SEQUENCE LISTING (40 sequences)

SEQ ID NO: 1 - protein, 523 aa, Homo sapiens
MESAPAAPDPAASEPGSSGADAAAGSRETPLNQESARKSEPPAPVRRQSYSSTSRGISVTKKTHTSQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 2 - DNA, 10827 bp, Homo sapiens
gtaccatagagttgctctgaaaacagaagatagagggagtctcggagctcgccatctccagcgatctctacattgggaaaaaacatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctaccttaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaaagttgaacttgtttttctcatgcatatgatttccattatgcctacagatatggaccctttttctgtcttgacttcttgatcattgacctctgtttacaacaggaggagggtactaaagtcggaggatttccttttcttgtagctcactgcccacagactttctacagagtcaccaatctgtcagtaacaacagagagtccagcaataatcggtgactggtgtgcatagcggaggttgcggcattactttgcacaactagctctttgtttcatgaaggaagtttttattttttcaccgattattgccagtccgcaggatggcatgaaaagggtccatagcagtagcaacaatagcattataatatattacagggtaaatgggcatgaagactatatatagctaaaagagatattgtttatatattgttttaagtaatataaaatgtagttactggtgtagcttttcctgttgaattgataaggcactttcattttgcacctttttctttaaattaaatgctagcgtgttcactgtcgtgtcgcatgtgcaccagaaacacaagtttaactgagaaggcttggaaggtacgttgggaggtatttatgctgctgtttacaaaattatttttaagagactggctggtcatatctagaaatcaccacgttggattttttttttaacatgtgaatttggaattagaaacggaactctccctaaattatactttgctttttggtaagtttaatgatagatgtgtttatgcttcatacaaagttgaatgattgattggcgtggtggacatataccatcatgctcattttttttttttaaagctttttaaaatgccacctcatggaggcgagggggaggagaagctcattttacacaattcagtagttaaatatggactcggtctcaacttggaattcttatgctttgagaacaaatcaacaaccagaatatttattggaatctagcttttattataagaaggacccaaagattatatcctgagcaaatgcacactccccatgtgaggacatgaagtatttactttgtgaatgtttatgttcttggtataatctaggaaccctatgagtttatctcagagtgaactaattctagatttgttgtcaatagatgctataattcaagaatgttgctctccatatttgaaaaacgatggataggagggtgagggaagcatacaatgttgaaccagtttctcttatttaaatattaaatactttaagcctttaaagtgaagttgatgctagctgcaaacatttactcttgtatttatcttcactaggaaactgtggactgtaatttattttattaaatatttagaagattatttggcttgtgtgttcaggtgagaaatactgagttgtttttgtttaatttcatggtttttttttttttaatgatccctagtgggggaaggggaaaggaatagtctgataaacagatgtgcatattttaaaaacaagtgaccttttgggaatgtaggcatttagacgatgattttagtcgcactaggggtgggattcaaactactggtcaaagaccattttgtacagaaaagggaacatctccgatgggtgttaaggtaggagtttccatgcagccctttatgtctgagaaatagtctcctctgccattggggtccctgcggaatcttctacaggaattgcagctcttcacgtcatgctaggttaccagcatgggcttcccagagcacttcactctgtttctaactccactgacttttctgacttgtttcttgagggacttggaaaaggggaaggtattattcacacagatgtgtgtatgaagcctaaataacatccaactttttttccaaaaacatagtaagagtttaaccataaatagaagagtaatattcctttaaatttcttacaggcatgtaaaattttatgtgtttatagagagatgctaactgtcagcatatagtatttatattggggcaagaagggttaaatcaaatctttaatttaagtaagcatagttcctttaaagatcagtagtatttatactctgaaaagagtaccaagcttagttcagttatttattcatccattttttttctattgtttctcctctggggggaaatggtgttttgtttttgtttgagttttgtgttttagttttttggttttggttttgttttgtttttttgtcattcagattcactaaatttggggatggttttattaaagtatattaacttctttttaaccaaagctttctgaatatgaccagcctcaggtgctagcgcattaaagagcaactaaacctaattccagtgtcgatttgtgaaataataaaagctaattgaatttctctaagggtgagagagaacataatgatgaacttaaaaagaaattccttttcccagaagggattctgcatgtacctacaaacaatcatgctctaaccacagtaatagtcatgccatggtgacattgcttttacggtaaaccaagataggtaatatgatgcttcttcccaggtgtttctgaaagaaaagcagcggtggagcttaagagccaagtccactgattgacataataggatggaattgtagacagagacatgctccatgaaacaaggaaacaactgactactatttggatctaagttgggatctgatgttaaacaataaattcagtttaaaaaaaatatggagctcagaaaaggatgtgaaaaactttgcattttcctttcttcattattacaaaaacacctatgtgatgacataaaatacttgggtgatatcagaacaattatccttaatagtttttataattaaagtttcctaaacttcagtctccaatagtcttttaaggattggaaccacatcactgtcagccccgctgctacaatgcctttgtacaatttttttacataaggcaaataaggcttttgtacaaagcctgaataccttctgttccaatggtgtccaaatggttattatattgtgaaaaacctggccttggtcacaatgcaaacaaaagtacagatgaaagtgctttttggacagtttgcaaattgtgttaaagctacggattttttttaaagtgttcagcatcttaacgtgtattaaagctatggatttttttttaaagtcttcagcatcctaattcaccctttctaacttaagaaaaacatgacttaagacactgcttctaagtttggttgttctttatagtgtggatacccagttatctgtgagcgtatgggggtgggctgagggtcaggtgaggaaggagtgtgtgtgtgtgtgtgtgtgtgtgtgtgtgtgtgtgtgatttgcatgtgtatgatgtgtgtgcgtcggaccgcttctaggctactaagtgtcaatggaaaagaaaatgtattcaaaatacttaaatcaaaactagaagatggggaaaaaaagatttattctatacaaagccttgtctggaccactttagagagacttctatttttttaacccttctataaatatttgatggcacttgaaatattcctgcaataaaatgtgatttgtgtaaagaaaaaaagattttgtaatgtgaaacaaagaaagaaagtaatgtaattttctaaaaaaaaaaatacaaacaaacaaactttgtattattttcttgatggaatttgtctatctgtctttggaaaactttttatttcattgaatgtgccatagtagaaatgtgtgtttttagttttagactaaggaatagctagttgttgtgttccgacattccaaaatgcaaaacaacctagtagtatctttcatgaaaaggtttaagtagtatgtaagcttctctcattgttgcttttttgcacatgttcttcattcctctctagtgcaatatgtacatagagcacttgcgggtgtaccttgatccctcagggaaaaatacatatttgtacagttttttggggtttttttgttttttggttttttttgttttgccttttgtttttggctaaggaatgtcgatcgaatcacttgttattgttgaggggcagccagataataatcctaaagccactgtttccaacattgattgtttaaatcatatgtccttccaatgctattattttaagataataataaaaagttattttctgacagttctttgtgctgactggtgaaaaacaagggtaaataagcaccttataattgacttactgtgaatgacaatccatcttggtatcaacgatagaagccctatcattttggagttggggttaagagtcagaaacaatgtgctcagggatctcctaaaactcttaaaacagggtggccagtactactgggacaaattgtgttttttattattaataataatgataataatactatccctccaaggcacaagtgaactatatagaactgcgtgtgtgtaaactctttactctcgtctcattttgttgagtttagaacttgatgtgctcgtcagtcttgtgtttcaaaacactgaataacttccaaagcaaagttatgccagtgtgttcaaaagataaattaataatgtaccagcaaagagcatcattcaaagtatagtccttgcatattccagttaccatttctctaataattaaaatattcatgataaatatatatatagcatcatatgttaaaaactatttcaaattctacatattaaggatgaaaattttaaaatccagtaataagaggagagacctgcctatcagtacagtgatataggtaaaagatgaaatatgtttttaaaataccagcaattaactattgttttcatgggttctcctctagaagcaaaccaaaaattccttcatggaaaacaattcttacttctacatgtgtagtttatatctgatgcattacaagagctaatgtttaaagacaaaacaaaacctgcctgtatgacagcagcaactcgagccaacatttagtgttacatgttatatttttgaaataccgttttgttatcatattccacatattactttccataaagtcagagaagttcaatgtaattgttggctctgattcttccaccttgggatacacattcacaagaagatttatatattttcttactatgatagaggaacataatctgggaaaacttcccatgtctgtaagatgaaaaggatacctttaccatgttgtttttgataataaagaatatggaaaatggtagaaatctctcctctctatgtgtatttgtatatgtgtgtatacacacacacgttgacatttttacataacatgtgattgccacttcttataaagttatgatatagaaccccatgaaaacaactttatattatagatcaaataatgtgcccagaaacgcaattgcaacagtaaatcttgatctattggtaagagtctatgatacaggtcttcattctatccctaaacataatgttagtaaagaggttccatcagattgtattatagagaccttcctattgctatttatttttaagagatgagaagactgactagcaatgtctccacagtgcaattggtttcacttctgggtctgtctgtctgtttgtgtaggtgagatcagtgttgtcaggctctcagaataatttttaaaagaatccagaagcctgacttcacaccaagtagccatcctagatgggcgggggggatctccatgttcaaccaaaccctcagaacttagagcaataaatacattcagtcatttatttataaatgaatgacagaatattcaatccaaatagaaacaaccttttgtcagtgatgcaacataacatggagtcttatattgagaaagtgaatatgaatattttaaatagatgcctaggaaatctgtgtttgctgtttacatttaatgtactttgccacattagcagtaccacacttcttttactttcatccttctaagaactaatgaaagatagcttgctattagctttgacatgttgcacatgccattatgttgttctctaagaacaactaaattctctccagtgttcatgtgtgattcctttttcatcattattactttaatgtggatgaatactatttctaggaacttgattatcactgaacagattgcatatgtaaatgcagataattcttgagcaatatagtgaaaatgatttcacaaaaaaaatccatatgtactgtatacatcttcctgaatccaaaattctcctgttatccacacagtttgaaatctcatattaaacagtggtaaatttttaaacattaggacattagctggctgcttctataaatgtacctgtattgtctgcctgctccttttgtggagatgtgtttttgtattggatgtttgggccaatgggaggcttcaagctacaacatattttcccactttaaataatatttaacatatgacaaaccccagacaaggcttttaatatgtataaagaactgcagtgttaggtggtttatttgcaaactgttttcagtgttgtccatttttatggcacctttaacaatattcttgtttccaaagtacaaaaaaaataggttaaataatctagccataactgaatgtcaagcaataaaacaaatgacttttgtgcatagacttaaaaaatacaaattttagacatctgcatgtaaatgcaatctcttgtttactgctttcttcaacttgagcaatcgattccttatttactattaacttaagaacttgtaacggcctgtaaacattttctctcttactcttctttcaaatttacctgtccctgcttttgagtcataatatttaatgttgttctgcacatctctatacagttaactttttggctttcattctgtatagataagaaaatgttatattataaacagcctactcagtgcaaatatttatctgtttatcaaatccacaatatgctgtataataccggttttactatataatctattttagacatagctgtttagaactagagtgtgctatttttgtgtttttctgatgtgtggtgctagacaagttacttttgtgaacaacaaaaattatcccttttattcctagacaataccacctttgggtcttgttaatttcactgagtataactatatatttgtatatatatacatatatatatatatctacctatgcccaactggcagctgtatcagagtgctggatttgggacatgcttttctctttaaatacataatatcattatataaattattctagagtgtatttaattaggataaaattacttccttagtatggatatttgacatctatagggtgaatttgtttataaatatggctatatggaaacttattagcatttactttatgtttgctacttggctttacagcatatctcctaagctgaaaaataatttgccaggccttcaagatcctaaagaaacttgtttaatggagtaatatacttttttttcttattaaggaattgtattactggcacctaacacagttgtattcttagctcctattatagataatgggcatttacataaaatatcctagatggcttgatggcagaataaacctttcccctcctacctgagtcatgagaaggatggagacgtcctctgccataacatgggccataaagcaaattcgacatgggatgttctgtttcagtatgacctcaaccagttccatgaactgagtgaaggaccttcattttcaaagttatttaataagtagcttaattaagcctttctacccattctcccaagatctactggcattattgaaaagcaaagtttatcaaatatctaactaaggatgtagttaaccttattaaatattgattagaattgttctgtaatattactgaatttgtaagatctttagcaaagatttttgagcaatttataaatgtagagcaaatgtttctgtttactgcactttttgtaactgaaggtgataaattctcaagccatgattattggcttccatgcactgcaatatttatccacaattctagacattttccatttttgtggaagagttgctgttaccttaattataaatgcaattgtgtggttaatgagagctaatgctagtagttaaccttttaaagtggattggctacagttgagggagaaatctcttttaatataaatcacatcattccttaactgcctctcttggaaagagattgaaaccttttttttaaagcacgatttagcatcctaagcttcctgagggtagagattgtatctttttgcgtctgcacaatggctagcacatgtcagcatttgacaattgttaaatgataacaagtgtgccccaattaaaacgtttttcctgggttgttttgttaaatttacaaagtaagccaagccttacggttaacattctcctctacaaccaagtattaaagccacatttaaaaagaccacatgaaatgctgattctaattgtgtgtaggtcttgaggattaagcacacaaatttcacaaacttctgtttgagtaaacaaactcagccttctgtaaatatacatgcaagtttggaaacagtaatactgtacctataaatatatgctgtctgttttgtgtacagtatgtaaaaactccttttctgccacactaaaaatgcaagccatttatgggaatcctaaaactagtattgaactaaaactttgctaatgatctttattagaggatcgtccaacttttcacttaccttgggttttcttttcaattcactcttacactagtctgcttatttccagctgtttattttattgagtcctgaatttaaaaaaaaaatattttgattcattttgtaaatacaagctgtacaaaaaagagagatttaatgttgtcttttaaatactccaattttcattctaatatgaatgttgttatattgtacttagaaactgtacctttaatattacattacctttattaaaagtgcattgaacacatcaattttagatgtgctttatgtactgttatcctataataaaacttcagcttctaatggaa

SEQ ID NO: 3 - DNA, 500 bp, Homo sapiens
  misc_feature (251)..(251): v=SNP position
aggcgcacctcacacggcacacaggcacatctcacacatggcacacatgcacacctcacacagatggcacacatgcacacctcacacacacggcacgcatgcacacctcacacacacggcacgcatgcacacctcacacacggcacacatgcacacctcacacacgacacacgggcacacctcacacacatggcacacgggcacacctcccacacacggcacacgggcacacctcccacacacggcacacvggcacacctcaaacgacacacggcacacctcacacacaagtctattcagctgcaagtcctgcctccacttgctgagaacctgcatgactgggcaccaaggatacggcacacacacgcacccaccccacatacatacagtccacacacacacaacacatatacaccacacgcaccacagatgcacaccacacatgccacacacacatacactgcacacgcaccctacacacaccccccacatgcttacac

SEQ ID NO: 4 - protein, 638 aa, Homo sapiens
MDLWQLLLTLALAGSSDAFSGSEATAAILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYFPWLLIIIFGIFGLTVMLFVFLFSKQQRIKMLILPPVPVPKIKGIDPDLLKEGKLEEVNTILAIHDSYKPEFHSDDSWVEFIELDIDEPDEKTEESDTDRLLSSDHEKSHSNLGVKDGDSGRTSCCEPDILETDFNANDIHEGTSEVAQPQRLKGEADLLCLDQKNQNNSPYHDACPATQQPSVIQAEKNKPQPLPTEGAESTHQAAHIQLSNPSSLSNIDFYAQVSDITPAGSVVLSPGQKNKAGMSQCDMHPEMVSLCQENFLMDNAYFCEADAKKCIPVAPHIKVESHIQPSLNQEDIYITTESLTTAAGRPGTGEHVPGSEMPVPDYTSIHIVQSPQGLILNATALPLPDKEFLSSCGYVSTDQLNKIMP

SEQ ID NO: 5 - DNA, 4560 bp, Homo sapiens
aactagcaatggtggtacagtggatgaaaagtgtttctctgttgatgaaataggcggcggcggcggcagcggcagcagcagctgctacagtggcggtggcggcggcggctgctgctgagcccgggcggcggcggggaccccgggctggggccacgcgggccggaggccccggcaccattggccccagcgcagacgcgaacccgcgctctctgatcagaggcgaagctcggaggtcctacaggtatggatctctggcagctgctgttgaccttggcactggcaggatcaagtgatgctttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcatcatggtacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagcttgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaatttacatgtgaagaagatttctactttccatggctcttaattattatctttggaatatttgggctaacagtgatgctatttgtattcttattttctaaacagcaaaggattaaaatgctgattctgcccccagttccagttccaaagattaaaggaatcgatccagatctcctcaaggaaggaaaattagaggaggtgaacacaatcttagccattcatgatagctataaacccgaattccacagtgatgactcttgggttgaatttattgagctagatattgatgagccagatgaaaagactgaggaatcagacacagacagacttctaagcagtgaccatgagaaatcacatagtaacctaggggtgaaggatggcgactctggacgtaccagctgttgtgaacctgacattctggagactgatttcaatgccaatgacatacatgagggtacctcagaggttgctcagccacagaggttaaaaggggaagcagatctcttatgccttgaccagaagaatcaaaataactcaccttatcatgatgcttgccctgctactcagcagcccagtgttatccaagcagagaaaaacaaaccacaaccacttcctactgaaggagctgagtcaactcaccaagctgcccatattcagctaagcaatccaagttcactgtcaaacatcgacttttatgcccaggtgagcgacattacaccagcaggtagtgtggtcctttccccgggccaaaagaataaggcagggatgtcccaatgtgacatgcacccggaaatggtctcactctgccaagaaaacttccttatggacaatgcctacttctgtgaggcagatgccaaaaagtgcatccctgtggctcctcacatcaaggttgaatcacacatacagccaagcttaaaccaagaggacatttacatcaccacagaaagccttaccactgctgctgggaggcctgggacaggagaacatgttccaggttctgagatgcctgtcccagactatacctccattcatatagtacagtccccacagggcctcatactcaatgcgactgccttgcccttgcctgacaaagagtttctctcatcatgtggctatgtgagcacagaccaactgaacaaaatcatgccttagcctttctttggtttcccaagagctacgtatttaatagcaaagaattgactggggcaataacgtttaagccaaaacaatgtttaaaccttttttgggggagtgacaggatggggtatggattctaaaatgccttttcccaaaatgttgaaatatgatgttaaaaaaataagaagaatgcttaatcagatagatattcctattgtgcaatgtaaatattttaaagaattgtgtcagactgtttagtagcagtgattgtcttaatattgtgggtgttaatttttgatactaagcattgaatggctatgtttttaatgtatagtaaatcacgctttttgaaaaagcgaaaaaatcaggtggcttttgcggttcaggaaaattgaatgcaaaccatagcacaggctaattttttgttgtttcttaaataagaaacttttttatttaaaaaactaaaaactagaggtgagaaatttaaactataagcaagaaggcaaaaatagtttggatatgtaaaacatttattttgacataaagttgataaagattttttaataatttagacttcaagcatggctattttatattacactacacactgtgtactgcagttggtatgacccctctaaggagtgtagcaactacagtctaaagctggtttaatgttttggccaatgcacctaaagaaaaacaaactcgttttttacaaagcccttttatacctccccagactccttcaacaattctaaaatgattgtagtaatctgcattattggaatataattgttttatctgaatttttaaacaagtatttgttaatttagaaaactttaaagcgtttgcacagatcaacttaccaggcaccaaaagaagtaaaagcaaaaaagaaaacctttcttcaccaaatcttggttgatgccaaaaaaaaatacatgctaagagaagtagaaatcatagctggttcacactgaccaagatacttaagtgctgcaattgcacgcggagtgagttttttagtgcgtgcagatggtgagagataagatctatagcctctgcagcggaatctgttcacacccaacttggttttgctacataattatccaggaagggaataaggtacaagaagcattttgtaagttgaagcaaatcgaatgaaattaactgggtaatgaaacaaagagttcaagaaataagtttttgtttcacagcctataaccagacacatactcatttttcatgataatgaacagaacatagacagaagaaacaaggttttcagtccccacagataactgaaaattatttaaaccgctaaaagaaactttctttctcactaaatcttttataggatttatttaaaatagcaaaagaagaagtttcatcattttttacttcctctctgagtggactggcctcaaagcaagcattcagaagaaaaagaagcaacctcagtaatttagaaatcattttgcaatcccttaatatcctaaacatcattcatttttgttgttgttgttgttgttgagacagagtctcgctctgtcgccaggctagagtgcggtggcgcgatcttgactcactgcaatctccacctcccacaggttcaggcgattcccgtgcctcagcctcctgagtagctgggactacaggcacgcaccaccatgccaggctaatttttttgtattttagcagagacggggtttcaccatgttggccaggatggtctcgatctcctgacctcgtgatccacccgactcggcctcccaaagtgctgggattacaggtgtaagccaccgtgcccagccctaaacatcattcttgagagcattgggatatctcctgaaaaggtttatgaaaaagaagaatctcatctcagtgaagaatacttctcattttttaaaaaagcttaaaactttgaagttagctttaacttaaatagtatttcccatttatcgcagaccttttttaggaagcaagcttaatggctgataattttaaattctctctcttgcaggaaggactatgaaaagctagaattgagtgtttaaagttcaacatgttatttgtaatagatgtttgatagattttctgctactttgctgctatggttttctccaagagctacataatttagtttcatataaagtatcatcagtgtagaacctaattcaattcaaagctgtgtgtttggaagactatcttactatttcacaacagcctgacaacatttctatagccaaaaatagctaaatacctcaatcagtctcagaatgtcattttggtactttggtggccacataagccattattcactagtatgactagttgtgtctggcagtttatatttaactctctttatgtctgtggattttttccttcaaagtttaataaatttattttcttggattcctgatagtgtgcttctgttatcaaacaccaacataaaaatgatctaaacc

SEQ ID NO: 6 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 7 - DNA, 1678 bp, Homo sapiens
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 8 - protein, 222 aa, Homo sapiens
MAEKPKLHYFNARGRMESTRWLLAAAGVEFEEKFIKSAEDLDKLRNDGYLMFQQVPMVEIDGMKLVQTRAILNYIASKYNLYGKDIKERALIDMYIEGIADLGEMILLLPVCPPEEKDAKLALIKEKIKNRYFPAFEKVLKSHGQDYLVGNKLSRADIHLVELLYYVEELDSSLISSFPLLKALKTRISNLPTVKKFLQPGSPRKPPMDEKSLEEARKIFRF

SEQ ID NO: 9 - DNA, 1218 bp, Homo sapiens
gtcgagccaggacggtgacagcgtttaacaaagcttagagaaacctccaggagactgctatcatggcagagaagcccaagctccactacttcaatgcacggggcagaatggagtccacccggtggctcctggctgcagctggagtagagtttgaagagaaatttataaaatctgcagaagatttggacaagttaagaaatgatggatatttgatgttccagcaagtgccaatggttgagattgatgggatgaagctggtgcagaccagagccattctcaactacattgccagcaaatacaacctctatgggaaagacataaaggagagagccctgattgatatgtatatagaaggtatagcagatttgggtgaaatgatcctccttctgcccgtatgtccacctgaggaaaaagatgccaagcttgccttgatcaaagagaaaataaaaaatcgctacttccctgcctttgaaaaagtcttaaagagccatggacaagactaccttgttggcaacaagctgagccgggctgacattcatctggtggaacttctctactacgtcgaggagcttgactccagtcttatctccagcttccctctgctgaaggccctgaaaaccagaatcagcaacctgcccacagtgaagaagtttctacagcctggcagcccaaggaagcctcccatggatgagaaatctttagaagaagcaaggaagattttcaggttttaataacgcagtcatggaggccaagaacttgcaataccaatgttctaaagttttgcaacaataaagtactttacctaagtgttgattgtgcctgttgtgaagctaatgaactctttcaaattatatgctaattaaataatacaactcctattcgctgacttagttaaaattgatttgttttcattaggatctgatgtgaattcagatttccaatcttctcctagccaaccattttcctggaattaaaaattcagtaaaaaaggaaactatagattatgtggtttgtttgacttttccaagaattgtcccgtaacatacaatttgtcatacaatctattaaaatgtcaatgtagaaatgcacttctgacattttcaggtatgcacaggagaagagttaccatcctggataatggcataaagacattttcttcttttcctggacagtcattttatttctgataaaagcgttctttcttatgcatttgcaaaa

SEQ ID NO: 10 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 11 - DNA, 708 bp, Homo sapiens
aggaggagcaccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaagaggaagtcctggctgcagacacctgcttctgattccacaaggggctttttcctcaaccctgtggccgcctttgaagtgactcatttttttaatgtatttatgtatttatttgattgttttatataagatggtttcttacctttgagcacaaaatttccacggtgaaataaagtcaacattataagcttta

SEQ ID NO: 12 - protein, 2201 aa, Homo sapiens
MGAMTQLLAGVFLAFLALATEGGVLKKVIRHKRQSGVNATLPEENQPVVFNHVYNIKLPVGSQCSVDLESASGEKDLAPPSEPSESFQEHTVDGENQIVFTHRINIPRRACGCAAAPDVKELLSRLEELENLVSSLREQCTAGAGCCLQPATGRLDTRPFCSGRGNFSTEGCGCVCEPGWKGPNCSEPECPGNCHLRGRCIDGQCICDDGFTGEDCSQLACPSDCNDQGKCVNGVCICFEGYAGADCSREICPVPCSEEHGTCVDGLCVCHDGFAGDDCNKPLCLNNCYNRGRCVENECVCDEGFTGEDCSELICPNDCFDRGRCINGTCYCEEGFTGEDCGKPTCPHACHTQGRCEEGQCVCDEGFAGVDCSEKRCPADCHNRGRCVDGRCECDDGFTGADCGELKCPNGCSGHGRCVNGQCVCDEGYTGEDCSQLRCPNDCHSRGRCVEGKCVCEQGFKGYDCSDMSCPNDCHQHGRCVNGMCVCDDGYTGEDCRDRQCPRDCSNRGLCVDGQCVCEDGFTGPDCAELSCPNDCHGQGRCVNGQCVCHEGFMGKDCKEQRCPSDCHGQGRCVDGQCICHEGFTGLDCGQHSCPSDCNNLGQCVSGRCICNEGYSGEDCSEVSPPKDLVVTEVTEETVNLAWDNEMRVTEYLVVYTPTHEGGLEMQFRVPGDQTSTIIQELEPGVEYFIRVFAILENKKSIPVSARVATYLPAPEGLKFKSIKETSVEVEWDPLDIAFETWEIIFRNMNKEDEGEITKSLRRPETSYRQTGLAPGQEYEISLHIVKNNTRGPGLKRVTTTRLDAPSQIEVKDVTDTTALITWFKPLAEIDGIELTYGIKDVPGDRTTIDLTEDENQYSIGNLKPDTEYEVSLISRRGDMSSNPAKETFTTGLDAPRNLRRVSQTDNSITLEWRNGKAAIDSYRIKYAPISGGDHAEVDVPKSQQATTKTTLTGLRPGTEYGIGVSAVKEDKESNPATINAATELDTPKDLQVSETAETSLTLLWKTPLAKFDRYRLNYSLPTGQWVGVQLPRNTTSYVLRGLEPGQEYNVLLTAEKGRHKSKPARVKASTEQAPELENLTVTEVGWDGLRLNWTAADQAYEHFIIQVQEANKVEAARNLTVPGSLRAVDIPGLKAATPYTVSIYGVIQGYRTPVLSAEASTGETPNLGEVVVAEVGWDALKLNWTAPEGAYEYFFIQVQEADTVEAAQNLTVPGGLRSTDLPGLKAATHYTITIRGVTQDFSTTPLSVEVLTEEVPDMGNLTVTEVSWDALRLNWTTPDGTYDQFTIQVQEADQVEEAHNLTVPGSLRSMEIPGLRAGTPYTVTLHGEVRGHSTRPLAVEVVTEDLPQLGDLAVSEVGWDGLRLNWTAADNAYEHFVIQVQEVNKVEAAQNLTLPGSLRAVDIPGLEAATPYRVSIYGVIRGYRTPVLSAEASTAKEPEIGNLNVSDITPESFNLSWMATDGIFETFTIEIIDSNRLLETVEYNISGAERTAHISGLPPSTDFIVYLSGLAPSIRTKTISATATTEALPLLENLTISDINPYGFTVSWMASENAFDSFLVTVVDSGKLLDPQEFTLSGTQRKLELRGLITGIGYEVMVSGFTQGHQTKPLRAEIVTEAEPEVDNLLVSDATPDGFRLSWTADEGVFDNFVLKIRDTKKQSEPLEITLLAPERTRDITGLREATEYEIELYGISKGRRSQTVSAIATTAMGSPKEVIFSDITENSATVSWRAPTAQVESFRITYVPITGGTPSMVTVDGTKTQTRLVKLIPGVEYLVSIIAMKGFEESEPVSGSFTTALDGPSGLVTANITDSEALARWQPAIATVDSYVISYTGEKVPEITRTVSGNTVEYALTDLEPATEYTLRIFAEKGPQKSSTITAKFTTDLDSPRDLTATEVQSETALLTWRPPRASVTGYLLVYESVDGTVKEVIVGPDTTSYSLADLSPSTHYTAKIQALNGPLRSNMIQTIFTTIGLLYPFPKDCSQAMLNGDTTSGLYTIYLNGDKAEALEVFCDMTSDGGGWIVFLRRKNGRENFYQNWKAYAAGFGDRREEFWLGLDNLNKITAQGQYELRVDLRDHGETAFAVYDKFSVGDAKTRYKLKVEGYSGTAGDSMAYHNGRSFSTFDKDTDSAITNCALSYKGAFWYRNCHRVNLMGRYGDNNHSQGVNWFHWKGHEHSIQFAEMKLRPSNFRNLEGRRKRA

SEQ ID NO: 13 - DNA, 8500 bp, Homo sapiens
ggccacagcctgcctactgtcacccgcctctcccgcgcgcagatacacgcccccgcctccgtgggcacaaaggcagcgctgctggggaactcgggggaacgcgcacgtgggaaccgccgcagctccacactccaggtacttcttccaaggacctaggtctctcgcccatcggaaagaaaataattctttcaagaagatcagggacaactgatttgaagtctactctgtgcttctaaatccccaattctgctgaaagtgagataccctagagccctagagccccagcagcacccagccaaacccacctccaccatgggggccatgactcagctgttggcaggtgtctttcttgctttccttgccctcgctaccgaaggtggggtcctcaagaaagtcatccggcacaagcgacagagtggggtgaacgccaccctgccagaagagaaccagccagtggtgtttaaccacgtttacaacatcaagctgccagtgggatcccagtgttcggtggatctggagtcagccagtggggagaaagacctggcaccgccttcagagcccagcgaaagctttcaggagcacacagtggatggggaaaaccagattgtcttcacacatcgcatcaacatcccccgccgggcctgtggctgtgccgcagcccctgatgttaaggagctgctgagcagactggaggagctggagaacctggtgtcttccctgagggagcaatgtactgcaggagcaggctgctgtctccagcctgccacaggccgcttggacaccaggcccttctgtagcggtcggggcaacttcagcactgaaggatgtggctgtgtctgcgaacctggctggaaaggccccaactgctctgagcccgaatgtccaggcaactgtcaccttcgaggccggtgcattgatgggcagtgcatctgtgacgacggcttcacgggcgaggactgcagccagctggcttgccccagcgactgcaatgaccagggcaagtgcgtaaatggagtctgcatctgtttcgaaggctacgccggggctgactgcagccgtgaaatctgcccagtgccctgcagtgaggagcacggcacatgtgtagatggcttgtgtgtgtgccacgatggctttgcaggcgatgactgcaacaagcctctgtgtctcaacaattgctacaaccgtggacgatgcgtggagaatgagtgcgtgtgtgatgagggtttcacgggcgaagactgcagtgagctcatctgccccaatgactgcttcgaccggggccgctgcatcaatggcacctgctactgcgaagaaggcttcacaggtgaagactgcgggaaacccacctgcccacatgcctgccacacccagggccggtgtgaggaggggcagtgtgtatgtgatgagggctttgccggtgtggactgcagcgagaagaggtgtcctgctgactgtcacaatcgtggccgctgtgtagacgggcggtgtgagtgtgatgatggtttcactggagctgactgtggggagctcaagtgtcccaatggctgcagtggccatggccgctgtgtcaatgggcagtgtgtgtgtgatgagggctatactggggaggactgcagccagctacggtgccccaatgactgtcacagtcggggccgctgtgtcgagggcaaatgtgtatgtgagcaaggcttcaagggctatgactgcagtgacatgagctgccctaatgactgtcaccagcacggccgctgtgtgaatggcatgtgtgtttgtgatgacggctacacaggggaagactgccgggatcgccaatgccccagggactgcagcaacaggggcctctgtgtggacggacagtgcgtctgtgaggacggcttcaccggccctgactgtgcagaactctcctgtccaaatgactgccatggccagggtcgctgtgtgaatgggcagtgcgtgtgccatgaaggatttatgggcaaagactgcaaggagcaaagatgtcccagtgactgtcatggccagggccgctgcgtggacggccagtgcatctgccacgagggcttcacaggcctggactgtggccagcactcctgccccagtgactgcaacaacttaggacaatgcgtctcgggccgctgcatctgcaacgagggctacagcggagaagactgctcagaggtgtctcctcccaaagacctcgttgtgacagaagtgacggaagagacggtcaacctggcctgggacaatgagatgcgggtcacagagtaccttgtcgtgtacacgcccacccacgagggtggtctggaaatgcagttccgtgtgcctggggaccagacgtccaccatcatccaggagctggagcctggtgtggagtactttatccgtgtatttgccatcctggagaacaagaagagcattcctgtcagcgccagggtggccacgtacttacctgcacctgaaggcctgaaattcaagtccatcaaggagacatctgtggaagtggagtgggatcctctagacattgcttttgaaacctgggagatcatcttccggaatatgaataaagaagatgagggagagatcaccaaaagcctgaggaggccagagacctcttaccggcaaactggtctagctcctgggcaagagtatgagatatctctgcacatagtgaaaaacaatacccggggccctggcctgaagagggtgaccaccacacgcttggatgcccccagccagatcgaggtgaaagatgtcacagacaccactgccttgatcacctggttcaagcccctggctgagatcgatggcattgagctgacctacggcatcaaagacgtgccaggagaccgtaccaccatcgatctcacagaggacgagaaccagtactccatcgggaacctgaagcctgacactgagtacgaggtgtccctcatctcccgcagaggtgacatgtcaagcaacccagccaaagagaccttcacaacaggcctcgatgctcccaggaatcttcgacgtgtttcccagacagataacagcatcaccctggaatggaggaatggcaaggcagctattgacagttacagaattaagtatgcccccatctctggaggggaccacgctgaggttgatgttccaaagagccaacaagccacaaccaaaaccacactcacaggtctgaggccgggaactgaatatgggattggagtttctgctgtgaaggaagacaaggagagcaatccagcgaccatcaacgcagccacagagttggacacgcccaaggaccttcaggtttctgaaactgcagagaccagcctgaccctgctctggaagacaccgttggccaaatttgaccgctaccgcctcaattacagtctccccacaggccagtgggtgggagtgcagcttccaagaaacaccacttcctatgtcctgagaggcctggaaccaggacaggagtacaatgtcctcctgacagccgagaaaggcagacacaagagcaagcccgcacgtgtgaaggcatccactgaacaagcccctgagctggaaaacctcaccgtgactgaggttggctgggatggcctcagactcaactggaccgcagctgaccaggcctatgagcactttatcattcaggtgcaggaggccaacaaggtggaggcagctcggaacctcaccgtgcctggcagccttcgggctgtggacataccgggcctcaaggctgctacgccttatacagtctccatctatggggtgatccagggctatagaacaccagtgctctctgctgaggcctccacaggggaaactcccaatttgggagaggtcgtggtggccgaggtgggctgggatgccctcaaactcaactggactgctccagaaggggcctatgagtactttttcattcaggtgcaggaggctgacacagtagaggcagcccagaacctcaccgtcccaggaggactgaggtccacagacctgcctgggctcaaagcagccactcattataccatcaccatccgcggggtcactcaggacttcagcacaacccctctctctgttgaagtcttgacagaggaggttccagatatgggaaacctcacagtgaccgaggttagctgggatgctctcagactgaactggaccacgccagatggaacctatgaccagtttactattcaggtccaggaggctgaccaggtggaagaggctcacaatctcacggttcctggcagcctgcgttccatggaaatcccaggcctcagggctggcactccttacacagtcaccctgcacggcgaggtcaggggccacagcactcgaccccttgctgtagaggtcgtcacagaggatctcccacagctgggagatttagccgtgtctgaggttggctgggatggcctcagactcaactggaccgcagctgacaatgcctatgagcactttgtcattcaggtgcaggaggtcaacaaagtggaggcagcccagaacctcacgttgcctggcagcctcagggctgtggacatcccgggcctcgaggctgccacgccttatagagtctccatctatggggtgatccggggctatagaacaccagtactctctgctgaggcctccacagccaaagaacctgaaattggaaacttaaatgtttctgacataactcccgagagcttcaatctctcctggatggctaccgatgggatcttcgagacctttaccattgaaattattgattccaataggttgctggagactgtggaatataatatctctggtgctgaacgaactgcccatatctcagggctaccccctagtactgattttattgtctacctctctggacttgctcccagcatccggaccaaaaccatcagtgccacagccacgacagaggccctgccccttctggaaaacctaaccatttccgacattaatccctacgggttcacagtttcctggatggcatcggagaatgcctttgacagctttctagtaacggtggtggattctgggaagctgctggacccccaggaattcacactttcaggaacccagaggaagctggagcttagaggcctcataactggcattggctatgaggttatggtctctggcttcacccaagggcatcaaaccaagcccttgagggctgagattgttacagaagccgaaccggaagttgacaaccttctggtttcagatgccaccccagacggtttccgtctgtcctggacagctgatgaaggggtcttcgacaattttgttctcaaaatcagagataccaaaaagcagtctgagccactggaaataaccctacttgcccccgaacgtaccagggacataacaggtctcagagaggctactgaatacgaaattgaactctatggaataagcaaaggaaggcgatcccagacagtcagtgctatagcaacaacagccatgggctccccaaaggaagtcattttctcagacatcactgaaaattcggctactgtcagctggagggcacccacagcccaagtggagagcttccggattacctatgtgcccattacaggaggtacaccctccatggtaactgtggacggaaccaagactcagaccaggctggtgaaactcatacctggcgtggagtaccttgtcagcatcatcgccatgaagggctttgaggaaagtgaacctgtctcagggtcattcaccacagctctggatggcccatctggcctggtgacagccaacatcactgactcagaagccttggccaggtggcagccagccattgccactgtggacagttatgtcatctcctacacaggcgagaaagtgccagaaattacacgcacggtgtccgggaacacagtggagtatgctctgaccgacctcgagcctgccacggaatacacactgagaatctttgcagagaaagggccccagaagagctcaaccatcactgccaagttcacaacagacctcgattctccaagagacttgactgctactgaggttcagtcggaaactgccctccttacctggcgacccccccgggcatcagtcaccggttacctgctggtctatgaatcagtggatggcacagtcaaggaagtcattgtgggtccagataccacctcctacagcctggcagacctgagcccatccacccactacacagccaagatccaggcactcaatgggcccctgaggagcaatatgatccagaccatcttcaccacaattggactcctgtaccccttccccaaggactgctcccaagcaatgctgaatggagacacgacctctggcctctacaccatttatctgaatggtgataaggctgaggcgctggaagtcttctgtgacatgacctctgatgggggtggatggattgtgttcctgagacgcaaaaacggacgcgagaacttctaccaaaactggaaggcatatgctgctggatttggggaccgcagagaagaattctggcttgggctggacaacctgaacaaaatcacagcccaggggcagtacgagctccgggtggacctgcgggaccatggggagacagcctttgctgtctatgacaagttcagcgtgggagatgccaagactcgctacaagctgaaggtggaggggtacagtgggacagcaggtgactccatggcctaccacaatggcagatccttctccacctttgacaaggacacagattcagccatcaccaactgtgctctgtcctacaaaggggctttctggtacaggaactgtcaccgtgtcaacctgatggggagatatggggacaataaccacagtcagggcgttaactggttccactggaagggccacgaacactcaatccagtttgctgagatgaagctgagaccaagcaacttcagaaatcttgaaggcaggcgcaaacgggcataaattccagggaccactgggtgagagaggaataaggcccagagcgaggaaaggattttaccaaagcatcaatacaaccagcccaaccatcggtccacacctgggcatttggtgagagtcaaagctgaccatggatccctggggccaacggcaacagcatgggcctcacctcctctgtgatttctttctttgcaccaaagacatcagtctccaacatgtttctgttttgttgtttgattcagcaaaaatctcccagtgacaacatcgcaatagttttttacttctcttaggtggctctgggaatgggagaggggtaggatgtacaggggtagtttgttttagaaccagccgtattttacatgaagctgtataattaattgtcattatttttgttagcaaagattaaatgtgtcattggaagccatccctttttttacatttcatacaacagaaaccagaaaagcaatactgtttccattttaaggatatgattaatattattaatataataatgatgatgatgatgatgaaaactaaggatttttcaagagatctttctttccaaaacatttctggacagtacctgattgtattttttttttaaataaaagcacaagtacttttgagtttgttattttgctttgaattgttgagtctgaatttcaccaaagccaatcatttgaacaaagcggggaatgttgggataggaaaggtaagtagggatagtggtcaagtgggaggggtggaaaggagactaaagactgggagagagggaagcactttttttaaataaagttgaacacacttgggaaaagcttacaggccaggcctgtaatcccaacactttgggaggccaaggtgggaggatagcttaaccccaggagtttgagaccagcctgagcaacatagtgagaacttgtctctacagaaaaaaaaaaaaaaaaaaatttaattaggcaagcgtggtagtgcgcacctgtcgtcccagctactcaggaggctgaggtaggaaaatcactggagcccaggagttagaggttacagtgagctatgatcacactactgcactccagcctgggcaacagagggagaccctgtctctaaataaaaaaagaaaagaaaaaaaaagcttacaacttgagattcagcatcttgctcagtatttccaagactaatagattatggtttaaaagatgcttttatactcattttctaatgcaactcctagaaactctatgatatagttgaggtaagtattgttaccacacatgggctaagatccccagaggcagactgcctgagttcaattcttggctccaccattcccaagttccctaacctctctatgcctcagtttcctcttctgtaaagtagggacactcatacttctcatttcagaacatttttgtgaagaataaattatgttatccatttgaggcccttagaatggtacccggtgtatattaagtgctagtacatgttagctatcatcattatcactttatatgagatggactggggttcatagaaacccaatgacttgattgtggctactactcaataaataatagaatttggatttaaa

SEQ ID NO: 14 - protein, 1474 aa, Homo sapiens
MGKNKLLHPSLVLLLLVLLPTDASVSGKPQYMVLVPSLLHTETTEKGCVLLSYLNETVTVSASLESVRGNRSLFTDLEAENDVLHCVAFAVPKSSSNEEVMFLTVQVKGPTQEFKKRTTVMVKNEDSLVFVQTDKSIYKPGQTVKFRVVSMDENFHPLNELIPLVYIQDPKGNRIAQWQSFQLEGGLKQFSFPLSSEPFQGSYKVVVQKKSGGRTEHPFTVEEFVLPKFEVQVTVPKIITILEEEMNVSVCGLYTYGKPVPGHVTVSICRKYSDASDCHGEDSQAFCEKFSGQLNSHGCFYQQVKTKVFQLKRKEYEMKLHTEAQIQEEGTVVELTGRQSSEITRTITKLSFVKVDSHFRQGIPFFGQVRLVDGKGVPIPNKVIFIRGNEANYYSNATTDEHGLVQFSINTTNVMGTSLTVRVNYKDRSPCYGYQWVSEEHEEAHHTAYLVFSPSKSFVHLEPMSHELPCGHTQTVQAHYILNGGTLLGLKKLSFYYLIMAKGGIVRTGTHGLLVKQEDMKGHFSISIPVKSDIAPVARLLIYAVLPTGDVIGDSAKYDVENCLANKVDLSFSPSQSLPASHAHLRVTAAPQSVCALRAVDQSVLLMKPDAELSASSVYNLLPEKDLTGFPGPLNDQDNEDCINRHNVYINGITYTPVSSTNEKDMYSFLEDMGLKAFTNSKIRKPKMCPQLQQYEMHGPEGLRVGFYESDVMGRGHARLVHVEEPHTETVRKYFPETWIWDLVVVNSAGVAEVGVTVPDTITEWKAGAFCLSEDAGLGISSTASLRAFQPFFVELTMPYSVIRGEAFTLKATVLNYLPKCIRVSVQLEASPAFLAVPVEKEQAPHCICANGRQTVSWAVTPKSLGNVNFTVSAEALESQELCGTEVPSVPEHGRKDTVIKPLLVEPEGLEKETTFNSLLCPSGGEVSEELSLKLPPNVVEESARASVSVLGDILGSAMQNTQNLLQMPYGCGEQNMVLFAPNIYVLDYLNETQQLTPEIKSKAIGYLNTGYQRQLNYKHYDGSYSTFGERYGRNQGNTWLTAFVLKTFAQARAYIFIDEAHITQALIWLSQRQKDNGCFRSSGSLLNNAIKGGVEDEVTLSAYITIALLEIPLTVTHPVVRNALFCLESAWKTAQEGDHGSHVYTKALLAYAFALAGNQDKRKEVLKSLNEEAVKKDNSVHWERPQKPKAPVGHFYEPQAPSAEVEMTSYVLLAYLTAQPAPTSEDLTSATNIVKWITKQQNAQGGFSSTQDTVVALHALSKYGAATFTRTGKAAQVTIQSSGTFSSKFQVDNNNRLLLQQVSLPELPGEYSMKVTGEGCVYLQTSLKYNILPEKEEFPFALGVQTLPQTCDEPKAHTSFQISLSVSYTGSRSASNMAIVDVKMVSGFIPLKPTVKMLERSNHVSRTEVSSNHVLIYLDKVSNQTLSLFFTVLQDVPVRDLKPAIVKVYDYYETDEFAIAEYNAPCSKDLGNA

SEQ ID NO: 15 - DNA, 4955 bp, Homo sapiens
gaaaagcttattagctgctgtacggtaaaagtgagctcttacgggaatgggaatgtagttttagccctccagggattctatttagcccgccaggaattaaccttgactataaataggccatcaatgacctttccagagaatgttcagagacctcaactttgtttagagatcttgtgtgggtggaacttcctgtttgcacacagagcagcataaagcccagttgctttgggaagtgtttgggaccagatggattgtagggagtagggtacaatacagtctgttctcctccagctccttctttctgcaacatggggaagaacaaactccttcatccaagtctggttcttctcctcttggtcctcctgcccacagacgcctcagtctctggaaaaccgcagtatatggttctggtcccctccctgctccacactgagaccactgagaagggctgtgtccttctgagctacctgaatgagacagtgactgtaagtgcttccttggagtctgtcaggggaaacaggagcctcttcactgacctggaggcggagaatgacgtactccactgtgtcgccttcgctgtcccaaagtcttcatccaatgaggaggtaatgttcctcactgtccaagtgaaaggaccaacccaagaatttaagaagcggaccacagtgatggttaagaacgaggacagtctggtctttgtccagacagacaaatcaatctacaaaccagggcagacagtgaaatttcgtgttgtctccatggatgaaaactttcaccccctgaatgagttgattccactagtatacattcaggatcccaaaggaaatcgcatcgcacaatggcagagtttccagttagagggtggcctcaagcaattttcttttcccctctcatcagagcccttccagggctcctacaaggtggtggtacagaagaaatcaggtggaaggacagagcaccctttcaccgtggaggaatttgttcttcccaagtttgaagtacaagtaacagtgccaaagataatcaccatcttggaagaagagatgaatgtatcagtgtgtggcctatacacatatgggaagcctgtccctggacatgtgactgtgagcatttgcagaaagtatagtgacgcttccgactgccacggtgaagattcacaggctttctgtgagaaattcagtggacagctaaacagccatggctgcttctatcagcaagtaaaaaccaaggtcttccagctgaagaggaaggagtatgaaatgaaacttcacactgaggcccagatccaagaagaaggaacagtggtggaattgactggaaggcagtccagtgaaatcacaagaaccataaccaaactctcatttgtgaaagtggactcacactttcgacagggaattcccttctttgggcaggtgcgcctagtagatgggaaaggcgtccctataccaaataaagtcatattcatcagaggaaatgaagcaaactattactccaatgctaccacggatgagcatggccttgtacagttctctatcaacaccaccaatgttatgggtacctctcttactgttagggtcaattacaaggatcgtagtccctgttacggctaccagtgggtgtcagaagaacacgaagaggcacatcacactgcttatcttgtgttctccccaagcaagagctttgtccaccttgagcccatgtctcatgaactaccctgtggccatactcagacagtccaggcacattatattctgaatggaggcaccctgctggggctgaagaagctctccttctattatctgataatggcaaagggaggcattgtccgaactgggactcatggactgcttgtgaagcaggaagacatgaagggccatttttccatctcaatccctgtgaagtcagacattgctcctgtcgctcggttgctcatctatgctgttttacctaccggggacgtgattggggattctgcaaaatatgatgttgaaaattgtctggccaacaaggtggatttgagcttcagcccatcacaaagtctcccagcctcacacgcccacctgcgagtcacagcggctcctcagtccgtctgcgccctccgtgctgtggaccaaagcgtgctgctcatgaagcctgatgctgagctctcggcgtcctcggtttacaacctgctaccagaaaaggacctcactggcttccctgggcctttgaatgaccaggacaatgaagactgcatcaatcgtcataatgtctatattaatggaatcacatatactccagtatcaagtacaaatgaaaaggatatgtacagcttcctagaggacatgggcttaaaggcattcaccaactcaaagattcgtaaacccaaaatgtgtccacagcttcaacagtatgaaatgcatggacctgaaggtctacgtgtaggtttttatgagtcagatgtaatgggaagaggccatgcacgcctggtgcatgttgaagagcctcacacggagaccgtacgaaagtacttccctgagacatggatctgggatttggtggtggtaaactcagcaggtgtggctgaggtaggagtaacagtccctgacaccatcaccgagtggaaggcaggggccttctgcctgtctgaagatgctggacttggtatctcttccactgcctctctccgagccttccagcccttctttgtggagctcacaatgccttactctgtgattcgtggagaggccttcacactcaaggccacggtcctaaactaccttcccaaatgcatccgggtcagtgtgcagctggaagcctctcccgccttcctagctgtcccagtggagaaggaacaagcgcctcactgcatctgtgcaaacgggcggcaaactgtgtcctgggcagtaaccccaaagtcattaggaaatgtgaatttcactgtgagcgcagaggcactagagtctcaagagctgtgtgggactgaggtgccttcagttcctgaacacggaaggaaagacacagtcatcaagcctctgttggttgaacctgaaggactagagaaggaaacaacattcaactccctactttgtccatcaggtggtgaggtttctgaagaattatccctgaaactgccaccaaatgtggtagaagaatctgcccgagcttctgtctcagttttgggagacatattaggctctgccatgcaaaacacacaaaatcttctccagatgccctatggctgtggagagcagaatatggtcctctttgctcctaacatctatgtactggattatctaaatgaaacacagcagcttactccagagatcaagtccaaggccattggctatctcaacactggttaccagagacagttgaactacaaacactatgatggctcctacagcacctttggggagcgatatggcaggaaccagggcaacacctggctcacagcctttgttctgaagacttttgcccaagctcgagcctacatcttcatcgatgaagcacacattacccaagccctcatatggctctcccagaggcagaaggacaatggctgtttcaggagctctgggtcactgctcaacaatgccataaagggaggagtagaagatgaagtgaccctctccgcctatatcaccatcgcccttctggagattcctctcacagtcactcaccctgttgtccgcaatgccctgttttgcctggagtcagcctggaagacagcacaagaaggggaccatggcagccatgtatataccaaagcactgctggcctatgcttttgccctggcaggtaaccaggacaagaggaaggaagtactcaagtcacttaatgaggaagctgtgaagaaagacaactctgtccattgggagcgccctcagaaacccaaggcaccagtggggcatttttacgaaccccaggctccctctgctgaggtggagatgacatcctatgtgctcctcgcttatctcacggcccagccagccccaacctcggaggacctgacctctgcaaccaacatcgtgaagtggatcacgaagcagcagaatgcccagggcggtttctcctccacccaggacacagtggtggctctccatgctctgtccaaatatggagcagccacatttaccaggactgggaaggctgcacaggtgactatccagtcttcagggacattttccagcaaattccaagtggacaacaacaaccgcctgttactgcagcaggtctcattgccagagctgcctggggaatacagcatgaaagtgacaggagaaggatgtgtctacctccagacatccttgaaatacaatattctcccagaaaaggaagagttcccctttgctttaggagtgcagactctgcctcaaacttgtgatgaacccaaagcccacaccagcttccaaatctccctaagtgtcagttacacagggagccgctctgcctccaacatggcgatcgttgatgtgaagatggtctctggcttcattcccctgaagccaacagtgaaaatgcttgaaagatctaaccatgtgagccggacagaagtcagcagcaaccatgtcttgatttaccttgataaggtgtcaaatcagacactgagcttgttcttcacggttctgcaagatgtcccagtaagagatctgaaaccagccatagtgaaagtctatgattactacgagacggatgagtttgcaattgctgagtacaatgctccttgcagcaaagatcttggaaatgcttgaagaccacaaggctgaaaagtgctttgctggagtcctgttctcagagctccacagaagacacgtgtttttgtatctttaaagacttgatgaataaacactttttctggtcaaagaccacaaggctgaaaagtgctttgctggagtcctgttctcagagctccacagaagacacgtgtttttgtatctttaaagacttgatgaataaacactttttctggtcaa

SEQ ID NO: 16 - protein, 325 aa, Homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 17 - DNA, 1414 bp, Homo sapiens
gaggaagaagcggaggaggcggctcccgcgctcgcagggccgtgccacctgcccgcccgcccgctcgctcgctcgcccgccgcgccgcgctgccgaccgccagcatgctgccgagagtgggctgccccgcgctgccgctgccgccgccgccgctgctgccgctgctgctgctgctactgggcgcgagtggcggcggcggcggggcgcgcgcggaggtgctgttccgctgcccgccctgcacacccgagcgcctggccgcctgcgggcccccgccggttgcgccgcccgccgcggtggccgcagtggccggaggcgcccgcatgccatgcgcggagctcgtccgggagccgggctgcggctgctgctcggtgtgcgcccggctggagggcgaggcgtgcggcgtctacaccccgcgctgcggccaggggctgcgctgctatccccacccgggctccgagctgcccctgcaggcgctggtcatgggcgagggcacttgtgagaagcgccgggacgccgagtatggcgccagcccggagcaggttgcagacaatggcgatgaccactcagaaggaggcctggtggagaaccacgtggacagcaccatgaacatgttgggcgggggaggcagtgctggccggaagcccctcaagtcgggtatgaaggagctggccgtgttccgggagaaggtcactgagcagcaccggcagatgggcaagggtggcaagcatcaccttggcctggaggagcccaagaagctgcgaccaccccctgccaggactccctgccaacaggaactggaccaggtcctggagcggatctccaccatgcgccttccggatgagcggggccctctggagcacctctactccctgcacatccccaactgtgacaagcatggcctgtacaacctcaaacagtgcaagatgtctctgaacgggcagcgtggggagtgctggtgtgtgaaccccaacaccgggaagctgatccagggagcccccaccatccggggggaccccgagtgtcatctcttctacaatgagcagcaggaggctcgcggggtgcacacccagcggatgcagtagaccgcagccagccggtgcctggcgcccctgccccccgcccctctccaaacaccggcagaaaacggagagtgcttgggtggtgggtgctggaggattttccagttctgacacacgtatttatatttggaaagagaccagcaccgagctcggcacctccccggcctctctcttcccagctgcagatgccacacctgctccttcttgctttccccgggggaggaagggggttgtggtcggggagctggggtacaggtttggggagggggaagagaaatttttatttttgaacccctgtgtcccttttgcataagattaaaggaaggaaaagtaaa

SEQ ID NO: 18 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSCDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 19 - DNA, 1957 bp, Homo sapiens
attcccaccgggacctgcggggctgagtgcccttctcggttgctgccgctgaggagcccgcccagccagccagggccgcgaggccgaggccaggccgcagcccaggagccgccccaccgcagctggcgatggacccgccgaggcccgcgctgctggcgctgctggcgctgcctgcgctgctgctgctgctgctggcgggcgccagggccgaagaggaaatgctggaaaatgtcagcctggtctgtccaaaagatgcgacccgattcaagcacctccggaagtacacatacaactatgaggctgagagttccagtggagtccctgggactgctgattcaagaagtgccaccaggatcaactgcaaggttgagctggaggttccccagctctgcagcttcatcctgaagaccagccagtgcaccctgaaagaggtgtatggcttcaaccctgagggcaaagccttgctgaagaaaaccaagaactctgaggagtttgctgcagccatgtccaggtatgagctcaagctggccattccagaagggaagcaggttttcctttacccggagaaagatgaacctacttacatcctgaacatcaagaggggcatcatttctgccctcctggttcccccagagacagaagaagccaagcaagtgttgtttctggataccgtgtatggaaactgctccactcactttaccgtcaagacgaggaagggcaatgtggcaacagaaatatccactgaaagagacctggggcagtgtgatcgcttcaagcccatccgcacaggcatcagcccacttgctctcatcaaaggcatgacccgccccttgtcaactctgatcagcagcagccagtcctgtcagtacacactggacgctaagaggaagcatgtggcagaagccatctgcaaggagcaacacctcttcctgcctttctcctacaagaataagtatgggatggtagcacaagtgacacagactttgaaacttgaagacacaccaaagatcaacagccgcttctttggtgaaggtactaagaagatgggcctcgcatttgagagcaccaaatccacatcacctccaaagcaggccgaagctgttttgaagactctccaggaactgaaaaaactaaccatctctgagcaaaatatccagagagctaatctcttcaataagctggttactgagctgagaggcctcagtgatgaagcagtcacatctctcttgccacagctgattgaggtgtccagccccatcactttacaagccttggttcagtgtggacagcctcagtgctccactcacatcctccagtggctgaaacgtgtgcatgccaacccccttctgatagatgtggtcacctacctggtggccctgatccccgagccctcagcacagcagctgcgagagatcttcaacatggcgagggatcagcgcagccgagccaccttgtatgcgctgagccacgcggtcaacaactatcataagacaaaccctacagggacccaggagctgctggacattgctaattacctgatggaacagattcaagatgactgcactggggatgaagattacacctatttgattctgcgggtcattggaaatatgggccaaaccatggagcagttaactccagaactcaagtcttcaatcctgaaatgtgtccaaagtacaaagccatcactgatgatccagaaagctgccatccaggctctgcggaaaatggagcctaaagacaaggaccaggaggttcttcttcagactttccttgatgatgcttctccgggagataagcgactggctgcctatcttatgttgatgaggagtccttcacaggcagatattaacaaaattgtccaaattctaccatgggaacagaatgagcaagtgaagaactttgtggcttcccatattgccaatatcttgaactcagaagaattggatatccaaga

SEQ ID NO: 20 - protein, 381 aa, Homo sapiens
  MISC_FEATURE (59)..(59): x=Selenocysteine
  MISC_FEATURE (300)..(300): x=Selenocysteine
  MISC_FEATURE (318)..(318): x=Selenocysteine
  MISC_FEATURE (330)..(330): x=Selenocysteine
  MISC_FEATURE (345)..(345): x=Selenocysteine
  MISC_FEATURE (352)..(352): x=Selenocysteine
  MISC_FEATURE (367)..(367): x=Selenocysteine
  MISC_FEATURE (369)..(369): x=Selenocysteine
  MISC_FEATURE (376)..(376): x=Selenocysteine
  MISC_FEATURE (378)..(378): x=Selenocysteine
MWRSLGLALALCLLPSGGTESQDQSSLCKQPPAWSIRDQDPMLNSNGSVTVVALLQASXYLCILQASKLEDLRVKLKKEGYSNISYIVVNHQGISSRLKYTHLKNKVSEHIPVYQQEENQTDVWTLLNGSKDDFLIYDRCGRLVYHLGLPFSFLTFPYVEEAIKIAYCEKKCGNCSLTTLKDEDFCKRVSLATVDKTVETPSPHYHHEHHHNHGHQHLGSSELSENQQPGAPNAPTHPAPPGLHHHHKHKGQHRQGHPENRDMPASEDLQDLQKKLCRKRCINQLLCKLPTDSELAPRSXCCHCRHLIFEKTGSAITXQCKENLPSLCSXQGLRAEENITESCQXRLPPAAXQISQQLIPTEASASXRXKNQAKKXEXPSN

SEQ ID NO: 21 - DNA, 2204 bp, Homo sapiens
acagacaggcaggtgcaggagacggggtgagcgcttttgggctctagccccatggcagcatctaggggtgtgttacaattaatgctcttttagcagttgctgttcgcggatggctaagtgttaaaccagctcagtggagagtcagggtggcagccttttacgccctgtcctcttggtacccgggtctttgtctagcatccaggaagaatcaggtcctacagacttgaaggatggtgaatacagagattttattgaatgatggaggaggctttcagcaggatggatggggagctggaaagcggatggagtgagaagataatcttcccctggggtttggccatcccatggctgatctctctgacagtccccagccgaactcctctcgatgttcagacactctttctcctctctcctctgccacactgctctgctgctcttctgctcatggagcctgaggacaaccccagcaatgtggagaagcctggggcttgccctggctctctgtctcctcccatcgggaggaacagagagccaggaccaaagctccttatgtaagcaacccccagcctggagcataagagatcaagatccaatgctaaactccaatggttcagtgactgtggttgctcttcttcaagccagctgatacctgtgcatactgcaggcatctaaattagaagacctgcgagtaaaactgaagaaagaaggatattctaatatttcttatattgttgttaatcatcaaggaatctcttctcgattaaaatacacacatcttaagaataaggtttcagagcatattcctgtttatcaacaagaagaaaaccaaacagatgtctggactcttttaaatggaagcaaagatgacttcctcatatatgatagatgtggccgtcttgtatatcatcttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccaggccttcatcaccaccataagcacaagggtcagcataggcagggtcacccagagaaccgagatatgccagcaagtgaagatttacaagatttacaaaagaagctctgtcgaaagagatgtataaatcaattactctgtaaattgcccacagattcagagttggctcctaggagctgatgctgccattgtcgacatctgatatttgaaaaaacagggtctgcaatcacctgacagtgtaaagaaaacctcccatctttatgtagctgacagggacttcgggcagaggagaacataactgaatcttgtcagtgacgtttgcctccagctgcctgacaaataagtcagcagcttatacccacagaagccagtgccagttgacgctgaaagaatcaggcaaaaaagtgagaatgaccttcaaactaaatatttaaaataggacatactccccaatttagtctagacacaatttcatttccagcatttttataaactaccaaattagtgaaccaaaaatagaaattagatttgtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggtttctgttggataattagcagtttagaatggaggaagaacaacaaagacatgctttccatttttttctttacttatctctcaaaacaatattactttgtcttttcaatcttctacttttaactaataaaataagtggattttgtattttaagatccagaaatacttaacacgtgaatattttgctaaaaaagcatatataactattttaaatatccatttatcttttgtatatctaagactcatcctgatttttactatcacacatgaataaagcctttgtatcttt

SEQ ID NO: 22 - protein, 94 aa, Homo sapiens
MKRVLSCVPEPTVVMAARALCMLGLVLALLSSSSAEEYVGLSANQCAVPAKDRVDCGYPHVTPKECNNRGCCFDSRIPGVPWCFKPLQEAECTF

SEQ ID NO: 23 - DNA, 1109 bp, Homo sapiens
cgctccccagtagaggacccggaaccagaactggaatccgcccttaccgcttgctgccaaaacagtgggggctgaactgacctctcccctttgggagagaaaaactgtctgggagcttgacaaaggcatgcaggagagaacaggagcagccacagccaggagggagagccttccccaagcaaacaatccagagcagctgtgcaaacaacggtgcataaatgaggcctcctggaccatgaagcgagtcctgagctgcgtcccggagcccacggtggtcatggctgccagagcgctctgcatgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccgaggacagttcttcgtgcctgagactttctgaggttgtgctttatttctgctgcgtcgtgggagagggcgggagggtgtcaggggagagtctgcccaggcctcaagggcaggaaaagactccctaaggagctgcagtgcatgcaaggatattttgaatccagactggcacccacgtcacaggaaagcctaggaacactgtaagtgccgcttcctcgggaaagcagaaaaaatacatttcaggtagaagttttcaaaaatcacaagtctttcttggtgaagacagcaagccaataaaactgtcttccaaagtggtcctttatttcacaaccactctcgctactgttcaatacttgtactattcctgggttttgtttctttgtacagtaaacattatgaacaaacaggca

SEQ ID NO: 24 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 25 - DNA, 1526 bp, Homo sapiens
aggtaacaccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagca

SEQ ID NO: 26 - protein, 383 aa, Homo sapiens
MGVKASQTGFVVLVLLQCCSAYKLVCYYTSWSQYREGDGSCFPDALDRFLCTHIIYSFANISNDHIDTWEWNDVTLYGMLNTLKNRNPNLKTLLSVGGWNFGSQRFSKIASNTQSRRTFIKSVPPFLRTHGFDGLDLAWLYPGRRDKQHFTTLIKEMKAEFIKEAQPGKKQLLLSAALSAGKVTIDSSYDIAKISQHLDFISIMTYDFHGAWRGTTGHHSPLFRGQEDASPDRFSNTDYAVGYMLRLGAPASKLVMGIPTFGRSFTLASSETGVGAPISGPGIPGRFTKEAGTLAYYEICDFLRGATVHRILGQQVPYATKGNQWVGYDDQESVKSKVQYLKDRQLAGAMVWALDLDDFQGSFCGQDLRFPLTNAIKDALAAT

SEQ ID NO: 27 - DNA, 16143 bp, Homo sapiens
gaggccctgtctaggtagctggcaccaggagccgtgggcaagggaagaggccacaccctgccctgctctgctgcagccagaatgggtgtgaaggcgtctcaaacaggctttgtggtcctggtgctgctccagtgctgctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggaaccccaacctgaagactctcttgtctgtcggaggatggaactttgggtctcaaagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggaaatgaaggccgaatttataaaggaagcccagccagggaaaaagcagctcctgctcagcgcagcactgtctgcggggaaggtcaccattgacagcagctatgacattgccaagatatcccaacacctggatttcattagcatcatgacctacgattttcatggagcctggcgtgggaccacaggccatcacagtcccctgttccgaggtcaggaggatgcaagtcctgacagattcagcaacactgactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgagatctgtgacttcctccgcggagccacagtccatagaatcctcggccagcaggtcccctatgccaccaagggcaaccagtgggtaggatacgacgaccaggaaagcgtcaaaagcaaggtgcagtacctgaaggacaggcagctggcgggcgccatggtatgggccctggacctggatgacttccagggctccttctgtggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtggtatctgggctagccaaggttaatccatcagagttgtgggttttcaggcccagacagcccgcagagccatctgcctgctgggtgagggactaagggagtgggcagagggggaggagaagcagagccaggggagggactgaggctgcaaccaggaggtgggggtgggggaggtgggtctcagttgcttgggggagggagcagggcggaagggcaggatgcacttgcaggggtctcatcctggatttctcttcaggtgagtaatccctccacctccacttttaagtccagaggcgtggcgagggcacagggcaggtgtggaggaggtcttagctccaagggaacactttgccaggttcttctgtgcttccaatgacttcgaaatagtcacgtttgctaaactggagtgaggagtatatggatgtttattttgctatactctttggtatatgtgaaaattctcaaaataagaaactttaaaaggtcctggttcagtgaaggctttgactaacagcccctgggagccgctagtacagttgcccagtagctgcttggatgagtgatgcccacattgtttgcagcaggagaaacagaattagaggcgaggaaggatttcctggtccattgtgatattgtgccccagcctgggggacatgccgagggagcacaggactgccattccgggtgggttacaagttagaggcactctcaccaggaggcagagaaaggtgggccagagtccctcatggatgaaggacctcactgcaggtcactaaaggtgccagcctgaaagccagggccatctgtgacacaggctatcttgggccttcccctcccacaaagccactgcttccaggaagcctgctgtgctggcaccaagtcccatttcatccttattccccagctcctacccttcccccacacagtgcctgtagcgtattcatcccctgcattggatcttttctaagcattttcagaataggccttattttcaccagtcagggaactccccagaggcagggggacctgaaccagctcctttcatttaagaattctgactttgctcacaatctgcaccccactcctccctacccaaccccacaccccattggtgctcagctctgggctcagcctgaggtctcttgccgaatcctggcccgggccccatcccagacttctccttcaggttgggccagggctggccaggagaggagggaccagctgggctggtcccagggcagggcaaccccacccaggaggaagggcgaggccccaccctgctcacctatgtttggagagttagaggggcttggcttaaatgggtggaggggatgactgtaggtgctgggcaatatttggggtgatgagagactgtagacggaagcgcagctgagcccctctgggagaggggaaccctctcctgcccctaacgtgctctccagggagcatggaaaatgagcaagactctactttgccatactctgtcttctccactggggaaaacaaaaatgggcagcagacctcagagtaactcccatgaagcctgtgacccctcagagatccaccactatgaatctctatgggattcccatgatgtcctatgggaggacatcagcggcctaacccagcctctcacccaaagcaggaaaccttctatggcctctcagacatggggccacccagtgtcatgacaatgtcattccactcctgccctccccacctccctgtgctccacaggtaggagcctctccccaggggcaggacggcagaggatgatggcataggagtgaggagcttgggtctcccgcatccactgtatggatgttccaggggctccagactagataggtacaaggcctacctgtttgtcaagggcctgcctgatgtgtagcaaagaaagcagagcccagagagagagcaggacttgccaagagtcacacagcaagttaacagtggatctcccaattccctgccaatgctctatttactacctcacaggcccgaaaatatgggacttctggggctaccaccattagggctggaaagagagagatggaaaccaatggggacattgagaagtggggagaccctgggaggagtctttggattaggtggggttggagcagggcttgaaatgggggtggttatggccatcgttggcacccatgtgctgggtactctttctgtgtccagcaccctgtctccccatgatcccatgttgtcctcacaacagcaaggtgagctgttatatttgtccccattttacagatgatgaaactgagactcaggttataacctttcacagtggctggctagtaagtgatagagccagacttggaaacctggaatgcctggctctaaggcacatgcatgcctggaggcgacccctgtcccaatcatgccctcccagagctgtgtggcctcaggatcccagctctgcaggtcctggaaaccccaccagaggcccaaggcacctagcatatcagtgctgagcatgctacagggctgattttggtcctaggtgctggtcaggtcagggacagggagggaggtgggcaggcatgagggaatggggtgggctagaagccggcgtcagctgctgtcctcctgaggacaggtaaagagggacttcagcctcagggcagtgtcctgggaccctgtgccctgaagatctcacatagcaaggaaggcttcttgtgaccatggtgggaggtgggaatggggttctaagaggtggaaggttgtgactgagcagagcacccacttataactacccacttagtgcattgcccattgcccacccttcaatcccatactgatgccacccatacccagcatgcactgtgtccagcagctctcagctctgcctgggggcagcctaagtatctccagttaccaggggcagaaggagagtctaaacaaattgttcacaataccagcagcaatcactttaactttgggatttcatgcaccagctagaacaaagcacagaccgaaaggcaaatgtctcccaaagtcatctgtgggccaataggggtcacccactgtccgatgctccctccaggacagggagtaattgagtgctgatgggtgtgcatgggtttggggagaagattagtcagttttttaggaaaaggatgggtgacttgaggatgggaccactgatgagccactttatcacttccactaggccccaggcaggttggggagtacagcaaatgggttgcgcagagacctagtccgccctcgatgagtctacctctcatgccacttgggaccctttctctcacctgcctgtctcccatctaggtgagtctcctgccccaggcagcctccccagaacctctgccaacactcctgtttccccgccacccccagccctagcactccatgtagactgaggacactgggcttccagtcccagttcttccaaagccttgatgtgtgaccttgagcaagtcacttccctgccctgtgcctcagtttccccatctgtcaaatgaggcataacaatccttgcctttcctttttcacctgggctgttggctgagcagttgagagagctgctatgaaagtattttggaaagggaagtggaaaaagctattcaaaccctccagctattgcagagtattttctccacaccaggcagagcagagtgctgggccctggagaggccacacagcagcctctttctagagtgctggggagtcctcaaggctctctcatacacacaggcctccccctttcctgttggccccacgcctcatcctcaccccacccctcatgcctgtgaggaacaaggaacagccaagcagcctcatctctcctgagagcagagccatgggtctcgggaaacccaggagtaaggaacaaagactcttcaaaatgacttcagagctttctttaggatcccagggaggtgtaaactcagtgcttaattaaatggattctttagagggtgggaaacaggtggatgtcaaccatttgccccacatacccgtattcatgcaatccacccccagtgggctcaccggtgcgggtgtgcaaaacctcctcccaccccagtcatcttaggttcaggtcatcctttggtcctgctctttccccgccaggctgtctgttgatgctacttttaatctgctttcactaagataagcctggcagaagtggtgggggtaaggtgggctgtaggccagctcccaaatgtgtgctgggcataacagaagacccattcttgactgaagtgcccttgtgggaccctgagcccgtgccctggagtggcacagggaggtgtgccagcaatggggaccgaggtcactggggactgctggggttcgggctagtggcctgtctggctgctgtctgcttccctcgttcacatcctgctggagccttaaataggagcccaaaagcttttctttcttacttttttttgagacaaaatctcactctgttgcccaggccggagtgcagtggcacaacctctgccgcctgggttcaagcgattctcctgcctcagcctcccgagtagctgggattacaggtgcctgccaccatgcctggctaattttcgtagttagagtagagacggggtttcaccatcttggccaggctggtcttgaactcctcacctcatgatccacctgcctcggcctcccaaagtgctgggattccaggtatgagccaccacgcccggcctaaagcttttctattaataatttcctgcctcaccctccatccccttcctcctcaggtgagttcccagaaggagggagggccaggagggggccgcaagacctgccatctgccagtgctcacacaccaatctctctagccctcagtacagtcctgcaagaggggggtcatgaggcccattccacagataaggaaactgaggcccaaagtctgggcatgctgcgttgctctgggaaggtgatctgcagggtaaatggagtgagggcagggggccgaatggggagaggctgggagccgaggaggtaggagtcattgtgccctcagagccaaccacctgatttctgcatctgtcaaatagtaatagccccttcctatgcctcaaaggatttttttcaagaatgaaactgtaaaattcactttaaagtgacatgatccgttcccggagggacaggggaatccccagtgcaccatacaccaataacccctgctaaggcagcagtattaattgctcaaccttccgcacctgtgcactaactgctcacgttgttcccaccctaccccacccaggtaatggagttggggagaaggtaggattcccccacaccaccagcagccctggggaaggtgattccccaccacgttcttgctttttctcctttgggaataaagaaaatgtccggttgccccagcatgcctgaggaagtggaagggagaggttaggacatttgttgctgaaaatctccagcaggtacaggcacatgggcctgcaccactaggcacctgggatagccctctggctatggggctgaggtcttccttccagcccaggaaagagcagaggtcaagaggcagattttttgtttcactctagcctctgctactctgtgtggccttgggcctgtcctcagtgtcaaccagcaggcctcacatctctgtttaaatggaagaagctaagcagggccaaggcagccaccatctctgggtcatttgcctctggtttgtataaacttgtgtgatgcatgcagcctgcagaccctgcagagagtgaggctgcaggatggagcaggagctaaaagagatttggagagtggcgtctcctggtgacctgcaaggtctcggcacgactccccacactgccttttccctgttatctgctcaggtaggtttccccaaggccacacctcaggacaaagagaaagaaggaggccccgctcccagcagccagattcctgtcccttgcactgaggtctgggctgggctcacagagcacatgtgccctgtacacactctgggtcagggaacccagccctgctcctctgggcctcccctgccagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtggaggccctgtctaggtagctggcaccaggagccgtgggcaagggaagaggccacaccctgccctgctctgctgcagccagagaggccctgtctaggtagctggcaccaggagccgtgggcaagggaagaggccacaccctgccctgctctgctgcagccagaatgggtgtgaaggcgtctcaaacaggtatctgggctagccaaggttaatccatcagagttgtgggttttcaggcccagacagcccgcagagccatctgcctgctgggtgagggactaagggagtgggcagagggggaggagaagcagagccaggggagggactgaggctgcaaccaggaggtgggggtgggggaggtgggtctcagttgcttgggggagggagcagggcggaagggcaggatgcacttgcaggggtctcatcctggatttctcttcaggctttgtggtcctggtgctgctccagtgctgtgagtaatccctccacctccacttttaagtccagaggcgtggcgagggcacagggcaggtgtggaggaggtcttagctccaagggaacactttgccaggttcttctgtgcttccaatgacttcgaaatagtcacgtttgctaaactggagtgaggagtatatggatgtttattttgctatactctttggtatatgtgaaaattctcaaaataagaaactttaaaaggtcctggttcagtgaaggctttgactaacagcccctgggagccgctagtacagttgcccagtagctgcttggatgagtgatgcccacattgtttgcagcaggagaaacagaattagaggcgaggaaggatttcctggtccattgtgatattgtgccccagcctgggggacatgccgagggagcacaggactgccattccgggtgggttacaagttagaggcactctcaccaggaggcagagaaaggtgggccagagtccctcatggatgaaggacctcactgcaggtcactaaaggtgccagcctgaaagccagggccatctgtgacacaggctatcttgggccttcccctcccacaaagccactgcttccaggaagcctgctgtgctggcaccaagtcccatttcatccttattccccagctcctacccttcccccacacagtgcctgtagcgtattcatcccctgcattggatcttttctaagcattttcagaataggccttattttcaccagtcagggaactccccagaggcagggggacctgaaccagctcctttcatttaagaattctgactttgctcacaatctgcaccccactcctccctacccaaccccacaccccattggtgctcagctctgggctcagcctgaggtctcttgccgaatcctggcccgggccccatcccagacttctccttcaggctctgcatacaaactggtctgctactacaccagctggtcccagtaccgggaaggcgatgggagctgcttcccagatgcccttgaccgcttcctctgtacccacatcatctacagctttgccaatataagcaacgatcacatcgacacctgggagtggaatgatgtgacgctctacggcatgctcaacacactcaagaacaggttgggccagggctggccaggagaggagggaccagctgggctggtcccagggcagggcaaccccacccaggaggaagggcgaggccccaccctgctcacctatgtttggagagttagaggggcttggcttaaatgggtggaggggatgactgtaggtgctgggcaatatttggggtgatgagagactgtagacggaagcgcagctgagcccctctgggagaggggaaccctctcctgcccctaacgtgctctccagggagcatggaaaatgagcaagactctactttgccatactctgtcttctccactggggaaaacaaaaatgggcagcagacctcagagtaactcccatgaagcctgtgacccctcagagatccaccactatgaatctctatgggattcccatgatgtcctatgggaggacatcagcggcctaacccagcctctcacccaaagcaggaaaccttctatggcctctcagacatggggccacccagtgtcatgacaatgtcattccactcctgccctccccacctccctgtgctccacaggaaccccaacctgaagactctcttgtctgtcggaggatggaactttgggtctcaaaggtaggagcctctccccaggggcaggacggcagaggatgatggcataggagtgaggagcttgggtctcccgcatccactgtatggatgttccaggggctccagactagataggtacaaggcctacctgtttgtcaagggcctgcctgatgtgtagcaaagaaagcagagcccagagagagagcaggacttgccaagagtcacacagcaagttaacagtggatctcccaattccctgccaatgctctatttactacctcacaggcccgaaaatatgggacttctggggctaccaccattagggctggaaagagagagatggaaaccaatggggacattgagaagtggggagaccctgggaggagtctttggattaggtggggttggagcagggcttgaaatgggggtggttatggccatcgttggcacccatgtgctgggtactctttctgtgtccagcaccctgtctccccatgatcccatgttgtcctcacaacagcaaggtgagctgttatatttgtccccattttacagatgatgaaactgagactcaggttataacctttcacagtggctggctagtaagtgatagagccagacttggaaacctggaatgcctggctctaaggcacatgcatgcctggaggcgacccctgtcccaatcatgccctcccagagctgtgtggcctcaggatcccagctctgcaggtcctggaaaccccaccagaggcccaaggcacctagcatatcagtgctgagcatgctacagggctgattttggtcctagattttccaagatagcctccaacacccagagtcgccggactttcatcaagtcagtaccgccatttctgcgcacccatggctttgatgggctggaccttgcctggctctaccctggacggagagacaaacagcattttaccaccctaatcaaggtgctggtcaggtcagggacagggagggaggtgggcaggcatgagggaatggggtgggctagaagccggcgtcagctgctgtcctcctgaggacaggtaaagagggacttcagcctcagggcagtgtcctgggaccctgtgccctgaagatctcacatagcaaggaaggcttcttgtgaccatggtgggaggtgggaatggggttctaagaggtggaaggttgtgactgagcagagcacccacttataactacccacttagtgcattgcccattgcccacccttcaatcccatactgatgccacccatacccagcatgcactgtgtccagcagctctcagctctgcctgggggcagcctaagtatctccagttaccaggggcagaaggagagtctaaacaaattgttcacaataccagcagcaatcactttaactttgggatttcatgcaccagctagaacaaagcacagaccgaaaggcaaatgtctcccaaagtcatctgtgggccaataggggtcacccactgtccgatgctccctccaggacagggagtaattgagtgctgatgggtgtgcatgggtttggggagaagattagtcagttttttaggaaaaggatgggtgacttgaggatgggaccactgatgagccactttatcacttccactaggccccaggcaggttggggagtacagcaaatgggttgcgcagagacctagtccgccctcgatgagtctacctctcatgccacttgggaccctttctctcacctgcctgtctcccatctaggaaatgaaggccgaatttataaaggaagcccagccagggaaaaagcagctcctgctcagcgcagcactgtctgcggggaaggtcaccattgacagcagctatgacattgccaagatatcccagtgagtctcctgccccaggcagcctccccagaacctctgccaacactcctgtttccccgccacccccagccctagcactccatgtagactgaggacactgggcttccagtcccagttcttccaaagccttgatgtgtgaccttgagcaagtcacttccctgccctgtgcctcagtttccccatctgtcaaatgaggcataacaatccttgcctttcctttttcacctgggctgttggctgagcagttgagagagctgctatgaaagtattttggaaagggaagtggaaaaagctattcaaaccctccagctattgcagagtattttctccacaccaggcagagcagagtgctgggccctggagaggccacacagcagcctctttctagagtgctggggagtcctcaaggctctctcatacacacaggcctccccctttcctgttggccccacgcctcatcctcaccccacccctcatgcctgtgaggaacaaggaacagccaagcagcctcatctctcctgagagcagagccatgggtctcgggaaacccaggagtaaggaacaaagactcttcaaaatgacttcagagctttctttaggatcccagggaggtgtaaactcagtgcttaattaaatggattctttagagggtgggaaacaggtggatgtcaaccatttgccccacatacccgtattcatgcaatccacccccagtgggctcaccggtgcgggtgtgcaaaacctcctcccaccccagtcatcttaggttcaggtcatcctttggtcctgctctttccccgccaggctgtctgttgatgctacttttaatctgctttcactaagataagcctggcagaagtggtgggggtaaggtgggctgtaggccagctcccaaatgtgtgctgggcataacagaagacccattcttgactgaagtgcccttgtgggaccctgagcccgtgccctggagtggcacagggaggtgtgccagcaatggggaccgaggtcactggggactgctggggttcgggctagtggcctgtctggctgctgtctgcttccctcgttcacatcctgctggagccttaaataggagcccaaaagcttttctttcttacttttttttgagacaaaatctcactctgttgcccaggccggagtgcagtggcacaacctctgccgcctgggttcaagcgattctcctgcctcagcctcccgagtagctgggattacaggtgcctgccaccatgcctggctaattttcgtagttagagtagagacggggtttcaccatcttggccaggctggtcttgaactcctcacctcatgatccacctgcctcggcctcccaaagtgctgggattccaggtatgagccaccacgcccggcctaaagcttttctattaataatttcctgcctcaccctccatccccttcctcctcagacacctggatttcattagcatcatgacctacgattttcatggagcctggcgtgggaccacaggccatcacagtcccctgttccgaggtcaggaggatgcaagtcctgacagattcagcaacactgtgagttcccagaaggagggagggccaggagggggccgcaagacctgccatctgccagtgctcacacaccaatctctctagccctcagtacagtcctgcaagaggggggtcatgaggcccattccacagataaggaaactgaggcccaaagtctgggcatgctgcgttgctctgggaaggtgatctgcagggtaaatggagtgagggcagggggccgaatggggagaggctgggagccgaggaggtaggagtcattgtgccctcagagccaaccacctgatttctgcatctgtcaaatagtaatagccccttcctatgcctcaaaggatttttttcaagaatgaaactgtaaaattcactttaaagtgacatgatccgttcccggagggacaggggaatccccagtgcaccatacaccaataacccctgctaaggcagcagtattaattgctcaaccttccgcacctgtgcactaactgctcacgttgttcccaccctaccccacccaggactatgctgtggggtacatgttgaggctgggggctcctgccagtaagctggtgatgggcatccccaccttcgggaggagcttcactctggcttcttctgagactggtgttggagccccaatctcaggaccgggaattccaggccggttcaccaaggaggcagggacccttgcctactatgaggtaatggagttggggagaaggtaggattcccccacaccaccagcagccctggggaaggtgattccccaccacgttcttgctttttctcctttgggaataaagaaaatgtccggttgccccagcatgcctgaggaagtggaagggagaggttaggacatttgttgctgaaaatctccagcaggtacaggcacatgggcctgcaccactaggcacctgggatagccctctggctatggggctgaggtcttccttccagcccaggaaagagcagaggtcaagaggcagattttttgtttcactctagcctctgctactctgtgtggccttgggcctgtcctcagtgtcaaccagcaggcctcacatctctgtttaaatggaagaagctaagcagggccaaggcagccaccatctctgggtcatttgcctctggtttgtataaacttgtgtgatgcatgcagcctgcagaccctgcagagagtgaggctgcaggatggagcaggagctaaaagagatttggagagtggcgtctcctggtgacctgcaaggtctcggcacgactccccacactgccttttccctgttatctgctcagatctgtgacttcctccgcggagccacagtccatagaatcctcggccagcaggtcccctatgccaccaagggcaaccagtgggtaggatacgacgaccaggaaagcgtcaaaagcaaggtaggtttccccaaggccacacctcaggacaaagagaaagaaggaggccccgctcccagcagccagattcctgtcccttgcactgaggtctgggctgggctcacagagcacatgtgccctgtacacactctgggtcagggaacccagccctgctcctctgggcctcccctgccaggtgcagtacctgaaggacaggcagctggcgggcgccatggtatgggccctggacctggatgacttccagggctccttctgtggccaggatctgcgcttccctctcaccaatgccatcaaggatgcactcgctgcaacgtagccctctgttctgcacacagcacgggggccaaggatgccccgtccccctctggctccagctggccgggagcctgatcacctgccctgctgagtcccaggctgagcctcagtctccctcccttggggcctatgcagaggtccacaacacacagatttgagctcagccctggtgggcagagaggtagggatggggctgtggggatagtgaggcatcgcaatgtaagactcgggattagtacacacttgttgattaatggaaatgtttacagatccccaagcctggcaagggaatttcttcaactccctgccccccagccctccttatcaaaggacaccattttggcaagctctatcaccaaggagccaaacatcctacaagacacagtgaccatactaattataccccctgcaaagcccagcttgaaaccttcacttaggaacgtaatcgtgtcccctatcctacttccccttcctaattccacagctgctcaataaagtacaagagcttaacagtg

SEQ ID NO: 28 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 29 - DNA, 6876 bp, Homo sapiens
agacacgtgctggggcgggcaggcgagcgcctcagtctggtcgcctggcggtgcctccggccccaacgcgcccgggccgccgcgggccgcgcgcgccgatgcccggctgagtcactggcagggcagcgcgcgtgtgggaaggggcggagggagtgcggccggcgggcgggcggggcgctgggctcagcccggccgcaggtgacccggaggccctcgccgcccgcggcgccccgagcgctttgtgagcagatgcggagccgagtggagggcgcgagccagatgcggggcgacagctgacttgctgagaggaggcggggaggcgcggagcgcgcgtgtggtccttgcgccgctgacttctccactggttcctgggcaccgaaagataaacctctcataatgaaggcccccgctgtgcttgcacctggcatcctcgtgctcctgtttaccttggtgcagaggagcaatggggagtgtaaagaggcactagcaaagtccgagatgaatgtgaatatgaagtatcagcttcccaacttcaccgcggaaacacccatccagaatgtcattctacatgagcatcacattttccttggtgccactaactacatttatgttttaaatgaggaagaccttcagaaggttgctgagtacaagactgggcctgtgctggaacacccagattgtttcccatgtcaggactgcagcagcaaagccaatttatcaggaggtgtttggaaagataacatcaacatggctctagttgtcgacacctactatgatgatcaactcattagctgtggcagcgtcaacagagggacctgccagcgacatgtctttccccacaatcatactgctgacatacagtcggaggttcactgcatattctccccacagatagaagagcccagccagtgtcctgactgtgtggtgagcgccctgggagccaaagtcctttcatctgtaaaggaccggttcatcaacttctttgtaggcaataccataaattcttcttatttcccagatcatccattgcattcgatatcagtgagaaggctaaaggaaacgaaagatggttttatgtttttgacggaccagtcctacattgatgttttacctgagttcagagattcttaccccattaagtatgtccatgcctttgaaagcaacaattttatttacttcttgacggtccaaagggaaactctagatgctcagacttttcacacaagaataatcaggttctgttccataaactctggattgcattcctacatggaaatgcctctggagtgtattctcacagaaaagagaaaaaagagatccacaaagaaggaagtgtttaatatacttcaggctgcgtatgtcagcaagcctggggcccagcttgctagacaaataggagccagcctgaatgatgacattcttttcggggtgttcgcacaaagcaagccagattctgccgaaccaatggatcgatctgccatgtgtgcattccctatcaaatatgtcaacgacttcttcaacaagatcgtcaacaaaaacaatgtgagatgtctccagcatttttacggacccaatcatgagcactgctttaataggacacttctgagaaattcatcaggctgtgaagcgcgccgtgatgaatatcgaacagagtttaccacagctttgcagcgcgttgacttattcatgggtcaattcagcgaagtcctcttaacatctatatccaccttcattaaaggagacctcaccatagctaatcttgggacatcagagggtcgcttcatgcaggttgtggtttctcgatcaggaccatcaacccctcatgtgaattttctcctggactcccatccagtgtctccagaagtgattgtggagcatacattaaaccaaaatggctacacactggttatcactgggaagaagatcacgaagatcccattgaatggcttgggctgcagacatttccagtcctgcagtcaatgcctctctgccccaccctttgttcagtgtggctggtgccacgacaaatgtgtgcgatcggaggaatgcctgagcgggacatggactcaacagatctgtctgcctgcaatctacaaggttttcccaaatagtgcaccccttgaaggagggacaaggctgaccatatgtggctgggactttggatttcggaggaataataaatttgatttaaagaaaactagagttctccttggaaatgagagctgcaccttgactttaagtgagagcacgatgaatacattgaaatgcacagttggtcctgccatgaataagcatttcaatatgtccataattatttcaaatggccacgggacaacacaatacagtacattctcctatgtggatcctgtaataacaagtatttcgccgaaatacggtcctatggctggtggcactttacttactttaactggaaattacctaaacagtgggaattctagacacatttcaattggtggaaaaacatgtactttaaaaagtgtgtcaaacagtattcttgaatgttataccccagcccaaaccatttcaactgagtttgctgttaaattgaaaattgacttagccaaccgagagacaagcatcttcagttaccgtgaagatcccattgtctatgaaattcatccaaccaaatcttttattagtacttggtggaaagaacctctcaacattgtcagttttctattttgctttgccagtggtgggagcacaataacaggtgttgggaaaaacctgaattcagttagtgtcccgagaatggtcataaatgtgcatgaagcaggaaggaactttacagtggcatgtcaacatcgctctaattcagagataatctgttgtaccactccttccctgcaacagctgaatctgcaactccccctgaaaaccaaagcctttttcatgttagatgggatcctttccaaatactttgatctcatttatgtacataatcctgtgtttaagccttttgaaaagccagtgatgatctcaatgggcaatgaaaatgtactggaaattaagggaaatgatattgaccctgaagcagttaaaggtgaagtgttaaaagttggaaataagagctgtgagaatatacacttacattctgaagccgttttatgcacggtccccaatgacctgctgaaattgaacagcgagctaaatatagagtggaagcaagcaatttcttcaaccgtccttggaaaagtaatagttcaaccagatcagaatttcacaggattgattgctggtgttgtctcaatatcaacagcactgttattactacttgggtttttcctgtggctgaaaaagagaaagcaaattaaagatctgggcagtgaattagttcgctacgatgcaagagtacacactcctcatttggataggcttgtaagtgcccgaagtgtaagcccaactacagaaatggtttcaaatgaatctgtagactaccgagctacttttccagaagatcagtttcctaattcatctcagaacggttcatgccgacaagtgcagtatcctctgacagacatgtcccccatcctaactagtggggactctgatatatccagtccattactgcaaaatactgtccacattgacctcagtgctctaaatccagagctggtccaggcagtgcagcatgtagtgattgggcccagtagcctgattgtgcatttcaatgaagtcataggaagagggcattttggttgtgtatatcatgggactttgttggacaatgatggcaagaaaattcactgtgctgtgaaatccttgaacagaatcactgacataggagaagtttcccaatttctgaccgagggaatcatcatgaaagattttagtcatcccaatgtcctctcgctcctgggaatctgcctgcgaagtgaagggtctccgctggtggtcctaccatacatgaaacatggagatcttcgaaatttcattcgaaatgagactcataatccaactgtaaaagatcttattggctttggtcttcaagtagccaaaggcatgaaatatcttgcaagcaaaaagtttgtccacagagacttggctgcaagaaactgtatgctggatgaaaaattcacagtcaaggttgctgattttggtcttgccagagacatgtatgataaagaatactatagtgtacacaacaaaacaggtgcaaagctgccagtgaagtggatggctttggaaagtctgcaaactcaaaagtttaccaccaagtcagatgtgtggtcctttggcgtgctcctctgggagctgatgacaagaggagccccaccttatcctgacgtaaacacctttgatataactgtttacttgttgcaagggagaagactcctacaacccgaatactgcccagaccccttatatgaagtaatgctaaaatgctggcaccctaaagccgaaatgcgcccatccttttctgaactggtgtcccggatatcagcgatcttctctactttcattggggagcactatgtccatgtgaacgctacttatgtgaacgtaaaatgtgtcgctccgtatccttctctgttgtcatcagaagataacgctgatgatgaggtggacacacgaccagcctccttctgggagacatcatagtgctagtactatgtcaaagcaacagtccacactttgtccaatggttttttcactgcctgacctttaaaaggccatcgatattctttgctcttgccaaaattgcactattataggacttgtattgttatttaaattactggattctaaggaatttcttatctgacagagcatcagaaccagaggcttggtcccacaggccacggaccaatggcctgcagccgtgacaacactcctgtcatattggagtccaaaacttgaattctgggttgaattttttaaaaatcaggtaccacttgatttcatatgggaaattgaagcaggaaatattgagggcttcttgatcacagaaaactcagaagagatagtaatgctcaggacaggagcggcagccccagaacaggccactcatttagaattctagtgtttcaaaacacttttgtgtgttgtatggtcaataacatttttcattactgatggtgtcattcacccattaggtaaacattcccttttaaatgtttgtttgttttttgagacaggatctcactctgttgccagggctgtagtgcagtggtgtgatcatagctcactgcaacctccacctcccaggctcaagcctcccgaatagctgggactacaggcgcacaccaccatccccggctaatttttgtattttttgtagagacggggttttgccatgttgccaaggctggtttcaaactcctggactcaagaaatccacccacctcagcctcccaaagtgctaggattacaggcatgagccactgcgcccagcccttataaatttttgtatagacattcctttggttggaagaatatttataggcaatacagtcaaagtttcaaaatagcatcacacaaaacatgtttataaatgaacaggatgtaatgtacatagatgacattaagaaaatttgtatgaaataatttagtcatcatgaaatatttagttgtcatataaaaacccactgtttgagaatgatgctactctgatctaatgaatgtgaacatgtagatgttttgtgtgtatttttttaaatgaaaactcaaaataagacaagtaatttgttgataaatatttttaaagataactcagcatgtttgtaaagcaggatacattttactaaaaggttcattggttccaatcacagctcataggtagagcaaagaaagggtggatggattgaaaagattagcctctgtctcggtggcaggttcccacctcgcaagcaattggaaacaaaacttttggggagttttattttgcattagggtgtgttttatgttaagcaaaacatactttagaaacaaatgaaaaaggcaattgaaaatcccagctatttcacctagatggaatagccaccctgagcagaactttgtgatgcttcattctgtggaattttgtgcttgctactgtatagtgcatgtggtgtaggttactctaactggttttgtcgacgtaaacatttaaagtgttatattttttataaaaatgtttatttttaatgatatgagaaaaattttgttaggccacaaaaacactgcactgtgaacattttagaaaaggtatgtcagactgggattaatgacagcatgattttcaatgactgtaaattgcgataaggaaatgtactgattgccaatacaccccaccctcattacatcatcaggacttgaagccaagggttaacccagcaagctacaaagagggtgtgtcacactgaaactcaatagttgagtttggctgttgttgcaggaaaatgattataactaaaagctctctgatagtgcagagacttaccagaagacacaaggaattgtactgaagagctattacaatccaaatattgccgtttcataaatgtaataagtaatactaattcacagagtattgtaaatggtggatgacaaaagaaaatctgctctgtggaaagaaagaactgtctctaccagggtcaagagcatgaacgcatcaatagaaagaactcggggaaacatcccatcaacaggactacacacttgtatatacattcttgagaacactgcaatgtgaaaatcacgtttgctatttataaacttgtccttagattaatgtgtctggacagattgtgggagtaagtgattcttctaagaattagatacttgtcactgcctatacctgcagctgaactgaatggtacttcgtatgttaatagttgttctgataaatcatgcaattaaagtaaagtgatgcaa

SEQ ID NO: 30 - protein, 308 aa, Homo sapiens
MPGQELRTVNGSQMLLVLLVLSWLPHGGALSLAEASRASFPGPSELHSEDSRFRELRKRYEDLLTRLRANQSWEDSNTDLVPAPAVRILTPEVRLGSGGHLHLRISRAALPEGLPEASRLHRALFRLSPTASRSWDVTRPLRRQLSLARPQAPALHLRLSPPPSQSDQLLAESSSARPQLELHLRPQAARGRRRARARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 31 - DNA, 1200 bp, Homo sapiens
agtcccagctcagagccgcaacctgcacagccatgcccgggcaagaactcaggacggtgaatggctctcagatgctcctggtgttgctggtgctctcgtggctgccgcatgggggcgccctgtctctggccgaggcgagccgcgcaagtttcccgggaccctcagagttgcactccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgaccgctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcggaggacgcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgtt

SEQ ID NO: 32 - protein, 93 aa, Homo sapiens
MDRLQTALLVVLVLLAVALQATEAGPYGANMEDSVCCRDYVRYRLPLRVVKHFYWTSDSCPRPGVVLLTFRDKEICADPRVPWVKMILNKLSQ

SEQ ID NO: 33 - DNA, 2917 bp, Homo sapiens
gcagacacctgggctgagacatacaggacagagcatggatcgcctacagactgcactcctggttgtcctcgtcctccttgctgtggcgcttcaagcaactgaggcaggcccctacggcgccaacatggaagacagcgtctgctgccgtgattacgtccgttaccgtctgcccctgcgcgtggtgaaacacttctactggacctcagactcctgcccgaggcctggcgtggtgttgctaaccttcagggataaggagatctgtgccgatcccagagtgccctgggtgaagatgattctcaataagctgagccaatgaagagcctactctgatgaccgtggccttggctcctccaggaaggctcaggagccctacctccctgccattatagctgctccccgccagaagcctgtgccaactctctgcattccctgatctccatccctgtggctgtcacccttggtcacctccgtgctgtcactgccatctcccccctgacccctctaacccatcctctgcctccctccctgcagtcagagggtcctgttcccatcagcgattcccctgcttaaacccttccatgactccccactgccctaagctgaggtcagtctcccaagcctggcatgtggccctctggatctgggttccatctctgtctccagcctgcccacttcccttcatgaatgttgggttctagctccctgttctccaaacccatactacacatcccacttctgggtctttgcctgggatgttgctgacacccagaaagtcccaccacctgcacatgtgtagccccaccagccctccaaggcattgctcgcccaagcagctggtaattccatttcatgtattagatgtcccctggccctctgtcccctcttaataaccctagtcacagtctccgcagattcttgggatttgggggttttctcccccacctctccactagttggaccaaggtttctagctaagttactctagtctccaagcctctagcatagagcactgcagacaggccctggctcagaatcagagcccagaaagtggctgcagacaaaatcaataaaactaatgtccctcccctctccctgccaaaaggcagttacatatcaatacagagactcaaggtcactagaaatgggccagctgggtcaatgtgaagccccaaatttgcccagattcacctttcttcccccactccctttttttttttttttttgagatggagtttcgctcttgtcacccacgctggagtgcaatggtgtggtcttggcttattgaagcctctgcctcctgggttcaagtgattctcttgcctcagcctcctgagtagctgggattacaggttcctgctaccacgcccagctaatttttgtatttttagtagagacgaggcttcaccatgttggccaggctggtctcgaactcctgtcctcaggtaatccgcccacctcagcctcccaaagtgctgggattacaggcgtgagccacagtgcctggcctcttccctctccccaccccccccccaacttttttttttttttatggcagggtctcactctgtcgcccaggctggagtgcagtggcgtgatctcggctcactacaacctcgacctcctgggttcaagcgattctcccaccccagcctcccaagtagctgggattacaggtgtgtgccactacggctggctaatttttgtatttttagtagagacaggtttcaccatattggccaggctggtcttgaactcctgacctcaagtgatccaccttccttgtgctcccaaagtgctgagattacaggcgtgagctatcacacccagcctccccctttttttcctaataggagactcctgtacctttcttcgttttacctatgtgtcgtgtctgcttacatttccttctcccctcaggctttttttgggtggtcctccaacctccaatacccaggcctggcctcttcagagtaccccccattccactttccctgcctccttccttaaatagctgacaatcaaattcatgctatggtgtgaaagactacctttgacttggtattataagctggagttatatatgtatttgaaaacagagtaaatacttaagaggccaaatagatgaatggaagaattttaggaactgtgagagggggacaaggtggagctttcctggccctgggaggaagctggctgtggtagcgtagcgctctctctctctgtctgtggcaggaggcaaagagtagggtgtaattgagtgaaggaatcctgggtagagaccattctcaggtggttgggccaggctaaagactgggatttgggtctatctatgcctttctggctgatttttgtagagacggggttttgccatgttacccaggctggtctcaaactcctgggctcaagcgatcctcctggctcagcctcccaaagtgctgggattacaggcgtgagtcactgcgcctggcttcctcttcctcttgagaaatattcttttcatacagcaagtatgggacagcagtgtcccaggtaaaggacataaatgttacaagtgtctggtcctttctgagggaggctggtgccgctctgcagggtatttgaacctgtggaattggaggaggccatttcactccctgaacccagcctgacaaatcacagtgagaatgttcaccttataggcttgctgtggggctcaggttgaaagtgtggggagtgacactgcctaggcatccagctcagtgtcatccagggcctgtgtccctcccgaacccagggtcaacctgcctaccacaggcactagaaggacgaatctgcctactgcccatgaacggggccctcaagcgtcctgggatctccttctccctcctgtcctgtccttgcccctcaggactgctggaaaataaatcctttaaaatag

SEQ ID NO: 34 - protein, 401 aa, Homo sapiens
MNNLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSGNSESTQKCGIDVTLCEEAFFRFAVPTKFTPNWLSVLVDNLPGTKVNAESVERIKRQHSSQEQTFQLLKLWKHQNKDQDIVKKIIQDIDLCENSVQRHIGHANLTFEQLRSLMESLPGKKVGAEDIEKTIKACKPSDQILKLLSLWRIKNGDQDTLKGLMHALKHSKTYHFPKTVTQSLKKTIRFLHSFTMYKLYQKLFLEMIGNQVQSVKISCL

SEQ ID NO: 35 - DNA, 2087 bp, Homo sapiens
ggagacgcaccggagcgctcgcccagccgccgcctccaagcccctgaggtttccggggaccacaatgaacaacttgctgtgctgcgcgctcgtgtttctggacatctccattaagtggaccacccaggaaacgtttcctccaaagtaccttcattatgacgaagaaacctctcatcagctgttgtgtgacaaatgtcctcctggtacctacctaaaacaacactgtacagcaaagtggaagaccgtgtgcgccccttgccctgaccactactacacagacagctggcacaccagtgacgagtgtctatactgcagccccgtgtgcaaggagctgcagtacgtcaagcaggagtgcaatcgcacccacaaccgcgtgtgcgaatgcaaggaagggcgctaccttgagatagagttctgcttgaaacataggagctgccctcctggatttggagtggtgcaagctggaaccccagagcgaaatacagtttgcaaaagatgtccagatgggttcttctcaaatgagacgtcatctaaagcaccctgtagaaaacacacaaattgcagtgtctttggtctcctgctaactcagaaaggaaatgcaacacacgacaacatatgttccggaaacagtgaatcaactcaaaaatgtggaatagatgttaccctgtgtgaggaggcattcttcaggtttgctgttcctacaaagtttacgcctaactggcttagtgtcttggtagacaatttgcctggcaccaaagtaaacgcagagagtgtagagaggataaaacggcaacacagctcacaagaacagactttccagctgctgaagttatggaaacatcaaaacaaagaccaagatatagtcaagaagatcatccaagatattgacctctgtgaaaacagcgtgcagcggcacattggacatgctaacctcaccttcgagcagcttcgtagcttgatggaaagcttaccgggaaagaaagtgggagcagaagacattgaaaaaacaataaaggcatgcaaacccagtgaccagatcctgaagctgctcagtttgtggcgaataaaaaatggcgaccaagacaccttgaagggcctaatgcacgcactaaagcactcaaagacgtaccactttcccaaaactgtcactcagagtctaaagaagaccatcaggttccttcacagcttcacaatgtacaaattgtatcagaagttatttttagaaatgataggtaaccaggtccaatcagtaaaaataagctgcttataactggaaatggccattgagctgtttcctcacaattggcgagatcccatggatgagtaaactgtttctcaggcacttgaggctttcagtgatatctttctcattaccagtgactaattttgccacagggtactaaaagaaactatgatgtggagaaaggactaacatctcctccaataaaccccaaatggttaatccaactgtcagatctggatcgttatctactgactatattttcccttattactgcttgcagtaattcaactggaaattaaaaaaaaaaaactagactccattgtgccttactaaatatgggaatgtctaacttaaatagctttgagatttcagctatgctagaggcttttattagaaagccatatttttttctgtaaaagttactaatatatctgtaacactattacagtattgctatttatattcattcagatataagatttgtacatattatcatcctataaagaaacggtatgacttaattttagaaagaaaattatattctgtttattatgacaaatgaaagagaaaatatatatttttaatggaaagtttgtagcatttttctaataggtactgccatatttttctgtgtggagtatttttataattttatctgtataagctgtaatatcattttatagaaaatgcattatttagtcaattgtttaatgttggaaaacatatgaaatataaattatctgaatattagatgctctgagaaattgaatgtaccttatttaaaagattttatggttttataactatataaatgacattattaaagttttcaaattatttttta

SEQ ID NO: 36 - protein, 496 aa, Homo sapiens
MWQIVFFTLSCDLVLAAAYNNFRKSMDSIGKKQYQVQHGSCSYTFLLPEMDNCRSSSSPYVSNAVQRDAPLEYDDSVQRLQVLENIMENNTQWLMKLENYIQDNMKKEMVEIQQNAVQNQTAVMIEIGTNLLNQTAEQTRKLTDVEAQVLNQTTRLELQLLEHSLSTNKLEKQILDQTSEINKLQDKNSFLEKKVLAMEDKHIIQLQSIKEEKDQLQVLVSKQNSIIEELEKKIVTATVNNSVLQKQQHDLMETVNNLLTMMSTSNSAKDPTVAKEEQISFRDCAEVFKSGHTTNGIYTLTFPNSTEEIKAYCDMEAGGGGWTIIQRREDGSVDFQRTWKEYKVGFGNPSGEYWLGNEFVSQLTNQQRYVLKIHLKDWEGNEAYSLYEHFYLSSEELNYRIHLKGLTGTAGKISSISQPGNDFSTKDGDNDKCICKCSQMLTGGWWFDACGPSNLNGMYYPQRQNTNKFNGIKWYYWKGSGYSLKATTMMIRPADF

SEQ ID NO: 37 - DNA, 5416 bp, Homo sapiens
ctggttggagggcaggcattctgctctgatttttcctgttgcctggctagtgaccccctacaggaagataacggctaagccaggagggcggagcagcccactacacatgtctggctgctcttatcaacttatcatataaggaaaggaaagtgattgattcggatactgacactgtaggatctggggagagaggaacaaaggaccgtgaaagctgctctgtaaaagctgacacagccctcccaagtgagcaggactgttcttcccactgcaatctgacagtttactgcatgcctggagagaacacagcagtaaaaaccaggtttgctactggaaaaagaggaaagagaagactttcattgacggacccagccatggcagcgtagcagccctgcgttttagacggcagcagctcgggactctggacgtgtgtttgccctcaagtttgctaagctgctggtttattactgaagaaagaatgtggcagattgttttctttactctgagctgtgatcttgtcttggccgcagcctataacaactttcggaagagcatggacagcataggaaagaagcaatatcaggtccagcatgggtcctgcagctacactttcctcctgccagagatggacaactgccgctcttcctccagcccctacgtgtccaatgctgtgcagagggacgcgccgctcgaatacgatgactcggtgcagaggctgcaagtgctggagaacatcatggaaaacaacactcagtggctaatgaagcttgagaattatatccaggacaacatgaagaaagaaatggtagagatacagcagaatgcagtacagaaccagacggctgtgatgatagaaatagggacaaacctgttgaaccaaacagcggagcaaacgcggaagttaactgatgtggaagcccaagtattaaatcagaccacgagacttgaacttcagctcttggaacactccctctcgacaaacaaattggaaaaacagattttggaccagaccagtgaaataaacaaattgcaagataagaacagtttcctagaaaagaaggtgctagctatggaagacaagcacatcatccaactacagtcaataaaagaagagaaagatcagctacaggtgttagtatccaagcaaaattccatcattgaagaactagaaaaaaaaatagtgactgccacggtgaataattcagttcttcagaagcagcaacatgatctcatggagacagttaataacttactgactatgatgtccacatcaaactcagctaaggaccccactgttgctaaagaagaacaaatcagcttcagagactgtgctgaagtattcaaatcaggacacaccacgaatggcatctacacgttaacattccctaattctacagaagagatcaaggcctactgtgacatggaagctggaggaggcgggtggacaattattcagcgacgtgaggatggcagcgttgattttcagaggacttggaaagaatataaagtgggatttggtaacccttcaggagaatattggctgggaaatgagtttgtttcgcaactgactaatcagcaacgctatgtgcttaaaatacaccttaaagactgggaagggaatgaggcttactcattgtatgaacatttctatctctcaagtgaagaactcaattataggattcaccttaaaggacttacagggacagccggcaaaataagcagcatcagccaaccaggaaatgattttagcacaaaggatggagacaacgacaaatgtatttgcaaatgttcacaaatgctaacaggaggctggtggtttgatgcatgtggtccttccaacttgaacggaatgtactatccacagaggcagaacacaaataagttcaacggcattaaatggtactactggaaaggctcaggctattcgctcaaggccacaaccatgatgatccgaccagcagatttctaaacatcccagtccacctgaggaactgtctcgaactattttcaaagacttaagcccagtgcactgaaagtcacggctgcgcactgtgtcctcttccaccacagagggcgtgtgctcggtgctgacgggacccacatgctccagattagagcctgtaaactttatcacttaaacttgcatcacttaacggaccaaagcaagaccctaaacatccataattgtgattagacagaacacctatgcaaagatgaacccgaggctgagaatcagactgacagtttacagacgctgctgtcacaaccaagaatgttatgtgcaagtttatcagtaaataactggaaaacagaacacttatgttatacaatacagatcatcttggaactgcattcttctgagcactgtttatacactgtgtaaatacccatatgtcctgaattcaccatcactatcacaattaaaaggaagaaaaaaactctctaagccataaaaagacatattcagggatattctgagaaggggttactagaagtttaatatttggaaaaacagttagtgcatttttactccatctcttaggtgctttaaatttttatttcaaaaacagcgtatttacatttatgttgacagcttagttataagttaatgctcaaatacgtatttcaaatttatatggtagaaacttccagaatctctgaaattatcaacagaaacgtgccattttagtttatatgcagaccgtactatttttttctgcctgattgttaaatatgaaggtatttttagtaattaaatataacttattaggggatatgcctatgtttaacttttatgataatatttacaattttataatttgtttccaaaagacctaattgtgccttgtgataaggaaacttcttacttttaatgatgaggaaaattatacatttcattctatgacaaagaaactttactatcttctcactattctaaaacagaggtctgttttctttcctagtaagatatatttttatagaactagactacaatttaatttctggttgagaaaagccttctatttaagaaatttacaaagctatatgtctcaagattcacccttaaatttacttaaggaaaaaaataattgacactagtaagtttttttatgtcaatcagcaaactgaaaaaaaaaaaagggtttcaaagtgcaaaaacaaaatctgatgttcataatatatttaaatatttaccaaaaatttgagaacacagggctgggcgcagtggctcacacctataatcccagtacattggtaggcaaggtgggcagatcacctgaggtcaggagttcaagaccagcctggacaacatggtgaaaccctgtctctactaaataatacaaaaattagccaggcgtgctggcgggcacctgtaatcccagctactcgggaggctgaggcagggagaattgcttgcaccagggaggtagaggttgcagtgagccaagatcgcaccactgcactccagccggggcaacagagcaagactccatctcaaaaaaaaaaaaaaaaaaagaaagaaaagaaaatttgagaacacagctttatactcgggactacaaaaccataaactcctggagttttaactccttttgaaattttcatagtacaattaatactaatgaacatttgtgtaaagctttataatttaaaggcaatttctcatatattcttttctgaatcatttgcaaggaagttcagagtccagtctgtaactagcatctactatatgtctgtcttcaccttacagtgttctaccattattttttctttattccatttcaaaatctaatttattttaccccaacttctccccaccacttgacgtagttttagaacacacaggtgttgctacatatttggagtcaatgatggactctggcaaagtcaaggctctgttttatttccaccaaggtgcacttttccaacaactatttaactagttaagaacctccctatcttagaactgtatctactttatatttaagaaggttttatgaattcaacaacggtatcatggccttgtatcaagttgaaaaacaactgaaaataagaaaatttcacagcctcgaaagacaacaacaagtttctaggatatctcaatgacaagagtgatggatacttaggtagggaaacgctaatgcaggaaaaactggcaacaacacaatttatatcaattctctttgtaggcaggtgataaaaaattcaaggacaaatctcattatgtcattgtgcatcatatataatctcttatgagcgagaatggggggaatttgtgtttttactttacacttcaattccttacacggtatttcaaacaaacagttttgctgagaggagcttttgtctctccttaagaaaatgtttataaagctgaaaggaaatcaaacagtaatcttaaaaatgaaaacaaaacaacccaacaacctagataactacagtgatcagggagcacagttcaactccttgttatgttttagtcatatggcctactcaaacagctaaataacaacaccagtggcagataaaaatcaccatttatctttcagctattaatcttttgaatgaataaactgtgacaaacaaattaacatttttgaacatgaaaggcaacttctgcacaatcctgtatccaagcaaactttaaattatccacttaattattacttaatcttaaaaaaaattagaacccagaacttttcaatgaagcatttgaaagttgaagtggaatttaggaaagccataaaaatataaatactgttatcacagcaccagcaagccataatctttatacctatcagttctatttctattaacagtaaaaacattaagcaagatataagactacctgcccaagaattcagtcttttttcatttttgtttttctcagttctgaggatgttaatcgtcaaattttctttggactgcattcctcactactttttgcacaatggtctcacgttctcacatttgttctcgcgaataaattgataaaaggtgttaagttctgtgaatgtctttttaattatgggcataattgtgcttgactggataaaaacttaagtccacccttatgtttataataatttcttgagaacagcaaactgcatttaccatcgtaaaacaacatctgacttacgggagctgcagggaagtggtgagacagttcgaacggctcctcagaaatccagtgacccaattctaaagaccatagcacctgcaagtgacacaacaagcagatttattatacatttattagccttagcaggcaataaaccaagaatcactttgaagacacagcaaaaagtgatacactccgcagatctgaaatagatgtgttctcagacaacaaagtcccttcagaatcttcatgttgcataaatgttatgaatattaataaaaagttgattgagaaaaa

SEQ ID NO: 38 - protein, 548 aa, Homo sapiens
MDELFPLIFPAEPAQASGPYVEIIEQPKQRGMRFRYKCEGRSAGSIPGERSTDTTKTHPTIKINGYTGPGTVRISLVTKDPPHRPHPHELVGKDCRDGFYEAELCPDRCIHSFQNLGIQCVKKRDLEQAISQRIQTNNNPFQEEQRGDYDLNAVRLCFQVTVRDPSGRPLRLPPVLSHPIFDNRAPNTAELKICRVNRNSGSCLGGDEIFLLCDKVQKEDIEVYFTGPGWEARGSFSQADVHRQVAIVFRTPPYADPSLQAPVRVSMQLRRPSDRELSEPMEFQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 39 - DNA, 2681 bp, Homo sapiens
cgtcctcggcgaggcgcgcacttggccccgacccccggcagcggctgtgcgtgcagcctcttcgtcctccgcgcggcgtgcacttgctcccggcccctgcgccgggcggcggcggggcagcgcgcaggcgcggccggattccgggcagtgacgcgacggcgggccgcgcggcgcatttccgcctctggcgaatggctcgtctgtagtgcacgccgcgggcccagctgcgaccccggccccgcccccgggaccccggccatggacgaactgttccccctcatcttcccggcagagccagcccaggcctctggcccctatgtggagatcattgagcagcccaagcagcggggcatgcgcttccgctacaagtgcgaggggcgctccgcgggcagcatcccaggcgagaggagcacagataccaccaagacccaccccaccatcaagatcaatggctacacaggaccagggacagtgcgcatctccctggtcaccaaggaccctcctcaccggcctcacccccacgagcttgtaggaaaggactgccgggatggcttctatgaggctgagctctgcccggaccgctgcatccacagtttccagaacctgggaatccagtgtgtgaagaagcgggacctggagcaggctatcagtcagcgcatccagaccaacaacaaccccttccaagaagagcagcgtggggactacgacctgaatgctgtgcggctctgcttccaggtgacagtgcgggacccatcaggcaggcccctccgcctgccgcctgtcctttctcatcccatctttgacaatcgtgcccccaacactgccgagctcaagatctgccgagtgaaccgaaactctggcagctgcctcggtggggatgagatcttcctactgtgtgacaaggtgcagaaagaggacattgaggtgtatttcacgggaccaggctgggaggcccgaggctccttttcgcaagctgatgtgcaccgacaagtggccattgtgttccggacccctccctacgcagaccccagcctgcaggctcctgtgcgtgtctccatgcagctgcggcggccttccgaccgggagctcagtgagcccatggaattccagtacctgccagatacagacgatcgtcaccggattgaggagaaacgtaaaaggacatatgagaccttcaagagcatcatgaagaagagtcctttcagcggacccaccgacccccggcctccacctcgacgcattgctgtgccttcccgcagctcagcttctgtccccaagccagcaccccagccctatccctttacgtcatccctgagcaccatcaactatgatgagtttcccaccatggtgtttccttctgggcagatcagccaggcctcggccttggccccggcccctccccaagtcctgccccaggctccagcccctgcccctgctccagccatggtatcagctctggcccaggccccagcccctgtcccagtcctagccccaggccctcctcaggctgtggccccacctgcccccaagcccacccaggctggggaaggaacgctgtcagaggccctgctgcagctgcagtttgatgatgaagacctgggggccttgcttggcaacagcacagacccagctgtgttcacagacctggcatccgtcgacaactccgagtttcagcagctgctgaaccagggcatacctgtggccccccacacaactgagcccatgctgatggagtaccctgaggctataactcgcctagtgacaggggcccagaggccccccgacccagctcctgctccactgggggccccggggctccccaatggcctcctttcaggagatgaagacttctcctccattgcggacatggacttctcagccctgctgagtcagatcagctcctaagggggtgacgcctgccctccccagagcactgggttgcaggggattgaagccctccaaaagcacttacggattctggtggggtgtgttccaactgcccccaactttgtggatgtcttccttggaggggggagccatattttattcttttattgtcagtatctgtatctctctctctttttggaggtgcttaagcagaagcattaacttctctggaaaggggggagctggggaaactcaaacttttcccctgtcctgatggtcagctcccttctctgtagggaactctggggtcccccatccccatcctccagcttctggtactctcctagagacagaagcaggctggaggtaaggcctttgagcccacaaagccttatcaagtgtcttccatcatggattcattacagcttaatcaaaataacgccccagataccagcccctgtatggcactggcattgtccctgtgcctaacaccagcgtttgaggggctggccttcctgccctacagaggtctctgccggctctttccttgctcaaccatggctgaaggaaaccagtgcaacagcactggctctctccaggatccagaaggggtttggtctgggacttccttgctctccctcttctcaagtgccttaatagtagggtaagttgttaagagtgggggagagcaggctggcagctctccagtcaggaggcatagtttttactgaacaatcaaagcacttggactcttgctctttctactctgaactaataaatctgttgccaagctg

SEQ ID NO: 40 - DNA, 500 bp, Homo sapiens; identical to SEQ ID NO: 3
  misc_feature (251)..(251): v=SNP position

(19) United States
(12) Patent Application Publication
(10) Pub. No.: US 2024/0288448 A1
(43) Pub. Date: Aug. 29, 2024

(54) Cardiovascular Disease

(71) Applicant: Sanofi, PARIS (FR)

(72) Inventors: Karla Allebrandt, Frankfurt am Main (DE); Francesca Frau, Frankfurt am Main (DE)

(21) Appl. No.: 18/280,426

(22) PCT Filed: Mar. 8, 2022
(86) PCT No.: PCT/EP2022/055925

§ 371 Date: Sep. 5, 2023

(30) Foreign Application Priority Data
Mar. 8, 2021 (EP) 21315031.1

(51) Int. Cl. G01N 33/68; C12Q 1/6827

(52) U.S. Cl. CPC G01N 33/6893; C12Q 1/6827; G01N 2333/4745; G01N 2333/521; G01N 2333/525; G01N 2333/5412; G01N 2333/70567; G01N 2333/7151; G01N 2333/775; G01N 2333/91177; G01N 2800/2871; G01N 2800/324; G01N 2800/325; G01N 2800/50; G01N 2800/52

(57) ABSTRACT

The invention relates to cardiovascular disease, and particularly, although not exclusively, to cardiovascular disease biomarkers and their use in methods of diagnosis and prognosis. The invention also extends to diagnostic and prognostic kits utilising the biomarkers of the invention for diagnosing or prognosing cardiovascular disease.

The present invention relates to cardiovascular disease, and particularly, although not exclusively, to cardiovascular disease biomarkers and their use in methods of diagnosis and prognosis. The invention also extends to diagnostic and prognostic kits utilising the biomarkers of the invention for diagnosing or prognosing cardiovascular disease.

Due to the complexity of cardiovascular disease (CVD), the ability of single biomarkers to predict cardiovascular outcome (CVO) risk is limited (De Lemos et al 2017), though many genetic loci and protein biomarkers (BMKs) have been discovered. Combining genetic information with other types of omics data should have more predictive power to detect sub-groups of the population and the correspondent molecular signatures related to the clinical outcomes (Vilne et al., 2018). This should point to molecular signatures for CV risk and show their potential to stratify the population in relation to disease progression. A better understanding of the drivers of T2D-associated cardiovascular disease will help to define sub-populations that may differ with regard to disease progression.

The inventors developed and tested a workflow to identify substructure of a trial population by molecular signatures of protein and genetic biomarkers. They discovered signatures defining sub-populations that progressed differently towards CVD, suggesting that these signatures reflect different stages of disease progression. They identified combinations of biomarkers, reflecting differences in CVD progression, will lead to strategies to optimize clinical trials plans, drug efficacy, and so to optimize treatment.

Accordingly, in a first aspect of the invention, there is provided a method of determining, diagnosing and/or prognosing an individual's risk of suffering from cardiovascular disease, the method comprising;
a. detecting, in a sample obtained from an individual, the expression level, amount and/or activity of two or more biomarkers selected from a group consisting of: Tumour Necrosis Factor (TNF)-□□ Glutathione S-Transferase Alpha 1 (GSTA1); N-terminal-pro hormone BNP (NT-proBNP); Retinoic Acid Receptor-Related Orphan Receptor Alpha (RORA); Tenascin C (TNC); Growth Hormone Receptor (GHR); Alpha-2-Macroglobulin (A2M); Insulin Like Growth Factor Binding Protein 2 (IGFBP2); Apolipoprotein B (APOB); Selenoprotein P (SEPP1); Trefoil Factor (TFF3); Interleukin 6 (IL6); Chitinase 3 Like 1 (CHI3L1); Hepatocyte Growth Factor Receptor (MET); Growth Differentiation Factor 15 (GDF15); Chemokine (C-C Motif) Ligand 22 (CCL22); Tumour Necrosis Factor Receptor Superfamily, Member 11 (TNFRSF11); Angiopoietin 2 (ANGPT2); and v-Rel Avian Reticuloendotheliosis Viral Oncogene Homolog A Nuclear Factor-kappa B (ReLA NF-KB);
b. comparing the expression level, amount and/or activity of the biomarker with a reference from a healthy control population; and
c. determining, diagnosing an
d/or prognosing the risk of an individual suffering from cardiovascular disease if the expression level, amount, and/or activity of the biomarker deviates from the reference from a healthy control population.

Advantageously, the method of the invention enables the identification of individuals who are at risk from suffering from a CVD event, and thus enables early intervention to prevent, or reduce the risk of, the individual from suffering from a CVD event. In particular, detection of each biomarker in isolation enables the identification of individuals who are at risk from suffering from a CVD event, and detection of multiple biomarkers, i.e. the biomarker signature provides a particularly effective means of enabling early intervention to prevent, or reduce the risk of, the individual from suffering from a CVD event.

The skilled person would understand that the term prognosis may relate to predicting the rate of progression or improvement and/or the duration of cardiovascular disease with individual suffering from cardiovascular disease.

The method may be performed in vivo, in vitro or ex vivo. Preferably, the method is performed in vitro or ex vivo. Most preferably, the method is performed in vitro.

The expression level may relate to the level or concentration of a biomarker polynucleotide sequence. The polynucleotide sequence may be DNA or RNA.

The DNA may be genomic DNA. The RNA may be mRNA.

The amount of biomarker may relate to the concentration of biomarker polypeptide sequence.

The biomarker activity may relate to the activity of the biomarker protein, preferably activity in relation to interacting biomarkers.

Categorical Biomarker(s):

LLQQ=2→inactivation (−1)

LLQQ=from 3 to 4→no activation (0)

LLQQ=from 5 to higher levels→activation (+1)

For the quantitative biomarker(s) we used the tertiles of the distribution.

Accordingly, the expression level, amount, and/or activity of the biomarker may be detected by: sequencing methods (e.g., Sanger, Next Generation Sequencing, RNA-SEQ), hybridization-based methods, including those employed in biochip arrays, mass spectrometry (e.g., laser desorption/ionization mass spectrometry), fluorescence (e.g., sandwich immunoassay), surface plasmon resonance, ellipsometry and atomic force microscopy. Expression levels of markers (e.g., polynucleotides, polypeptides, or other analytes) may be compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SFMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero. Thus, the method of detection may relate to a probe that is capable of hybridizing to the biomarker DNA or RNA sequence. The term “probe” may defined to be an oligonucleotide. A probe can be single stranded at the time of hybridization to a target. Probes include but are not limited to primers, i.e., oligonucleotides that can be used to prime a reaction, for example at least in a PCR reaction.

Preferably, a decrease in expression, amount and/or activity of TNF-1, GSTA1, NT-proBNP, RORA and/or TNC, when compared to the reference, is indicative of an individual having a higher risk of suffering from cardiovascular disease or a negative prognosis.

Preferably, an increase in expression, amount and/or activity of GHR, A2M, IGFBP2, APOB, SEPP1, TFF3, IL6 and/or CHI3L1, when compared to the reference, is indicative of an individual having a higher risk of suffering from cardiovascular disease or a negative prognosis.

Preferably, a decrease in expression, amount and/or activity of MET, GDF15, CCL22, TNFRSF11, ANGPT2 and/or ReIA NF-KB, when compared to the reference, is indicative of an individual having a lower risk of suffering from cardiovascular disease or a positive prognosis.

Preferably, step a) comprises detecting, in a sample obtained from the individual, the expression levels, amount and/or activities of at least 3 of the biomarkers selected from the group consisting of: TNF-1; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2; and ReIA NF-KB. However, step a) may comprise detecting expression levels, amount and/or activities of at least 4 biomarkers or at least 5 biomarkers.

Step a) may comprise detecting expression levels, amount and/or activities of at least 6 biomarkers or at least 7 biomarkers. Alternatively, step a) may comprise detecting expression levels, amount and/or activities of at least 8 biomarkers or at least 9 biomarkers. In another embodiment, step a) may comprise detecting expression levels, amount and/or activities of at least 10 biomarkers, at least 11 biomarkers, at least 12 biomarkers, at least 13 biomarkers, at least 14 biomarkers or at least 15 biomarkers. In another embodiment, step a) may comprise detecting expression levels, amount and/or activities of at least 16 biomarkers, at least 17 biomarkers or at least 18 biomarkers.

Preferably, step a) comprises detecting, in a sample obtained from the individual, the expression levels, amount and/or activities of the biomarkers: TNF-□; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB.

The cardiovascular disease may be selected from the group consisting of: cardiovascular death; myocardial infarction; stroke; and heart failure.

In one embodiment, RORA is provided by gene bank locus ID: HGNC: 10258; Entrez Gene: 6095; and/or Ensembl: ENSG00000069667. The protein sequence may be represented by the GeneBank ID P35398-2, which is provided herein as SEQ ID No: 1, as follows:

[SEQ ID No: 1]
MESAPAAPDPAASEPGSSGADAAAGSRETPLNQESARKSEPPAPVRRQS
YSSTSRGISVTKKTHTSQIEIIPCKICGDKSSGIHYGVITCEGCKGFFR
RSQQSNATYSCPRQKNCLIDRTSRNRCQHCRLQKCLAVGMSRDAVKFGR
MSKKQRDSLYAEVQKHRMQQQQRDHQQQPGEAEPLTPTYNISANGLTEL
HDDLSNYIDGHTPEGSKADSAVSSFYLDIQPSPDQSGLDINGIKPEPIC
DYTPASGFFPYCSFTNGETSPTVSMAELEHLAQNISKSHLETCQYLREE
LQQITWQTFLQEEIENYQNKQREVMWQLCAIKITEAIQYVVEFAKRIDG
FMELCQNDQIVLLKAGSLEVVFIRMCRAFDSQNNTVYFDGKYASPDVFK
SLGCEDFISFVFEFGKSLCSMHLTEDEIALFSAFVLMSADRSWLQEKVK
IEKLQQKIQLALQHVLQKNHREDGILTKLICKVSTLRALCGRHTEKLMA
FKAIYPDIVRLHFPPLYKELFTSEFEPAMQIDG

Accordingly, preferably RORA comprises or consists of an amino acid sequence substantially as set out in SEQ ID NO: 1, or a fragment or variant thereof.

In one embodiment, RORA is encoded by a nucleotide sequence which is provided herein as SEQ ID No: 2, as follows:

[SEQ ID No: 2]
GTACCATAGAGTTGCTCTGAAAACAGAAGATAGAGGGAGTCTCGGAGCTC
GCCATCTCCAGCGATCTCTACATTGGGAAAAAACATGGAGTCAGCTCCGG
CAGCCCCCGACCCCGCCGCCAGCGAGCCAGGCAGCAGCGGCGCGGACG
CGGCCGCCGGCTCCAGGGAGACCCCGCTGAACCAGGAATCCGCCCGCAA
GAGCGAGCCGCCTGCCCCGGTGCGCAGACAGAGCTATTCCAGCACCAGC
AGAGGTATCTCAGTAACGAAGAAGACACATACATCTCAAATTGAAATTATTC
CATGCAAGATCTGTGGAGACAAATCATCAGGAATCCATTATGGTGTCATTA
CATGTGAAGGCTGCAAGGGCTTTTTCAGGAGAAGTCAGCAAAGCAATGCC
ACCTACTCCTGTCCTCGTCAGAAGAACTGTTTGATTGATCGAACCAGTAGA
AACCGCTGCCAACACTGTCGATTACAGAAATGCCTTGCCGTAGGGATGTC
TCGAGATGCTGTAAAATTTGGCCGAATGTCAAAAAAGCAGAGAGACAGCTT
GTATGCAGAAGTACAGAAACACCGGATGCAGCAGCAGCAGCGCGACCAC
CAGCAGCAGCCTGGAGAGGCTGAGCCGCTGACGCCCACCTACAACATCT
CGGCCAACGGGCTGACGGAACTTCACGACGACCTCAGTAACTACATTGAC
GGGCACACCCCTGAGGGGAGTAAGGCAGACTCCGCCGTCAGCAGCTTCT
ACCTGGACATACAGCCTTCCCCAGACCAGTCAGGTCTTGATATCAATGGAA
TCAAACCAGAACCAATATGTGACTACACACCAGCATCAGGCTTCTTTCCCT
ACTGTTCGTTCACCAACGGCGAGACTTCCCCAACTGTGTCCATGGCAGAA
TTAGAACACCTTGCACAGAATATATCTAAATCGCATCTGGAAACCTGCCAA
TACTTGAGAGAAGAGCTCCAGCAGATAACGTGGCAGACCTTTTTACAGGAA
GAAATTGAGAACTATCAAAACAAGCAGCGGGAGGTGATGTGGCAATTGTG
TGCCATCAAAATTACAGAAGCTATACAGTATGTGGTGGAGTTTGCCAAACG
CATTGATGGATTTATGGAACTGTGTCAAAATGATCAAATTGTGCTTCTAAAA
GCAGGTTCTCTAGAGGTGGTGTTTATCAGAATGTGCCGTGCCTTTGACTCT
CAGAACAACACCGTGTACTTTGATGGGAAGTATGCCAGCCCCGACGTCTT
CAAATCCTTAGGTTGTGAAGACTTTATTAGCTTTGTGTTTGAATTTGGAAAG
AGTTTATGTTCTATGCACCTGACTGAAGATGAAATTGCATTATTTTCTGCAT
TTGTACTGATGTCAGCAGATCGCTCATGGCTGCAAGAAAAGGTAAAAATTG
AAAAACTGCAACAGAAAATTCAGCTAGCTCTTCAACACGTCCTACAGAAGA
ATCACCGAGAAGATGGAATACTAACAAAGTTAATATGCAAGGTGTCTACCT
TAAGAGCCTTATGTGGACGACATACAGAAAAGCTAATGGCATTTAAAGCAA
TATACCCAGACATTGTGCGACTTCATTTTCCTCCATTATACAAGGAGTTGTT
CACTTCAGAATTTGAGCCAGCAATGCAAATTGATGGGTAAATGTTATCACC
TAAGCACTTCTAGAATGTCTGAAGTACAAACATGAAAAACAAACAAAAAAAT
TAACCGAGACACTTTATATGGCCCTGCACAGACCTGGAGCGCCACACACT
GCACATCTTTTGGTGATCGGGGTCAGGCAAAGGAGGGGAAACAATGAAAA
CAAATAAAAGTTGAACTTGTTTTTCTCATGCATATGATTTCCATTATGCCTAC
AGATATGGACCCTTTTTCTGTCTTGACTTCTTGATCATTGACCTCTGTTTAC
AACAGGAGGAGGGTACTAAAGTCGGAGGATTTCCTTTTCTTGTAGCTCACT
GCCCACAGACTTTCTACAGAGTCACCAATCTGTCAGTAACAACAGAGAGTC
CAGCAATAATCGGTGACTGGTGTGCATAGCGGAGGTTGCGGCATTACTTT
GCACAACTAGCTCTTTGTTTCATGAAGGAAGTTTTTATTTTTTCACCGATTA
TTGCCAGTCCGCAGGATGGCATGAAAAGGGTCCATAGCAGTAGCAACAAT
AGCATTATAATATATTACAGGGTAAATGGGCATGAAGACTATATATAGCTAA
AAGAGATATTGTTTATATATTGTTTTAAGTAATATAAAATGTAGTTACTGGTG
TAGCTTTTCCTGTTGAATTGATAAGGCACTTTCATTTTGCACCTTTTTCTTTA
AATTAAATGCTAGCGTGTTCACTGTCGTGTCGCATGTGCACCAGAAACACA
AGTTTAACTGAGAAGGCTTGGAAGGTACGTTGGGAGGTATTTATGCTGCTG
TTTACAAAATTATTTTTAAGAGACTGGCTGGTCATATCTAGAAATCACCACG
TTGGATTTTTTTTTTAACATGTGAATTTGGAATTAGAAACGGAACTCTCCCT
AAATTATACTTTGCTTTTTGGTAAGTTTAATGATAGATGTGTTTATGCTTCAT
ACAAAGTTGAATGATTGATTGGCGTGGTGGACATATACCATCATGCTCATT
TTTTTTTTTTAAAGCTTTTTAAAATGCCACCTCATGGAGGCGAGGGGGAGG
AGAAGCTCATTTTACACAATTCAGTAGTTAAATATGGACTCGGTCTCAACTT
GGAATTCTTATGCTTTGAGAACAAATCAACAACCAGAATATTTATTGGAATC
TAGCTTTTATTATAAGAAGGACCCAAAGATTATATCCTGAGCAAATGCACAC
TCCCCATGTGAGGACATGAAGTATTTACTTTGTGAATGTTTATGTTCTTGGT
ATAATCTAGGAACCCTATGAGTTTATCTCAGAGTGAACTAATTCTAGATTTG
TTGTCAATAGATGCTATAATTCAAGAATGTTGCTCTCCATATTTGAAAAACG
ATGGATAGGAGGGTGAGGGAAGCATACAATGTTGAACCAGTTTCTCTTATT
TAAATATTAAATACTTTAAGCCTTTAAAGTGAAGTTGATGCTAGCTGCAAAC
ATTTACTCTTGTATTTATCTTCACTAGGAAACTGTGGACTGTAATTTATTTTA
TTAAATATTTAGAAGATTATTTGGCTTGTGTGTTCAGGTGAGAAATACTGAG
TTGTTTTTGTTTAATTTCATGGTTTTTTTTTTTTTAATGATCCCTAGTGGGGG
AAGGGGAAAGGAATAGTCTGATAAACAGATGTGCATATTTTAAAAACAAGT
GACCTTTTGGGAATGTAGGCATTTAGACGATGATTTTAGTCGCACTAGGGG
TGGGATTCAAACTACTGGTCAAAGACCATTTTGTACAGAAAAGGGAACATC
TCCGATGGGTGTTAAGGTAGGAGTTTCCATGCAGCCCTTTATGTCTGAGAA
ATAGTCTCCTCTGCCATTGGGGTCCCTGCGGAATCTTCTACAGGAATTGCA
GCTCTTCACGTCATGCTAGGTTACCAGCATGGGCTTCCCAGAGCACTTCA
CTCTGTTTCTAACTCCACTGACTTTTCTGACTTGTTTCTTGAGGGACTTGGA
AAAGGGGAAGGTATTATTCACACAGATGTGTGTATGAAGCCTAAATAACAT
CCAACTTTTTTTCCAAAAACATAGTAAGAGTTTAACCATAAATAGAAGAGTA
ATATTCCTTTAAATTTCTTACAGGCATGTAAAATTTTATGTGTTTATAGAGAG
ATGCTAACTGTCAGCATATAGTATTTATATTGGGGCAAGAAGGGTTAAATC
AAATCTTTAATTTAAGTAAGCATAGTTCCTTTAAAGATCAGTAGTATTTATAC
TCTGAAAAGAGTACCAAGCTTAGTTCAGTTATTTATTCATCCATTTTTTTTCT
ATTGTTTCTCCTCTGGGGGGAAATGGTGTTTTGTTTTTGTTTGAGTTTTGTG
TTTTAGTTTTTTGGTTTTGGTTTTGTTTTGTTTTTTTGTCATTCAGATTCACTA
AATTTGGGGATGGTTTTATTAAAGTATATTAACTTCTTTTTAACCAAAGCTTT
CTGAATATGACCAGCCTCAGGTGCTAGCGCATTAAAGAGCAACTAAACCTA
ATTCCAGTGTCGATTTGTGAAATAATAAAAGCTAATTGAATTTCTCTAAGGG
TGAGAGAGAACATAATGATGAACTTAAAAAGAAATTCCTTTTCCCAGAAGG
GATTCTGCATGTACCTACAAACAATCATGCTCTAACCACAGTAATAGTCATG
CCATGGTGACATTGCTTTTACGGTAAACCAAGATAGGTAATATGATGCTTC
TTCCCAGGTGTTTCTGAAAGAAAAGCAGCGGTGGAGCTTAAGAGCCAAGT
CCACTGATTGACATAATAGGATGGAATTGTAGACAGAGACATGCTCCATGA
AACAAGGAAACAACTGACTACTATTTGGATCTAAGTTGGGATCTGATGTTA
AACAATAAATTCAGTTTAAAAAAAATATGGAGCTCAGAAAAGGATGTGAAAA
ACTTTGCATTTTCCTTTCTTCATTATTACAAAAACACCTATGTGATGACATAA
AATACTTGGGTGATATCAGAACAATTATCCTTAATAGTTTTTATAATTAAAGT
TTCCTAAACTTCAGTCTCCAATAGTCTTTTAAGGATTGGAACCACATCACTG
TCAGCCCCGCTGCTACAATGCCTTTGTACAATTTTTTTACATAAGGCAAATA
AGGCTTTTGTACAAAGCCTGAATACCTTCTGTTCCAATGGTGTCCAAATGG
TTATTATATTGTGAAAAACCTGGCCTTGGTCACAATGCAAACAAAAGTACAG
ATGAAAGTGCTTTTTGGACAGTTTGCAAATTGTGTTAAAGCTACGGATTTTT
TTTAAAGTGTTCAGCATCTTAACGTGTATTAAAGCTATGGATTTTTTTTTAAA
GTCTTCAGCATCCTAATTCACCCTTTCTAACTTAAGAAAAACATGACTTAAG
ACACTGCTTCTAAGTTTGGTTGTTCTTTATAGTGTGGATACCCAGTTATCTG
TGAGCGTATGGGGGTGGGCTGAGGGTCAGGTGAGGAAGGAGTGTGTGTG
TGTGTGTGTGTGTGTGTGTGTGTGTGTGTGATTTGCATGTGTATGATGTGT
GTGCGTCGGACCGCTTCTAGGCTACTAAGTGTCAATGGAAAAGAAAATGTA
TTCAAAATACTTAAATCAAAACTAGAAGATGGGGAAAAAAAGATTTATTCTA
TACAAAGCCTTGTCTGGACCACTTTAGAGAGACTTCTATTTTTTTAACCCTT
CTATAAATATTTGATGGCACTTGAAATATTCCTGCAATAAAATGTGATTTGT
GTAAAGAAAAAAAGATTTTGTAATGTGAAACAAAGAAAGAAAGTAATGTAAT
TTTCTAAAAAAAAAAATACAAACAAACAAACTTTGTATTATTTTCTTGATGGA
ATTTGTCTATCTGTCTTTGGAAAACTTTTTATTTCATTGAATGTGCCATAGTA
GAAATGTGTGTTTTTAGTTTTAGACTAAGGAATAGCTAGTTGTTGTGTTCCG
ACATTCCAAAATGCAAAACAACCTAGTAGTATCTTTCATGAAAAGGTTTAAG
TAGTATGTAAGCTTCTCTCATTGTTGCTTTTTTGCACATGTTCTTCATTCCTC
TCTAGTGCAATATGTACATAGAGCACTTGCGGGTGTACCTTGATCCCTCAG
GGAAAAATACATATTTGTACAGTTTTTTGGGGTTTTTTTGTTTTTTGGTTTTT
TTTGTTTTGCCTTTTGTTTTTGGCTAAGGAATGTCGATCGAATCACTTGTTA
TTGTTGAGGGGCAGCCAGATAATAATCCTAAAGCCACTGTTTCCAACATTG
ATTGTTTAAATCATATGTCCTTCCAATGCTATTATTTTAAGATAATAATAAAA
AGTTATTTTCTGACAGTTCTTTGTGCTGACTGGTGAAAAACAAGGGTAAATA
AGCACCTTATAATTGACTTACTGTGAATGACAATCCATCTTGGTATCAACGA
TAGAAGCCCTATCATTTTGGAGTTGGGGTTAAGAGTCAGAAACAATGTGCT
CAGGGATCTCCTAAAACTCTTAAAACAGGGTGGCCAGTACTACTGGGACA
AATTGTGTTTTTTATTATTAATAATAATGATAATAATACTATCCCTCCAAGGC
ACAAGTGAACTATATAGAACTGCGTGTGTGTAAACTCTTTACTCTCGTCTCA
TTTTGTTGAGTTTAGAACTTGATGTGCTCGTCAGTCTTGTGTTTCAAAACAC
TGAATAACTTCCAAAGCAAAGTTATGCCAGTGTGTTCAAAAGATAAATTAAT
AATGTACCAGCAAAGAGCATCATTCAAAGTATAGTCCTTGCATATTCCAGTT
ACCATTTCTCTAATAATTAAAATATTCATGATAAATATATATATAGCATCATA
TGTTAAAAACTATTTCAAATTCTACATATTAAGGATGAAAATTTTAAAATCCA
GTAATAAGAGGAGAGACCTGCCTATCAGTACAGTGATATAGGTAAAAGATG
AAATATGTTTTTAAAATACCAGCAATTAACTATTGTTTTCATGGGTTCTCCTC
TAGAAGCAAACCAAAAATTCCTTCATGGAAAACAATTCTTACTTCTACATGT
GTAGTTTATATCTGATGCATTACAAGAGCTAATGTTTAAAGACAAAACAAAA
CCTGCCTGTATGACAGCAGCAACTCGAGCCAACATTTAGTGTTACATGTTA
TATTTTTGAAATACCGTTTTGTTATCATATTCCACATATTACTTTCCATAAAG
TCAGAGAAGTTCAATGTAATTGTTGGCTCTGATTCTTCCACCTTGGGATAC
ACATTCACAAGAAGATTTATATATTTTCTTACTATGATAGAGGAACATAATCT
GGGAAAACTTCCCATGTCTGTAAGATGAAAAGGATACCTTTACCATGTTGT
TTTTGATAATAAAGAATATGGAAAATGGTAGAAATCTCTCCTCTCTATGTGT
ATTTGTATATGTGTGTATACACACACACGTTGACATTTTTACATAACATGTG
ATTGCCACTTCTTATAAAGTTATGATATAGAACCCCATGAAAACAACTTTAT
ATTATAGATCAAATAATGTGCCCAGAAACGCAATTGCAACAGTAAATCTTGA
TCTATTGGTAAGAGTCTATGATACAGGTCTTCATTCTATCCCTAAACATAAT
GTTAGTAAAGAGGTTCCATCAGATTGTATTATAGAGACCTTCCTATTGCTAT
TTATTTTTAAGAGATGAGAAGACTGACTAGCAATGTCTCCACAGTGCAATT
GGTTTCACTTCTGGGTCTGTCTGTCTGTTTGTGTAGGTGAGATCAGTGTTG
TCAGGCTCTCAGAATAATTTTTAAAAGAATCCAGAAGCCTGACTTCACACC
AAGTAGCCATCCTAGATGGGCGGGGGGGATCTCCATGTTCAACCAAACCC
TCAGAACTTAGAGCAATAAATACATTCAGTCATTTATTTATAAATGAATGAC
AGAATATTCAATCCAAATAGAAACAACCTTTTGTCAGTGATGCAACATAACA
TGGAGTCTTATATTGAGAAAGTGAATATGAATATTTTAAATAGATGCCTAGG
AAATCTGTGTTTGCTGTTTACATTTAATGTACTTTGCCACATTAGCAGTACC
ACACTTCTTTTACTTTCATCCTTCTAAGAACTAATGAAAGATAGCTTGCTATT
AGCTTTGACATGTTGCACATGCCATTATGTTGTTCTCTAAGAACAACTAAAT
TCTCTCCAGTGTTCATGTGTGATTCCTTTTTCATCATTATTACTTTAATGTGG
ATGAATACTATTTCTAGGAACTTGATTATCACTGAACAGATTGCATATGTAA
ATGCAGATAATTCTTGAGCAATATAGTGAAAATGATTTCACAAAAAAAATCC
ATATGTACTGTATACATCTTCCTGAATCCAAAATTCTCCTGTTATCCACACA
GTTTGAAATCTCATATTAAACAGTGGTAAATTTTTAAACATTAGGACATTAG
CTGGCTGCTTCTATAAATGTACCTGTATTGTCTGCCTGCTCCTTTTGTGGA
GATGTGTTTTTGTATTGGATGTTTGGGCCAATGGGAGGCTTCAAGCTACAA
CATATTTTCCCACTTTAAATAATATTTAACATATGACAAACCCCAGACAAGG
CTTTTAATATGTATAAAGAACTGCAGTGTTAGGTGGTTTATTTGCAAACTGT
TTTCAGTGTTGTCCATTTTTATGGCACCTTTAACAATATTCTTGTTTCCAAAG
TACAAAAAAAATAGGTTAAATAATCTAGCCATAACTGAATGTCAAGCAATAA
AACAAATGACTTTTGTGCATAGACTTAAAAAATACAAATTTTAGACATCTGC
ATGTAAATGCAATCTCTTGTTTACTGCTTTCTTCAACTTGAGCAATCGATTC
CTTATTTACTATTAACTTAAGAACTTGTAACGGCCTGTAAACATTTTCTCTCT
TACTCTTCTTTCAAATTTACCTGTCCCTGCTTTTGAGTCATAATATTTAATGT
TGTTCTGCACATCTCTATACAGTTAACTTTTTGGCTTTCATTCTGTATAGATA
AGAAAATGTTATATTATAAACAGCCTACTCAGTGCAAATATTTATCTGTTTAT
CAAATCCACAATATGCTGTATAATACCGGTTTTACTATATAATCTATTTTAGA
CATAGCTGTTTAGAACTAGAGTGTGCTATTTTTGTGTTTTTCTGATGTGTGG
TGCTAGACAAGTTACTTTTGTGAACAACAAAAATTATCCCTTTTATTCCTAG
ACAATACCACCTTTGGGTCTTGTTAATTTCACTGAGTATAACTATATATTTGT
ATATATATACATATATATATATATCTACCTATGCCCAACTGGCAGCTGTATC
AGAGTGCTGGATTTGGGACATGCTTTTCTCTTTAAATACATAATATCATTAT
ATAAATTATTCTAGAGTGTATTTAATTAGGATAAAATTACTTCCTTAGTATGG
ATATTTGACATCTATAGGGTGAATTTGTTTATAAATATGGCTATATGGAAAC
TTATTAGCATTTACTTTATGTTTGCTACTTGGCTTTACAGCATATCTCCTAAG
CTGAAAAATAATTTGCCAGGCCTTCAAGATCCTAAAGAAACTTGTTTAATGG
AGTAATATACTTTTTTTTCTTATTAAGGAATTGTATTACTGGCACCTAACACA
GTTGTATTCTTAGCTCCTATTATAGATAATGGGCATTTACATAAAATATCCTA
GATGGCTTGATGGCAGAATAAACCTTTCCCCTCCTACCTGAGTCATGAGAA
GGATGGAGACGTCCTCTGCCATAACATGGGCCATAAAGCAAATTCGACAT
GGGATGTTCTGTTTCAGTATGACCTCAACCAGTTCCATGAACTGAGTGAAG
GACCTTCATTTTCAAAGTTATTTAATAAGTAGCTTAATTAAGCCTTTCTACCC
ATTCTCCCAAGATCTACTGGCATTATTGAAAAGCAAAGTTTATCAAATATCT
AACTAAGGATGTAGTTAACCTTATTAAATATTGATTAGAATTGTTCTGTAATA
TTACTGAATTTGTAAGATCTTTAGCAAAGATTTTTGAGCAATTTATAAATGTA
GAGCAAATGTTTCTGTTTACTGCACTTTTTGTAACTGAAGGTGATAAATTCT
CAAGCCATGATTATTGGCTTCCATGCACTGCAATATTTATCCACAATTCTAG
ACATTTTCCATTTTTGTGGAAGAGTTGCTGTTACCTTAATTATAAATGCAATT
GTGTGGTTAATGAGAGCTAATGCTAGTAGTTAACCTTTTAAAGTGGATTGG
CTACAGTTGAGGGAGAAATCTCTTTTAATATAAATCACATCATTCCTTAACT
GCCTCTCTTGGAAAGAGATTGAAACCTTTTTTTTAAAGCACGATTTAGCATC
CTAAGCTTCCTGAGGGTAGAGATTGTATCTTTTTGCGTCTGCACAATGGCT
AGCACATGTCAGCATTTGACAATTGTTAAATGATAACAAGTGTGCCCCAATT
AAAACGTTTTTCCTGGGTTGTTTTGTTAAATTTACAAAGTAAGCCAAGCCTT
ACGGTTAACATTCTCCTCTACAACCAAGTATTAAAGCCACATTTAAAAAGAC
CACATGAAATGCTGATTCTAATTGTGTGTAGGTCTTGAGGATTAAGCACAC
AAATTTCACAAACTTCTGTTTGAGTAAACAAACTCAGCCTTCTGTAAATATA
CATGCAAGTTTGGAAACAGTAATACTGTACCTATAAATATATGCTGTCTGTT
TTGTGTACAGTATGTAAAAACTCCTTTTCTGCCACACTAAAAATGCAAGCCA
TTTATGGGAATCCTAAAACTAGTATTGAACTAAAACTTTGCTAATGATCTTTA
TTAGAGGATCGTCCAACTTTTCACTTACCTTGGGTTTTCTTTTCAATTCACT
CTTACACTAGTCTGCTTATTTCCAGCTGTTTATTTTATTGAGTCCTGAATTTA
AAAAAAAAATATTTTGATTCATTTTGTAAATACAAGCTGTACAAAAAAGAGA
GATTTAATGTTGTCTTTTAAATACTCCAATTTTCATTCTAATATGAATGTTGT
TATATTGTACTTAGAAACTGTACCTTTAATATTACATTACCTTTATTAAAAGT
GCATTGAACACATCAATTTTAGATGTGCTTTATGTACTGTTATCCTATAATAA
AACTTCAGCTTCTAATGGAA

Accordingly, preferably RORA comprises or consists of a nucleotide sequence substantially as set out in SEQ ID NO: 2, or a fragment or variant thereof.

→In one embodiment, GHR is provided by gene bank locus ID: HGNC: 4263; Entrez Gene: 2690; and/or Ensembl: ENSG00000112964. The protein sequence is represented by the GeneBank ID P10912, which is provided herein as SEQ ID No: 4, as follows:

[SEQ ID No: 4]
MDLWQLLLTLALAGSSDAFSGSEATAAILSRAPWSLQSVNPGLKTNSSK
EPKFTKCRSPERETFSCHWTDEVHHGTKNLGPIQLFYTRRNTQEWTQEW
KECPDYVSAGENSCYFNSSFTSIWIPYCIKLTSNGGTVDEKCFSVDEIV
QPDPPIALNWTLLNVSLTGIHADIQVRWEAPRNADIQKGWMVLEYELQY
KEVNETKWKMMDPILTTSVPVYSLKVDKEYEVRVRSKQRNSGNYGEFSE
VLYVTLPQMSQFTCEEDFYFPWLLIIIFGIFGLTVMLFVFLFSKQQRIK
MLILPPVPVPKIKGIDPDLLKEGKLEEVNTILAIHDSYKPEFHSDDSWV
EFIELDIDEPDEKTEESDTDRLLSSDHEKSHSNLGVKDGDSGRTSCCEP
DILETDFNANDIHEGTSEVAQPQRLKGEADLLCLDQKNQNNSPYHDACP
ATQQPSVIQAEKNKPQPLPTEGAESTHQAAHIQLSNPSSLSNIDFYAQV
SDITPAGSVVLSPGQKNKAGMSQCDMHPEMVSLCQENFLMDNAYFCEAD
AKKCIPVAPHIKVESHIQPSLNQEDIYITTESLTTAAGRPGTGEHVPGS
EMPVPDYTSIHIVQSPQGLILNATALPLPDKEFLSSCGYVSTDQLNKIM
P

Accordingly, preferably GHR comprises or consists of an amino acid sequence substantially as set out in SEQ ID NO: 4, or a fragment or variant thereof.

In one embodiment, GHR is encoded by a nucleotide sequence, which is provided herein as SEQ ID No: 5, as follows:

[SEQ ID No: 5]
AACTAGCAATGGTGGTACAGTGGATGAAAAGTGTTTCTCTGTTGATGAAAT
AGGCGGCGGCGGCGGCAGCGGCAGCAGCAGCTGCTACAGTGGCGGTGG
CGGCGGCGGCTGCTGCTGAGCCCGGGGGGGGGGGGACCCCGGGCT
GGGGCCACGCGGGCCGGAGGCCCCGGCACCATTGGCCCCAGCGCAGAC
GCGAACCCGCGCTCTCTGATCAGAGGCGAAGCTCGGAGGTCCTACAGGT
ATGGATCTCTGGCAGCTGCTGTTGACCTTGGCACTGGCAGGATCAAGTGA
TGCTTTTTCTGGAAGTGAGGCCACAGCAGCTATCCTTAGCAGAGCACCCT
GGAGTCTGCAAAGTGTTAATCCAGGCCTAAAGACAAATTCTTCTAAGGAGC
CTAAATTCACCAAGTGCCGTTCACCTGAGCGAGAGACTTTTTCATGCCACT
GGACAGATGAGGTTCATCATGGTACAAAGAACCTAGGACCCATACAGCTG
TTCTATACCAGAAGGAACACTCAAGAATGGACTCAAGAATGGAAAGAATGC
CCTGATTATGTTTCTGCTGGGGAAAACAGCTGTTACTTTAATTCATCGTTTA
CCTCCATCTGGATACCTTATTGTATCAAGCTTGCAACCAGATCCACCCATT
GCCCTCAACTGGACTTTACTGAACGTCAGTTTAACTGGGATTCATGCAGAT
ATCCAAGTGAGATGGGAAGCACCACGCAATGCAGATATTCAGAAAGGATG
GATGGTTCTGGAGTATGAACTTCAATACAAAGAAGTAAATGAAACTAAATG
GAAAATGATGGACCCTATATTGACAACATCAGTTCCAGTGTACTCATTGAA
AGTGGATAAGGAATATGAAGTGCGTGTGAGATCCAAACAACGAAACTCTG
GAAATTATGGCGAGTTCAGTGAGGTGCTCTATGTAACACTTCCTCAGATGA
GCCAATTTACATGTGAAGAAGATTTCTACTTTCCATGGCTCTTAATTATTAT
CTTTGGAATATTTGGGCTAACAGTGATGCTATTTGTATTCTTATTTTCTAAAC
AGCAAAGGATTAAAATGCTGATTCTGCCCCCAGTTCCAGTTCCAAAGATTA
AAGGAATCGATCCAGATCTCCTCAAGGAAGGAAAATTAGAGGAGGTGAAC
ACAATCTTAGCCATTCATGATAGCTATAAACCCGAATTCCACAGTGATGACT
CTTGGGTTGAATTTATTGAGCTAGATATTGATGAGCCAGATGAAAAGACTG
AGGAATCAGACACAGACAGACTTCTAAGCAGTGACCATGAGAAATCACATA
GTAACCTAGGGGTGAAGGATGGCGACTCTGGACGTACCAGCTGTTGTGAA
CCTGACATTCTGGAGACTGATTTCAATGCCAATGACATACATGAGGGTACC
TCAGAGGTTGCTCAGCCACAGAGGTTAAAAGGGGAAGCAGATCTCTTATG
CCTTGACCAGAAGAATCAAAATAACTCACCTTATCATGATGCTTGCCCTGC
TACTCAGCAGCCCAGTGTTATCCAAGCAGAGAAAAACAAACCACAACCACT
TCCTACTGAAGGAGCTGAGTCAACTCACCAAGCTGCCCATATTCAGCTAAG
CAATCCAAGTTCACTGTCAAACATCGACTTTTATGCCCAGGTGAGCGACAT
TACACCAGCAGGTAGTGTGGTCCTTTCCCCGGGCCAAAAGAATAAGGCAG
GGATGTCCCAATGTGACATGCACCCGGAAATGGTCTCACTCTGCCAAGAA
AACTTCCTTATGGACAATGCCTACTTCTGTGAGGCAGATGCCAAAAAGTGC
ATCCCTGTGGCTCCTCACATCAAGGTTGAATCACACATACAGCCAAGCTTA
AACCAAGAGGACATTTACATCACCACAGAAAGCCTTACCACTGCTGCTGG
GAGGCCTGGGACAGGAGAACATGTTCCAGGTTCTGAGATGCCTGTCCCAG
ACTATACCTCCATTCATATAGTACAGTCCCCACAGGGCCTCATACTCAATG
CGACTGCCTTGCCCTTGCCTGACAAAGAGTTTCTCTCATCATGTGGCTATG
TGAGCACAGACCAACTGAACAAAATCATGCCTTAGCCTTTCTTTGGTTTCC
CAAGAGCTACGTATTTAATAGCAAAGAATTGACTGGGGCAATAACGTTTAA
GCCAAAACAATGTTTAAACCTTTTTTGGGGGAGTGACAGGATGGGGTATG
GATTCTAAAATGCCTTTTCCCAAAATGTTGAAATATGATGTTAAAAAAATAA
GAAGAATGCTTAATCAGATAGATATTCCTATTGTGCAATGTAAATATTTTAA
AGAATTGTGTCAGACTGTTTAGTAGCAGTGATTGTCTTAATATTGTGGGTGT
TAATTTTTGATACTAAGCATTGAATGGCTATGTTTTTAATGTATAGTAAATCA
CGCTTTTTGAAAAAGCGAAAAAATCAGGTGGCTTTTGCGGTTCAGGAAAAT
TGAATGCAAACCATAGCACAGGCTAATTTTTTGTTGTTTCTTAAATAAGAAA
CTTTTTTATTTAAAAAACTAAAAACTAGAGGTGAGAAATTTAAACTATAAGCA
AGAAGGCAAAAATAGTTTGGATATGTAAAACATTTATTTTGACATAAAGTTG
ATAAAGATTTTTTAATAATTTAGACTTCAAGCATGGCTATTTTATATTACACT
ACACACTGTGTACTGCAGTTGGTATGACCCCTCTAAGGAGTGTAGCAACTA
CAGTCTAAAGCTGGTTTAATGTTTTGGCCAATGCACCTAAAGAAAAACAAA
CTCGTTTTTTACAAAGCCCTTTTATACCTCCCCAGACTCCTTCAACAATTCT
AAAATGATTGTAGTAATCTGCATTATTGGAATATAATTGTTTTATCTGAATTT
TTAAACAAGTATTTGTTAATTTAGAAAACTTTAAAGCGTTTGCACAGATCAA
CTTACCAGGCACCAAAAGAAGTAAAAGCAAAAAAGAAAACCTTTCTTCACC
AAATCTTGGTTGATGCCAAAAAAAAATACATGCTAAGAGAAGTAGAAATCAT
AGCTGGTTCACACTGACCAAGATACTTAAGTGCTGCAATTGCACGCGGAG
TGAGTTTTTTAGTGCGTGCAGATGGTGAGAGATAAGATCTATAGCCTCTGC
AGCGGAATCTGTTCACACCCAACTTGGTTTTGCTACATAATTATCCAGGAA
GGGAATAAGGTACAAGAAGCATTTTGTAAGTTGAAGCAAATCGAATGAAAT
TAACTGGGTAATGAAACAAAGAGTTCAAGAAATAAGTTTTTGTTTCACAGCC
TATAACCAGACACATACTCATTTTTCATGATAATGAACAGAACATAGACAGA
AGAAACAAGGTTTTCAGTCCCCACAGATAACTGAAAATTATTTAAACCGCTA
AAAGAAACTTTCTTTCTCACTAAATCTTTTATAGGATTTATTTAAAATAGCAA
AAGAAGAAGTTTCATCATTTTTTACTTCCTCTCTGAGTGGACTGGCCTCAAA
GCAAGCATTCAGAAGAAAAAGAAGCAACCTCAGTAATTTAGAAATCATTTT
GCAATCCCTTAATATCCTAAACATCATTCATTTTTGTTGTTGTTGTTGTTGTT
GAGACAGAGTCTCGCTCTGTCGCCAGGCTAGAGTGCGGTGGCGCGATCT
TGACTCACTGCAATCTCCACCTCCCACAGGTTCAGGCGATTCCCGTGCCT
CAGCCTCCTGAGTAGCTGGGACTACAGGCACGCACCACCATGCCAGGCTA
ATTTTTTTGTATTTTAGCAGAGACGGGGTTTCACCATGTTGGCCAGGATGG
TCTCGATCTCCTGACCTCGTGATCCACCCGACTCGGCCTCCCAAAGTGCT
GGGATTACAGGTGTAAGCCACCGTGCCCAGCCCTAAACATCATTCTTGAG
AGCATTGGGATATCTCCTGAAAAGGTTTATGAAAAAGAAGAATCTCATCTC
AGTGAAGAATACTTCTCATTTTTTAAAAAAGCTTAAAACTTTGAAGTTAGCTT
TAACTTAAATAGTATTTCCCATTTATCGCAGACCTTTTTTAGGAAGCAAGCT
TAATGGCTGATAATTTTAAATTCTCTCTCTTGCAGGAAGGACTATGAAAAGC
TAGAATTGAGTGTTTAAAGTTCAACATGTTATTTGTAATAGATGTTTGATAG
ATTTTCTGCTACTTTGCTGCTATGGTTTTCTCCAAGAGCTACATAATTTAGT
TTCATATAAAGTATCATCAGTGTAGAACCTAATTCAATTCAAAGCTGTGTGT
TTGGAAGACTATCTTACTATTTCACAACAGCCTGACAACATTTCTATAGCCA
AAAATAGCTAAATACCTCAATCAGTCTCAGAATGTCATTTTGGTACTTTGGT
GGCCACATAAGCCATTATTCACTAGTATGACTAGTTGTGTCTGGCAGTTTA
TATTTAACTCTCTTTATGTCTGTGGATTTTTTCCTTCAAAGTTTAATAAATTTA
TTTTCTTGGATTCCTGATAGTGTGCTTCTGTTATCAAACACCAACATAAAAA
TGATCTAAACC

Accordingly, preferably GHR comprises or consists of a nucleotide sequence substantially as set out in SEQ ID NO: 5, or a fragment or variant thereof.

In one embodiment, TNF-□ is provided by gene bank locus ID: HGNC: 11892; Entrez Gene: 7124; and/or Ensembl: ENSG00000232810. The protein sequence may be represented by the GeneBank ID P01375, which is provided herein as SEQ ID No: 6, as follows:

[SEQ ID No: 6]
MSTESMIRDVELAEEALPKKTGGPQGSRRCLFLSLFSFLIVAGAT
TLFCLLHFGVIGPQREEFPRDLSLISPLAQAVRSSSRTPSDKPVA
HVVANPQAEGQLQWLNRRANALLANGVELRDNQLVVPSEGLYLIY
SQVLFKGQGCPSTHVLLTHTISRIAVSYQTKVNLLSAIKSPCQRE
TPEGAEAKPWYEPIYLGGVFQLEKGDRLSAEINRPDYLDFAESGQ
VYFGIIAL

Accordingly, preferably TNF-□ comprises or consists of an amino acid sequence as substantially as set out in SEQ ID NO: 6, or a fragment or variant thereof.

In one embodiment TNF-□ is encoded by a nucleotide sequence which is provided herein as SEQ ID No: 7, as follows:

[SEQ ID No: 7]
AGCAGACGCTCCCTCAGCAAGGACAGCAGAGGACCAGCTAAGAGG
GAGAGAAGCAACTACAGACCCCCCCTGAAAACAACCCTCAGACGC
CACATCCCCTGACAAGCTGCCAGGCAGGTTCTCTTCCTCTCACAT
ACTGACCCACGGCTCCACCCTCTCTCCCCTGGAAAGGACACCATG
AGCACTGAAAGCATGATCCGGGACGTGGAGCTGGCCGAGGAGGCG
CTCCCCAAGAAGACAGGGGGGCCCCAGGGCTCCAGGCGGTGCTTG
TTCCTCAGCCTCTTCTCCTTCCTGATCGTGGCAGGCGCCACCACG
CTCTTCTGCCTGCTGCACTTTGGAGTGATCGGCCCCCAGAGGGAA
GAGTTCCCCAGGGACCTCTCTCTAATCAGCCCTCTGGCCCAGGCA
GTCAGATCATCTTCTCGAACCCCGAGTGACAAGCCTGTAGCCCAT
GTTGTAGCAAACCCTCAAGCTGAGGGGCAGCTCCAGTGGCTGAAC
CGCCGGGCCAATGCCCTCCTGGCCAATGGCGTGGAGCTGAGAGAT
AACCAGCTGGTGGTGCCATCAGAGGGCCTGTACCTCATCTACTCC
CAGGTCCTCTTCAAGGGCCAAGGCTGCCCCTCCACCCATGTGCTC
CTCACCCACACCATCAGCCGCATCGCCGTCTCCTACCAGACCAAG
GTCAACCTCCTCTCTGCCATQAAGAGCCCCTGCCAGAGGGAGACC
CCAGAGGGGGCTGAGGCCAAGCCCTGGTATGAGCCCATCTATCTG
GGAGGGGTCTTCCAGCTGGAGAAGGGTGACCGACTCAGCGCTGAG
ATCAATCGGCCCGACTATCTCGACTTTGCCGAGTCTGGGCAGGTC
TACTTTGGGATCATTGCCCTGTGAGGAGGACGAACATCCAACCTT
CCCAAACGCCTCCCCTGCCCCAATCCCTTTATTACCCCCTCCTTC
AGACACCCTCAACCTCTTCTGGCTCAAAAAGAGAATTGGGGGCTT
AGGGTCGGAACCCAAGCTTAGAACTTTAAGCAACAAGACCACCAC
TTCGAAACCTGGGATTCAGGAATGTGTGGCCTGCACAGTGAAGTG
CTGGCAACCACTAAGAATTCAAACTGGGGCCTCCAGAACTCACTG
GGGCCTACAGCTTTGATCCCTGACATCTGGAATCTGGAGACCAGG
GAGCCTTTGGTTCTGGCCAGAATGCTGCAGGACTTGAGAAGACCT
CACCTAGAAATTGACACAAGTGGACCTTAGGCCTTCCTCTCTCCA
GATGTTTCCAGACTTCCTTGAGACACGGAGCCCAGCCCTCCCCAT
GGAGCCAGCTCCCTCTATTTATGTTTGCACTTGTGATTATTTATT
ATTTATTTATTATTTATTTATTTACAGATGAATGTATTTATTTGG
GAGACCGGGGTATCCTGGGGGACCCAATGTAGGAGCTGCCTTGGC
TCAGACATGTTTTCCGTGAAAACGGAGCTGAACAATAGGCTGTTC
CCATGTAGCCCCCTGGCCTCTGTGCCTTCTTTTGATTATGTTTTT
TAAAATATTTATCTGATTAAGTTGTCTAAACAATGCTGATTTGGT
GACCAACTGTCACTCATTGCTGAGCCTCTGCTCCCCAGGGGAGTT
GTGTCTGTAATCGCCCTACTATTCAGTGGCGAGAAATAAAGTTTG
QTTAGAAAAGAAA

Accordingly, preferably TNF-□ comprises or consists of a nucleotide sequence as substantially as set out in SEQ ID NO: 7, or a fragment or variant thereof.

In one embodiment, GSTA1 is provided by gene bank locus ID: HGNC: 4626; Entrez Gene: 2938; Ensembl: ENSG00000243955; OMIM: 138359; and/or UniProtKB: P08263. The protein sequence may be represented by the GeneBank ID: ENST00000334575.6, which is provided herein as SEQ ID No: 8, as follows:

[SEQ ID No: 8]
MAEKPKLHYFNARGRMESTRWLLAAAGVEFEEKFIKSAEDLDKLR
NDGYLMFQQVPMVEIDGMKLVQTRAILNYIASKYNLYGKDIKERA
LIDMYIEGIADLGEMILLLPVCPPEEKDAKLALIKEKIKNRYFPA
FEKVLKSHGQDYLVGNKLSRADIHLVELLYYVEELDSSLISSFPL
LKALKTRISNLPTVKKFLQPGSPRKPPMDEKSLEEARKIFRF

Accordingly, preferably GSTA1 comprises or consists of an amino acid sequence substantially as set out in SEQ ID NO: 8, or a fragment or variant thereof.

In one embodiment GSTA1 is encoded by a nucleotide sequence which is provided herein as SEQ ID No: 9, as follows:

[SEQ ID No: 9]
GTCGAGCCAGGACGGTGACAGCGTTTAACAAAGCTTAGAGAAACC
TCCAGGAGACTGCTATCATGGCAGAGAAGCCCAAGCTCCACTACT
TCAATGCACGGGGCAGAATGGAGTCCACCCGGTGGCTCCTGGCTG
CAGCTGGAGTAGAGTTTGAAGAGAAATTTATAAAATCTGCAGAAG
ATTTGGACAAGTTAAGAAATGATGGATATTTGATGTTCCAGCAAG
TGCCAATGGTTGAGATTGATGGGATGAAGCTGGTGCAGACCAGAG
CCATTCTCAACTACATTGCCAGCAAATACAACCTCTATGGGAAAG
ACATAAAGGAGAGAGCCCTGATTGATATGTATATAGAAGGTATAG
CAGATTTGGGTGAAATGATCCTCCTTCTGCCCGTATGTCCACCTG
AGGAAAAAGATGCCAAGCTTGCCTTGATCAAAGAGAAAATAAAAA
ATCGCTACTTCCCTGCCTTTGAAAAAGTCTTAAAGAGCCATGGAC
AAGACTACCTTGTTGGCAACAAGCTGAGCCGGGCTGACATTCATC
TGGTGGAACTTCTCTACTACGTCGAGGAGCTTGACTCCAGTCTTA
TCTCCAGCTTCCCTCTGCTGAAGGCCCTGAAAACCAGAATCAGCA
ACCTGCCCACAGTGAAGAAGTTTCTACAGCCTGGCAGCCCAAGGA
AGCCTCCCATGGATGAGAAATCTTTAGAAGAAGCAAGGAAGATTT
TCAGGTTTTAATAACGCAGTCATGGAGGCCAAGAACTTGCAATAC
CAATGTTCTAAAGTTTTGCAACAATAAAGTACTTTACCTAAGTGT
TGATTGTGCCTGTTGTGAAGCTAATGAACTCTTTCAAATTATATG
CTAATTAAATAATACAACTCCTATTCGCTGACTTAGTTAAAATTG
ATTTGTTTTCATTAGGATCTGATGTGAATTCAGATTTCCAATCTT
CTCCTAGCCAACCATTTTCCTGGAATTAAAAATTCAGTAAAAAAG
GAAACTATAGATTATGTGGTTTGTTTGACTTTTCCAAGAATTGTC
CCGTAACATACAATTTGTCATACAATCTATTAAAATGTCAATGTA
GAAATGCACTTCTGACATTTTCAGGTATGCACAGGAGAAGAGTTA
CCATCCTGGATAATGGCATAAAGACATTTTCTTCTTTTCCTGGAC
AGTCATTTTATTTCTGATAAAAGCGTTCTTTCTTATGCATTTGCA
AAA

Accordingly, preferably GSTA1 comprises or consists of a nucleotide sequence as substantially as set out in SEQ ID NO: 9, or a fragment or variant thereof.

In one embodiment, NT-proBNP is provided by gene bank locus ID: HGNC: 7940; Entrez Gene: 4879; Ensembl: ENSG00000120937; OMIM: 600295; and/or UniProtKB: P16860. The protein sequence may be represented by the GeneBank ID P16860, which is provided herein as SEQ ID No: 10, as follows:

[SEQ ID No: 10]
MDPQTAPSRALLLLLFLHLAFLGGRSHPLGSPGSASDLETSGLQE
QRNHLQGKLSELQVEQTSLEPLQESPRPTGVWKSREVATEGIRGH
RKMVLYTLRAPRSPKMVQGSGCFGRKMDRISSSSGLGCKVLRRH

Accordingly, preferably NT-proBNP comprises or consists of an amino acid sequence substantially as set out in SEQ ID NO: 10, or a fragment or variant thereof.

In one embodiment NT-proBNP is encoded by a nucleotide sequence which is provided herein as SEQ ID No: 11, as follows:

[SEQ ID No: 11]
AGGAGGAGCACCCCGCAGGCTGAGGGCAGGTGGGAAGCAAACCCG
GACGCATCGCAGCAGCAGCAGCAGCAGCAGAAGCAGCAGCAGCAG
CCTCCGCAGTCCCTCCAGAGACATGGATCCCCAGACAGCACCTTC
CCGGGCGCTCCTGCTCCTGCTCTTCTTGCATCTGGCTTTCCTGGG
AGGTCGTTCCCACCCGCTGGGCAGCCCCGGTTCAGCCTCGGACTT
GGAAACGTCCGGGTTACAGGAGCAGCGCAACCATTTGCAGGGCAA
ACTGTCGGAGCTGCAGGTGGAGCAGACATCCCTGGAGCCCCTCCA
GGAGAGCCCCCGTCCCACAGGTGTCTGGAAGTCCCGGGAGGTAGC
CACCGAGGGCATCCGTGGGCACCGCAAAATGGTCCTCTACACCCT
GCGGGCACCACGAAGCCCCAAGATGGTGCAAGGGTCTGGCTGCTT
TGGGAGGAAGATGGACCGGATCAGCTCCTCCAGTGGCCTGGGCTG
CAAAGTGCTGAGGCGGCATTAAGAGGAAGTCCTGGCTGCAGACAC
CTGCTTCTGATTCCACAAGGGGCTTTTTCCTCAACCCTGTGGCCG
CCTTTGAAGTGACTCATTTTTTTAATGTATTTATGTATTTATTTG
ATTGTTTTATATAAGATGGTTTCTTACCTTTGAGCACAAAATTTC
CACGGTGAAATAAAGTCAACATTATAAGCTTTA

Accordingly, preferably NT-proBNP comprises or consists of a nucleotide sequence substantially as set out in SEQ ID NO: 11, or a fragment or variant thereof.

In one embodiment, TNC is provided by gene bank locus ID: HGNC: 5318; Entrez Gene: 3371; Ensembl: ENSG00000041982; OMIM: 187380; and/or UniProtKB: P24821. The protein sequence may be represented by the GeneBank ID P24821, which is provided herein as SEQ ID No: 12, as follows:

[SEQ ID No: 12]
MGAMTQLLAGVFLAFLALATEGGVLKKVIRHKRQSGVNATLPEEN
QPVVFNHVYNIKLPVGSQCSVDLESASGEKDLAPPSEPSESFQEH
TVDGENQIVFTHRINIPRRACGCAAAPDVKELLSRLEELENLVSS
LREQCTAGAGCCLQPATGRLDTRPFCSGRGNFSTEGCGCVCEPGW
KGPNCSEPECPGNCHLRGRCIDGQCICDDGFTGEDCSQLACPSDC
NDQGKCVNGVCICFEGYAGADCSREICPVPCSEEHGTCVDGLCVC
HDGFAGDDCNKPLCLNNCYNRGRCVENECVCDEGFTGEDCSELIC
PNDCFDRGRCINGTCYCEEGFTGEDCGKPTCPHACHTQGRCEEGQ
CVCDEGFAGVDCSEKRCPADCHNRGRCVDGRCECDDGFTGADCGE
LKCPNGCSGHGRCVNGQCVCDEGYTGEDCSQLRCPNDCHSRGRCV
EGKCVCEQGFKGYDCSDMSCPNDCHQHGRCVNGMCVCDDGYTGED
CRDRQCPRDCSNRGLCVDGQCVCEDGFTGPDCAELSCPNDCHGQG
RCVNGQCVCHEGFMGKDCKEQRCPSDCHGQGRCVDGQCICHEGFT
GLDCGQHSCPSDCNNLGQCVSGRCICNEGYSGEDCSEVSPPKDLV
VTEVTEETVNLAWDNEMRVTEYLVVYTPTHEGGLEMQFRVPGDQT
STIIQELEPGVEYFIRVFAILENKKSIPVSARVATYLPAPEGLKF
KSIKETSVEVEWDPLDIAFETWEIIFRNMNKEDEGEITKSLRRPE
TSYRQTGLAPGQEYEISLHIVKNNTRGPGLKRVTTTRLDAPSQIE
VKDVTDTTALITWFKPLAEIDGIELTYGIKDVPGDRTTIDLTEDE
NQYSIGNLKPDTEYEVSLISRRGDMSSNPAKETFTTGLDAPRNLR
RVSQTDNSITLEWRNGKAAIDSYRIKYAPISGGDHAEVDVPKSQQ
ATTKTTLTGLRPGTEYGIGVSAVKEDKESNPATINAATELDTPKD
LQVSETAETSLTLLWKTPLAKFDRYRLNYSLPTGQWVGVQLPRNT
TSYVLRGLEPGQEYNVLLTAEKGRHKSKPARVKASTEQAPELENL
TVTEVGWDGLRLNWTAADQAYEHFIIQVQEANKVEAARNLTVPGS
LRAVDIPGLKAATPYTVSIYGVIQGYRTPVLSAEASTGETPNLGE
VVVAEVGWDALKLNWTAPEGAYEYFFIQVQEADTVEAAQNLTVPG
GLRSTDLPGLKAATHYTITIRGVTQDFSTTPLSVEVLTEEVPDMG
NLTVTEVSWDALRLNWTTPDGTYDQFTIQVQEADQVEEAHNLTVP
GSLRSMEIPGLRAGTPYTVTLHGEVRGHSTRPLAVEVVTEDLPQL
GDLAVSEVGWDGLRLNWTAADNAYEHFVIQVQEVNKVEAAQNLTL
PGSLRAVDIPGLEAATPYRVSIYGVIRGYRTPVLSAEASTAKEPE
IGNLNVSDITPESFNLSWMATDGIFETFTIEIIDSNRLLETVEYN
ISGAERTAHISGLPPSTDFIVYLSGLAPSIRTKTISATATTEALP
LLENLTISDINPYGFTVSWMASENAFDSFLVTVVDSGKLLDPQEF
TLSGTQRKLELRGLITGIGYEVMVSGFTQGHQTKPLRAEIVTEAE
PEVDNLLVSDATPDGFRLSWTADEGVFDNFVLKIRDTKKQSEPLE
ITLLAPERTRDITGLREATEYEIELYGISKGRRSQTVSAIATTAM
GSPKEVIFSDITENSATVSWRAPTAQVESFRITYVPITGGTPSMV
TVDGTKTQTRLVKLIPGVEYLVSIIAMKGFEESEPVSGSFTTALD
GPSGLVTANITDSEALARWQPAIATVDSYVISYTGEKVPEITRTV
SGNTVEYALTDLEPATEYTLRIFAEKGPQKSSTITAKFTTDLDSP
RDLTATEVQSETALLTWRPPRASVTGYLLVYESVDGTVKEVIVGP
DTTSYSLADLSPSTHYTAKIQALNGPLRSNMIQTIFTTIGLLYPF
PKDCSQAMLNGDTTSGLYTIYLNGDKAEALEVFCDMTSDGGGWIV
FLRRKNGRENFYQNWKAYAAGFGDRREEFWLGLDNLNKITAQGQY
ELRVDLRDHGETAFAVYDKFSVGDAKTRYKLKVEGYSGTAGDSMA
YHNGRSFSTFDKDTDSAITNCALSYKGAFWYRNCHRVNLMGRYGD
NNHSQGVNWFHWKGHEHSIQFAEMKLRPSNFRNLEGRRKRA

Accordingly, preferably TNC comprises or consists of an amino acid sequence substantially as set out in SEQ ID NO: 12, or a fragment or variant thereof.

In one embodiment TNC is encoded by a nucleotide sequence which is provided herein as SEQ ID No: 13, as follows:

[SEQ ID No: 13]
GGCCACAGCCTGCCTACTGTCACCCGCCTCTCCCGCGCGCAGATA
CACGCCCCCGCCTCCGTGGGCACAAAGGCAGCGCTGCTGGGGAAC
TCGGGGGAACGCGCACGTGGGAACCGCCGCAGCTCCACACTCCAG
GTACTTCTTCCAAGGACCTAGGTCTCTCGCCCATCGGAAAGAAAA
TAATTCTTTCAAGAAGATCAGGGACAACTGATTTGAAGTCTACTC
TGTGCTTCTAAATCCCCAATTCTGCTGAAAGTGAGATACCCTAGA
GCCCTAGAGCCCCAGCAGCACCCAGCCAAACCCACCTCCACCATG
GGGGCCATGACTCAGCTGTTGGCAGGTGTCTTTCTTGCTTTCCTT
GCCCTCGCTACCGAAGGTGGGGTCCTCAAGAAAGTCATCCGGCAC
AAGCGACAGAGTGGGGTGAACGCCACCCTGCCAGAAGAGAACCAG
CCAGTGGTGTTTAACCACGTTTACAACATCAAGCTGCCAGTGGGA
TCCCAGTGTTCGGTGGATCTGGAGTCAGCCAGTGGGGAGAAAGAC
CTGGCACCGCCTTCAGAGCCCAGCGAAAGCTTTCAGGAGCACACA
GTGGATGGGGAAAACCAGATTGTCTTCACACATCGCATCAACATC
CCCCGCCGGGCCTGTGGCTGTGCCGCAGCCCCTGATGTTAAGGAG
CTGCTGAGCAGACTGGAGGAGCTGGAGAACCTGGTGTCTTCCCTG
AGGGAGCAATGTACTGCAGGAGCAGGCTGCTGTCTCCAGCCTGCC
ACAGGCCGCTTGGACACCAGGCCCTTCTGTAGCGGTCGGGGCAAC
TTCAGCACTGAAGGATGTGGCTGTGTCTGCGAACCTGGCTGGAAA
GGCCCCAACTGCTCTGAGCCCGAATGTCCAGGCAACTGTCACCTT
CGAGGCCGGTGCATTGATGGGCAGTGCATCTGTGACGACGGCTTC
ACGGGCGAGGACTGCAGCCAGCTGGCTTGCCCCAGCGACTGCAAT
GACCAGGGCAAGTGCGTAAATGGAGTCTGCATCTGTTTCGAAGGC
TACGCCGGGGCTGACTGCAGCCGTGAAATCTGCCCAGTGCCCTGC
AGTGAGGAGCACGGCACATGTGTAGATGGCTTGTGTGTGTGCCAC
GATGGCTTTGCAGGCGATGACTGCAACAAGCCTCTGTGTCTCAAC
AATTGCTACAACCGTGGACGATGCGTGGAGAATGAGTGCGTGTGT
GATGAGGGTTTCACGGGCGAAGACTGCAGTGAGCTCATCTGCCCC
AATGACTGCTTCGACCGGGGCCGCTGCATCAATGGCACCTGCTAC
TGCGAAGAAGGCTTCACAGGTGAAGACTGCGGGAAACCCACCTGC
CCACATGCCTGCCACACCCAGGGCCGGTGTGAGGAGGGGCAGTGT
GTATGTGATGAGGGCTTTGCCGGTGTGGACTGCAGCGAGAAGAGG
TGTCCTGCTGACTGTCACAATCGTGGCCGCTGTGTAGACGGGCGG
TGTGAGTGTGATGATGGTTTCACTGGAGCTGACTGTGGGGAGCTC
AAGTGTCCCAATGGCTGCAGTGGCCATGGCCGCTGTGTCAATGGG
CAGTGTGTGTGTGATGAGGGCTATACTGGGGAGGACTGCAGCCAG
CTACGGTGCCCCAATGACTGTCACAGTCGGGGCCGCTGTGTCGAG
GGCAAATGTGTATGTGAGCAAGGCTTCAAGGGCTATGACTGCAGT
GACATGAGCTGCCCTAATGACTGTCACCAGCACGGCCGCTGTGTG
AATGGCATGTGTGTTTGTGATGACGGCTACACAGGGGAAGACTGC
CGGGATCGCCAATGCCCCAGGGACTGCAGCAACAGGGGCCTCTGT
GTGGACGGACAGTGCGTCTGTGAGGACGGCTTCACCGGCCCTGAC
TGTGCAGAACTCTCCTGTCCAAATGACTGCCATGGCCAGGGTCGC
TGTGTGAATGGGCAGTGCGTGTGCCATGAAGGATTTATGGGCAAA
GACTGCAAGGAGCAAAGATGTCCCAGTGACTGTCATGGCCAGGGC
CGCTGCGTGGACGGCCAGTGCATCTGCCACGAGGGCTTCACAGGC
CTGGACTGTGGCCAGCACTCCTGCCCCAGTGACTGCAACAACTTA
GGACAATGCGTCTCGGGCCGCTGCATCTGCAACGAGGGCTACAGC
GGAGAAGACTGCTCAGAGGTGTCTCCTCCCAAAGACCTCGTTGTG
ACAGAAGTGACGGAAGAGACGGTCAACCTGGCCTGGGACAATGAG
ATGCGGGTCACAGAGTACCTTGTCGTGTACACGCCCACCCACGAG
GGTGGTCTGGAAATGCAGTTCCGTGTGCCTGGGGACCAGACGTCC
ACCATCATCCAGGAGCTGGAGCCTGGTGTGGAGTACTTTATCCGT
GTATTTGCCATCCTGGAGAACAAGAAGAGCATTCCTGTCAGCGCC
AGGGTGGCCACGTACTTACCTGCACCTGAAGGCCTGAAATTCAAG
TCCATCAAGGAGACATCTGTGGAAGTGGAGTGGGATCCTCTAGAC
ATTGCTTTTGAAACCTGGGAGATCATCTTCCGGAATATGAATAAA
GAAGATGAGGGAGAGATCACCAAAAGCCTGAGGAGGCCAGAGACC
TCTTACCGGCAAACTGGTCTAGCTCCTGGGCAAGAGTATGAGATA
TCTCTGCACATAGTGAAAAACAATACCCGGGGCCCTGGCCTGAAG
AGGGTGACCACCACACGCTTGGATGCCCCCAGCCAGATCGAGGTG
AAAGATGTCACAGACACCACTGCCTTGATCACCTGGTTCAAGCCC
CTGGCTGAGATCGATGGCATTGAGCTGACCTACGGCATCAAAGAC
GTGCCAGGAGACCGTACCACCATCGATCTCACAGAGGACGAGAAC
CAGTACTCCATCGGGAACCTGAAGCCTGACACTGAGTACGAGGTG
TCCCTCATCTCCCGCAGAGGTGACATGTCAAGCAACCCAGCCAAA
GAGACCTTCACAACAGGCCTCGATGCTCCCAGGAATCTTCGACGT
GTTTCCCAGACAGATAACAGCATCACCCTGGAATGGAGGAATGGC
AAGGCAGCTATTGACAGTTACAGAATTAAGTATGCCCCCATCTCT
GGAGGGGACCACGCTGAGGTTGATGTTCCAAAGAGCCAACAAGCC
ACAACCAAAACCACACTCACAGGTCTGAGGCCGGGAACTGAATAT
GGGATTGGAGTTTCTGCTGTGAAGGAAGACAAGGAGAGCAATCCA
GCGACCATCAACGCAGCCACAGAGTTGGACACGCCCAAGGACCTT
CAGGTTTCTGAAACTGCAGAGACCAGCCTGACCCTGCTCTGGAAG
ACACCGTTGGCCAAATTTGACCGCTACCGCCTCAATTACAGTCTC
CCCACAGGCCAGTGGGTGGGAGTGCAGCTTCCAAGAAACACCACT
TCCTATGTCCTGAGAGGCCTGGAACCAGGACAGGAGTACAATGTC
CTCCTGACAGCCGAGAAAGGCAGACACAAGAGCAAGCCCGCACGT
GTGAAGGCATCCACTGAACAAGCCCCTGAGCTGGAAAACCTCACC
GTGACTGAGGTTGGCTGGGATGGCCTCAGACTCAACTGGACCGCA
GCTGACCAGGCCTATGAGCACTTTATCATTCAGGTGCAGGAGGCC
AACAAGGTGGAGGCAGCTCGGAACCTCACCGTGCCTGGCAGCCTT
CGGGCTGTGGACATACCGGGCCTCAAGGCTGCTACGCCTTATACA
GTCTCCATCTATGGGGTGATCCAGGGCTATAGAACACCAGTGCTC
TCTGCTGAGGCCTCCACAGGGGAAACTCCCAATTTGGGAGAGGTC
GTGGTGGCCGAGGTGGGCTGGGATGCCCTCAAACTCAACTGGACT
GCTCCAGAAGGGGCCTATGAGTACTTTTTCATTCAGGTGCAGGAG
GCTGACACAGTAGAGGCAGCCCAGAACCTCACCGTCCCAGGAGGA
CTGAGGTCCACAGACCTGCCTGGGCTCAAAGCAGCCACTCATTAT
ACCATCACCATCCGCGGGGTCACTCAGGACTTCAGCACAACCCCT
CTCTCTGTTGAAGTCTTGACAGAGGAGGTTCCAGATATGGGAAAC
CTCACAGTGACCGAGGTTAGCTGGGATGCTCTCAGACTGAACTGG
ACCACGCCAGATGGAACCTATGACCAGTTTACTATTCAGGTCCAG
GAGGCTGACCAGGTGGAAGAGGCTCACAATCTCACGGTTCCTGGC
AGCCTGCGTTCCATGGAAATCCCAGGCCTCAGGGCTGGCACTCCT
TACACAGTCACCCTGCACGGCGAGGTCAGGGGCCACAGCACTCGA
CCCCTTGCTGTAGAGGTCGTCACAGAGGATCTCCCACAGCTGGGA
GATTTAGCCGTGTCTGAGGTTGGCTGGGATGGCCTCAGACTCAAC
TGGACCGCAGCTGACAATGCCTATGAGCACTTTGTCATTCAGGTG
CAGGAGGTCAACAAAGTGGAGGCAGCCCAGAACCTCACGTTGCCT
GGCAGCCTCAGGGCTGTGGACATCCCGGGCCTCGAGGCTGCCACG
CCTTATAGAGTCTCCATCTATGGGGTGATCCGGGGCTATAGAACA
CCAGTACTCTCTGCTGAGGCCTCCACAGCCAAAGAACCTGAAATT
GGAAACTTAAATGTTTCTGACATAACTCCCGAGAGCTTCAATCTC
TCCTGGATGGCTACCGATGGGATCTTCGAGACCTTTACCATTGAA
ATTATTGATTCCAATAGGTTGCTGGAGACTGTGGAATATAATATC
TCTGGTGCTGAACGAACTGCCCATATCTCAGGGCTACCCCCTAGT
ACTGATTTTATTGTCTACCTCTCTGGACTTGCTCCCAGCATCCGG
ACCAAAACCATCAGTGCCACAGCCACGACAGAGGCCCTGCCCCTT
CTGGAAAACCTAACCATTTCCGACATTAATCCCTACGGGTTCACA
GTTTCCTGGATGGCATCGGAGAATGCCTTTGACAGCTTTCTAGTA
ACGGTGGTGGATTCTGGGAAGCTGCTGGACCCCCAGGAATTCACA
CTTTCAGGAACCCAGAGGAAGCTGGAGCTTAGAGGCCTCATAACT
GGCATTGGCTATGAGGTTATGGTCTCTGGQTTCACCCAAGGGCAT
CAAACCAAGCCCTTGAGGGCTGAGATTGTTACAGAAGCCGAACCG
GAAGTTGACAACCTTCTGGTTTCAGATGCCACCCCAGACGGTTTC
CGTCTGTCCTGGACAGCTGATGAAGGGGTCTTCGACAATTTTGTT
CTCAAAATCAGAGATACCAAAAAGCAGTCTGAGCCACTGGAAATA
ACCCTACTTGCCCCCGAACGTACCAGGGACATAACAGGTCTCAGA
GAGGCTACTGAATACGAAATTGAACTCTATGGAATAAGCAAAGGA
AGGCGATCCCAGACAGTCAGTGCTATAGCAACAACAGCCATGGGC
TCCCCAAAGGAAGTCATTTTCTCAGACATCACTGAAAATTCGGCT
ACTGTCAGCTGGAGGGCACCCACAGCCCAAGTGGAGAGCTTCCGG
ATTACCTATGTGCCCATTACAGGAGGTACACCCTCCATGGTAACT
GTGGACGGAACCAAGACTCAGACCAGGCTGGTGAAACTCATACCT
GGCGTGGAGTACCTTGTCAGCATCATCGCCATGAAGGGCTTTGAG
GAAAGTGAACCTGTCTCAGGGTCATTCACCACAGCTCTGGATGGC
CCATCTGGCCTGGTGACAGCCAACATCACTGACTCAGAAGCCTTG
GCCAGGTGGCAGCCAGCCATTGCCACTGTGGACAGTTATGTCATC
TCCTACACAGGCGAGAAAGTGCCAGAAATTACACGCACGGTGTCC
GGGAACACAGTGGAGTATGCTCTGACCGACCTCGAGCCTGCCACG
GAATACACACTGAGAATCTTTGCAGAGAAAGGGCCCCAGAAGAGC
TCAACCATCACTGCCAAGTTCACAACAGACCTCGATTCTCCAAGA
GACTTGACTGCTACTGAGGTTCAGTCGGAAACTGCCCTCCTTACC
TGGCGACCCCCCCGGGCATCAGTCACCGGTTACCTGCTGGTCTAT
GAATCAGTGGATGGCACAGTCAAGGAAGTCATTGTGGGTCCAGAT
ACCACCTCCTACAGCCTGGCAGACCTGAGCCCATCCACCCACTAC
ACAGCCAAGATCCAGGCACTCAATGGGCCCCTGAGGAGCAATATG
ATCCAGACCATCTTCACCACAATTGGACTCCTGTACCCCTTCCCC
AAGGACTGCTCCCAAGCAATGCTGAATGGAGACACGACCTCTGGC
CTCTACACCATTTATCTGAATGGTGATAAGGCTGAGGCGCTGGAA
GTCTTCTGTGACATGACCTCTGATGGGGGTGGATGGATTGTGTTC
CTGAGACGCAAAAACGGACGCGAGAACTTCTACCAAAACTGGAAG
GCATATGCTGCTGGATTTGGGGACCGCAGAGAAGAATTCTGGQTT
GGGCTGGACAACCTGAACAAAATCACAGCCCAGGGGCAGTACGAG
CTCCGGGTGGACCTGCGGGACCATGGGGAGACAGCCTTTGCTGTC
TATGACAAGTTCAGCGTGGGAGATGCCAAGACTCGCTACAAGCTG
AAGGTGGAGGGGTACAGTGGGACAGCAGGTGACTCCATGGCCTAC
CACAATGGCAGATCCTTCTCCACCTTTGACAAGGACACAGATTCA
GCCATCACCAACTGTGCTCTGTCCTACAAAGGGGCTTTCTGGTAC
AGGAACTGTCACCGTGTCAACCTGATGGGGAGATATGGGGACAAT
AACCACAGTCAGGGCGTTAACTGGTTCCACTGGAAGGGCCACGAA
CACTCAATCCAGTTTGCTGAGATGAAGCTGAGACCAAGCAACTTC
AGAAATCTTGAAGGCAGGCGCAAACGGGCATAAATTCCAGGGACC
ACTGGGTGAGAGAGGAATAAGGCCCAGAGCGAGGAAAGGATTTTA
CCAAAGCATCAATACAACCAGCCCAACCATCGGTCCACACCTGGG
CATTTGGTGAGAGTCAAAGCTGACCATGGATCCCTGGGGCCAACG
GCAACAGCATGGGCCTCACCTCCTCTGTGATTTCTTTCTTTGCAC
CAAAGACATCAGTCTCCAACATGTTTCTGTTTTGTTGTTTGATTC
AGCAAAAATCTCCCAGTGACAACATCGCAATAGTTTTTTACTTCT
CTTAGGTGGCTCTGGGAATGGGAGAGGGGTAGGATGTACAGGGGT
AGTTTGTTTTAGAACCAGCCGTATTTTACATGAAGCTGTATAATT
AATTGTCATTATTTTTGTTAGCAAAGATTAAATGTGTCATTGGAA
GCCATCCCTTTTTTTACATTTCATACAACAGAAACCAGAAAAGCA
ATACTGTTTCCATTTTAAGGATATGATTAATATTATTAATATAAT
AATGATGATGATGATGATGAAAACTAAGGATTTTTCAAGAGATCT
TTCTTTCCAAAACATTTCTGGACAGTACCTGATTGTATTTTTTTT
TTAAATAAAAGCACAAGTACTTTTGAGTTTGTTATTTTGCTTTGA
ATTGTTGAGTCTGAATTTCACCAAAGCCAATCATTTGAACAAAGC
GGGGAATGTTGGGATAGGAAAGGTAAGTAGGGATAGTGGTCAAGT
GGGAGGGGTGGAAAGGAGACTAAAGACTGGGAGAGAGGGAAGCAC
TTTTTTTAAATAAAGTTGAACACACTTGGGAAAAGCTTACAGGCC
AGGCCTGTAATCCCAACACTTTGGGAGGCCAAGGTGGGAGGATAG
CTTAACCCCAGGAGTTTGAGACCAGCCTGAGCAACATAGTGAGAA
CTTGTCTCTACAGAAAAAAAAAAAAAAAAAAATTTAATTAGGCAA
GCGTGGTAGTGCGCACCTGTCGTCCCAGCTACTCAGGAGGCTGAG
GTAGGAAAATCACTGGAGCCCAGGAGTTAGAGGTTACAGTGAGCT
ATGATCACACTACTGCACTCCAGCCTGGGCAACAGAGGGAGACCC
TGTCTCTAAATAAAAAAAGAAAAGAAAAAAAAAGCTTACAACTTG
AGATTCAGCATCTTGCTCAGTATTTCCAAGACTAATAGATTATGG
TTTAAAAGATGCTTTTATACTCATTTTCTAATGCAACTCCTAGAA
ACTCTATGATATAGTTGAGGTAAGTATTGTTACCACACATGGGCT
AAGATCCCCAGAGGCAGACTGCCTGAGTTCAATTCTTGGCTCCAC
CATTCCCAAGTTCCCTAACCTCTCTATGCCTCAGTTTCCTCTTCT
GTAAAGTAGGGACACTCATACTTCTCATTTCAGAACATTTTTGTG
AAGAATAAATTATGTTATCCATTTGAGGCCCTTAGAATGGTACCC
GGTGTATATTAAGTGCTAGTACATGTTAGCTATCATCATTATCAC
TTTATATGAGATGGACTGGGGTTCATAGAAACCCAATGACTTGAT
TGTGGCTACTACTCAATAAATAATAGAATTTGGATTTAAA

Accordingly, preferably TNC comprises or consists of a nucleotide sequence substantially as set out in SEQ ID NO: 13, or a fragment or variant thereof.

In one embodiment, A2M is provided by gene bank locus ID: HGNC: 7; Entrez Gene: 2; Ensembl: ENSG00000175899; OMIM: 103950; and/or UniProtKB: P01023. The protein sequence may be represented by the GeneBank ID P01023, which is provided herein as SEQ ID No: 14, as follows:

[SEQ ID No: 14]
MGKNKLLHPSLVLLLLVLLPTDASVSGKPQYMVLVPSLLHTETTE
KGCVLLSYLNETVTVSASLESVRGNRSLFTDLEAENDVLHCVAFA
VPKSSSNEEVMFLTVQVKGPTQEFKKRTTVMVKNEDSLVFVQTDK
SIYKPGQTVKFRVVSMDENFHPLNELIPLVYIQDPKGNRIAQWQS
FQLEGGLKQFSFPLSSEPFQGSYKVVVQKKSGGRTEHPFTVEEFV
LPKFEVQVTVPKIITILEEEMNVSVCGLYTYGKPVPGHVTVSICR
KYSDASDCHGEDSQAFCEKFSGQLNSHGCFYQQVKTKVFQLKRKE
YEMKLHTEAQIQEEGTVVELTGRQSSEITRTITKLSFVKVDSHFR
QGIPFFGQVRLVDGKGVPIPNKVIFIRGNEANYYSNATTDEHGLV
QFSINTTNVMGTSLTVRVNYKDRSPCYGYQWVSEEHEEAHHTAYL
VFSPSKSFVHLEPMSHELPCGHTQTVQAHYILNGGTLLGLKKLSF
YYLIMAKGGIVRTGTHGLLVKQEDMKGHFSISIPVKSDIAPVARL
LIYAVLPTGDVIGDSAKYDVENCLANKVDLSFSPSQSLPASHAHL
RVTAAPQSVCALRAVDQSVLLMKPDAELSASSVYNLLPEKDLTGF
PGPLNDQDNEDCINRHNVYINGITYTPVSSTNEKDMYSFLEDMGL
KAFTNSKIRKPKMCPQLQQYEMHGPEGLRVGFYESDVMGRGHARL
VHVEEPHTETVRKYFPETWIWDLVVVNSAGVAEVGVTVPDTITEW
KAGAFCLSEDAGLGISSTASLRAFQPFFVELTMPYSVIRGEAFTL
KATVLNYLPKCIRVSVQLEASPAFLAVPVEKEQAPHCICANGRQT
VSWAVTPKSLGNVNFTVSAEALESQELCGTEVPSVPEHGRKDTVI
KPLLVEPEGLEKETTFNSLLCPSGGEVSEELSLKLPPNVVEESAR
ASVSVLGDILGSAMQNTQNLLQMPYGCGEQNMVLFAPNIYVLDYL
NETQQLTPEIKSKAIGYLNTGYQRQLNYKHYDGSYSTFGERYGRN
QGNTWLTAFVLKTFAQARAYIFIDEAHITQALIWLSQRQKDNGCF
RSSGSLLNNAIKGGVEDEVTLSAYITIALLEIPLTVTHPVVRNAL
FCLESAWKTAQEGDHGSHVYTKALLAYAFALAGNQDKRKEVLKSL
NEEAVKKDNSVHWERPQKPKAPVGHFYEPQAPSAEVEMTSYVLLA
YLTAQPAPTSEDLTSATNIVKWITKQQNAQGGFSSTQDTVVALHA
LSKYGAATFTRTGKAAQVTIQSSGTFSSKFQVDNNNRLLLQQVSL
PELPGEYSMKVTGEGCVYLQTSLKYNILPEKEEFPFALGVQTLPQ
TCDEPKAHTSFQISLSVSYTGSRSASNMAIVDVKMVSGFIPLKPT
VKMLERSNHVSRTEVSSNHVLIYLDKVSNQTLSLFFTVLQDVPVR
DLKPAIVKVYDYYETDEFAIAEYNAPCSKDLGNA

Accordingly, preferably A2M comprises or consists of an amino acid sequence substantially as set out in SEQ ID NO: 14, or a fragment or variant thereof.

In one embodiment A2M is encoded by a nucleotide sequence which is provided herein as SEQ ID No: 15, as follows:

[SEQ ID No: 15]
GAAAAGCTTATTAGCTGCTGTACGGTAAAAGTGAGCTCTTACGGG
AATGGGAATGTAGTTTTAGCCCTCCAGGGATTCTATTTAGCCCGC
CAGGAATTAACCTTGACTATAAATAGGCCATCAATGACCTTTCCA
GAGAATGTTCAGAGACCTCAACTTTGTTTAGAGATCTTGTGTGGG
TGGAACTTCCTGTTTGCACACAGAGCAGCATAAAGCCCAGTTGCT
TTGGGAAGTGTTTGGGACCAGATGGATTGTAGGGAGTAGGGTACA
ATACAGTCTGTTCTCCTCCAGCTCCTTCTTTCTGCAACATGGGGA
AGAACAAACTCCTTCATCCAAGTCTGGTTCTTCTCCTCTTGGTCC
TCCTGCCCACAGACGCCTCAGTCTCTGGAAAACCGCAGTATATGG
TTCTGGTCCCCTCCCTGCTCCACACTGAGACCACTGAGAAGGGCT
GTGTCCTTCTGAGCTACCTGAATGAGACAGTGACTGTAAGTGCTT
CCTTGGAGTCTGTCAGGGGAAACAGGAGCCTCTTCACTGACCTGG
AGGCGGAGAATGACGTACTCCACTGTGTCGCCTTCGCTGTCCCAA
AGTCTTCATCCAATGAGGAGGTAATGTTCCTCACTGTCCAAGTGA
AAGGACCAACCCAAGAATTTAAGAAGCGGACCACAGTGATGGTTA
AGAACGAGGACAGTCTGGTCTTTGTCCAGACAGACAAATCAATCT
ACAAACCAGGGCAGACAGTGAAATTTCGTGTTGTCTCCATGGATG
AAAACTTTCACCCCCTGAATGAGTTGATTCCACTAGTATACATTC
AGGATCCCAAAGGAAATCGCATCGCACAATGGCAGAGTTTCCAGT
TAGAGGGTGGCCTCAAGCAATTTTCTTTTCCCCTCTCATCAGAGC
CCTTCCAGGGCTCCTACAAGGTGGTGGTACAGAAGAAATCAGGTG
GAAGGACAGAGCACCCTTTCACCGTGGAGGAATTTGTTCTTCCCA
AGTTTGAAGTACAAGTAACAGTGCCAAAGATAATCACCATCTTGG
AAGAAGAGATGAATGTATCAGTGTGTGGCCTATACACATATGGGA
AGCCTGTCCCTGGACATGTGACTGTGAGCATTTGCAGAAAGTATA
GTGACGCTTCCGACTGCCACGGTGAAGATTCACAGGCTTTCTGTG
AGAAATTCAGTGGACAGCTAAACAGCCATGGCTGCTTCTATCAGC
AAGTAAAAACCAAGGTCTTCCAGCTGAAGAGGAAGGAGTATGAAA
TGAAACTTCACACTGAGGCCCAGATCCAAGAAGAAGGAACAGTGG
TGGAATTGACTGGAAGGCAGTCCAGTGAAATCACAAGAACCATAA
CCAAACTCTCATTTGTGAAAGTGGACTCACACTTTCGACAGGGAA
TTCCCTTCTTTGGGCAGGTGCGCCTAGTAGATGGGAAAGGCGTCC
CTATACCAAATAAAGTCATATTCATCAGAGGAAATGAAGCAAACT
ATTACTCCAATGCTACCACGGATGAGCATGGCCTTGTACAGTTCT
CTATCAACACCACCAATGTTATGGGTACCTCTCTTACTGTTAGGG
TCAATTACAAGGATCGTAGTCCCTGTTACGGCTACCAGTGGGTGT
CAGAAGAACACGAAGAGGCACATCACACTGCTTATCTTGTGTTCT
CCCCAAGCAAGAGCTTTGTCCACCTTGAGCCCATGTCTCATGAAC
TACCCTGTGGCCATACTCAGACAGTCCAGGCACATTATATTCTGA
ATGGAGGCACCCTGCTGGGGCTGAAGAAGCTCTCCTTCTATTATC
TGATAATGGCAAAGGGAGGCATTGTCCGAACTGGGACTCATGGAC
TGCTTGTGAAGCAGGAAGACATGAAGGGCCATTTTTCCATCTCAA
TCCCTGTGAAGTCAGACATTGCTCCTGTCGCTCGGTTGCTCATCT
ATGCTGTTTTACCTACCGGGGACGTGATTGGGGATTCTGCAAAAT
ATGATGTTGAAAATTGTCTGGCCAACAAGGTGGATTTGAGCTTCA
GCCCATCACAAAGTCTCCCAGCCTCACACGCCCACCTGCGAGTCA
CAGCGGCTCCTCAGTCCGTCTGCGCCCTCCGTGCTGTGGACCAAA
GCGTGCTGCTCATGAAGCCTGATGCTGAGCTCTCGGCGTCCTCGG
TTTACAACCTGCTACCAGAAAAGGACCTCACTGGCTTCCCTGGGC
CTTTGAATGACCAGGACAATGAAGACTGCATCAATCGTCATAATG
TCTATATTAATGGAATCACATATACTCCAGTATCAAGTACAAATG
AAAAGGATATGTACAGCTTCCTAGAGGACATGGGCTTAAAGGCAT
TCACCAACTCAAAGATTCGTAAACCCAAAATGTGTCCACAGCTTC
AACAGTATGAAATGCATGGACCTGAAGGTCTACGTGTAGGTTTTT
ATGAGTCAGATGTAATGGGAAGAGGCCATGCACGCCTGGTGCATG
TTGAAGAGCCTCACACGGAGACCGTACGAAAGTACTTCCCTGAGA
CATGGATCTGGGATTTGGTGGTGGTAAACTCAGCAGGTGTGGCTG
AGGTAGGAGTAACAGTCCCTGACACCATCACCGAGTGGAAGGCAG
GGGCCTTCTGCCTGTCTGAAGATGCTGGACTTGGTATCTCTTCCA
CTGCCTCTCTCCGAGCCTTCCAGCCCTTCTTTGTGGAGCTCACAA
TGCCTTACTCTGTGATTCGTGGAGAGGCCTTCACACTCAAGGCCA
CGGTCCTAAACTACCTTCCCAAATGCATCCGGGTCAGTGTGCAGC
TGGAAGCCTCTCCCGCCTTCCTAGCTGTCCCAGTGGAGAAGGAAC
AAGCGCCTCACTGCATCTGTGCAAACGGGCGGCAAACTGTGTCCT
GGGCAGTAACCCCAAAGTCATTAGGAAATGTGAATTTCACTGTGA
GCGCAGAGGCACTAGAGTCTCAAGAGCTGTGTGGGACTGAGGTGC
CTTCAGTTCCTGAACACGGAAGGAAAGACACAGTCATCAAGCCTC
TGTTGGTTGAACCTGAAGGACTAGAGAAGGAAACAACATTCAACT
CCCTACTTTGTCCATCAGGTGGTGAGGTTTCTGAAGAATTATCCC
TGAAACTGCCACCAAATGTGGTAGAAGAATCTGCCCGAGCTTCTG
TCTCAGTTTTGGGAGACATATTAGGCTCTGCCATGCAAAACACAC
AAAATCTTCTCCAGATGCCCTATGGCTGTGGAGAGCAGAATATGG
TCCTCTTTGCTCCTAACATCTATGTACTGGATTATCTAAATGAAA
CACAGCAGCTTACTCCAGAGATCAAGTCCAAGGCCATTGGCTATC
TCAACACTGGTTACCAGAGACAGTTGAACTACAAACACTATGATG
GCTCCTACAGCACCTTTGGGGAGCGATATGGCAGGAACCAGGGCA
ACACCTGGCTCACAGCCTTTGTTCTGAAGACTTTTGCCCAAGCTC
GAGCCTACATCTTCATCGATGAAGCACACATTACCCAAGCCCTCA
TATGGCTCTCCCAGAGGCAGAAGGACAATGGCTGTTTCAGGAGCT
CTGGGTCACTGCTCAACAATGCCATAAAGGGAGGAGTAGAAGATG
AAGTGACCCTCTCCGCCTATATCACCATCGCCCTTCTGGAGATTC
CTCTCACAGTCACTCACCCTGTTGTCCGCAATGCCCTGTTTTGCC
TGGAGTCAGCCTGGAAGACAGCACAAGAAGGGGACCATGGCAGCC
ATGTATATACCAAAGCACTGCTGGCCTATGCTTTTGCCCTGGCAG
GTAACCAGGACAAGAGGAAGGAAGTACTCAAGTCACTTAATGAGG
AAGCTGTGAAGAAAGACAACTCTGTCCATTGGGAGCGCCCTCAGA
AACCCAAGGCACCAGTGGGGCATTTTTACGAACCCCAGGCTCCCT
CTGCTGAGGTGGAGATGACATCCTATGTGCTCCTCGCTTATCTCA
CGGCCCAGCCAGCCCCAACCTCGGAGGACCTGACCTCTGCAACCA
ACATCGTGAAGTGGATCACGAAGCAGCAGAATGCCCAGGGCGGTT
TCTCCTCCACCCAGGACACAGTGGTGGCTCTCCATGCTCTGTCCA
AATATGGAGCAGCCACATTTACCAGGACTGGGAAGGCTGCACAGG
TGACTATCCAGTCTTCAGGGACATTTTCCAGCAAATTCCAAGTGG
ACAACAACAACCGCCTGTTACTGCAGCAGGTCTCATTGCCAGAGC
TGCCTGGGGAATACAGCATGAAAGTGACAGGAGAAGGATGTGTCT
ACCTCCAGACATCCTTGAAATACAATATTCTCCCAGAAAAGGAAG
AGTTCCCCTTTGCTTTAGGAGTGCAGACTCTGCCTCAAACTTGTG
ATGAACCCAAAGCCCACACCAGCTTCCAAATCTCCCTAAGTGTCA
GTTACACAGGGAGCCGCTCTGCCTCCAACATGGCGATCGTTGATG
TGAAGATGGTCTCTGGCTTCATTCCCCTGAAGCCAACAGTGAAAA
TGCTTGAAAGATCTAACCATGTGAGCCGGACAGAAGTCAGCAGCA
ACCATGTCTTGATTTACCTTGATAAGGTGTCAAATCAGACACTGA
GCTTGTTCTTCACGGTTCTGCAAGATGTCCCAGTAAGAGATCTGA
AACCAGCCATAGTGAAAGTCTATGATTACTACGAGACGGATGAGT
TTGCAATTGCTGAGTACAATGCTCCTTGCAGCAAAGATCTTGGAA
ATGCTTGAAGACCACAAGGCTGAAAAGTGCTTTGCTGGAGTCCTG
TTCTCAGAGCTCCACAGAAGACACGTGTTTTTGTATCTTTAAAGA
CTTGATGAATAAACACTTTTTCTGGTCAAAGACCACAAGGCTGAA
AAGTGCTTTGCTGGAGTCCTGTTCTCAGAGCTCCACAGAAGACAC
GTGTTTTTGTATCTTTAAAGACTTGATGAATAAACACTTTTTCTG
GTCAA

Accordingly, preferably A2M comprises or consists of a nucleotide sequence substantially as set out in SEQ ID NO: 15, or a fragment or variant thereof.

In one embodiment, IGFBP2 is provided by gene bank locus ID HGNC: 5471; Entrez Gene: 3485; Ensembl: ENSG00000115457; OMIM: 146731 and/or UniProtKB: P18065.

The protein sequence may be represented by the GeneBank ID P18065, which is provided herein as SEQ ID No: 16, as follows:

[SEQ ID No: 16]
MLPRVGCPALPLPPPPLLPLLLLLLGASGGGGGARAEVLFRCPPC
TPERLAACGPPPVAPPAAVAAVAGGARMPCAELVREPGCGCCSVC
ARLEGEACGVYTPRCGQGLRCYPHPGSELPLQALVMGEGTCEKRR
DAEYGASPEQVADNGDDHSEGGLVENHVDSTMNMLGGGGSAGRKP
LKSGMKELAVFREKVTEQHRQMGKGGKHHLGLEEPKKLRPPPART
PCQQELDQVLERISTMRLPDERGPLEHLYSLHIPNCDKHGLYNLK
QCKMSLNGQRGECWCVNPNTGKLIQGAPTIRGDPECHLFYNEQQE
ARGVHTQRMQ

Accordingly, preferably IGFBP2 comprises or consists of an amino acid sequence substantially as set out in SEQ ID NO: 16 or a fragment or variant thereof.

In one embodiment IGFBP2 is encoded by a nucleotide sequence which is provided herein as SEQ ID No: 17, as follows:

[SEQ ID No: 17]
GAGGAAGAAGCGGAGGAGGCGGCTCCCGCGCTCGCAGGGCCGTGC
CACCTGCCCGCCCGCCCGCTCGCTCGCTCGCCCGCCGCGCCGCGC
TGCCGACCGCCAGCATGCTGCCGAGAGTGGGCTGCCCCGCGCTGC
CGCTGCCGCCGCCGCCGCTGCTGCCGCTGCTGCTGCTGCTACTGG
GCGCGAGTGGCGGCGGCGGCGGGGCGCGCGCGGAGGTGCTGTTCC
GCTGCCCGCCCTGCACACCCGAGCGCCTGGCCGCCTGCGGGCCCC
CGCCGGTTGCGCCGCCCGCCGCGGTGGCCGCAGTGGCCGGAGGCG
CCCGCATGCCATGCGCGGAGCTCGTCCGGGAGCCGGGCTGCGGCT
GCTGCTCGGTGTGCGCCCGGCTGGAGGGCGAGGCGTGCGGCGTCT
ACACCCCGCGCTGCGGCCAGGGGCTGCGCTGCTATCCCCACCCGG
GCTCCGAGCTGCCCCTGCAGGCGCTGGTCATGGGCGAGGGCACTT
GTGAGAAGCGCCGGGACGCCGAGTATGGCGCCAGCCCGGAGCAGG
TTGCAGACAATGGCGATGACCACTCAGAAGGAGGCCTGGTGGAGA
ACCACGTGGACAGCACCATGAACATGTTGGGGGGGGAGGCAGTGC
TGGCCGGAAGCCCCTCAAGTCGGGTATGAAGGAGCTGGCCGTGTT
CCGGGAGAAGGTCACTGAGCAGCACCGGCAGATGGGCAAGGGTGG
CAAGCATCACCTTGGCCTGGAGGAGCCCAAGAAGCTGCGACCACC
CCCTGCCAGGACTCCCTGCCAACAGGAACTGGACCAGGTCCTGGA
GQGGATCTCCACCATGCGCCTTCCGGATGAGCGGGGCCCTCTGGA
GCACCTCTACTCCCTGCACATCCCCAACTGTGACAAGCATGGCCT
GTACAACCTCAAACAGTGCAAGATGTCTCTGAACGGGCAGCGTGG
GGAGTGCTGGTGTGTGAACCCCAACACCGGGAAGCTGATCCAGGG
AGCCCCCACCATCCGGGGGGACCCCGAGTGTCATCTCTTCTACAA
TGAGCAGCAGGAGGCTCGCGGGGTGCACACCCAGCGGATGCAGTA
GACCGCAGCCAGCCGGTGCCTGGCGCCCCTGCCCCCCGCCCCTCT
CCAAACACCGGCAGAAAACGGAGAGTGCTTGGGTGGTGGGTGCTG
GAGGATTTTCCAGTTCTGACACACGTATTTATATTTGGAAAGAGA
CCAGCACCGAGCTCGGCACCTCCCCGGCCTCTCTCTTCCCAGCTG
CAGATGCCACACCTGCTCCTTCTTGCTTTCCCCGGGGGAGGAAGG
GGGTTGTGGTCGGGGAGCTGGGGTACAGGTTTGGGGAGGGGGAAG
AGAAATTTTTATTTTTGAACCCCTGTGTCCCTTTTGCATAAGATT
AAAGGAAGGAAAAGTAAA

Accordingly, preferably IGFBP2 comprises or consists of a nucleotide sequence substantially as set out in SEQ ID NO: 17, or a fragment or variant thereof.

In one embodiment, APOB is provided by gene bank locus ID: HGNC: 603; Entrez Gene: 338; Ensembl: ENSG00000084674; OMIM: 107730; and/or UniProtKB: P04114. The protein sequence may be represented by the GeneBank ID P04114, which is provided herein as SEQ ID No: 18, as follows:

[SEQ ID No: 18]
MDPPRPALLALLALPALLLLLLAGARAEEEMLENVSLVCPKDATR
FKHLRKYTYNYEAESSSGVPGTADSRSATRINCKVELEVPQLCSF
ILKTSQCTLKEVYGFNPEGKALLKKTKNSEEFAAAMSRYELKLAI
PEGKQVFLYPEKDEPTYILNIKRGIISALLVPPETEEAKQVLFLD
TVYGNCSTHFTVKTRKGNVATEISTERDLGQCDRFKPIRTGISPL
ALIKGMTRPLSTLISSSQSCQYTLDAKRKHVAEAICKEQHLFLPF
SYKNKYGMVAQVTQTLKLEDTPKINSRFFGEGTKKMGLAFESTKS
TSPPKQAEAVLKTLQELKKLTISEQNIQRANLFNKLVTELRGLSD
EAVTSLLPQLIEVSSPITLQALVQCGQPQCSTHILQWLKRVHANP
LLIDVVTYLVALIPEPSAQQLREIFNMARDQRSRATLYALSHAVN
NYHKTNPTGTQELLDIANYLMEQIQDDCTGDEDYTYLILRVIGNM
GQTMEQLTPELKSSILKCVQSTKPSLMIQKAAIQALRKMEPKDKD
QEVLLQTFLDDASPGDKRLAAYLMLMRSPSQADINKIVQILPWEQ
NEQVKNFVASHIANILNSEELDIQDLKKLVKEALKESQLPTVMDF
RKFSRNYQLYKSVSLPSLDPASAKIEGNLIFDPNNYLPKESMLKT
TLTAFGFASADLIEIGLEGKGFEPTLEALFGKQGFFPDSVNKALY
WVNGQVPDGVSKVLVDHFGYTKDDKHEQDMVNGIMLSVEKLIKDL
KSKEVPEARAYLRILGEELGFASLHDLQLLGKLLLMGARTLQGIP
QMIGEVIRKGSKNDFFLHYIFMENAFELPTGAGLQLQISSSGVIA
PGAKAGVKLEVANMQAELVAKPSVSVEFVTNMGIIIPDFARSGVQ
MNTNFFHESGLEAHVALKAGKLKFIIPSPKRPVKLLSGGNTLHLV
STTKTEVIPPLIENRQSWSVCKQVFPGLNYCTSGAYSNASSTDSA
SYYPLTGDTRLELELRPTGEIEQYSVSATYELQREDRALVDTLKF
VTQAEGAKQTEATMTFKYNRQSMTLSSEVQIPDFDVDLGTILRVN
DESTEGKTSYRLTLDIQNKKITEVALMGHLSCDTKEERKIKGVIS
IPRLQAEARSEILAHWSPAKLLLQMDSSATAYGSTVSKRVAWHYD
EEKIEFEWNTGTNVDTKKMTSNFPVDLSDYPKSLHMYANRLLDHR
VPQTDMTFRHVGSKLIVAMSSWLQKASGSLPYTQTLQDHLNSLKE
FNLQNMGLPDFHIPENLFLKSDGRVKYTLNKNSLKIEIPLPFGGK
SSRDLKMLETVRTPALHFKSVGFHLPSREFQVPTFTIPKLYQLQV
PLLGVLDLSTNVYSNLYNWSASYSGGNTSTDHFSLRARYHMKADS
VVDLLSYNVQGSGETTYDHKNTFTLSCDGSLRHKFLDSNIKFSHV
EKLGNNPVSKGLLIFDASSSWGPQMSASVHLDSKKKQHLFVKEVK
IDGQFRVSSFYAKGTYGLSCQRDPNTGRLNGESNLRFNSSYLQGT
NQITGRYEDGTLSLTSTSDLQSGIIKNTASLKYENYELTLKSDTN
GKYKNFATSNKMDMTFSKQNALLRSEYQADYESLRFFSLLSGSLN
SHGLELNADILGTDKINSGAHKATLRIGQDGISTSATTNLKCSLL
VLENELNAELGLSGASMKLTTNGRFREHNAKFSLDGKAALTELSL
GSAYQAMILGVDSKNIFNFKVSQEGLKLSNDMMGSYAEMKFDHTN
SLNIAGLSLDFSSKLDNIYSSDKFYKQTVNLQLQPYSLVTTLNSD
LKYNALDLTNNGKLRLEPLKLHVAGNLKGAYQNNEIKHIYAISSA
ALSASYKADTVAKVQGVEFSHRLNTDIAGLASAIDMSTNYNSDSL
HFSNVFRSVMAPFTMTIDAHTNGNGKLALWGEHTGQLYSKFLLKA
EPLAFTFSHDYKGSTSHHLVSRKSISAALEHKVSALLTPAEQTGT
WKLKTQFNNNEYSQDLDAYNTKDKIGVELTGRTLADLTLLDSPIK
VPLLLSEPINIIDALEMRDAVEKPQEFTIVAFVKYDKNQDVHSIN
LPFFETLQEYFERNRQTIIVVLENVQRNLKHINIDQFVRKYRAAL
GKLPQQANDYLNSFNWERQVSHAKEKLTALTKKYRITENDIQIAL
DDAKINFNEKLSQLQTYMIQFDQYIKDSYDLHDLKIAIANIIDEI
IEKLKSLDEHYHIRVNLVKTIHDLHLFIENIDFNKSGSSTASWIQ
NVDTKYQIRIQIQEKLQQLKRHIQNIDIQHLAGKLKQHIEAIDVR
VLLDQLGTTISFERINDILEHVKHFVINLIGDFEVAEKINAFRAK
VHELIERYEVDQQIQVLMDKLVELAHQYKLKETIQKLSNVLQQVK
IKDYFEKLVGFIDDAVKKLNELSFKTFIEDVNKFLDMLIKKLKSF
DYHQFVDETNDKIREVTQRLNGEIQALELPQKAEALKLFLEETKA
TVAVYLESLQDTKITLIINWLQEALSSASLAHMKAKFRETLEDTR
DRMYQMDIQQELQRYLSLVGQVYSTLVTYISDWWTLAAKNLTDFA
EQYSIQDWAKRMKALVEQGFTVPEIKTILGTMPAFEVSLQALQKA
TFQTPDFIVPLTDLRIPSVQINFKDLKNIKIPSRFSTPEFTILNT
FHIPSFTIDFVEMKVKIIRTIDQMLNSELQWPVPDIYLRDLKVED
IPLARITLPDFRLPEIAIPEFIIPTLNLNDFQVPDLHIPEFQLPH
ISHTIEVPTFGKLYSILKIQSPLFTLDANADIGNGTTSANEAGIA
ASITAKGESKLEVLNFDFQANAQLSNPKINPLALKESVKFSSKYL
RTEHGSEMLFFGNAIEGKSNTVASLHTEKNTLELSNGVIVKINNQ
LTLDSNTKYFHKLNIPKLDFSSQADLRNEIKTLLKAGHIAWTSSG
KGSWKWACPRFSDEGTHESQISFTIEGPLTSFGLSNKINSKHLRV
NQNLVYESGSLNFSKLEIQSQVDSQHVGHSVLTAKGMALFGEGKA
EFTGRHDAHLNGKVIGTLKNSLFFSAQPFEITASTNNEGNLKVRF
PLRLTGKIDFLNNYALFLSPSAQQASWQVSARFNQYKYNQNFSAG
NNENIMEAHVGINGEANLDFLNIPLTIPEMRLPYTIITTPPLKDF
SLWEKTGLKEFLKTTKQSFDLSVKAQYKKNKHRHSITNPLAVLCE
FISQSIKSFDRHFEKNRNNALDFVTKSYNETKIKFDKYKAEKSHD
ELPRTFQIPGYTVPVVNVEVSPFTIEMSAFGYVFPKAVSMPSFSI
LGSDVRVPSYTLILPSLELPVLHVPRNLKLSLPDFKELCTISHIF
IPAMGNITYDFSFKSSVITLNTNAELFNQSDIVAHLLSSSSSVID
ALQYKLEGTTRLTRKRGLKLATALSLSNKFVEGSHNSTVSLTTKN
MEVSVATTTKAQIPILRMNFKQELNGNTKSKPTVSSSMEFKYDFN
SSMLYSTAKGAVDHKLSLESLTSYFSIESSTKGDVKGSVLSREYS
GTIASEANTYLNSKSTRSSVKLQGTSKIDDIWNLEVKENFAGEAT
LQRIYSLWEHSTKNHLQLEGLFFTNGEHTSKATLELSPWQMSALV
QVHASQPSSFHDFPDLGQEVALNANTKNQKIRWKNEVRIHSGSFQ
SQVELSNDQEKAHLDIAGSLEGHLRFLKNIILPVYDKSLWDFLKL
DVTTSIGRRQHLRVSTAFVYTKNPNGYSFSIPVKVLADKFIIPGL
KLNDLNSVLVMPTFHVPFTDLQVPSCKLDFREIQIYKKLRTSSFA
LNLPTLPEVKFPEVDVLTKYSQPEDSLIPFFEITVPESQLTVSQF
TLPKSVSDGIAALDLNAVANKIADFELPTIIVPEQTIEIPSIKFS
VPAGIVIPSFQALTARFEVDSPVYNATWSASLKNKADYVETVLDS
TCSSTVQFLEYELNVLGTHKIEDGTLASKTKGTFAHRDFSAEYEE
DGKYEGLQEWEGKAHLNIKSPAFTDLHLRYQKDKKGISTSAASPA
VGTVGMDMDEDDDFSKWNFYYSPQSSPDKKLTIFKTELRVRESDE
ETQIKVNWEEEAASGLLTSLKDNVPKATGVLYDYVNKYHWEHTGL
TLREVSSKLRRNLQNNAEWVYQGAIRQIDDIDVRFQKAASGTTGT
YQEWKDKAQNLYQELLTQEGQASFQGLKDNVFDGLVRVTQEFHMK
VKHLIDSLIDFLNFPRFQFPGKPGIYTREELCTMFIREVGTVLSQ
VYSKVHNGSEILFSYFQDLVITLPFELRKHKLIDVISMYRELLKD
LSKEAQEVFKAIQSLKTTEVLRNLQDLLQFIFQLIEDNIKQLKEM
KFTYLINYIQDEINTIFSDYIPYVFKLLKENLCLNLHKFNEFIQN
ELQEASQELQQIHQYIMALREEYFDPSIVGWTVKYYELEEKIVSL
IKNLLVALKDFHSEYIVSASNFTSQLSSQVEQFLHRNIQEYLSIL
TDPDGKGKEKIAELSATAQEIIKSQAIATKKIISDYHQQFRYKLQ
DFSDQLSDYYEKFIAESKRLIDLSIQNYHTFLIYITELLKKLQST
TVMNPYMKLAPGELTIIL

Accordingly, preferably APOB comprises or consists of an amino acid sequence substantially as set out in SEQ ID NO: 18, or a fragment or variant thereof.

In one embodiment APOB is encoded by a nucleotide sequence which is provided herein as SEQ ID No: 19, as follows:

[SEQ ID No: 19]
ATTCCCACCGGGACCTGCGGGGCTGAGTGCCCTTCTCGGTTGCTG
CCGCTGAGGAGCCCGCCCAGCCAGCCAGGGCCGCGAGGCCGAGGC
CAGGCCGCAGCCCAGGAGCCGCCCCACCGCAGCTGGCGATGGACC
CGCCGAGGCCCGCGCTGCTGGCGCTGCTGGCGCTGCCTGCGCTGC
TGCTGCTGCTGCTGGCGGGCGCCAGGGCCGAAGAGGAAATGCTGG
AAAATGTCAGCCTGGTCTGTCCAAAAGATGCGACCCGATTCAAGC
ACCTCCGGAAGTACACATACAACTATGAGGCTGAGAGTTCCAGTG
GAGTCCCTGGGACTGCTGATTCAAGAAGTGCCACCAGGATCAACT
GCAAGGTTGAGCTGGAGGTTCCCCAGCTCTGCAGCTTCATCCTGA
AGACCAGCCAGTGCACCCTGAAAGAGGTGTATGGCTTCAACCCTG
AGGGCAAAGCCTTGCTGAAGAAAACCAAGAACTCTGAGGAGTTTG
CTGCAGCCATGTCCAGGTATGAGCTCAAGCTGGCCATTCCAGAAG
GGAAGCAGGTTTTCCTTTACCCGGAGAAAGATGAACCTACTTACA
TCCTGAACATCAAGAGGGGCATCATTTCTGCCCTCCTGGTTCCCC
CAGAGACAGAAGAAGCCAAGCAAGTGTTGTTTCTGGATACCGTGT
ATGGAAACTGCTCCACTCACTTTACCGTCAAGACGAGGAAGGGCA
ATGTGGCAACAGAAATATCCACTGAAAGAGACCTGGGGCAGTGTG
ATCGCTTCAAGCCCATCCGCACAGGCATCAGCCCACTTGCTCTCA
TCAAAGGCATGACCCGCCCCTTGTCAACTCTGATCAGCAGCAGCC
AGTCCTGTCAGTACACACTGGACGCTAAGAGGAAGCATGTGGCAG
AAGCCATCTGCAAGGAGCAACACCTCTTCCTGCCTTTCTCCTACA
AGAATAAGTATGGGATGGTAGCACAAGTGACACAGACTTTGAAAC
TTGAAGACACACCAAAGATCAACAGCCGCTTCTTTGGTGAAGGTA
CTAAGAAGATGGGCCTCGCATTTGAGAGCACCAAATCCACATCAC
CTCCAAAGCAGGCCGAAGCTGTTTTGAAGACTCTCCAGGAACTGA
AAAAACTAACCATCTCTGAGCAAAATATCCAGAGAGCTAATCTCT
TCAATAAGCTGGTTACTGAGCTGAGAGGCCTCAGTGATGAAGCAG
TCACATCTCTCTTGCCACAGCTGATTGAGGTGTCCAGCCCCATCA
CTTTACAAGCCTTGGTTCAGTGTGGACAGCCTCAGTGCTCCACTC
ACATCCTCCAGTGGCTGAAACGTGTGCATGCCAACCCCCTTCTGA
TAGATGTGGTCACCTACCTGGTGGCCCTGATCCCCGAGCCCTCAG
CACAGCAGCTGCGAGAGATCTTCAACATGGCGAGGGATCAGCGCA
GCCGAGCCACCTTGTATGCGCTGAGCCACGCGGTCAACAACTATC
ATAAGACAAACCCTACAGGGACCCAGGAGCTGCTGGACATTGCTA
ATTACCTGATGGAACAGATTCAAGATGACTGCACTGGGGATGAAG
ATTACACCTATTTGATTCTGCGGGTCATTGGAAATATGGGCCAAA
CCATGGAGCAGTTAACTCCAGAACTCAAGTCTTCAATCCTGAAAT
GTGTCCAAAGTACAAAGCCATCACTGATGATCCAGAAAGCTGCCA
TCCAGGCTCTGCGGAAAATGGAGCCTAAAGACAAGGACCAGGAGG
TTCTTCTTCAGACTTTCCTTGATGATGCTTCTCCGGGAGATAAGC
GACTGGCTGCCTATCTTATGTTGATGAGGAGTCCTTCACAGGCAG
ATATTAACAAAATTGTCCAAATTCTACCATGGGAACAGAATGAGC
AAGTGAAGAACTTTGTGGCTTCCCATATTGCCAATATCTTGAACT
CAGAAGAATTGGATATCCAAGA

Accordingly, preferably APOB comprises or consists of a nucleotide sequence substantially as set out in SEQ ID NO: 19, or a fragment or variant thereof.

In one embodiment, SEPP1 is provided by gene bank locus ID: HGNC: 10751; Entrez Gene: 6414; Ensembl: ENSG00000250722; OMIM: 601484; and/or UniProtKB: P49908. The protein sequence may be represented by the GeneBank ID P49908, which is provided herein as SEQ ID No: 20, as follows:

[SEQ ID No: 20]
MWRSLGLALALCLLPSGGTESQDQSSLCKQPPAWSIRDQDPMLNS
NGSVTVVALLQASUYLCILQASKLEDLRVKLKKEGYSNISYIVVN
HQGISSRLKYTHLKNKVSEHIPVYQQEENQTDVWTLLNGSKDDFL
IYDRCGRLVYHLGLPFSFLTFPYVEEAIKIAYCEKKCGNCSLTTL
KDEDFCKRVSLATVDKTVETPSPHYHHEHHHNHGHQHLGSSELSE
NQQPGAPNAPTHPAPPGLHHHHKHKGQHRQGHPENRDMPASEDLQ
DLQKKLCRKRCINQLLCKLPTDSELAPRSUCCHCRHLIFEKTGSA
ITUQCKENLPSLCSUQGLRAEENITESCQURLPPAAUQISQQLIP
TEASASURUKNQAKKUEUPSN

Accordingly, preferably SEPP1 comprises or consists of an amino acid sequence substantially as set out in SEQ ID NO: 20, or a fragment or variant thereof.

In one embodiment SEPP1 is encoded by a nucleotide sequence which is provided herein as SEQ ID No: 21 as follows:

[SEQ ID No: 21]
ACAGACAGGCAGGTGCAGGAGACGGGGTGAGCGCTTTTGGGCTCT
AGCCCCATGGCAGCATCTAGGGGTGTGTTACAATTAATGCTCTTT
TAGCAGTTGCTGTTCGCGGATGGCTAAGTGTTAAACCAGCTCAGT
GGAGAGTCAGGGTGGCAGCCTTTTACGCCCTGTCCTCTTGGTACC
CGGGTCTTTGTCTAGCATCCAGGAAGAATCAGGTCCTACAGACTT
GAAGGATGGTGAATACAGAGATTTTATTGAATGATGGAGGAGGCT
TTCAGCAGGATGGATGGGGAGCTGGAAAGCGGATGGAGTGAGAAG
ATAATCTTCCCCTGGGGTTTGGCCATCCCATGGCTGATCTCTCTG
ACAGTCCCCAGCCGAACTCCTCTCGATGTTCAGACACTCTTTCTC
CTCTCTCCTCTGCCACACTGCTCTGCTGCTCTTCTGCTCATGGAG
CCTGAGGACAACCCCAGCAATGTGGAGAAGCCTGGGGCTTGCCCT
GGCTCTCTGTCTCCTCCCATCGGGAGGAACAGAGAGCCAGGACCA
AAGCTCCTTATGTAAGCAACCCCCAGCCTGGAGCATAAGAGATCA
AGATCCAATGCTAAACTCCAATGGTTCAGTGACTGTGGTTGCTCT
TCTTCAAGCCAGCTGATACCTGTGCATACTGCAGGCATCTAAATT
AGAAGACCTGCGAGTAAAACTGAAGAAAGAAGGATATTCTAATAT
TTCTTATATTGTTGTTAATCATCAAGGAATCTCTTCTCGATTAAA
ATACACACATCTTAAGAATAAGGTTTCAGAGCATATTCCTGTTTA
TCAACAAGAAGAAAACCAAACAGATGTCTGGACTCTTTTAAATGG
AAGCAAAGATGACTTCCTCATATATGATAGATGTGGCCGTCTTGT
ATATCATCTTGGTTTGCCTTTTTCCTTCCTAACTTTCCCATATGT
AGAAGAAGCCATTAAGATTGCTTACTGTGAAAAGAAATGTGGAAA
CTGCTCTCTCACGACTCTCAAAGATGAAGACTTTTGTAAACGTGT
ATCTTTGGCTACTGTGGATAAAACAGTTGAAACTCCATCGCCTCA
TTACCATCATGAGCATCATCACAATCATGGACATCAGCACCTTGG
CAGCAGTGAGCTTTCAGAGAATCAGCAACCAGGAGCACCAAATGC
TCCTACTCATCCTGCTCCTCCAGGCCTTCATCACCACCATAAGCA
CAAGGGTCAGCATAGGCAGGGTCACCCAGAGAACCGAGATATGCC
AGCAAGTGAAGATTTACAAGATTTACAAAAGAAGCTCTGTCGAAA
GAGATGTATAAATCAATTACTCTGTAAATTGCCCACAGATTCAGA
GTTGGCTCCTAGGAGCTGATGCTGCCATTGTCGACATCTGATATT
TGAAAAAACAGGGTCTGCAATCACCTGACAGTGTAAAGAAAACCT
CCCATCTTTATGTAGCTGACAGGGACTTCGGGCAGAGGAGAACAT
AACTGAATCTTGTCAGTGACGTTTGCCTCCAGCTGCCTGACAAAT
AAGTCAGCAGCTTATACCCACAGAAGCCAGTGCCAGTTGACGCTG
AAAGAATCAGGCAAAAAAGTGAGAATGACCTTCAAACTAAATATT
TAAAATAGGACATACTCCCCAATTTAGTCTAGACACAATTTCATT
TCCAGCATTTTTATAAACTACCAAATTAGTGAACCAAAAATAGAA
ATTAGATTTGTGCAAACATGGAGAAATCTACTGAATTGGCTTCCA
GATTTTAAATTTTATGTCATAGAAATATTGACTCAAACCATATTT
TTTATGATGGAGCAACTGAAAGGTGATTGCAGCTTTTGGTTAATA
TGTCTTTTTTTTTCTTTTTCCAGTGTTCTATTTGCTTTAATGAGA
ATAGAAACGTAAACTATGACCTAGGGGTTTCTGTTGGATAATTAG
CAGTTTAGAATGGAGGAAGAACAACAAAGACATGCTTTCCATTTT
TTTCTTTACTTATCTCTCAAAACAATATTACTTTGTCTTTTCAAT
CTTCTACTTTTAACTAATAAAATAAGTGGATTTTGTATTTTAAGA
TCCAGAAATACTTAACACGTGAATATTTTGCTAAAAAAGCATATA
TAACTATTTTAAATATCCATTTATCTTTTGTATATCTAAGACTCA
TCCTGATTTTTACTATCACACATGAATAAAGCCTTTGTATCTT
T

Accordingly, preferably SEPP1 comprises or consists of a nucleotide sequence substantially as set out in SEQ ID NO: 21, or a fragment or variant thereof.

In one embodiment, TFF3 is provided by gene bank locus ID: HGNC: 11757; Entrez Gene: 7033; Ensembl: ENSG00000160180; OMIM: 600633; and/or UniProtKB: Q07654. The protein sequence may be represented by the GeneBank ID 007654, which is provided herein as SEQ ID No: 22, as follows:

[SEQ ID No: 22]
MKRVLSCVPEPTVVMAARALCMLGLVLALLSSSSAEEYVGLSANQ
CAVPAKDRVDCGYPHVTPKECNNRGCCFDSRIPGVPWCFKPLQEA
ECTF

Accordingly, preferably TFF3 comprises or consists of an amino acid sequence substantially as set out in SEQ ID NO: 22, or a fragment or variant thereof.

In one embodiment TFF3 is encoded by a nucleotide sequence which is provided herein as SEQ ID No: 23, as follows:

[SEQ ID No: 23]
CGCTCCCCAGTAGAGGACCCGGAACCAGAACTGGAATCCGCCCTT
ACCGCTTGCTGCCAAAACAGTGGGGGCTGAACTGACCTCTCCCCT
TTGGGAGAGAAAAACTGTCTGGGAGCTTGACAAAGGCATGCAGGA
GAGAACAGGAGCAGCCACAGCCAGGAGGGAGAGCCTTCCCCAAGC
AAACAATCCAGAGCAGCTGTGCAAACAACGGTGCATAAATGAGGC
CTCCTGGACCATGAAGCGAGTCCTGAGCTGCGTCCCGGAGCCCAC
GGTGGTCATGGCTGCCAGAGCGCTCTGCATGCTGGGGCTGGTCCT
GGCCTTGCTGTCCTCCAGCTCTGCTGAGGAGTACGTGGGCCTGTC
TGCAAACCAGTGTGCCGTGCCAGCCAAGGACAGGGTGGACTGCGG
CTACCCCCATGTCACCCCCAAGGAGTGCAACAACCGGGGCTGCTG
CTTTGACTCCAGGATCCCTGGAGTGCCTTGGTGTTTCAAGCCCCT
GCAGGAAGCAGAATGCACCTTCTGAGGCACCTCCAGCTGCCCCCG
GCCGGGGGATGCGAGGCTCGGAGCACCCTTGCCCGGCTGTGATTG
CTGCCAGGCACTGTTCATCTCAGCTTTTCTGTCCCTTTGCTCCCG
GCAAGCGCTTCTGCTGAAAGTTCATATCTGGAGCCTGATGTCTTA
ACGAATAAAGGTCCCATGCTCCACCCGAGGACAGTTCTTCGTGCC
TGAGACTTTCTGAGGTTGTGCTTTATTTCTGCTGCGTCGTGGGAG
AGGGCGGGAGGGTGTCAGGGGAGAGTCTGCCCAGGCCTCAAGGGC
AGGAAAAGACTCCCTAAGGAGCTGCAGTGCATGCAAGGATATTTT
GAATCCAGACTGGCACCCACGTCACAGGAAAGCCTAGGAACACTG
TAAGTGCCGCTTCCTCGGGAAAGCAGAAAAAATACATTTCAGGTA
GAAGTTTTCAAAAATCACAAGTCTTTCTTGGTGAAGACAGCAAGC
CAATAAAACTGTCTTCCAAAGTGGTCCTTTATTTCACAACCACTC
TCGCTACTGTTCAATACTTGTACTATTCCTGGGTTTTGTTTCTTT
GTACAGTAAACATTATGAACAAACAGGCA

Accordingly, preferably TFF3 comprises or consists of a nucleotide sequence substantially as set out in SEQ ID NO: 23, or a fragment or variant thereof.

In one embodiment, IL6 is provided by gene bank locus ID; HGNC: 6018; Entrez Gene: 3569; Ensembl: ENSG00000136244; OMIM: 147620; and/or UniProtKB: P05231. The protein sequence may be represented by the GeneBank ID P05231, which is provided herein as SEQ ID No: 24, as follows:

[SEQ ID No: 24]
MNSFSTSAFGPVAFSLGLLLVLPAAFPAPVPPGEDSKDVAAPHRQ
PLTSSERIDKQIRYILDGISALRKETCNKSNMCESSKEALAENNL
NLPKMAEKDGCFQSGFNEETCLVKIITGLLEFEVYLEYLQNRFES
SEEQARAVQMSTKVLIQFLQKKAKNLDAITTPDPTTNASLLTKLQ
AQNQWLQDMTTHLILRSFKEFLQSSLRALRQM

Accordingly, preferably IL6 comprises or consists of an amino acid sequence substantially as set out in SEQ ID NO: 24 or a fragment or variant thereof.

In one embodiment IL6 is encoded by a nucleotide sequence which is provided herein as SEQ ID No: 25, as follows:

[SEQ ID No: 25]
AGGTAACACCATGTTTGGTAAATAAGTGTTTTGGTGTTGTGCAAG
GGTCTGGTTTCAGCCTGAAGCCATCTCAGAGCTGTCTGGGTCTCT
GGAGACTGGAGGGACAACCTAGTCTAGAGCCCATTTGCATGAGAC
CAAGGATCCTCCTGCAAGAGACACCATCCTGAGGGAAGAGGGCTT
CTGAACCAGCTTGACCCAATAAGAAATTCTTGGGTGCCGACGCGG
AAGCAGATTCAGAGCCTAGAGCCGTGCCTGCGTCCGTAGTTTCCT
TCTAGCTTCTTTTGATTTCAAATCAAGACTTACAGGGAGAGGGAG
CGATAAACACAAACTCTGCAAGATGCCACAAGGTCCTCCTTTGAC
ATCCCCAACAAAGAGGACTGGAGATGTCTGAGGCTCATTCTGCCC
TCGAGCCCACCGGGAACGAAAGAGAAGCTCTATCTCCCCTCCAGG
AGCCCAGCTATGAACTCCTTCTCCACAAGCGCCTTCGGTCCAGTT
GCCTTCTCCCTGGGGCTGCTCCTGGTGTTGCCTGCTGCCTTCCCT
GCCCCAGTACCCCCAGGAGAAGATTCCAAAGATGTAGCCGCCCCA
CACAGACAGCCACTCACCTCTTCAGAACGAATTGACAAACAAATT
CGGTACATCCTCGACGGCATCTCAGCCCTGAGAAAGGAGACATGT
AACAAGAGTAACATGTGTGAAAGCAGCAAAGAGGCACTGGCAGAA
AACAACCTGAACCTTCCAAAGATGGCTGAAAAAGATGGATGCTTC
CAATCTGGATTCAATGAGGAGACTTGCCTGGTGAAAATCATCACT
GGTCTTTTGGAGTTTGAGGTATACCTAGAGTACCTCCAGAACAGA
TTTGAGAGTAGTGAGGAACAAGCCAGAGCTGTGCAGATGAGTACA
AAAGTCCTGATCCAGTTCCTGCAGAAAAAGGCAAAGAATCTAGAT
GCAATAACCACCCCTGACCCAACCACAAATGCCAGCCTGCTGACG
AAGCTGCAGGCACAGAACCAGTGGCTGCAGGACATGACAACTCAT
CTCATTCTGCGCAGCTTTAAGGAGTTCCTGCAGTCCAGCCTGAGG
GCTCTTCGGCAAATGTAGCATGGGCACCTCAGATTGTTGTTGTTA
ATGGGCATTCCTTCTTCTGGTCAGAAACCTGTCCACTGGGCACAG
AACTTATGTTGTTCTCTATGGAGAACTAAAAGTATGAGCGTTAGG
ACACTATTTTAATTATTTTTAATTTATTAATATTTAAATATGTGA
AGCTGAGTTAATTTATGTAAGTCATATTTATATTTTTAAGAAGTA
CCACTTGAAACATTTTATGTATTAGTTTTGAAATAATAATGGAAA
GTGGCTATGCAGTTTGAATATCCTTTGTTTCAGAGCCAGATCATT
TCTTGGAAAGTGTAGGCTTACCTCAAATAAATGGCTAACTTATAC
ATATTTTTAAAGAAATATTTATATTGTATTTATATAATGTATAAA
TGGTTTTTATACCAATAAATGGCATTTTAAAAAATTCAGCA

Accordingly, preferably IL6 comprises or consists of a nucleotide sequence substantially as set out in SEQ ID NO: 25, or a fragment or variant thereof.

In one embodiment, CHI3L1 is provided by gene bank locus ID; HGNC: 1932; Entrez Gene: 1116; Ensembl: ENSG00000133048; OMIM: 601525; and/or UniProtKB: P36222. The protein sequence may be represented by the GeneBank ID P36222, which is provided herein as SEQ ID No: 26 as follows:

[SEQ ID No: 26]
MGVKASQTGFVVLVLLQCCSAYKLVCYYTSWSQYREGDGSCFPDA
LDRFLCTHIIYSFANISNDHIDTWEWNDVTLYGMLNTLKNRNPNL
KTLLSVGGWNFGSQRFSKIASNTQSRRTFIKSVPPFLRTHGFDGL
DLAWLYPGRRDKQHFTTLIKEMKAEFIKEAQPGKKQLLLSAALSA
GKVTIDSSYDIAKISQHLDFISIMTYDFHGAWRGTTGHHSPLFRG
QEDASPDRFSNTDYAVGYMLRLGAPASKLVMGIPTFGRSFTLASS
ETGVGAPISGPGIPGRFTKEAGTLAYYEICDFLRGATVHRILGQQ
VPYATKGNQWVGYDDQESVKSKVQYLKDRQLAGAMVWALDLDDFQ
GSFCGQDLRFPLTNAIKDALAAT

Accordingly, preferably CHI3L1 comprises or consist of an amino acid sequence substantially as set out in SEQ ID NO: 26, or a fragment or variant thereof.

In one embodiment CHI3L1 is encoded by a nucleotide sequence which is provided herein as SEQ ID No: 27, as follows:

[SEQ ID No: 27]
GAGGCCCTGTCTAGGTAGCTGGCACCAGGAGCCGTGGGCAAGGGA
AGAGGCCACACCCTGCCCTGCTCTGCTGCAGCCAGAATGGGTGTG
AAGGCGTCTCAAACAGGCTTTGTGGTCCTGGTGCTGCTCCAGTGC
TGCTCTGCATACAAACTGGTCTGCTACTACACCAGCTGGTCCCAG
TACCGGGAAGGCGATGGGAGCTGCTTCCCAGATGCCCTTGACCGC
TTCCTCTGTACCCACATCATCTACAGCTTTGCCAATATAAGCAAC
GATCACATCGACACCTGGGAGTGGAATGATGTGACGCTCTACGGC
ATGCTCAACACACTCAAGAACAGGAACCCCAACCTGAAGACTCTC
TTGTCTGTCGGAGGATGGAACTTTGGGTCTCAAAGATTTTCCAAG
ATAGCCTCCAACACCCAGAGTCGCCGGACTTTCATCAAGTCAGTA
CCGCCATTTCTGCGCACCCATGGCTTTGATGGGCTGGACCTTGCC
TGGCTCTACCCTGGACGGAGAGACAAACAGCATTTTACCACCCTA
ATCAAGGAAATGAAGGCCGAATTTATAAAGGAAGCCCAGCCAGGG
AAAAAGCAGCTCCTGCTCAGCGCAGCACTGTCTGCGGGGAAGGTC
ACCATTGACAGCAGCTATGACATTGCCAAGATATCCCAACACCTG
GATTTCATTAGCATCATGACCTACGATTTTCATGGAGCCTGGCGT
GGGACCACAGGCCATCACAGTCCCCTGTTCCGAGGTCAGGAGGAT
GCAAGTCCTGACAGATTCAGCAACACTGACTATGTGTGGGGTACA
TGTTGAGGCTGGGGGCTCCTGCCAGTAAGCTGGTGATGGGCATCC
CCACCTTCGGGAGGAGCTTCACTCTGGCTTCTTCTGAGACTGGTG
TTGGAGCCCCAATCTCAGGACCGGGAATTCCAGGCCGGTTCACCA
AGGAGGCAGGGACCCTTGCCTACTATGAGATCTGTGACTTCCTCC
GCGGAGCCACAGTCCATAGAATCCTCGGCCAGCAGGTCCCCTATG
CCACCAAGGGCAACCAGTGGGTAGGATACGACGACCAGGAAAGCG
TCAAAAGCAAGGTGCAGTACCTGAAGGACAGGCAGCTGGCGGGCG
CCATGGTATGGGCCCTGGACCTGGATGACTTCCAGGGCTCCTTCT
GTGGCCAGGATCTGCGCTTCCCTCTCACCAATGCCATCAAGGATG
CACTCGCTGCAACGTAGCCCTCTGTTCTGCACACAGCACGGGGGC
CAAGGATGCCCCGTCCCCCTCTGGCTCCAGCTGGCCGGGAGCCTG
ATCACCTGCCCTGCTGAGTCCCAGGCTGAGCCTCAGTCTCCCTCC
CTTGGGGCCTATGCAGAGGTCCACAACACACAGATTTGAGCTCAG
CCCTGGTGGGCAGAGAGGTAGGGATGGGGCTGTGGGGATAGTGAG
GCATCGCAATGTAAGACTCGGGATTAGTACACACTTGTTGATTAA
TGGAAATGTTTACAGATCCCCAAGCCTGGCAAGGGAATTTCTTCA
ACTCCCTGCCCCCCAGCCCTCCTTATCAAAGGACACCATTTTGGC
AAGCTCTATCACCAAGGAGCCAAACATCCTACAAGACACAGTGAC
CATACTAATTATACCCCCTGCAAAGCCCAGCTTGAAACCTTCACT
TAGGAACGTAATCGTGTCCCCTATCCTACTTCCCCTTCCTAATTC
CACAGCTGCTCAATAAAGTACAAGAGCTTAACAGTGGTATCTGGG
CTAGCCAAGGTTAATCCATCAGAGTTGTGGGTTTTCAGGCCCAGA
CAGCCCGCAGAGCCATCTGCCTGCTGGGTGAGGGACTAAGGGAGT
GGGCAGAGGGGGAGGAGAAGCAGAGCCAGGGGAGGGACTGAGGCT
GCAACCAGGAGGTGGGGGTGGGGGAGGTGGGTCTCAGTTGCTTGG
GGGAGGGAGCAGGGCGGAAGGGCAGGATGCACTTGCAGGGGTCTC
ATCCTGGATTTCTCTTCAGGTGAGTAATCCCTCCACCTCCACTTT
TAAGTCCAGAGGCGTGGCGAGGGCACAGGGCAGGTGTGGAGGAGG
TCTTAGCTCCAAGGGAACACTTTGCCAGGTTCTTCTGTGCTTCCA
ATGACTTCGAAATAGTCACGTTTGCTAAACTGGAGTGAGGAGTAT
ATGGATGTTTATTTTGCTATACTCTTTGGTATATGTGAAAATTCT
CAAAATAAGAAACTTTAAAAGGTCCTGGTTCAGTGAAGGCTTTGA
CTAACAGCCCCTGGGAGCCGCTAGTACAGTTGCCCAGTAGCTGCT
TGGATGAGTGATGCCCACATTGTTTGCAGCAGGAGAAACAGAATT
AGAGGCGAGGAAGGATTTCCTGGTCCATTGTGATATTGTGCCCCA
GCCTGGGGGACATGCCGAGGGAGCACAGGACTGCCATTCCGGGTG
GGTTACAAGTTAGAGGCACTCTCACCAGGAGGCAGAGAAAGGTGG
GCCAGAGTCCCTCATGGATGAAGGACCTCACTGCAGGTCACTAAA
GGTGCCAGCCTGAAAGCCAGGGCCATCTGTGACACAGGCTATCTT
GGGCCTTCCCCTCCCACAAAGCCACTGCTTCCAGGAAGCCTGCTG
TGCTGGCACCAAGTCCCATTTCATCCTTATTCCCCAGCTCCTACC
CTTCCCCCACACAGTGCCTGTAGCGTATTCATCCCCTGCATTGGA
TQTTTTCTAAGCATTTTCAGAATAGGCCTTATTTTCACCAGTCAG
GGAACTCCCCAGAGGCAGGGGGACCTGAACCAGCTCCTTTCATTT
AAGAATTCTGACTTTGCTCACAATCTGCACCCCACTCCTCCCTAC
CCAACCCCACACCCCATTGGTGCTCAGCTCTGGGCTCAGCCTGAG
GTCTCTTGCCGAATCCTGGCCCGGGCCCCATCCCAGACTTCTCCT
TCAGGTTGGGCCAGGGCTGGCCAGGAGAGGAGGGACCAGCTGGGC
TGGTCCCAGGGCAGGGCAACCCCACCCAGGAGGAAGGGCGAGGCC
CCACCCTGCTCACCTATGTTTGGAGAGTTAGAGGGGCTTGGCTTA
AATGGGTGGAGGGGATGACTGTAGGTGCTGGGCAATATTTGGGGT
GATGAGAGACTGTAGACGGAAGCGCAGCTGAGCCCCTCTGGGAGA
GGGGAACCCTCTCCTGCCCCTAACGTGCTCTCCAGGGAGCATGGA
AAATGAGCAAGACTCTACTTTGCCATACTCTGTCTTCTCCACTGG
GGAAAACAAAAATGGGCAGCAGACCTCAGAGTAACTCCCATGAAG
CCTGTGACCCCTCAGAGATCCACCACTATGAATCTCTATGGGATT
CCCATGATGTCCTATGGGAGGACATCAGCGGCCTAACCCAGCCTC
TCACCCAAAGCAGGAAACCTTCTATGGCCTCTCAGACATGGGGCC
ACCCAGTGTCATGACAATGTCATTCCACTCCTGCCCTCCCCACCT
CCCTGTGCTCCACAGGTAGGAGCCTCTCCCCAGGGGCAGGACGGC
AGAGGATGATGGCATAGGAGTGAGGAGCTTGGGTCTCCCGCATCC
ACTGTATGGATGTTCCAGGGGCTCCAGACTAGATAGGTACAAGGC
CTACCTGTTTGTCAAGGGCCTGCCTGATGTGTAGCAAAGAAAGCA
GAGCCCAGAGAGAGAGCAGGACTTGCCAAGAGTCACACAGCAAGT
TAACAGTGGATCTCCCAATTCCCTGCCAATGCTCTATTTACTACC
TCACAGGCCCGAAAATATGGGACTTCTGGGGCTACCACCATTAGG
GCTGGAAAGAGAGAGATGGAAACCAATGGGGACATTGAGAAGTGG
GGAGACCCTGGGAGGAGTCTTTGGATTAGGTGGGGTTGGAGCAGG
GCTTGAAATGGGGGTGGTTATGGCCATCGTTGGCACCCATGTGCT
GGGTACTCTTTCTGTGTCCAGCACCCTGTCTCCCCATGATCCCAT
GTTGTCCTCACAACAGCAAGGTGAGCTGTTATATTTGTCCCCATT
TTACAGATGATGAAACTGAGACTCAGGTTATAACCTTTCACAGTG
GCTGGCTAGTAAGTGATAGAGCCAGACTTGGAAACCTGGAATGCC
TGGCTCTAAGGCACATGCATGCCTGGAGGCGACCCCTGTCCCAAT
CATGCCCTCCCAGAGCTGTGTGGCCTCAGGATCCCAGCTCTGCAG
GTCCTGGAAACCCCACCAGAGGCCCAAGGCACCTAGCATATCAGT
GCTGAGCATGCTACAGGGCTGATTTTGGTCCTAGGTGCTGGTCAG
GTCAGGGACAGGGAGGGAGGTGGGCAGGCATGAGGGAATGGGGTG
GGCTAGAAGCCGGCGTCAGCTGCTGTCCTCCTGAGGACAGGTAAA
GAGGGACTTCAGCCTCAGGGCAGTGTCCTGGGACCCTGTGCCCTG
AAGATCTCACATAGCAAGGAAGGCTTCTTGTGACCATGGTGGGAG
GTGGGAATGGGGTTCTAAGAGGTGGAAGGTTGTGACTGAGCAGAG
CACCCACTTATAACTACCCACTTAGTGCATTGCCCATTGCCCACC
CTTCAATCCCATACTGATGCCACCCATACCCAGCATGCACTGTGT
CCAGCAGCTCTCAGCTCTGCCTGGGGGCAGCCTAAGTATCTCCAG
TTACCAGGGGCAGAAGGAGAGTCTAAACAAATTGTTCACAATACC
AGCAGCAATCACTTTAACTTTGGGATTTCATGCACCAGCTAGAAC
AAAGCACAGACCGAAAGGCAAATGTCTCCCAAAGTCATCTGTGGG
CCAATAGGGGTCACCCACTGTCCGATGCTCCCTCCAGGACAGGGA
GTAATTGAGTGCTGATGGGTGTGCATGGGTTTGGGGAGAAGATTA
GTCAGTTTTTTAGGAAAAGGATGGGTGACTTGAGGATGGGACCAC
TGATGAGCCACTTTATCACTTCCACTAGGCCCCAGGCAGGTTGGG
GAGTACAGCAAATGGGTTGCGCAGAGACCTAGTCCGCCCTCGATG
AGTCTACCTCTCATGCCACTTGGGACCCTTTCTCTCACCTGCCTG
TCTCCCATCTAGGTGAGTCTCCTGCCCCAGGCAGCCTCCCCAGAA
CCTCTGCCAACACTCCTGTTTCCCCGCCACCCCCAGCCCTAGCAC
TCCATGTAGACTGAGGACACTGGGCTTCCAGTCCCAGTTCTTCCA
AAGCCTTGATGTGTGACCTTGAGCAAGTCACTTCCCTGCCCTGTG
CCTCAGTTTCCCCATCTGTCAAATGAGGCATAACAATCCTTGCCT
TTCCTTTTTCACCTGGGCTGTTGGCTGAGCAGTTGAGAGAGCTGC
TATGAAAGTATTTTGGAAAGGGAAGTGGAAAAAGCTATTCAAACC
CTCCAGCTATTGCAGAGTATTTTCTCCACACCAGGCAGAGCAGAG
TGCTGGGCCCTGGAGAGGCCACACAGCAGCCTCTTTCTAGAGTGC
TGGGGAGTCCTCAAGGCTCTCTCATACACACAGGCCTCCCCCTTT
CCTGTTGGCCCCACGCCTCATCCTCACCCCACCCCTCATGCCTGT
GAGGAACAAGGAACAGCCAAGCAGCCTCATCTCTCCTGAGAGCAG
AGCCATGGGTCTCGGGAAACCCAGGAGTAAGGAACAAAGACTCTT
CAAAATGACTTCAGAGCTTTCTTTAGGATQCCAGGGAGGTGTAAA
CTCAGTGCTTAATTAAATGGATTCTTTAGAGGGTGGGAAACAGGT
GGATGTCAACCATTTGCCCCACATACCCGTATTCATGCAATCCAC
CCCCAGTGGGCTCACCGGTGCGGGTGTGCAAAACCTCCTCCCACC
CCAGTCATCTTAGGTTCAGGTCATCCTTTGGTCCTGCTCTTTCCC
CGCCAGGCTGTCTGTTGATGCTACTTTTAATCTGCTTTCACTAAG
ATAAGCCTGGCAGAAGTGGTGGGGGTAAGGTGGGCTGTAGGCCAG
CTCCCAAATGTGTGCTGGGCATAACAGAAGACCCATTCTTGACTG
AAGTGCCCTTGTGGGACCCTGAGCCCGTGCCCTGGAGTGGCACAG
GGAGGTGTGCCAGCAATGGGGACCGAGGTCACTGGGGACTGCTGG
GGTTCGGGCTAGTGGCCTGTCTGGCTGCTGTCTGCTTCCCTCGTT
CACATCCTGCTGGAGCCTTAAATAGGAGCCCAAAAGCTTTTCTTT
CTTACTTTTTTTTGAGACAAAATCTCACTCTGTTGCCCAGGCCGG
AGTGCAGTGGCACAACCTCTGCCGCCTGGGTTCAAGCGATTCTCC
TGCCTCAGCCTCCCGAGTAGCTGGGATTACAGGTGCCTGCCACCA
TGCCTGGCTAATTTTCGTAGTTAGAGTAGAGACGGGGTTTCACCA
TCTTGGCCAGGCTGGTCTTGAACTCCTCACCTCATGATCCACCTG
CCTCGGCCTCCCAAAGTGCTGGGATTCCAGGTATGAGCCACCACG
CCCGGCCTAAAGCTTTTCTATTAATAATTTCCTGCCTCACCCTCC
ATCCCCTTCCTCCTCAGGTGAGTTCCCAGAAGGAGGGAGGGCCAG
GAGGGGGCCGCAAGACCTGCCATCTGCCAGTGCTCACACACCAAT
CTCTCTAGCCCTCAGTACAGTCCTGCAAGAGGGGGGTCATGAGGC
CCATTCCACAGATAAGGAAACTGAGGCCCAAAGTCTGGGCATGCT
GCGTTGCTCTGGGAAGGTGATCTGCAGGGTAAATGGAGTGAGGGC
AGGGGGCCGAATGGGGAGAGGCTGGGAGCCGAGGAGGTAGGAGTC
ATTGTGCCCTCAGAGCCAACCACCTGATTTCTGCATCTGTCAAAT
AGTAATAGCCCCTTCCTATGCCTCAAAGGATTTTTTTCAAGAATG
AAACTGTAAAATTCACTTTAAAGTGACATGATCCGTTCCCGGAGG
GACAGGGGAATCCCCAGTGCACCATACACCAATAACCCCTGCTAA
GGCAGCAGTATTAATTGCTCAACCTTCCGCACCTGTGCACTAACT
GCTCACGTTGTTCCCACCCTACCCCACCCAGGTAATGGAGTTGGG
GAGAAGGTAGGATTCCCCCACACCACCAGCAGCCCTGGGGAAGGT
GATTCCCCACCACGTTCTTGCTTTTTCTCCTTTGGGAATAAAGAA
AATGTCCGGTTGCCCCAGCATGCCTGAGGAAGTGGAAGGGAGAGG
TTAGGACATTTGTTGCTGAAAATCTCCAGCAGGTACAGGCACATG
GGCCTGCACCACTAGGCACCTGGGATAGCCCTCTGGCTATGGGGC
TGAGGTCTTCCTTCCAGCCCAGGAAAGAGCAGAGGTCAAGAGGCA
GATTTTTTGTTTCACTCTAGCCTCTGCTACTCTGTGTGGCCTTGG
GCCTGTCCTCAGTGTCAACCAGCAGGCCTCACATCTCTGTTTAAA
TGGAAGAAGCTAAGCAGGGCCAAGGCAGCCACCATCTCTGGGTCA
TTTGCCTCTGGTTTGTATAAACTTGTGTGATGCATGCAGCCTGCA
GACCCTGCAGAGAGTGAGGCTGCAGGATGGAGCAGGAGCTAAAAG
AGATTTGGAGAGTGGCGTCTCCTGGTGACCTGCAAGGTCTCGGCA
CGACTCCCCACACTGCCTTTTCCCTGTTATCTGCTCAGGTAGGTT
TCCCCAAGGCCACACCTCAGGACAAAGAGAAAGAAGGAGGCCCCG
CTCCCAGCAGCCAGATTCCTGTCCCTTGCACTGAGGTCTGGGCTG
GGCTCACAGAGCACATGTGCCCTGTACACACTCTGGGTCAGGGAA
CCCAGCCCTGCTCCTCTGGGCCTCCCCTGCCAGCCCTCTGTTCTG
CACACAGCACGGGGGCCAAGGATGCCCCGTCCCCCTCTGGCTCCA
GCTGGCCGGGAGCCTGATCACCTGCCCTGCTGAGTCCCAGGCTGA
GCCTCAGTCTCCCTCCCTTGGGGCCTATGCAGAGGTCCACAACAC
ACAGATTTGAGCTCAGCCCTGGTGGGCAGAGAGGTAGGGATGGGG
CTGTGGGGATAGTGAGGCATCGCAATGTAAGACTCGGGATTAGTA
CACACTTGTTGATTAATGGAAATGTTTACAGATCCCCAAGCCTGG
CAAGGGAATTTCTTCAACTCCCTGCCCCCCAGCCCTCCTTATCAA
AGGACACCATTTTGGCAAGCTCTATCACCAAGGAGCCAAACATCC
TACAAGACACAGTGACCATACTAATTATACCCCCTGCAAAGCCCA
GCTTGAAACCTTCACTTAGGAACGTAATCGTGTCCCCTATCCTAC
TTCCCCTTCCTAATTCCACAGCTGCTCAATAAAGTACAAGAGCTT
AACAGTGGAGGCCCTGTCTAGGTAGCTGGCACCAGGAGCCGTGGG
CAAGGGAAGAGGCCACACCCTGCCCTGCTCTGCTGCAGCCAGAGA
GGCCCTGTCTAGGTAGCTGGCACCAGGAGCCGTGGGCAAGGGAAG
AGGCCACACCCTGCCCTGCTCTGCTGCAGCCAGAATGGGTGTGAA
GGCGTCTCAAACAGGTATCTGGGCTAGCCAAGGTTAATCCATCAG
AGTTGTGGGTTTTCAGGCCCAGACAGCCCGCAGAGCCATCTGCCT
GCTGGGTGAGGGACTAAGGGAGTGGGCAGAGGGGGAGGAGAAGCA
GAGCCAGGGGAGGGACTGAGGCTGCAACCAGGAGGTGGGGGTGGG
GGAGGTGGGTCTCAGTTGCTTGGGGGAGGGAGCAGGGCGGAAGGG
CAGGATGCACTTGCAGGGGTCTCATCCTGGATTTCTCTTCAGGCT
TTGTGGTCCTGGTGCTGCTCCAGTGCTGTGAGTAATCCCTCCACC
TCCACTTTTAAGTCCAGAGGCGTGGCGAGGGCACAGGGCAGGTGT
GGAGGAGGTCTTAGCTCCAAGGGAACACTTTGCCAGGTTCTTCTG
TGCTTCCAATGACTTCGAAATAGTCACGTTTGCTAAACTGGAGTG
AGGAGTATATGGATGTTTATTTTGCTATACTCTTTGGTATATGTG
AAAATTCTCAAAATAAGAAACTTTAAAAGGTCCTGGTTCAGTGAA
GGCTTTGACTAACAGCCCCTGGGAGCCGCTAGTACAGTTGCCCAG
TAGCTGCTTGGATGAGTGATGCCCACATTGTTTGCAGCAGGAGAA
ACAGAATTAGAGGCGAGGAAGGATTTCCTGGTCCATTGTGATATT
GTGCCCCAGCCTGGGGGACATGCCGAGGGAGCACAGGACTGCCAT
TCCGGGTGGGTTACAAGTTAGAGGCACTCTCACCAGGAGGCAGAG
AAAGGTGGGCCAGAGTCCCTCATGGATGAAGGACCTCACTGCAGG
TCACTAAAGGTGCCAGCCTGAAAGCCAGGGCCATCTGTGACACAG
GCTATCTTGGGCCTTCCCCTCCCACAAAGCCACTGCTTCCAGGAA
GCCTGCTGTGCTGGCACCAAGTCCCATTTCATCCTTATTCCCCAG
CTCCTACCCTTCCCCCACACAGTGCCTGTAGCGTATTCATCCCCT
GCATTGGATQTTTTCTAAGCATTTTCAGAATAGGCCTTATTTTCA
CCAGTCAGGGAACTCCCCAGAGGCAGGGGGACCTGAACCAGCTCC
TTTCATTTAAGAATTCTGACTTTGCTCACAATCTGCACCCCACTC
CTCCCTACCCAACCCCACACCCCATTGGTGCTCAGCTCTGGGCTC
AGCCTGAGGTCTCTTGCCGAATCCTGGCCCGGGCCCCATCCCAGA
CTTCTCCTTCAGGCTCTGCATACAAACTGGTCTGCTACTACACCA
GCTGGTCCCAGTACCGGGAAGGCGATGGGAGCTGCTTCCCAGATG
CCCTTGACCGCTTCCTCTGTACCCACATCATCTACAGCTTTGCCA
ATATAAGCAACGATCACATCGACACCTGGGAGTGGAATGATGTGA
CGCTCTACGGCATGCTCAACACACTCAAGAACAGGTTGGGCCAGG
GCTGGCCAGGAGAGGAGGGACCAGCTGGGCTGGTCCCAGGGCAGG
GCAACCCCACCCAGGAGGAAGGGCGAGGCCCCACCCTGCTCACCT
ATGTTTGGAGAGTTAGAGGGGCTTGGCTTAAATGGGTGGAGGGGA
TGACTGTAGGTGCTGGGCAATATTTGGGGTGATGAGAGACTGTAG
ACGGAAGCGCAGCTGAGCCCCTCTGGGAGAGGGGAACCCTCTCCT
GCCCCTAACGTGCTCTCCAGGGAGCATGGAAAATGAGCAAGACTC
TACTTTGCCATACTCTGTCTTCTCCACTGGGGAAAACAAAAATGG
GCAGCAGACCTCAGAGTAACTCCCATGAAGCCTGTGACCCCTCAG
AGATCCACCACTATGAATCTCTATGGGATTCCCATGATGTCCTAT
GGGAGGACATCAGCGGCCTAACCCAGCCTCTCACCCAAAGCAGGA
AACCTTCTATGGCCTCTCAGACATGGGGCCACCCAGTGTCATGAC
AATGTCATTCCACTCCTGCCCTCCCCACCTCCCTGTGCTCCACAG
GAACCCCAACCTGAAGACTCTCTTGTCTGTCGGAGGATGGAACTT
TGGGTCTCAAAGGTAGGAGCCTCTCCCCAGGGGCAGGACGGCAGA
GGATGATGGCATAGGAGTGAGGAGCTTGGGTCTCCCGCATCCACT
GTATGGATGTTCCAGGGGCTCCAGACTAGATAGGTACAAGGCCTA
CCTGTTTGTCAAGGGCCTGCCTGATGTGTAGCAAAGAAAGCAGAG
CCCAGAGAGAGAGCAGGACTTGCCAAGAGTCACACAGCAAGTTAA
CAGTGGATCTCCCAATTCCCTGCCAATGCTCTATTTACTACCTCA
CAGGCCCGAAAATATGGGACTTCTGGGGCTACCACCATTAGGGCT
GGAAAGAGAGAGATGGAAACCAATGGGGACATTGAGAAGTGGGGA
GACCCTGGGAGGAGTCTTTGGATTAGGTGGGGTTGGAGCAGGGCT
TGAAATGGGGGTGGTTATGGCCATCGTTGGCACCCATGTGCTGGG
TACTCTTTCTGTGTCCAGCACCCTGTCTCCCCATGATCCCATGTT
GTCCTCACAACAGCAAGGTGAGCTGTTATATTTGTCCCCATTTTA
CAGATGATGAAACTGAGACTCAGGTTATAACCTTTCACAGTGGCT
GGCTAGTAAGTGATAGAGCCAGACTTGGAAACCTGGAATGCCTGG
CTCTAAGGCACATGCATGCCTGGAGGCGACCCCTGTQCCAATCAT
GCCCTCCCAGAGCTGTGTGGCCTCAGGATCCCAGCTCTGCAGGTC
CTGGAAACCCCACCAGAGGCCCAAGGCACCTAGCATATCAGTGCT
GAGCATGCTACAGGGCTGATTTTGGTCCTAGATTTTCCAAGATAG
CCTCCAACACCCAGAGTCGCCGGACTTTCATCAAGTCAGTACCGC
CATTTCTGCGCACCCATGGCTTTGATGGGCTGGACCTTGCCTGGC
TCTACCCTGGACGGAGAGACAAACAGCATTTTACCACCCTAATCA
AGGTGCTGGTCAGGTCAGGGACAGGGAGGGAGGTGGGCAGGCATG
AGGGAATGGGGTGGGCTAGAAGCCGGCGTCAGCTGCTGTCCTCCT
GAGGACAGGTAAAGAGGGACTTCAGCCTCAGGGCAGTGTCCTGGG
ACCCTGTGCCCTGAAGATCTCACATAGCAAGGAAGGCTTCTTGTG
ACCATGGTGGGAGGTGGGAATGGGGTTCTAAGAGGTGGAAGGTTG
TGACTGAGCAGAGCACCCACTTATAACTACCCACTTAGTGCATTG
CCCATTGCCCACCCTTCAATCCCATACTGATGCCACCCATACCCA
GCATGCACTGTGTCCAGCAGCTCTCAGCTCTGCCTGGGGGCAGCC
TAAGTATCTCCAGTTACCAGGGGCAGAAGGAGAGTCTAAACAAAT
TGTTCACAATACCAGCAGCAATCACTTTAACTTTGGGATTTCATG
CACCAGCTAGAACAAAGCACAGACCGAAAGGCAAATGTCTCCCAA
AGTCATCTGTGGGCCAATAGGGGTCACCCACTGTCCGATGCTCCC
TCCAGGACAGGGAGTAATTGAGTGCTGATGGGTGTGCATGGGTTT
GGGGAGAAGATTAGTCAGTTTTTTAGGAAAAGGATGGGTGACTTG
AGGATGGGACCACTGATGAGCCACTTTATCACTTCCACTAGGCCC
CAGGCAGGTTGGGGAGTACAGCAAATGGGTTGCGCAGAGACCTAG
TCCGCCCTCGATGAGTCTACCTCTCATGCCACTTGGGACCCTTTC
TCTCACCTGCCTGTCTCCCATCTAGGAAATGAAGGCCGAATTTAT
AAAGGAAGCCCAGCCAGGGAAAAAGCAGCTCCTGCTCAGCGCAGC
ACTGTCTGCGGGGAAGGTCACCATTGACAGCAGCTATGACATTGC
CAAGATATCCCAGTGAGTCTCCTGCCCCAGGCAGCCTCCCCAGAA
CCTCTGCCAACACTCCTGTTTCCCCGCCACCCCCAGCCCTAGCAC
TCCATGTAGACTGAGGACACTGGGCTTCCAGTCCCAGTTCTTCCA
AAGCCTTGATGTGTGACCTTGAGCAAGTCACTTCCCTGCCCTGTG
CCTCAGTTTCCCCATCTGTCAAATGAGGCATAACAATCCTTGCCT
TTCCTTTTTCACCTGGGCTGTTGGCTGAGCAGTTGAGAGAGCTGC
TATGAAAGTATTTTGGAAAGGGAAGTGGAAAAAGCTATTCAAACC
CTCCAGCTATTGCAGAGTATTTTCTCCACACCAGGCAGAGCAGAG
TGCTGGGCCCTGGAGAGGCCACACAGCAGCCTCTTTCTAGAGTGC
TGGGGAGTCCTCAAGGCTCTCTCATACACACAGGCCTCCCCCTTT
CCTGTTGGCCCCACGCCTCATCCTCACCCCACCCCTCATGCCTGT
GAGGAACAAGGAACAGCCAAGCAGCCTCATCTCTCCTGAGAGCAG
AGCCATGGGTCTCGGGAAACCCAGGAGTAAGGAACAAAGACTCTT
CAAAATGACTTCAGAGCTTTCTTTAGGATCCCAGGGAGGTGTAAA
CTCAGTGQTTAATTAAATGGATTCTTTAGAGGGTGGGAAACAGGT
GGATGTCAACCATTTGCCCCACATACCCGTATTCATGCAATCCAC
CCCCAGTGGGCTCACCGGTGCGGGTGTGCAAAACCTCCTCCCACC
CCAGTCATCTTAGGTTCAGGTCATCCTTTGGTCCTGCTCTTTCCC
CGCCAGGCTGTCTGTTGATGCTACTTTTAATCTGCTTTCACTAAG
ATAAGCCTGGCAGAAGTGGTGGGGGTAAGGTGGGCTGTAGGCCAG
CTCCCAAATGTGTGCTGGGCATAACAGAAGACCCATTCTTGACTG
AAGTGCCCTTGTGGGACCCTGAGCCCGTGCCCTGGAGTGGCACAG
GGAGGTGTGCCAGCAATGGGGACCGAGGTCACTGGGGACTGCTGG
GGTTCGGGCTAGTGGCCTGTCTGGCTGCTGTCTGCTTCCCTCGTT
CACATCCTGCTGGAGCCTTAAATAGGAGCCCAAAAGCTTTTCTTT
CTTACTTTTTTTTGAGACAAAATCTCACTCTGTTGCCCAGGCCGG
AGTGCAGTGGCACAACCTCTGCCGCCTGGGTTCAAGCGATTCTCC
TGCCTCAGCCTCCCGAGTAGCTGGGATTACAGGTGCCTGCCACCA
TGCCTGGCTAATTTTCGTAGTTAGAGTAGAGACGGGGTTTCACCA
TCTTGGCCAGGCTGGTCTTGAACTCCTCACCTCATGATCCACCTG
CCTCGGCCTCCCAAAGTGCTGGGATTCCAGGTATGAGCCACCACG
CCCGGCCTAAAGCTTTTCTATTAATAATTTCCTGCCTCACCCTCC
ATCCCCTTCCTCCTCAGACACCTGGATTTCATTAGCATCATGACC
TACGATTTTCATGGAGCCTGGCGTGGGACCACAGGCCATCACAGT
CCCCTGTTCCGAGGTCAGGAGGATGCAAGTCCTGACAGATTCAGC
AACACTGTGAGTTCCCAGAAGGAGGGAGGGCCAGGAGGGGGCCGC
AAGACCTGCCATCTGCCAGTGCTCACACACCAATCTCTCTAGCCC
TCAGTACAGTCCTGCAAGAGGGGGGTCATGAGGCCCATTCCACAG
ATAAGGAAACTGAGGCCCAAAGTCTGGGCATGCTGCGTTGCTCTG
GGAAGGTGATCTGCAGGGTAAATGGAGTGAGGGCAGGGGGCCGAA
TGGGGAGAGGCTGGGAGCCGAGGAGGTAGGAGTCATTGTGCCCTC
AGAGCCAACCACCTGATTTCTGCATCTGTCAAATAGTAATAGCCC
CTTCCTATGCCTCAAAGGATTTTTTTCAAGAATGAAACTGTAAAA
TTCACTTTAAAGTGACATGATCCGTTCCCGGAGGGACAGGGGAAT
CCCCAGTGCACCATACACCAATAACCCCTGCTAAGGCAGCAGTAT
TAATTGCTCAACCTTCCGCACCTGTGCACTAACTGCTCACGTTGT
TCCCACCCTACCCCACCCAGGACTATGCTGTGGGGTACATGTTGA
GGCTGGGGGCTCCTGCCAGTAAGCTGGTGATGGGCATCCCCACCT
TCGGGAGGAGCTTCACTCTGGCTTCTTCTGAGACTGGTGTTGGAG
CCCCAATCTCAGGACCGGGAATTCCAGGCCGGTTCACCAAGGAGG
CAGGGACCCTTGCCTACTATGAGGTAATGGAGTTGGGGAGAAGGT
AGGATTCCCCCACACCACCAGCAGCCCTGGGGAAGGTGATTCCCC
ACCACGTTCTTGCTTTTTCTCCTTTGGGAATAAAGAAAATGTCCG
GTTGCCCCAGCATGCCTGAGGAAGTGGAAGGGAGAGGTTAGGACA
TTTGTTGCTGAAAATCTCCAGCAGGTACAGGCACATGGGCCTGCA
CCACTAGGCACCTGGGATAGCCCTCTGGCTATGGGGCTGAGGTCT
TCCTTCCAGCCCAGGAAAGAGCAGAGGTCAAGAGGCAGATTTTTT
GTTTCACTCTAGCCTCTGCTACTCTGTGTGGCCTTGGGCCTGTCC
TCAGTGTCAACCAGCAGGCCTCACATCTCTGTTTAAATGGAAGAA
GCTAAGCAGGGCCAAGGCAGCCACCATCTCTGGGTCATTTGCCTC
TGGTTTGTATAAACTTGTGTGATGCATGCAGCCTGCAGACCCTGC
AGAGAGTGAGGCTGCAGGATGGAGCAGGAGCTAAAAGAGATTTGG
AGAGTGGCGTCTCCTGGTGACCTGCAAGGTCTCGGCACGACTCCC
CACACTGCCTTTTCCCTGTTATCTGCTCAGATCTGTGACTTCCTC
CGCGGAGCCACAGTCCATAGAATCCTCGGCCAGCAGGTCCCCTAT
GCCACCAAGGGCAACCAGTGGGTAGGATACGACGACCAGGAAAGC
GTCAAAAGCAAGGTAGGTTTCCCCAAGGCCACACCTCAGGACAAA
GAGAAAGAAGGAGGCCCCGCTCCCAGCAGCCAGATTCCTGTCCCT
TGCACTGAGGTCTGGGCTGGGCTCACAGAGCACATGTGCCCTGTA
CACACTCTGGGTCAGGGAACCCAGCCCTGCTCCTCTGGGCCTCCC
CTGCCAGGTGCAGTACCTGAAGGACAGGCAGCTGGCGGGCGCCAT
GGTATGGGCCCTGGACCTGGATGACTTCCAGGGCTCCTTCTGTGG
CCAGGATCTGCGCTTCCCTCTCACCAATGCCATCAAGGATGCACT
CGCTGCAACGTAGCCCTCTGTTCTGCACACAGCACGGGGGCCAAG
GATGCCCCGTCCCCCTCTGGCTCCAGCTGGCCGGGAGCCTGATCA
CCTGCCCTGCTGAGTCCCAGGCTGAGCCTCAGTCTCCCTCCCTTG
GGGCCTATGCAGAGGTCCACAACACACAGATTTGAGCTCAGCCCT
GGTGGGCAGAGAGGTAGGGATGGGGCTGTGGGGATAGTGAGGCAT
CGCAATGTAAGACTCGGGATTAGTACACACTTGTTGATTAATGGA
AATGTTTACAGATCCCCAAGCCTGGCAAGGGAATTTCTTCAACTC
CCTGCCCCCCAGCCCTCCTTATCAAAGGACACCATTTTGGCAAGC
TCTATCACCAAGGAGCCAAACATCCTACAAGACACAGTGACCATA
CTAATTATACCCCCTGCAAAGCCCAGCTTGAAACCTTCACTTAGG
AACGTAATCGTGTCCCCTATCCTACTTCCCCTTCCTAATTCCACA
GCTGCTCAATAAAGTACAAGAGCTTAACAGTG

Accordingly, preferably CHI3L1 comprises or consists of a nucleotide sequence substantially as set out in SEQ ID NO: 27, or a fragment or variant thereof.

In one embodiment, MET is provided by gene bank locus ID: HGNC: 7029; Entrez Gene: 4233; Ensembl: ENSG00000105976; OMIM: 164860; and/or UniProtKB: P08581. The protein sequence may be represented by the GeneBank ID P08581, which is provided herein as SEQ ID No: 28, as follows:

[SEQ ID No: 28]
MKAPAVLAPGILVLLFTLVQRSNGECKEALAKSEMNVNMKYQLPN
FTAETPIQNVILHEHHIFLGATNYIYVLNEEDLQKVAEYKTGPVL
EHPDCFPCQDCSSKANLSGGVWKDNINMALVVDTYYDDQLISCGS
VNRGTCQRHVFPHNHTADIQSEVHCIFSPQIEEPSQCPDCVVSAL
GAKVLSSVKDRFINFFVGNTINSSYFPDHPLHSISVRRLKETKDG
FMFLTDQSYIDVLPEFRDSYPIKYVHAFESNNFIYFLTVQRETLD
AQTFHTRIIRFCSINSGLHSYMEMPLECILTEKRKKRSTKKEVFN
ILQAAYVSKPGAQLARQIGASLNDDILFGVFAQSKPDSAEPMDRS
AMCAFPIKYVNDFFNKIVNKNNVRCLQHFYGPNHEHCFNRTLLRN
SSGCEARRDEYRTEFTTALQRVDLFMGQFSEVLLTSISTFIKGDL
TIANLGTSEGRFMQVVVSRSGPSTPHVNFLLDSHPVSPEVIVEHT
LNQNGYTLVITGKKITKIPLNGLGCRHFQSCSQCLSAPPFVQCGW
CHDKCVRSEECLSGTWTQQICLPAIYKVFPNSAPLEGGTRLTICG
WDFGFRRNNKFDLKKTRVLLGNESCTLTLSESTMNTLKCTVGPAM
NKHFNMSIIISNGHGTTQYSTFSYVDPVITSISPKYGPMAGGTLL
TLTGNYLNSGNSRHISIGGKTCTLKSVSNSILECYTPAQTISTEF
AVKLKIDLANRETSIFSYREDPIVYEIHPTKSFISTWWKEPLNIV
SFLFCFASGGSTITGVGKNLNSVSVPRMVINVHEAGRNFTVACQH
RSNSEIICCTTPSLQQLNLQLPLKTKAFFMLDGILSKYFDLIYVH
NPVFKPFEKPVMISMGNENVLEIKGNDIDPEAVKGEVLKVGNKSC
ENIHLHSEAVLCTVPNDLLKLNSELNIEWKQAISSTVLGKVIVQP
DQNFTGLIAGVVSISTALLLLLGFFLWLKKRKQIKDLGSELVRYD
ARVHTPHLDRLVSARSVSPTTEMVSNESVDYRATFPEDQFPNSSQ
NGSCRQVQYPLTDMSPILTSGDSDISSPLLQNTVHIDLSALNPEL
VQAVQHVVIGPSSLIVHFNEVIGRGHFGCVYHGTLLDNDGKKIHC
AVKSLNRITDIGEVSQFLTEGIIMKDFSHPNVLSLLGICLRSEGS
PLVVLPYMKHGDLRNFIRNETHNPTVKDLIGFGLQVAKGMKYLAS
KKFVHRDLAARNCMLDEKFTVKVADFGLARDMYDKEYYSVHNKTG
AKLPVKWMALESLQTQKFTTKSDVWSFGVLLWELMTRGAPPYPDV
NTFDITVYLLQGRRLLQPEYCPDPLYEVMLKCWHPKAEMRPSFSE
LVSRISAIFSTFIGEHYVHVNATYVNVKCVAPYPSLLSSEDNADD
EVDTRPASFWETS

Accordingly, preferably MET comprises or consist of an amino acid sequence substantially as set out in SEQ ID NO: 28, or a fragment or variant thereof.

In one embodiment MET is encoded by a nucleotide sequence which is provided herein as SEQ ID No: 29, as follows:

[SEQ ID No: 29]
AGACACGTGCTGGGGGGGGCAGGCGAGCGCCTCAGTCTGGTCGCC
TGGCGGTGCCTCCGGCCCCAACGCGCCCGGGCCGCCGCGGGCCGC
GCGCGCCGATGCCCGGCTGAGTCACTGGCAGGGCAGCGCGCGTGT
GGGAAGGGGCGGAGGGAGTGCGGCCGGCGGGGGGGGGGGGCGCTG
GGCTCAGCCCGGCCGCAGGTGACCCGGAGGCCCTCGCCGCCCGCG
GCGCCCCGAGCGCTTTGTGAGCAGATGCGGAGCCGAGTGGAGGGC
GCGAGCCAGATGCGGGGCGACAGCTGACTTGCTGAGAGGAGGCGG
GGAGGCGCGGAGCGCGCGTGTGGTCCTTGCGCCGCTGACTTCTCC
ACTGGTTCCTGGGCACCGAAAGATAAACCTCTCATAATGAAGGCC
CCCGCTGTGCTTGCACCTGGCATCCTCGTGCTCCTGTTTACCTTG
GTGCAGAGGAGCAATGGGGAGTGTAAAGAGGCACTAGCAAAGTCC
GAGATGAATGTGAATATGAAGTATCAGCTTCCCAACTTCACCGCG
GAAACACCCATCCAGAATGTCATTCTACATGAGCATCACATTTTC
CTTGGTGCCACTAACTACATTTATGTTTTAAATGAGGAAGACCTT
CAGAAGGTTGCTGAGTACAAGACTGGGCCTGTGCTGGAACACCCA
GATTGTTTCCCATGTCAGGACTGCAGCAGCAAAGCCAATTTATCA
GGAGGTGTTTGGAAAGATAACATCAACATGGCTCTAGTTGTCGAC
ACCTACTATGATGATCAACTCATTAGCTGTGGCAGCGTCAACAGA
GGGACCTGCCAGCGACATGTCTTTCCCCACAATCATACTGCTGAC
ATACAGTCGGAGGTTCACTGCATATTCTCCCCACAGATAGAAGAG
CCCAGCCAGTGTCCTGACTGTGTGGTGAGCGCCCTGGGAGCCAAA
GTCCTTTCATCTGTAAAGGACCGGTTCATCAACTTCTTTGTAGGC
AATACCATAAATTCTTCTTATTTCCCAGATCATCCATTGCATTCG
ATATCAGTGAGAAGGCTAAAGGAAACGAAAGATGGTTTTATGTTT
TTGACGGACCAGTCCTACATTGATGTTTTACCTGAGTTCAGAGAT
TCTTACCCCATTAAGTATGTCCATGCCTTTGAAAGCAACAATTTT
ATTTACTTCTTGACGGTCCAAAGGGAAACTCTAGATGCTCAGACT
TTTCACACAAGAATAATCAGGTTCTGTTCCATAAACTCTGGATTG
CATTCCTACATGGAAATGCCTCTGGAGTGTATTCTCACAGAAAAG
AGAAAAAAGAGATCCACAAAGAAGGAAGTGTTTAATATACTTCAG
GCTGCGTATGTCAGCAAGCCTGGGGCCCAGCTTGCTAGACAAATA
GGAGCCAGCCTGAATGATGACATTCTTTTCGGGGTGTTCGCACAA
AGCAAGCCAGATTCTGCCGAACCAATGGATCGATCTGCCATGTGT
GCATTCCCTATCAAATATGTCAACGACTTCTTCAACAAGATCGTC
AACAAAAACAATGTGAGATGTCTCCAGCATTTTTACGGACCCAAT
CATGAGCACTGCTTTAATAGGACACTTCTGAGAAATTCATCAGGC
TGTGAAGCGCGCCGTGATGAATATCGAACAGAGTTTACCACAGCT
TTGCAGCGCGTTGACTTATTCATGGGTCAATTCAGCGAAGTCCTC
TTAACATCTATATCCACCTTCATTAAAGGAGACCTCACCATAGCT
AATCTTGGGACATCAGAGGGTCGCTTCATGCAGGTTGTGGTTTCT
CGATCAGGACCATCAACCCCTCATGTGAATTTTCTCCTGGACTCC
CATCCAGTGTCTCCAGAAGTGATTGTGGAGCATACATTAAACCAA
AATGGCTACACACTGGTTATCACTGGGAAGAAGATCACGAAGATC
CCATTGAATGGCTTGGGCTGCAGACATTTCCAGTCCTGCAGTCAA
TGCCTCTCTGCCCCACCCTTTGTTCAGTGTGGCTGGTGCCACGAC
AAATGTGTGCGATCGGAGGAATGCCTGAGCGGGACATGGACTCAA
CAGATCTGTCTGCCTGCAATCTACAAGGTTTTCCCAAATAGTGCA
CCCCTTGAAGGAGGGACAAGGCTGACCATATGTGGCTGGGACTTT
GGATTTCGGAGGAATAATAAATTTGATTTAAAGAAAACTAGAGTT
CTCCTTGGAAATGAGAGCTGCACCTTGACTTTAAGTGAGAGCACG
ATGAATACATTGAAATGCACAGTTGGTCCTGCCATGAATAAGCAT
TTCAATATGTCCATAATTATTTCAAATGGCCACGGGACAACACAA
TACAGTACATTCTCCTATGTGGATCCTGTAATAACAAGTATTTCG
CCGAAATACGGTCCTATGGCTGGTGGCACTTTACTTACTTTAACT
GGAAATTACCTAAACAGTGGGAATTCTAGACACATTTCAATTGGT
GGAAAAACATGTACTTTAAAAAGTGTGTCAAACAGTATTCTTGAA
TGTTATACCCCAGCCCAAACCATTTCAACTGAGTTTGCTGTTAAA
TTGAAAATTGACTTAGCCAACCGAGAGACAAGCATCTTCAGTTAC
CGTGAAGATCCCATTGTCTATGAAATTCATCCAACCAAATCTTTT
ATTAGTACTTGGTGGAAAGAACCTCTCAACATTGTCAGTTTTCTA
TTTTGCTTTGCCAGTGGTGGGAGCACAATAACAGGTGTTGGGAAA
AACCTGAATTCAGTTAGTGTCCCGAGAATGGTCATAAATGTGCAT
GAAGCAGGAAGGAACTTTACAGTGGCATGTCAACATCGCTCTAAT
TCAGAGATAATCTGTTGTACCACTCCTTCCCTGCAACAGCTGAAT
CTGCAACTCCCCCTGAAAACCAAAGCCTTTTTCATGTTAGATGGG
ATCCTTTCCAAATACTTTGATCTCATTTATGTACATAATCCTGTG
TTTAAGCCTTTTGAAAAGCCAGTGATGATCTCAATGGGCAATGAA
AATGTACTGGAAATTAAGGGAAATGATATTGACCCTGAAGCAGTT
AAAGGTGAAGTGTTAAAAGTTGGAAATAAGAGCTGTGAGAATATA
CACTTACATTCTGAAGCCGTTTTATGCACGGTCCCCAATGACCTG
CTGAAATTGAACAGCGAGCTAAATATAGAGTGGAAGCAAGCAATT
TCTTCAACCGTCCTTGGAAAAGTAATAGTTCAACCAGATCAGAAT
TTCACAGGATTGATTGCTGGTGTTGTCTCAATATCAACAGCACTG
TTATTACTACTTGGGTTTTTCCTGTGGCTGAAAAAGAGAAAGCAA
ATTAAAGATCTGGGCAGTGAATTAGTTCGCTACGATGCAAGAGTA
CACACTCCTCATTTGGATAGGCTTGTAAGTGCCCGAAGTGTAAGC
CCAACTACAGAAATGGTTTCAAATGAATCTGTAGACTACCGAGCT
ACTTTTCCAGAAGATCAGTTTCCTAATTCATCTCAGAACGGTTCA
TGCCGACAAGTGCAGTATCCTCTGACAGACATGTCCCCCATCCTA
ACTAGTGGGGACTCTGATATATCCAGTCCATTACTGCAAAATACT
GTCCACATTGACCTCAGTGCTCTAAATCCAGAGCTGGTCCAGGCA
GTGCAGCATGTAGTGATTGGGCCCAGTAGCCTGATTGTGCATTTC
AATGAAGTCATAGGAAGAGGGCATTTTGGTTGTGTATATCATGGG
ACTTTGTTGGACAATGATGGCAAGAAAATTCACTGTGCTGTGAAA
TCCTTGAACAGAATCACTGACATAGGAGAAGTTTCCCAATTTCTG
ACCGAGGGAATCATCATGAAAGATTTTAGTCATCCCAATGTCCTC
TCGCTCCTGGGAATCTGCCTGCGAAGTGAAGGGTCTCCGCTGGTG
GTCCTACCATACATGAAACATGGAGATCTTCGAAATTTCATTCGA
AATGAGACTCATAATCCAACTGTAAAAGATCTTATTGGCTTTGGT
CTTCAAGTAGCCAAAGGCATGAAATATCTTGCAAGCAAAAAGTTT
GTCCACAGAGACTTGGCTGCAAGAAACTGTATGCTGGATGAAAAA
TTCACAGTCAAGGTTGCTGATTTTGGTCTTGCCAGAGACATGTAT
GATAAAGAATACTATAGTGTACACAACAAAACAGGTGCAAAGCTG
CCAGTGAAGTGGATGGCTTTGGAAAGTCTGCAAACTCAAAAGTTT
ACCACCAAGTCAGATGTGTGGTCCTTTGGCGTGCTCCTCTGGGAG
CTGATGACAAGAGGAGCCCCACCTTATCCTGACGTAAACACCTTT
GATATAACTGTTTACTTGTTGCAAGGGAGAAGACTCCTACAACCC
GAATACTGCCCAGACCCCTTATATGAAGTAATGCTAAAATGCTGG
CACCCTAAAGCCGAAATGCGCCCATCCTTTTCTGAACTGGTGTCC
CGGATATCAGCGATCTTCTCTACTTTCATTGGGGAGCACTATGTC
CATGTGAACGCTACTTATGTGAACGTAAAATGTGTCGCTCCGTAT
CCTTCTCTGTTGTCATCAGAAGATAACGCTGATGATGAGGTGGAC
ACACGACCAGCCTCCTTCTGGGAGACATCATAGTGCTAGTACTAT
GTCAAAGCAACAGTCCACACTTTGTCCAATGGTTTTTTCACTGCC
TGACCTTTAAAAGGCCATCGATATTCTTTGCTCTTGCCAAAATTG
CACTATTATAGGACTTGTATTGTTATTTAAATTACTGGATTCTAA
GGAATTTCTTATCTGACAGAGCATCAGAACCAGAGGCTTGGTCCC
ACAGGCCACGGACCAATGGCCTGCAGCCGTGACAACACTCCTGTC
ATATTGGAGTCCAAAACTTGAATTCTGGGTTGAATTTTTTAAAAA
TCAGGTACCACTTGATTTCATATGGGAAATTGAAGCAGGAAATAT
TGAGGGCTTCTTGATCACAGAAAACTCAGAAGAGATAGTAATGCT
CAGGACAGGAGCGGCAGCCCCAGAACAGGCCACTCATTTAGAATT
CTAGTGTTTCAAAACACTTTTGTGTGTTGTATGGTCAATAACATT
TTTCATTACTGATGGTGTCATTCACCCATTAGGTAAACATTCCCT
TTTAAATGTTTGTTTGTTTTTTGAGACAGGATCTCACTCTGTTGC
CAGGGCTGTAGTGCAGTGGTGTGATCATAGCTCACTGCAACCTCC
ACCTCCCAGGCTCAAGCCTCCCGAATAGCTGGGACTACAGGCGCA
CACCACCATCCCCGGCTAATTTTTGTATTTTTTGTAGAGACGGGG
TTTTGCCATGTTGCCAAGGCTGGTTTCAAACTCCTGGACTCAAGA
AATCCACCCACCTCAGCCTCCCAAAGTGCTAGGATTACAGGCATG
AGCCACTGCGCCCAGCCCTTATAAATTTTTGTATAGACATTCCTT
TGGTTGGAAGAATATTTATAGGCAATACAGTCAAAGTTTCAAAAT
AGCATCACACAAAACATGTTTATAAATGAACAGGATGTAATGTAC
ATAGATGACATTAAGAAAATTTGTATGAAATAATTTAGTCATCAT
GAAATATTTAGTTGTCATATAAAAACCCACTGTTTGAGAATGATG
CTACTCTGATCTAATGAATGTGAACATGTAGATGTTTTGTGTGTA
TTTTTTTAAATGAAAACTCAAAATAAGACAAGTAATTTGTTGATA
AATATTTTTAAAGATAACTCAGCATGTTTGTAAAGCAGGATACAT
TTTACTAAAAGGTTCATTGGTTCCAATCACAGCTCATAGGTAGAG
CAAAGAAAGGGTGGATGGATTGAAAAGATTAGCCTCTGTCTCGGT
GGCAGGTTCCCACCTCGCAAGCAATTGGAAACAAAACTTTTGGGG
AGTTTTATTTTGCATTAGGGTGTGTTTTATGTTAAGCAAAACATA
CTTTAGAAACAAATGAAAAAGGCAATTGAAAATCCCAGCTATTTC
ACCTAGATGGAATAGCCACCCTGAGCAGAACTTTGTGATGCTTCA
TTCTGTGGAATTTTGTGCTTGCTACTGTATAGTGCATGTGGTGTA
GGTTACTCTAACTGGTTTTGTCGACGTAAACATTTAAAGTGTTAT
ATTTTTTATAAAAATGTTTATTTTTAATGATATGAGAAAAATTTT
GTTAGGCCACAAAAACACTGCACTGTGAACATTTTAGAAAAGGTA
TGTCAGACTGGGATTAATGACAGCATGATTTTCAATGACTGTAAA
TTGCGATAAGGAAATGTACTGATTGCCAATACACCCCACCCTCAT
TACATCATCAGGACTTGAAGCCAAGGGTTAACCCAGCAAGCTACA
AAGAGGGTGTGTCACACTGAAACTCAATAGTTGAGTTTGGCTGTT
GTTGCAGGAAAATGATTATAACTAAAAGCTCTCTGATAGTGCAGA
GACTTACCAGAAGACACAAGGAATTGTACTGAAGAGCTATTACAA
TCCAAATATTGCCGTTTCATAAATGTAATAAGTAATACTAATTCA
CAGAGTATTGTAAATGGTGGATGACAAAAGAAAATCTGCTCTGTG
GAAAGAAAGAACTGTCTCTACCAGGGTCAAGAGCATGAACGCATC
AATAGAAAGAACTCGGGGAAACATCCCATCAACAGGACTACACAC
TTGTATATACATTCTTGAGAACACTGCAATGTGAAAATCACGTTT
GCTATTTATAAACTTGTCCTTAGATTAATGTGTCTGGACAGATTG
TGGGAGTAAGTGATTCTTCTAAGAATTAGATACTTGTCACTGCCT
ATACCTGCAGCTGAACTGAATGGTACTTCGTATGTTAATAGTTGT
TCTGATAAATCATGCAATTAAAGTAAAGTGATGCAA

Accordingly, preferably MET comprises or consist of a nucleotide sequence substantially as set out in SEQ ID NO: 29, or a fragment or variant thereof.

In one embodiment, GDF15 is provided by gene bank locus ID: HGNC: 30142; Entrez Gene: 9518; Ensembl: ENSG00000130513; OMIM: 605312; and/or UniProtKB: Q99988. The protein sequence may be represented by the GeneBank ID Q99988, which is provided herein as SEQ ID No: 30, as follows:

[SEQ ID No: 30]
MPGQELRTVNGSQMLLVLLVLSWLPHGGALSLAEASRASFPGPSELHSE
DSRFRELRKRYEDLLTRLRANQSWEDSNTDLVPAPAVRILTPEVRLGSG
GHLHLRISRAALPEGLPEASRLHRALFRLSPTASRSWDVTRPLRRQLSL
ARPQAPALHLRLSPPPSQSDQLLAESSSARPQLELHLRPQAARGRRRAR
ARNGDHCPLGPGRCCRLHTVRASLEDLGWADWVLSPREVQVTMCIGACP
SQFRAANMHAQIKTSLHRLKPDTVPAPCCVPASYNPMVLIQKTDTGVSL
QTYDDLLAKDCHCI

Accordingly, preferably GDF15 comprises or consists of an amino acid sequence substantially as set out in SEQ ID NO: 30, or a fragment or variant thereof.

In one embodiment GDF15 is encoded by a nucleotide sequence which is provided herein as SEQ ID No: 31, as follows:

[SEQ ID No: 31]
AGTCCCAGCTCAGAGCCGCAACCTGCACAGCCATGCCCGGGCAAGAACT
CAGGACGGTGAATGGCTCTCAGATGCTCCTGGTGTTGCTGGTGCTCTCGT
GGCTGCCGCATGGGGGCGCCCTGTCTCTGGCCGAGGCGAGCCGCGCAA
GTTTCCCGGGACCCTCAGAGTTGCACTCCGAAGACTCCAGATTCCGAGAG
TTGCGGAAACGCTACGAGGACCTGCTAACCAGGCTGCGGGCCAACCAGA
GCTGGGAAGATTCGAACACCGACCTCGTCCCGGCCCCTGCAGTCCGGAT
ACTCACGCCAGAAGTGCGGCTGGGATCCGGCGGCCACCTGCACCTGCGT
ATCTCTCGGGCCGCCCTTCCCGAGGGGCTCCCCGAGGCCTCCCGCCTTC
ACCGGGCTCTGTTCCGGCTGTCCCCGACGGCGTCAAGGTCGTGGGACGT
GACACGACCGCTGCGGCGTCAGCTCAGCCTTGCAAGACCCCAGGCGCCC
GCGCTGCACCTGCGACTGTCGCCGCCGCCGTCGCAGTCGGACCAACTGC
TGGCAGAATCTTCGTCCGCACGGCCCCAGCTGGAGTTGCACTTGCGGCC
GCAAGCCGCCAGGGGGCGCCGCAGAGCGCGTGCGCGCAACGGGGACCA
CTGTCCGCTCGGGCCCGGGCGTTGCTGCCGTCTGCACACGGTCCGCGCG
TCGCTGGAAGACCTGGGCTGGGCCGATTGGGTGCTGTCGCCACGGGAGG
TGCAAGTGACCATGTGCATCGGCGCGTGCCCGAGCCAGTTCCGGGCGGC
AAACATGCACGCGCAGATCAAGACGAGCCTGCACCGCCTGAAGCCCGAC
ACGGTGCCAGCGCCCTGCTGCGTGCCCGCCAGCTACAATCCCATGGTGC
TCATTCAAAAGACCGACACCGGGGTGTCGCTCCAGACCTATGATGACTTG
TTAGCCAAAGACTGCCACTGCATATGAGCAGTCCTGGTCCTTCCACTGTG
CACCTGCGCGGAGGACGCGACCTCAGTTGTCCTGCCCTGTGGAATGGGCT
CAAGGTTCCTGAGACACCCGATTCCTGCCCAAACAGCTGTATTTATATAA
GTCTGTTATTTATTATTAATTTATTGGGGTGACCTTCTTGGGGACTCGGG
GGCTGGTCTGATGGAACTGTGTATTTATTTAAAACTCTGGTGATAAAAAT
AAAGCTGTCTGAACTGTT

Accordingly, preferably GDF15 comprises or consists of a nucleotide sequence substantially as set out in SEQ ID NO: 31, or a fragment or variant thereof.

In one embodiment, CCL22 is provided by gene bank locus ID: HGNC: 10621; Entrez Gene: 6367; Ensembl: ENSG00000102962; OMIM: 602957; and/or UniProtKB: 000626. The protein sequence may be represented by the GeneBank ID 000626, which is provided herein as SEQ ID No: 32, as follows:

[SEQ ID No: 32]
MDRLQTALLVVLVLLAVALQATEAGPYGANMEDSVCCRDYVRYRLPLRVV
KHFYWTSDSC
PRPGVVLLTFRDKEICADPRVPWVKMILNKLSQ

Accordingly, preferably CCL22 comprises or consists of an amino acid sequence substantially as set out in SEQ ID NO: 32, or a fragment or variant thereof.

In one embodiment CCL22 is encoded by a nucleotide sequence which is provided herein as SEQ ID No: 33, as follows:

[SEQ ID No: 33]
GCAGACACCTGGGCTGAGACATACAGGACAGAGCATGGATCGCCTACAGA
CTGCACTCCTGGTTGTCCTCGTCCTCCTTGCTGTGGCGCTTCAAGCAACT
GAGGCAGGCCCCTACGGCGCCAACATGGAAGACAGCGTCTGCTGCCGTG
ATTACGTCCGTTACCGTCTGCCCCTGCGCGTGGTGAAACACTTCTACTGG
ACCTCAGACTCCTGCCCGAGGCCTGGCGTGGTGTTGCTAACCTTCAGGGA
TAAGGAGATCTGTGCCGATCCCAGAGTGCCCTGGGTGAAGATGATTCTCA
ATAAGCTGAGCCAATGAAGAGCCTACTCTGATGACCGTGGCCTTGGCTCC
TCCAGGAAGGCTCAGGAGCCCTACCTCCCTGCCATTATAGCTGCTCCCCG
CCAGAAGCCTGTGCCAACTCTCTGCATTCCCTGATCTCCATCCCTGTGGC
TGTCACCCTTGGTCACCTCCGTGCTGTCACTGCCATCTCCCCCCTGACCC
CTCTAACCCATCCTCTGCCTCCCTCCCTGCAGTCAGAGGGTCCTGTTCCC
ATCAGCGATTCCCCTGCTTAAACCCTTCCATGACTCCCCACTGCCCTAAG
CTGAGGTCAGTCTCCCAAGCCTGGCATGTGGCCCTCTGGATCTGGGTTCC
ATCTCTGTCTCCAGCCTGCCCACTTCCCTTCATGAATGTTGGGTTCTAGC
TCCCTGTTCTCCAAACCCATACTACACATCCCACTTCTGGGTCTTTGCCT
GGGATGTTGCTGACACCCAGAAAGTCCCACCACCTGCACATGTGTAGCCC
CACCAGCCCTCCAAGGCATTGCTCGCCCAAGCAGCTGGTAATTCCATTTC
ATGTATTAGATGTCCCCTGGCCCTCTGTCCCCTCTTAATAACCCTAGTCA
CAGTCTCCGCAGATTCTTGGGATTTGGGGGTTTTCTCCCCCACCTCTCCA
CTAGTTGGACCAAGGTTTCTAGCTAAGTTACTCTAGTCTCCAAGCCTCTA
GCATAGAGCACTGCAGACAGGCCCTGGCTCAGAATCAGAGCCCAGAAAGT
GGCTGCAGACAAAATCAATAAAACTAATGTCCCTCCCCTCTCCCTGCCAA
AAGGCAGTTACATATCAATACAGAGACTCAAGGTCACTAGAAATGGGCCA
GCTGGGTCAATGTGAAGCCCCAAATTTGCCCAGATTCACCTTTCTTCCCC
CACTCCCTTTTTTTTTTTTTTTTTGAGATGGAGTTTCGCTCTTGTCACCC
ACGCTGGAGTGCAATGGTGTGGTCTTGGCTTATTGAAGCCTCTGCCTCCT
GGGTTCAAGTGATTCTCTTGCCTCAGCCTCCTGAGTAGCTGGGATTACAG
GTTCCTGCTACCACGCCCAGCTAATTTTTGTATTTTTAGTAGAGACGAGG
CTTCACCATGTTGGCCAGGCTGGTCTCGAACTCCTGTCCTCAGGTAATCC
GCCCACCTCAGCCTCCCAAAGTGCTGGGATTACAGGCGTGAGCCACAGTG
CCTGGCCTCTTCCCTCTCCCCACCCCCCCCCCAACTTTTTTTTTTTTTTA
TGGCAGGGTCTCACTCTGTCGCCCAGGCTGGAGTGCAGTGGCGTGATCTC
GGCTCACTACAACCTCGACCTCCTGGGTTCAAGCGATTCTCCCACCCCAG
CCTCCCAAGTAGCTGGGATTACAGGTGTGTGCCACTACGGCTGGCTAATT
TTTGTATTTTTAGTAGAGACAGGTTTCACCATATTGGCCAGGCTGGTCTT
GAACTCCTGACCTCAAGTGATCCACCTTCCTTGTGCTCCCAAAGTGCTGA
GATTACAGGCGTGAGCTATCACACCCAGCCTCCCCCTTTTTTTCCTAATA
GGAGACTCCTGTACCTTTCTTCGTTTTACCTATGTGTCGTGTCTGCTTAC
ATTTCCTTCTCCCCTCAGGCTTTTTTTGGGTGGTCCTCCAACCTCCAATA
CCCAGGCCTGGCCTCTTCAGAGTACCCCCCATTCCACTTTCCCTGCCTCC
TTCCTTAAATAGCTGACAATCAAATTCATGCTATGGTGTGAAAGACTACC
TTTGACTTGGTATTATAAGCTGGAGTTATATATGTATTTGAAAACAGAGT
AAATACTTAAGAGGCCAAATAGATGAATGGAAGAATTTTAGGAACTGTGA
GAGGGGGACAAGGTGGAGCTTTCCTGGCCCTGGGAGGAAGCTGGCTGTGG
TAGCGTAGCGCTCTCTCTCTCTGTCTGTGGCAGGAGGCAAAGAGTAGGGT
GTAATTGAGTGAAGGAATCCTGGGTAGAGACCATTCTCAGGTGGTTGGGC
CAGGCTAAAGACTGGGATTTGGGTCTATCTATGCCTTTCTGGCTGATTTT
TGTAGAGACGGGGTTTTGCCATGTTACCCAGGCTGGTCTCAAACTCCTGG
GCTCAAGCGATCCTCCTGGCTCAGCCTCCCAAAGTGCTGGGATTACAGGC
GTGAGTCACTGCGCCTGGCTTCCTCTTCCTCTTGAGAAATATTCTTTTCA
TACAGCAAGTATGGGACAGCAGTGTCCCAGGTAAAGGACATAAATGTTAC
AAGTGTCTGGTCCTTTCTGAGGGAGGCTGGTGCCGCTCTGCAGGGTATTT
GAACCTGTGGAATTGGAGGAGGCCATTTCACTCCCTGAACCCAGCCTGAC
AAATCACAGTGAGAATGTTCACCTTATAGGCTTGCTGTGGGGCTCAGGTT
GAAAGTGTGGGGAGTGACACTGCCTAGGCATCCAGCTCAGTGTCATCCAG
GGCCTGTGTCCCTCCCGAACCCAGGGTCAACCTGCCTACCACAGGCACTA
GAAGGACGAATCTGCCTACTGCCCATGAACGGGGCCCTCAAGCGTCCTGG
GATCTCCTTCTCCCTCCTGTCCTGTCCTTGCCCCTCAGGACTGCTGGAAA
ATAAATCCTTTAAAATAG

Accordingly, preferably CCL22 comprises or consists of a nucleotide sequence substantially as set out in SEQ ID NO: 33 or a fragment or variant thereof.

In one embodiment, TNFRSF11 is provided by gene bank locus ID: HGNC: 11909; Entrez Gene: 4982; Ensembl: ENSG00000164761; OMIM: 602643; and/or UniProtKB: 000300. The protein sequence may be represented by the GeneBank ID 000300, which is provided herein as SEQ ID No: 34, as follows:

[SEQ ID No: 34]
MNNLLCCALVFLDISIKWTTQETFPPKYLHYDEETSHQLLCDKCPPGTY
LKQHCTAKWKTVCAPCPDHYYTDSWHTSDECLYCSPVCKELQYVKQECNR
THNRVCECKEGRYLEIEFCLKHRSCPPGFGVVQAGTPERNTVCKRCPDGF
FSNETSSKAPCRKHTNCSVFGLLLTQKGNATHDNICSGNSESTQKCGIDV
TLCEEAFFRFAVPTKFTPNWLSVLVDNLPGTKVNAESVERIKRQHSSQEQ
TFQLLKLWKHQNKDQDIVKKIIQDIDLCENSVQRHIGHANLTFEQLRSLM
ESLPGKKVGAEDIEKTIKACKPSDQILKLLSLWRIKNGDQDTLKGLMHAL
KHSKTYHFPKTVTQSLKKTIRFLHSFTMYKLYQKLFLEMIGNQVQSVKIS
CL

Accordingly, preferably TNFRSF11 comprises or consists of an amino acid sequence substantially as set out in SEQ ID NO: 34, or a fragment or variant thereof.

In one embodiment TNFRSF11 is be encoded by a nucleotide sequence which is provided herein as SEQ ID No: 35, as follows:

[SEQ ID No: 35]
GGAGACGCACCGGAGCGCTCGCCCAGCCGCCGCCTCCAAGCCCCTGAG
GTTTCCGGGGACCACAATGAACAACTTGCTGTGCTGCGCGCTCGTGTTTC
TGGACATCTCCATTAAGTGGACCACCCAGGAAACGTTTCCTCCAAAGTAC
CTTCATTATGACGAAGAAACCTCTCATCAGCTGTTGTGTGACAAATGTCC
TCCTGGTACCTACCTAAAACAACACTGTACAGCAAAGTGGAAGACCGTGT
GCGCCCCTTGCCCTGACCACTACTACACAGACAGCTGGCACACCAGTGAC
GAGTGTCTATACTGCAGCCCCGTGTGCAAGGAGCTGCAGTACGTCAAGCA
GGAGTGCAATCGCACCCACAACCGCGTGTGCGAATGCAAGGAAGGGCGCT
ACCTTGAGATAGAGTTCTGCTTGAAACATAGGAGCTGCCCTCCTGGATTT
GGAGTGGTGCAAGCTGGAACCCCAGAGCGAAATACAGTTTGCAAAAGATG
TCCAGATGGGTTCTTCTCAAATGAGACGTCATCTAAAGCACCCTGTAGAA
AACACACAAATTGCAGTGTCTTTGGTCTCCTGCTAACTCAGAAAGGAAAT
GCAACACACGACAACATATGTTCCGGAAACAGTGAATCAACTCAAAAATG
TGGAATAGATGTTACCCTGTGTGAGGAGGCATTCTTCAGGTTTGCTGTTC
CTACAAAGTTTACGCCTAACTGGCTTAGTGTCTTGGTAGACAATTTGCCT
GGCACCAAAGTAAACGCAGAGAGTGTAGAGAGGATAAAACGGCAACACAG
CTCACAAGAACAGACTTTCCAGCTGCTGAAGTTATGGAAACATCAAAACA
AAGACCAAGATATAGTCAAGAAGATCATCCAAGATATTGACCTCTGTGAA
AACAGCGTGCAGCGGCACATTGGACATGCTAACCTCACCTTCGAGCAGCT
TCGTAGCTTGATGGAAAGCTTACCGGGAAAGAAAGTGGGAGCAGAAGACA
TTGAAAAAACAATAAAGGCATGCAAACCCAGTGACCAGATCCTGAAGCTG
CTCAGTTTGTGGCGAATAAAAAATGGCGACCAAGACACCTTGAAGGGCCT
AATGCACGCACTAAAGCACTCAAAGACGTACCACTTTCCCAAAACTGTCA
CTCAGAGTCTAAAGAAGACCATCAGGTTCCTTCACAGCTTCACAATGTAC
AAATTGTATCAGAAGTTATTTTTAGAAATGATAGGTAACCAGGTCCAATC
AGTAAAAATAAGCTGCTTATAACTGGAAATGGCCATTGAGCTGTTTCCTC
ACAATTGGCGAGATCCCATGGATGAGTAAACTGTTTCTCAGGCACTTGAG
GCTTTCAGTGATATCTTTCTCATTACCAGTGACTAATTTTGCCACAGGGT
ACTAAAAGAAACTATGATGTGGAGAAAGGACTAACATCTCCTCCAATAAA
CCCCAAATGGTTAATCCAACTGTCAGATCTGGATCGTTATCTACTGACTA
TATTTTCCCTTATTACTGCTTGCAGTAATTCAACTGGAAATTAAAAAAAA
AAAACTAGACTCCATTGTGCCTTACTAAATATGGGAATGTCTAACTTAAA
TAGCTTTGAGATTTCAGCTATGCTAGAGGCTTTTATTAGAAAGCCATATT
TTTTTCTGTAAAAGTTACTAATATATCTGTAACACTATTACAGTATTGCT
ATTTATATTCATTCAGATATAAGATTTGTACATATTATCATCCTATAAAG
AAACGGTATGACTTAATTTTAGAAAGAAAATTATATTCTGTTTATTATGA
CAAATGAAAGAGAAAATATATATTTTTAATGGAAAGTTTGTAGCATTTTT
CTAATAGGTACTGCCATATTTTTCTGTGTGGAGTATTTTTATAATTTTAT
CTGTATAAGCTGTAATATCATTTTATAGAAAATGCATTATTTAGTCAATT
GTTTAATGTTGGAAAACATATGAAATATAAATTATCTGAATATTAGATGC
TCTGAGAAATTGAATGTACCTTATTTAAAAGATTTTATGGTTTTATAACT
ATATAAATGACATTATTAAAGTTTTCAAATTATTTTTTA

Accordingly, preferably TNFRSF11 comprises or consists of a nucleotide sequence substantially as set out in SEQ ID NO: 35, or a fragment or variant thereof.

In one embodiment, ANGPT2 is provided by gene bank locus ID is HGNC: 485; Entrez Gene: 285; Ensembl: ENSG00000091879; OMIM: 601922; and/or UniProtKB: 015123. The protein sequence may be represented by the GeneBank ID 015123, which is provided herein as SEQ ID No: 36, as follows:

[SEQ ID No: 36]
MWQIVFFTLSCDLVLAAAYNNFRKSMDSIGKKQYQVQHGSCSYTFLLPEM
DNCRSSSSPYVSNAVQRDAPLEYDDSVQRLQVLENIMENNTQWLMKLENY
IQDNMKKEMVEIQQNAVQNQTAVMIEIGTNLLNQTAEQTRKLTDVEAQVL
NQTTRLELQLLEHSLSTNKLEKQILDQTSEINKLQDKNSFLEKKVLAMED
KHIIQLQSIKEEKDQLQVLVSKQNSIIEELEKKIVTATVNNSVLQKQQHD
LMETVNNLLTMMSTSNSAKDPTVAKEEQISFRDCAEVFKSGHTTNGIYTL
TFPNSTEEIKAYCDMEAGGGGWTIIQRREDGSVDFQRTWKEYKVGFGNPS
GEYWLGNEFVSQLTNQQRYVLKIHLKDWEGNEAYSLYEHFYLSSEELNYR
IHLKGLTGTAGKISSISQPGNDFSTKDGDNDKCICKCSQMLTGGWWFDAC
GPSNLNGMYYPQRQNTNKFNGIKWYYWKGSGYSLKATTMMIRPADF

Accordingly, preferably ANGPT2 comprises or consists of an amino acid sequence substantially as set out in SEQ ID NO: 36, or a fragment or variant thereof.

In one embodiment ANGPT2 is encoded by a nucleotide sequence which is provided herein as SEQ ID No: 37, as follows:

[SEQ ID No: 37]
CTGGTTGGAGGGCAGGCATTCTGCTCTGATTTTTCCTGTTGCCTGGCTAG
TGACCCCCTACAGGAAGATAACGGCTAAGCCAGGAGGGCGGAGCAGCCCA
CTACACATGTCTGGCTGCTCTTATCAACTTATCATATAAGGAAAGGAAAG
TGATTGATTCGGATACTGACACTGTAGGATCTGGGGAGAGAGGAACAAAG
GACCGTGAAAGCTGCTCTGTAAAAGCTGACACAGCCCTCCCAAGTGAGCA
GGACTGTTCTTCCCACTGCAATCTGACAGTTTACTGCATGCCTGGAGAGA
ACACAGCAGTAAAAACCAGGTTTGCTACTGGAAAAAGAGGAAAGAGAAGA
CTTTCATTGACGGACCCAGCCATGGCAGCGTAGCAGCCCTGCGTTTTAGA
CGGCAGCAGCTCGGGACTCTGGACGTGTGTTTGCCCTCAAGTTTGCTAAG
CTGCTGGTTTATTACTGAAGAAAGAATGTGGCAGATTGTTTTCTTTACTC
TGAGCTGTGATCTTGTCTTGGCCGCAGCCTATAACAACTTTCGGAAGAGC
ATGGACAGCATAGGAAAGAAGCAATATCAGGTCCAGCATGGGTCCTGCAG
CTACACTTTCCTCCTGCCAGAGATGGACAACTGCCGCTCTTCCTCCAGCC
CCTACGTGTCCAATGTGTGCAGAGGGACGCGCCGCTCGAATACGATGACT
CGGTGCAGAGGCTGCAAGTGCTGGAGAACATCATGGAAAACAACACTCAG
TGGCTAATGAAGCTTGAGAATTATATCCAGGACAACATGAAGAAAGAAAT
GGTAGAGATACAGCAGAATGCAGTACAGAACCAGACGGCTGTGATGATAG
AAATAGGGACAAACCTGTTGAACCAAACAGCGGAGCAAACGCGGAAGTTA
ACTGATGTGGAAGCCCAAGTATTAAATCAGACCACGAGACTTGAACTTCA
GCTCTTGGAACACTCCCTCTCGACAAACAAATTGGAAAAACAGATTTTGG
ACCAGACCAGTGAAATAAACAAATTGCAAGATAAGAACAGTTTCCTAGAA
AAGAAGGTGCTAGCTATGGAAGACAAGCACATCATCCAACTACAGTCAAT
AAAAGAAGAGAAAGATCAGCTACAGGTGTTAGTATCCAAGCAAAATTCCA
TCATTGAAGAACTAGAAAAAAAAATAGTGACTGCCACGGTGAATAATTCA
GTTCTTCAGAAGCAGCAACATGATCTCATGGAGACAGTTAATAACTTACT
GACTATGATGTCCACATCAAACTCAGCTAAGGACCCCACTGTTGCTAAAG
AAGAACAAATCAGCTTCAGAGACTGTGCTGAAGTATTCAAATCAGGACAC
ACCACGAATGGCATCTACACGTTAACATTCCCTAATTCTACAGAAGAGAT
CAAGGCCTACTGTGACATGGAAGCTGGAGGAGGCGGGTGGACAATTATTC
AGCGACGTGAGGATGGCAGCGTTGATTTTCAGAGGACTTGGAAAGAATAT
AAAGTGGGATTTGGTAACCCTTCAGGAGAATATTGGCTGGGAAATGAGTT
TGTTTCGCAACTGACTAATCAGCAACGCTATGTGCTTAAAATACACCTTA
AAGACTGGGAAGGGAATGAGGCTTACTCATTGTATGAACATTTCTATCTC
TCAAGTGAAGAACTCAATTATAGGATTCACCTTAAAGGACTTACAGGGAC
AGCCGGCAAAATAAGCAGCATCAGCCAACCAGGAAATGATTTTAGCACAA
AGGATGGAGACAACGACAAATGTATTTGCAAATGTTCACAAATGCTAACA
GGAGGCTGGTGGTTTGATGCATGTGGTCCTTCCAACTTGAACGGAATGTA
CTATCCACAGAGGCAGAACACAAATAAGTTCAACGGCATTAAATGGTACT
ACTGGAAAGGCTCAGGCTATTCGCTCAAGGCCACAACCATGATGATCCGA
CCAGCAGATTTCTAAACATCCCAGTCCACCTGAGGAACTGTCTCGAACTA
TTTTCAAAGACTTAAGCCCAGTGCACTGAAAGTCACGGCTGCGCACTGTG
TCCTCTTCCACCACAGAGGGCGTGTGCTCGGTGCTGACGGGACCCACATG
CTCCAGATTAGAGCCTGTAAACTTTATCACTTAAACTTGCATCACTTAAC
GGACCAAAGCAAGACCCTAAACATCCATAATTGTGATTAGACAGAACACC
TATGCAAAGATGAACCCGAGGCTGAGAATCAGACTGACAGTTTACAGACG
CTGCTGTCACAACCAAGAATGTTATGTGCAAGTTTATCAGTAAATAACTG
GAAAACAGAACACTTATGTTATACAATACAGATCATCTTGGAACTGCATT
CTTCTGAGCACTGTTTATACACTGTGTAAATACCCATATGTCCTGAATTC
ACCATCACTATCACAATTAAAAGGAAGAAAAAAACTCTCTAAGCCATAAA
AAGACATATTCAGGGATATTCTGAGAAGGGGTTACTAGAAGTTTAATATT
TGGAAAAACAGTTAGTGCATTTTTACTCCATCTCTTAGGTGCTTTAAATT
TTTATTTCAAAAACAGCGTATTTACATTTATGTTGACAGCTTAGTTATAA
GTTAATGCTCAAATACGTATTTCAAATTTATATGGTAGAAACTTCCAGAA
TCTCTGAAATTATCAACAGAAACGTGCCATTTTAGTTTATATGCAGACCG
TACTATTTTTTTCTGCCTGATTGTTAAATATGAAGGTATTTTTAGTAATT
AAATATAACTTATTAGGGGATATGCCTATGTTTAACTTTTATGATAATAT
TTACAATTTTATAATTTGTTTCCAAAAGACCTAATTGTGCCTTGTGATAA
GGAAACTTCTTACTTTTAATGATGAGGAAAATTATACATTTCATTCTATG
ACAAAGAAACTTTACTATCTTCTCACTATTCTAAAACAGAGGTCTGTTTT
CTTTCCTAGTAAGATATATTTTTATAGAACTAGACTACAATTTAATTTCT
GGTTGAGAAAAGCCTTCTATTTAAGAAATTTACAAAGCTATATGTCTCAA
GATTCACCCTTAAATTTACTTAAGGAAAAAAATAATTGACACTAGTAAGT
TTTTTTATGTCAATCAGCAAACTGAAAAAAAAAAAAGGGTTTCAAAGTGC
AAAAACAAAATCTGATGTTCATAATATATTTAAATATTTACCAAAAATTT
GAGAACACAGGGCTGGGCGCAGTGGCTCACACCTATAATCCCAGTACATT
GGTAGGCAAGGTGGGCAGATCACCTGAGGTCAGGAGTTCAAGACCAGCCT
GGACAACATGGTGAAACCCTGTCTCTACTAAATAATACAAAAATTAGCCA
GGCGTGCTGGCGGGCACCTGTAATCCCAGCTACTCGGGAGGCTGAGGCAG
GGAGAATTGCTTGCACCAGGGAGGTAGAGGTTGCAGTGAGCCAAGATCGC
ACCACTGCACTCCAGCCGGGGCAACAGAGCAAGACTCCATCTCAAAAAAA
AAAAAAAAAAAAGAAAGAAAAGAAAATTTGAGAACACAGCTTTATACTCG
GGACTACAAAACCATAAACTCCTGGAGTTTTAACTCCTTTTGAAATTTTC
ATAGTACAATTAATACTAATGAACATTTGTGTAAAGCTTTATAATTTAAA
GGCAATTTCTCATATATTCTTTTCTGAATCATTTGCAAGGAAGTTCAGAG
TCCAGTCTGTAACTAGCATCTACTATATGTCTGTCTTCACCTTACAGTGT
TCTACCATTATTTTTTCTTTATTCCATTTCAAAATCTAATTTATTTTACC
CCAACTTCTCCCCACCACTTGACGTAGTTTTAGAACACACAGGTGTTGCT
ACATATTTGGAGTCAATGATGGACTCTGGCAAAGTCAAGGCTCTGTTTTA
TTTCCACCAAGGTGCACTTTTCCAACAACTATTTAACTAGTTAAGAACCT
CCCTATCTTAGAACTGTATCTACTTTATATTTAAGAAGGTTTTATGAATT
CAACAACGGTATCATGGCCTTGTATCAAGTTGAAAAACAACTGAAAATAA
GAAAATTTCACAGCCTCGAAAGACAACAACAAGTTTCTAGGATATCTCAA
TGACAAGAGTGATGGATACTTAGGTAGGGAAACGCTAATGCAGGAAAAAC
TGGCAACAACACAATTTATATCAATTCTCTTTGTAGGCAGGTGATAAAAA
ATTCAAGGACAAATCTCATTATGTCATTGTGCATCATATATAATCTCTTA
TGAGCGAGAATGGGGGGAATTTGTGTTTTTACTTTACACTTCAATTCCTT
ACACGGTATTTCAAACAAACAGTTTTGCTGAGAGGAGCTTTTGTCTCTCC
TTAAGAAAATGTTTATAAAGCTGAAAGGAAATCAAACAGTAATCTTAAAA
ATGAAAACAAAACAACCCAACAACCTAGATAACTACAGTGATCAGGGAGC
ACAGTTCAACTCCTTGTTATGTTTTAGTCATATGGCCTACTCAAACAGCT
AAATAACAACACCAGTGGCAGATAAAAATCACCATTTATCTTTCAGCTAT
TAATCTTTTGAATGAATAAACTGTGACAAACAAATTAACATTTTTGAACA
TGAAAGGCAACTTCTGCACAATCCTGTATCCAAGCAAACTTTAAATTATC
CACTTAATTATTACTTAATCTTAAAAAAAATTAGAACCCAGAACTTTTCA
ATGAAGCATTTGAAAGTTGAAGTGGAATTTAGGAAAGCCATAAAAATATA
AATACTGTTATCACAGCACCAGCAAGCCATAATCTTTATACCTATCAGTT
CTATTTCTATTAACAGTAAAAACATTAAGCAAGATATAAGACTACCTGCC
CAAGAATTCAGTCTTTTTTCATTTTTGTTTTTCTCAGTTCTGAGGATGTT
AATCGTCAAATTTTCTTTGGACTGCATTCCTCACTACTTTTTGCACAATG
GTCTCACGTTCTCACATTTGTTCTCGCGAATAAATTGATAAAAGGTGTTA
AGTTCTGTGAATGTCTTTTTAATTATGGGCATAATTGTGCTTGACTGGAT
AAAAACTTAAGTCCACCCTTATGTTTATAATAATTTCTTGAGAACAGCAA
ACTGCATTTACCATCGTAAAACAACATCTGACTTACGGGAGCTGCAGGGA
AGTGGTGAGACAGTTCGAACGGCTCCTCAGAAATCCAGTGACCCAATTCT
AAAGACCATAGCACCTGCAAGTGACACAACAAGCAGATTTATTATACATT
TATTAGCCTTAGCAGGCAATAAACCAAGAATCACTTTGAAGACACAGCAA
AAAGTGATACACTCCGCAGATCTGAAATAGATGTGTTCTCAGACAACAAA
GTCCCTTCAGAATCTTCATGTTGCATAAATGTTATGAATATTAATAAAAA
GTTGATTGAGAAAAA

Accordingly, preferably ANGPT2 comprises or consists of a nucleotide sequence substantially as set out in SEQ ID NO: 37, or a fragment or variant thereof.

In one embodiment, ReIA NF-KB is provided by gene bank locus ID: HGNC: 9955; Entrez Gene: 5970; Ensembl: ENSG00000173039; OMIM: 164014; and/or UniProtKB: Q04206. The protein sequence may be represented by the GeneBank ID Q04206, which is provided herein as SEQ ID No: 38, as follows:

[SEQ ID No: 38]
MDELFPLIFPAEPAQASGPYVEIIEQPKQRGMRFRYKCEGRSAGSIPGER
STDTTKTHPTIKINGYTGPGTVRISLVTKDPPHRPHPHELVGKDCRDGFY
EAELCPDRCIHSFQNLGIQCVKKRDLEQAISQRIQTNNNPFQEEQRGDYD
LNAVRLCFQVTVRDPSGRPLRLPPVLSHPIFDNRAPNTAELKICRVNRNS
GSCLGGDEIFLLCDKVQKEDIEVYFTGPGWEARGSFSQADVHRQVAIVFR
TPPYADPSLQAPVRVSMQLRRPSDRELSEPMEFQYLPDTDDRHRIEEKRK
RTYETFKSIMKKSPFSGPTDPRPPPRRIAVPSRSSASVPKPAPQPYPFTS
SLSTINYDEFPTMVFPSGQISQASALAPAPPQVLPQAPAPAPAPAMVSAL
AQAPAPVPVLAPGPPQAVAPPAPKPTQAGEGTLSEALLQLQFDDEDLGAL
LGNSTDPAVFTDLASVDNSEFQQLLNQGIPVAPHTTEPMLMEYPEAITRL
VTGAQRPPDPAPAPLGAPGLPNGLLSGDEDFSSIADMDFSALLSQISS

Accordingly, preferably ReIA NF-KB comprises or consists of an amino acid sequence substantially as set out in SEQ ID NO: 38, or a fragment or variant thereof.

In one embodiment ReIA NF-KB is be encoded by a nucleotide sequence which is provided herein as SEQ ID No: 39, as follows:

[SEQ ID No: 39]
CGTCCTCGGCGAGGCGCGCACTTGGCCCCGACCCCCGGCAGCGGCTGT
GCGTGCAGCCTCTTCGTCCTCCGCGCGGCGTGCACTTGCTCCCGGCCCC
TGCGCCGGGCGGCGGCGGGGCAGCGCGCAGGCGCGGCCGGATTCCGG
GCAGTGACGCGACGGCGGGCCGCGCGGCGCATTTCCGCCTCTGGCGAAT
GGCTCGTCTGTAGTGCACGCCGCGGGCCCAGCTGCGACCCCGGCCCCG
CCCCCGGGACCCCGGCCATGGACGAACTGTTCCCCCTCATCTTCCCGGC
AGAGCCAGCCCAGGCCTCTGGCCCCTATGTGGAGATCATTGAGCAGCCCA
AGCAGCGGGGCATGCGCTTCCGCTACAAGTGCGAGGGGCGCTCCGCGG
GCAGCATCCCAGGCGAGAGGAGCACAGATACCACCAAGACCCACCCCAC
CATCAAGATCAATGGCTACACAGGACCAGGGACAGTGCGCATCTCCCTGG
TCACCAAGGACCCTCCTCACCGGCCTCACCCCCACGAGCTTGTAGGAAAG
GACTGCCGGGATGGCTTCTATGAGGCTGAGCTCTGCCCGGACCGCTGCA
TCCACAGTTTCCAGAACCTGGGAATCCAGTGTGTGAAGAAGCGGGACCTG
GAGCAGGCTATCAGTCAGCGCATCCAGACCAACAACAACCCCTTCCAAGA
AGAGCAGCGTGGGGACTACGACCTGAATGCTGTGCGGCTCTGCTTCCAG
GTGACAGTGCGGGACCCATCAGGCAGGCCCCTCCGCCTGCCGCCTGTCC
TTTCTCATCCCATCTTTGACAATCGTGCCCCCAACACTGCCGAGCTCAAG
ATCTGCCGAGTGAACCGAAACTCTGGCAGCTGCCTCGGTGGGGATGAGAT
CTTCCTACTGTGTGACAAGGTGCAGAAAGAGGACATTGAGGTGTATTTCA
CGGGACCAGGCTGGGAGGCCCGAGGCTCCTTTTCGCAAGCTGATGTGCAC
CGACAAGTGGCCATTGTGTTCCGGACCCCTCCCTACGCAGACCCCAGCCT
GCAGGCTCCTGTGCGTGTCTCCATGCAGCTGCGGCGGCCTTCCGACCGGG
AGCTCAGTGAGCCCATGGAATTCCAGTACCTGCCAGATACAGACGATCGT
CACCGGATTGAGGAGAAACGTAAAAGGACATATGAGACCTTCAAGAGCAT
CATGAAGAAGAGTCCTTTCAGCGGACCCACCGACCCCCGGCCTCCACCTC
GACGCATTGCTGTGCCTTCCCGCAGCTCAGCTTCTGTCCCCAAGCCAGCA
CCCCAGCCCTATCCCTTTACGTCATCCCTGAGCACCATCAACTATGATGA
GTTTCCCACCATGGTGTTTCCTTCTGGGCAGATCAGCCAGGCCTCGGCCT
TGGCCCCGGCCCCTCCCCAAGTCCTGCCCCAGGCTCCAGCCCCTGCCCCT
GCTCCAGCCATGGTATCAGCTCTGGCCCAGGCCCCAGCCCCTGTCCCAGT
CCTAGCCCCAGGCCCTCCTCAGGCTGTGGCCCCACCTGCCCCCAAGCCC
ACCCAGGCTGGGGAAGGAACGCTGTCAGAGGCCCTGCTGCAGCTGCAGT
TTGATGATGAAGACCTGGGGGCCTTGCTTGGCAACAGCACAGACCCAGCT
GTGTTCACAGACCTGGCATCCGTCGACAACTCCGAGTTTCAGCAGCTGCT
GAACCAGGGCATACCTGTGGCCCCCCACACAACTGAGCCCATGCTGATGG
AGTACCCTGAGGCTATAACTCGCCTAGTGACAGGGGCCCAGAGGCCCCC
CGACCCAGCTCCTGCTCCACTGGGGGCCCCGGGGCTCCCCAATGGCCTC
CTTTCAGGAGATGAAGACTTCTCCTCCATTGCGGACATGGACTTCTCAGC
CCTGCTGAGTCAGATCAGCTCCTAAGGGGGTGACGCCTGCCCTCCCCAGA
GCACTGGGTTGCAGGGGATTGAAGCCCTCCAAAAGCACTTACGGATTCTG
GTGGGGTGTGTTCCAACTGCCCCCAACTTTGTGGATGTCTTCCTTGGAGG
GGGGAGCCATATTTTATTCTTTTATTGTCAGTATCTGTATCTCTCTCTCT
TTTTGGAGGTGCTTAAGCAGAAGCATTAACTTCTCTGGAAAGGGGGGAGC
TGGGGAAACTCAAACTTTTCCCCTGTCCTGATGGTCAGCTCCCTTCTCTG
TAGGGAACTCTGGGGTCCCCCATCCCCATCCTCCAGCTTCTGGTACTCTC
CTAGAGACAGAAGCAGGCTGGAGGTAAGGCCTTTGAGCCCACAAAGCCTT
ATCAAGTGTCTTCCATCATGGATTCATTACAGCTTAATCAAAATAACGCC
CCAGATACCAGCCCCTGTATGGCACTGGCATTGTCCCTGTGCCTAACACC
AGCGTTTGAGGGGCTGGCCTTCCTGCCCTACAGAGGTCTCTGCCGGCTCT
TTCCTTGCTCAACCATGGCTGAAGGAAACCAGTGCAACAGCACTGGCTCT
CTCCAGGATCCAGAAGGGGTTTGGTCTGGGACTTCCTTGCTCTCCCTCTT
CTCAAGTGCCTTAATAGTAGGGTAAGTTGTTAAGAGTGGGGGAGAGCAGG
CTGGCAGCTCTCCAGTCAGGAGGCATAGTTTTTACTGAACAATCAAAGCA
CTTGGACTCTTGCTCTTTCTACTCTGAACTAATAAATCTGTTGCCAAGCT
G

Accordingly, preferably ReIA NF-KB comprises or consists of a nucleotide sequence substantially as set out in SEQ ID NO: 39, or a fragment or variant thereof.

In one embodiment, the biomarker is RORA, and a decrease in the expression, amount and/or activity of RORA when compared to a reference is indicative of an individual having a higher risk of suffering from cardiovascular disease.

In one embodiment, the biomarker is GHR, and an increase in the expression, amount and/or activity of GHR when compared to a reference is indicative of an individual having a higher risk of suffering from cardiovascular disease.

Preferably, the sample comprises a biological sample. The sample may be any material that is obtainable from the subject from which protein, RNA and/or DNA is obtainable. Furthermore, the sample may be blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, or combinations thereof.

Preferably, however, the sample comprises blood, urine or tissue.

In one embodiment, the sample comprises a blood sample. The blood may be venous or arterial blood. Blood samples may be assayed immediately.

Alternatively, the blood sample may be stored at low temperatures, for example in a fridge or even frozen before the method is conducted. Detection may be carried out on whole blood. Preferably, however, the blood sample comprises blood serum. Preferably, the blood sample comprises blood plasma.

The blood may be further processed before the method is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting. Alternatively, the blood sample may be centrifuged or filtered to prepare a plasma or serum fraction, which may be used for analysis. Hence, it is preferred that the method is performed in a blood plasma or a blood serum sample. It is preferred that the expression level, amount and/or activity of the biomarker is measured in vitro from a blood serum sample or a plasma sample taken from the individual.

The invention also provides for a kit for determining, diagnosing and/or prognosing CVD risk.

Accordingly, in a second aspect, there is provided a kit for determining, diagnosing and/or prognosing the risk of an individual suffering from cardiovascular disease, the kit comprising:
a. detection means for detecting, in a sample obtained from a test subject, the expression level, amount and/or activity of two or more biomarkers selected from the group consisting of TNF-a; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB; and
b. a reference value from a healthy control population for expression level, amount and/or activity of two or more a biomarkers selected from the group consisting of TNF-a; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB, wherein the kit is used to identify:
   i) a decrease in expression, amount and/or activity of TNF-α; GSTA1; NT-proBNP; RORA and/or TNC when compared to the reference; and/or an increase in expression, amount and/or activity of GHR; A2M; IGFBP2; APOB; SEPP1; TFF3; IL6 and/or CHI3L11; when compared to the reference to determine, diagnose and/or prognose that an individual has a higher risk of suffering from cardiovascular disease; and/or
   ii) a decrease expression, amount and/or activity of MET; GDF15; CCL22; TNFRSF11; ANGPT2 and/or ReIA NF-KB when compared to the reference to determine, diagnose and/or prognose that an individual has a lower risk of suffering from cardiovascular disease.

The cardiovascular disease, the biomarker, detection and the sample may be as defined in the first aspect.

Preferably, a decrease in expression, amount and/or activity of TNF-1, GSTA1, NT-proBNP, RORA and/or TNC, when compared to the reference, is indicative of an individual having a higher risk of suffering from cardiovascular disease or a negative prognosis.

Preferably, an increase in expression, amount and/or activity of GHR, A2M, IGFBP2, APOB, SEPP1, TFF3, IL6 and/or CHI3L1, when compared to the reference, is indicative of an individual having a higher risk of suffering from cardiovascular disease or a negative prognosis.

Preferably, a decrease in expression, amount and/or activity of MET, GDF15, CCL22, TNFRSF11, ANGPT2 and/or ReIA NF-KB, when compared to the reference, is indicative of an individual having a lower risk of suffering from cardiovascular disease or a positive prognosis.

The expression levels, amount and/or activities of the biomarkers may be as defined in the first aspect.

The kit may comprise detection means for detecting the expression levels, amount and/or activities of at least 3 biomarkers or at least 4 biomarkers. The kit may comprise detection means for detecting the expression levels, amount and/or activities of at least 5 biomarkers. The kit may comprise detection means for detecting the expression levels, amount and/or activities of at least 6 biomarkers or at least 7 biomarkers. Alternatively, the kit may comprise detection means for detecting the expression levels, amount and/or activities of at least 8 biomarkers or at least 9 biomarkers. In another embodiment, the kit may comprise detection means for detecting the expression levels, amount and/or activities of at least 10 biomarkers, at least 11 biomarkers, at least 12 biomarkers, at least 13 biomarkers, at least 14 biomarkers or at least 15 biomarkers. In another embodiment, the kit may comprise detection means for detecting the expression levels, amount and/or activities of at least 16 biomarkers, at least 17 biomarkers or at least 18 biomarkers.

Preferably, the kit of the second aspect may be for determining, diagnosing and prognosing the risk of an individual suffering from cardiovascular disease, the kit comprising:
a. detection means for detecting, in a sample obtained from a test subject, the expression level, amount and/or activity of: TNF-a; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB; and
b. a reference value from a healthy control population for expression level, amount and/or activity of TNF-a; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB, wherein the kit is used to identify:
   i) a decrease in expression, amount and/or activity of TNF-a, GSTA1, NT-proBNP, RORA and TNC when compared to the reference; and an increase in expression, amount and/or activity of GHR, A2M, IGFBP2, APOB, SEPP1, TFF3, IL6 and CHI3L1; when compared to the reference to determine, diagnose and/or prognose that an individual has a higher risk of suffering from cardiovascular disease; and
   ii) a decrease expression, amount and/or activity of MET, GDF15, CCL22, TNFRSF11, ANGPT2 and ReIA NF-KB when compared to the reference to determine, diagnose and/or prognose that an individual has a lower risk of suffering from cardiovascular disease.

The detection means may detect the expression level, for example the level or concentration, of a biomarker polynucleotide sequence, for example DNA or RNA. The DNA may be genomic DNA. The RNA may be mRNA. Alternatively, the detection means may detect polypeptide concentration and/or activity of the biomarker.

Accordingly, the detection means may include: sequencing methods (e.g., Sanger, Next Generation Sequencing, RNA-SEQ), hybridization-based methods, including those employed in biochip arrays, mass spectrometry (e.g., laser desorption/ionization mass spectrometry), fluorescence (e.g., sandwich immunoassay), surface plasmon resonance, ellipsometry and atomic force microscopy. Expression levels of markers (e.g., polynucleotides, polypeptides, or other analytes) may be compared by procedures well known in the art, such as RT-PCR, Northern blotting, Western blotting, flow cytometry, immunocytochemistry, binding to magnetic and/or antibody-coated beads, in situ hybridization, fluorescence in situ hybridization (FISH), flow chamber adhesion assay, ELISA, microarray analysis, or colorimetric assays. Methods may further include one or more of electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SFMS), quadrupole time-of-flight (Q-TOF), atmospheric pressure chemical ionization mass spectrometry (APCI-MS), APCI-MS/MS, APCI-(MS)n, atmospheric pressure photoionization mass spectrometry (APPI-MS), APPI-MS/MS, and APPI-(MS)n, quadrupole mass spectrometry, fourier transform mass spectrometry (FTMS), and ion trap mass spectrometry, where n is an integer greater than zero.

Preferably, the kit comprises detection means for detecting RORA present in a sample from a test subject, wherein a decrease in the expression, amount and/or activity of RORA indicative of an individual having a higher risk of suffering from cardiovascular disease.

Preferably, the kit comprises detection means for detecting GHR present in a sample from a test subject, wherein an increase in the expression, amount and/or activity of GHR indicative of an individual having a higher risk of suffering from cardiovascular disease.

Using the methods described herein, the inventors have been able to identify SNPs within RORA and GHR that may be used in diagnosis and prognosis, and in particular gene variants that are associated with CVD risk.

Accordingly, in a third aspect of the invention, there is provided a method of determining, diagnosing and/or prognosing an individual's risk of suffering from cardiovascular disease, the method comprising detecting, in a sample obtained from a individual, a single nucleotide polymorphism (SNP) in the RORA gene, wherein the presence of the SNP is indicative of an individual having an increased risk of suffering from cardiovascular disease.

Preferably, RORA, the sample, detection and the cardiovascular disease is as defined in the first aspect.

The method may be performed in vivo, in vitro or ex vivo. Preferably, the method is performed in vitro or ex vivo. Most preferably, the method is performed in vitro.

Preferably, the SNP is present in a region of chromosome 15, preferably at nucleic acid position 60542728 of the reference sequence NC_000015.10.

Preferably, the SNP comprises a substitution of Adenine (A) to Guanine (G) or Adenine (A) to Cytosine (C).

Preferably, the SNP comprises a substitution of Adenine (A) to Guanine (G) Thus, preferably, the SNP may be referred to by the sequence variant GRCh38.p12 chr 15; NC 000015.10:g.60542728A>G.

Preferably, the SNP comprises a substitution of Adenine (A) to Cytosine (C).

Thus, preferably, the SNP may be referred to by the sequence variant GRCh38.p12 chr 15; NC 000015.10:g.60542728A>C.

Preferably, the SNP may be Reference SNP cluster ID: rs73420079.

Thus, in one embodiment, SNP is present in the sequence represented by Reference SNP cluster ID rs73420079, referred to herein as SEQ ID No: 3, as follows:

[SEQ ID No: 3]
AGGCGCACCT CACACGGCAC ACAGGCACAT CTCACACATG
GCACACATGC ACACCTCACA CAGATGGCAC ACATGCACAC
CTCACACACA CGGCACGCAT GCACACCTCA CACACACGGC
ACGCATGCAC ACCTCACACA CGGCACACAT GCACACCTCA
CACACGACAC ACGGGCACAC CTCACACACA TGGCACACGG
GCACACCTCC CACACACGGC ACACGGGCAC ACCTCCCACA
CACGGCACAC
V GGCACACCTC AAACGACACA CGGCACACC TCACACACAA
GTCTATTCAG CTGCAAGTCC TGCCTCCACT TGCTGAGAAC
CTGCATGACT GGGCACCAAG GATACGGCAC ACACACGCAC
CCACCCCACA TACATACAGT CCACACACAC ACAACACATA
TACACCACAC GCACCACAGA TGCACACCAC ACATGCCACA
CACACATACA CTGCACACGC ACCCTACACA CACCCCCCAC
ATGCTTACAC

Where “V” represent the SNP position. Accordingly, in one embodiment, SNP may comprise or consist of the sequence as substantially set out in SEQ ID No: 3, or a fragment or variant thereof.

Preferably, the SNP comprises a substitution of nucleic acid position X in SEQ D No: 3.

Thus, preferably RORA comprises a single nucleotide polymorphism (SNP), the presence of which is associated with an individual having an increased risk of suffering from CVD.

Preferably, the method of detecting the presence of the SNP comprises a probe that is capable of hybridizing to the biomarker sequence. Preferably, the probe is capable of hybridizing to SEQ ID No 3 such that the SNP is detected.

In a fourth aspect of the invention, there is provided a method of determining, diagnosing and/or prognosing a individual's risk of suffering from cardiovascular disease, the method comprising detecting, in a sample obtained from an individual, a single nucleotide polymorphism (SNP) in the GHR gene, wherein the presence of the SNP is indicative of an individual having an increased risk of suffering from cardiovascular disease.

Preferably, GHR, the sample, detection and the cardiovascular disease is as defined in the first aspect.

Preferably, detecting the SNP in a subject is indicative of an increased risk of suffering from cardiovascular disease.

The method may be performed in vivo, in vitro or ex vivo. Preferably, the method is performed in vitro or ex vivo. Most preferably, the method is performed in vitro.

Preferably, the SNP is present in a region of chromosome 5, preferably at nucleic acid position 42546623 of the reference sequence NC_000005.10.

Preferably, the SNP comprises a substitution of Guanine (G) to Adenine (A).

Preferably, the SNP may be Reference SNP cluster ID rs4314405.

Thus, in one embodiment, SNP is present in the sequence represented by Reference SNP cluster ID: rs73420079, referred to herein as SEQ ID No: 40, as follows:

[SEQ ID No: 40]
AGGCGCACCT CACACGGCAC ACAGGCACAT CTCACACATG
GCACACATGC ACACCTCACA CAGATGGCAC ACATGCACAC
CTCACACACA CGGCACGCAT GCACACCTCA CACACACGGC
ACGCATGCAC ACCTCACACA CGGCACACAT GCACACCTCA
CACACGACAC ACGGGCACAC CTCACACACA TGGCACACGG
GCACACCTCC CACACACGGC ACACGGGCAC ACCTCCCACA
CACGGCACAC
V GGCACACCTC AAACGACACA CGGCACACC TCACACACAA
GTCTATTCAG CTGCAAGTCC TGCCTCCACT TGCTGAGAAC
CTGCATGACT GGGCACCAAG GATACGGCAC ACACACGCAC
CCACCCCACA TACATACAGT CCACACACAC ACAACACATA
TACACCACAC GCACCACAGA TGCACACCAC ACATGCCACA
CACACATACA CTGCACACGC ACCCTACACA CACCCCCCAC
ATGCTTACAC

Where “V” represent the SNP position. Accordingly, in one embodiment, SNP may comprise or consist of the sequence as substantially set out in SEQ ID No: 40, or a fragment or variant thereof.

Preferably, the SNP comprises a substitution of nucleic acid position X in SEQ ID No: 3.

Thus, preferably GHR comprises a single nucleotide polymorphism (SNP), the presence of which is associated with an individual having an increased risk of suffering from CVD.→

In a fifth aspect, there is provided GHR and/or RORA, for use in diagnosis or prognosis.

Preferably, RORA and GHR may comprise a SNP as defined in the third and fourth aspects. The cardiovascular disease may be as defined in the first aspect.

In a sixth aspect, there is provided GHR and/or RORA, for use in diagnosing or prognosing an individual's risk of suffering from cardiovascular disease.

Preferably, RORA and GHR may comprise a SNP as defined in the third and fourth aspects. The cardiovascular disease may be as defined in the first aspect.

Preferably, the method of detecting the presence of the SNP comprises a probe that is capable of hybridizing to the biomarker sequence. Preferably, the probe is capable of hybridizing to SEQ ID No 40 such that the SNP is detected.

In a seventh aspect, there is provided a kit for determining, diagnosing and/or prognosing an individual's risk of suffering from cardiovascular disease, the kit comprising a detection means for detecting, in a sample obtained from a test subject, a single nucleotide polymorphism (SNP) in the RORA gene and/or GHR gene, wherein the presence of the SNP is used to determine, diagnose and/or prognose that an individual has a higher risk of suffering from cardiovascular disease.

The RORA gene, the GHR gene, the sample, detection and the cardiovascular disease may be as defined in the first aspect.

The detection means may be as defined in the second aspect. The single nucleotide polymorphism (SNP) in the RORA gene and/or GHR gene may be as defined in the third and fourth aspects.

In an eighth aspect, there is provided a method of treating an individual having a higher risk of suffering from cardiovascular disease, the method comprising:—
(a) analysing, in a sample obtained from the subject, the expression level, amount and/or activity of two or more biomarkers selected from the group consisting of: TNF-□; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB;
(b) comparing the expression level, amount and/or activity of the biomarker with a reference from a healthy control population, where a decrease in expression, amount and/or activity of TNF-□, GSTA1, NT-proBNP, RORA and/or TNC when compared to the reference and an increase in expression, amount and/or activity of GHR, A2M, IGFBP2, APOB, SEPP1, TFF3, I1L6 and/or CHI3L1 when compared to the reference is suggests that the individual has a higher risk of suffering from cardiovascular disease; and
(c) administering, or having administered, to the individual, a therapeutic agent that prevents, or reduces the likelihood of, the individual suffering from cardiovascular disease.

Preferably, the biomarkers, detection of the biomarkers, the cardiovascular disease, the expression levels, amount and/or activities of the biomarkers and the sample are as defined in the first aspect.

Preferably, the method of treatment comprises analysing and comparing the expression levels, amount and/or activities of 3, 4, 5, 6, 7, 8, 9, 10, 11, 12, 13, 14, 15, 16, 17, 18 or more of the biomarkers: TNF-□; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB.

The method of treatment may comprise analysing and comparing the expression levels, amount and/or activities of at least 6 biomarkers or at least 7 biomarkers. Alternatively, the method of treatment may comprise analysing and comparing the expression levels, amount and/or activities of at least 8 biomarkers or at least 9 biomarkers. In another embodiment, the method of treatment may comprise analysing and comparing the expression levels, amount and/or activities of at least 10 biomarkers, at least 11 biomarkers, at least 12 biomarkers, at least 13 biomarkers, at least 14 biomarkers or at least 15 biomarkers. In another embodiment, the method of treatment may comprise analysing and comparing the expression levels, amount and/or activities of at least 16 biomarkers, at least 17 biomarkers or at least 18 biomarkers.

Preferably, the method of treatment comprises analysing and comparing the expression levels, amount and/or activities of the biomarkers: TNF-□; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB

A clinician would be able to make a decision as to the preferred course of treatment required, for example, the type and dosage of the therapeutic agent according to the eighth and ninth aspects to be administered.

Suitable therapeutic agents may include: statins, including the statins selected from the group consisting of: atorvastatin; simvastatin; rosuvastatin; and pravastatin, beta blockers, blood thinning agents including the blood thinning agents selected from the group consisting of: low-dose aspirin; clopidogrel; rivaroxaban; ticagrelor and prasugrel, nitrates, angiotensin-converting enzyme (ACE) inhibitors, angiotensin II receptor antagonists, calcium channel blockers, including the calcium channel blockers selected from the group consisting of: amlodipine; verapamil and diltiazem and/or diuretics.

Treatment may include enacting lifestyle changes.

In an ninth aspect, there is also provided a method of treating an individual having a higher risk of suffering from cardiovascular disease, the method comprising:—
(a) detecting, in a sample obtained from the subject, a single nucleotide polymorphism (SNP) in the RORA gene and/or the GHR gene, wherein the presence of the SNP suggests that the individual has a higher risk of suffering from cardiovascular disease; and
(b) administering, or having administered, to the individual, a therapeutic agent that prevents, or reduces the likelihood of, the individual suffering from cardiovascular disease.

Preferably, the biomarkers, detection of the biomarkers, the cardiovascular disease, the expression levels, amount and/or activities of the biomarkers and the sample are as defined in the first aspect.

Preferably, the single nucleotide polymorphism (SNP) RORA and/or GHR is as defined in the third and/or fourth aspect.

A clinician would be able to make a decision as to the preferred course of treatment required, for example, the type and dosage of the therapeutic agent according to the eighth and ninth aspects to be administered.

Suitable therapeutic agents may include: statins, including the statins selected from the group consisting of: atorvastatin; simvastatin; rosuvastatin; and pravastatin, beta blockers, blood thinning agents including the blood thinning agents selected from the group consisting of: low-dose aspirin; clopidogrel; rivaroxaban; ticagrelor and prasugrel, nitrates, angiotensin-converting enzyme (ACE) inhibitors, angiotensin II receptor antagonists, calcium channel blockers, including the calcium channel blockers selected from the group consisting of: amlodipine; verapamil and diltiazem and/or diuretics.

Treatment may include enacting lifestyle changes.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1 to 40 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value.

The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 1 to 40 and so on.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

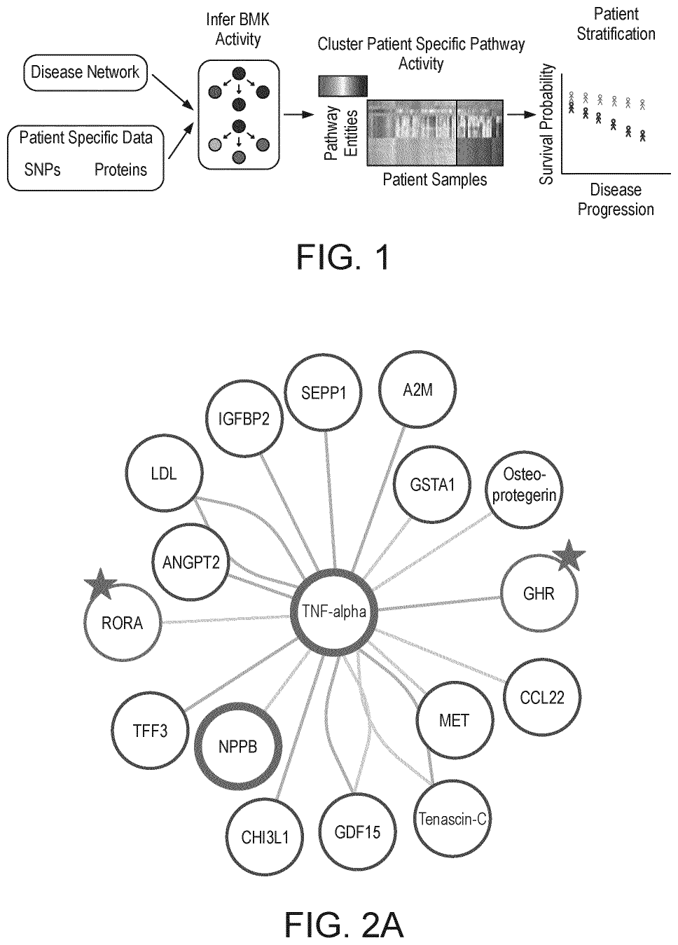

FIG. 1 shows after the disease network identification (upper left panel), single level patient data is integrated based on Bayesian probabilistic graphical models to inferred patient specific pathway activity for each of the BMKs entered in the model. Calculated activities per patient and molecule are them clustered by hierarchical clustering. Survival analysis is done in an independent step to define the relevance of the identified clusters for disease progression (patient stratification). BMKs=biomarkers; TF=transcription factor.

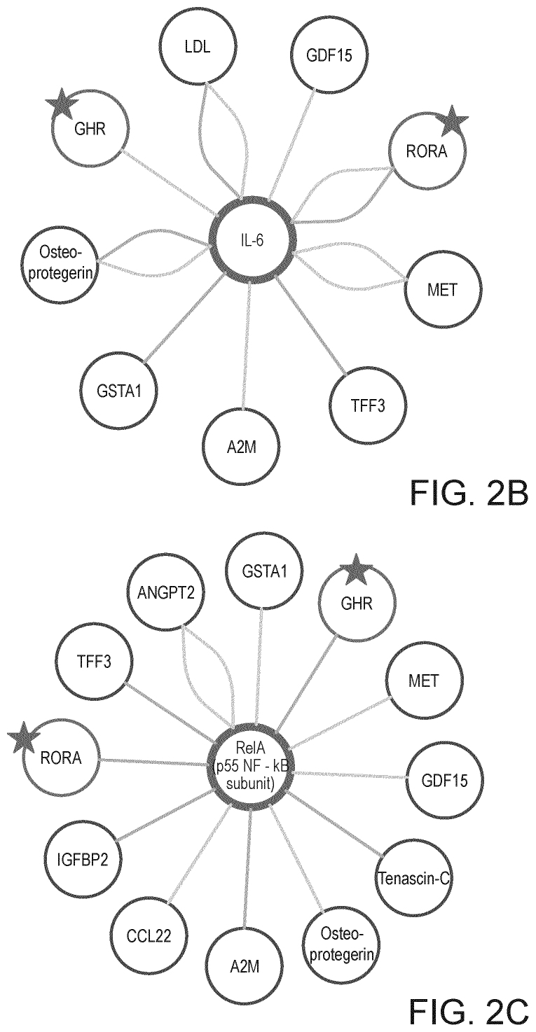

FIG. 2 a-c shows the networks of molecular entities that were prioritized in the overconnectivity analyses. To infer patient specific activity (Workflow FIG. 1), the inventors used patient data from: i) 13 proteins (nodes highlighted in blue), and ii) genotypes of 2 genes (nodes with stars). The activity of 4 nodes (red circles), was inferred by Bayesian statistics based on molecular interactions (connecting edges, red=inhibition, or green=activation, if bi-directional, right edge=from central node to neighbouring molecule, and left edge=from neighbouring molecule to central node) known from the literature (Supplementary Table S5).

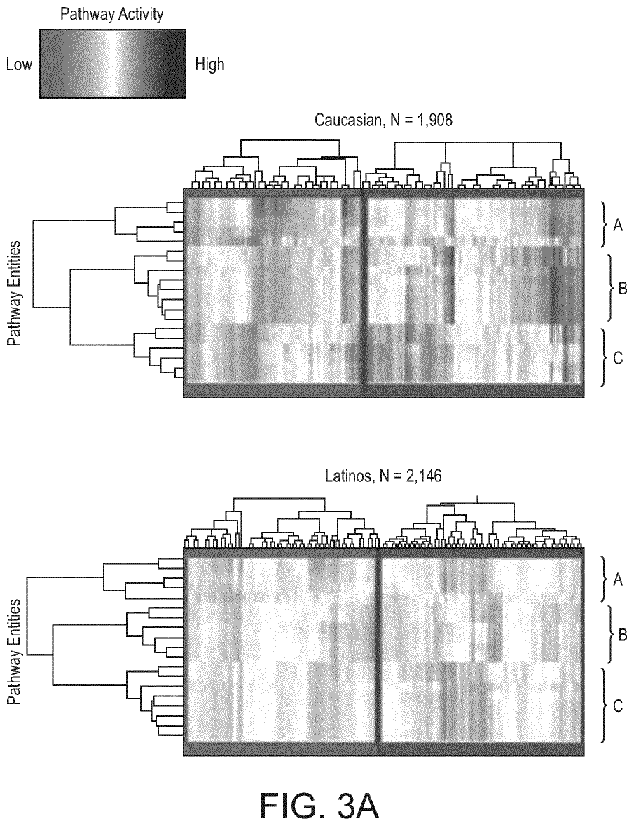

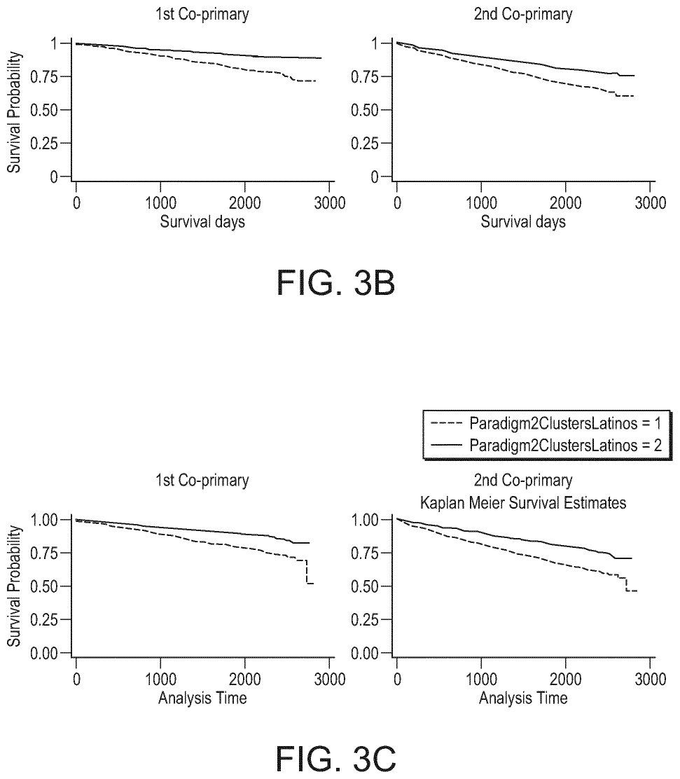

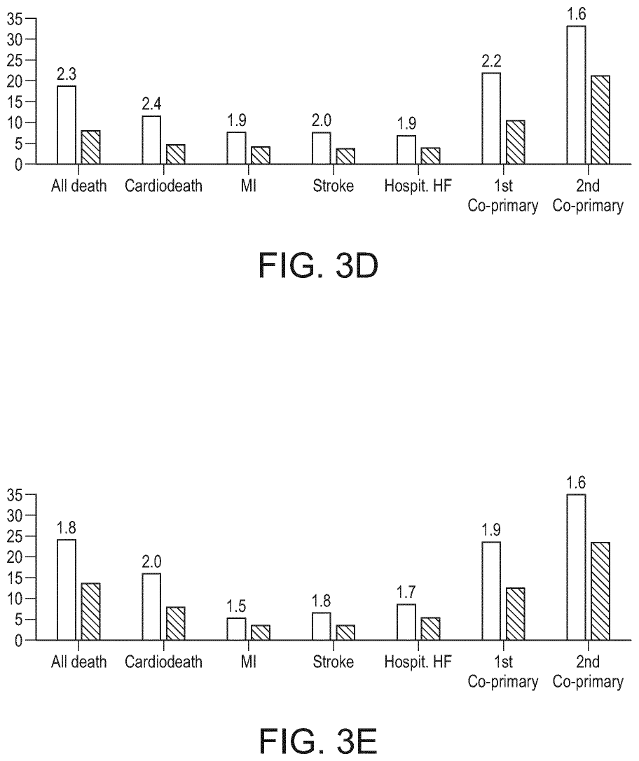

FIG. 3 shows that the molecular signatures of population sub-types relate to CVD progression. a, Two main patient clusters (highlighted in red and blue frames) were identified based on molecular signatures in Caucasians (left) and Latinos (right). b-c, Kaplan-Meier analyses comparing survival probabilities for the identified clusters (a, red and blue) related to 1st and 2^{nd} co-primary expanded CV composites in Caucasians (b) and Latinos (c). d,e, Proportion of CVO events or all death per cluster (blue=higher and blue=lower CVO risk clusters) with the respective calculated relative risk of event for patients in the blue versus red highlighted cluster (a). There were significant differences in survival between clusters (Log-rank Pᵢ 0.0000 in b-c) for each of the outcomes adjusted for CVO risk factors (Cox regression models Pᵢ 0.0000 in b-c). A, C, B represent the main clusters of pathway entities (Table 1). PA=pathway activity; MI=myocardial infarction; Hospit. HF=hospitalization by heart failure.

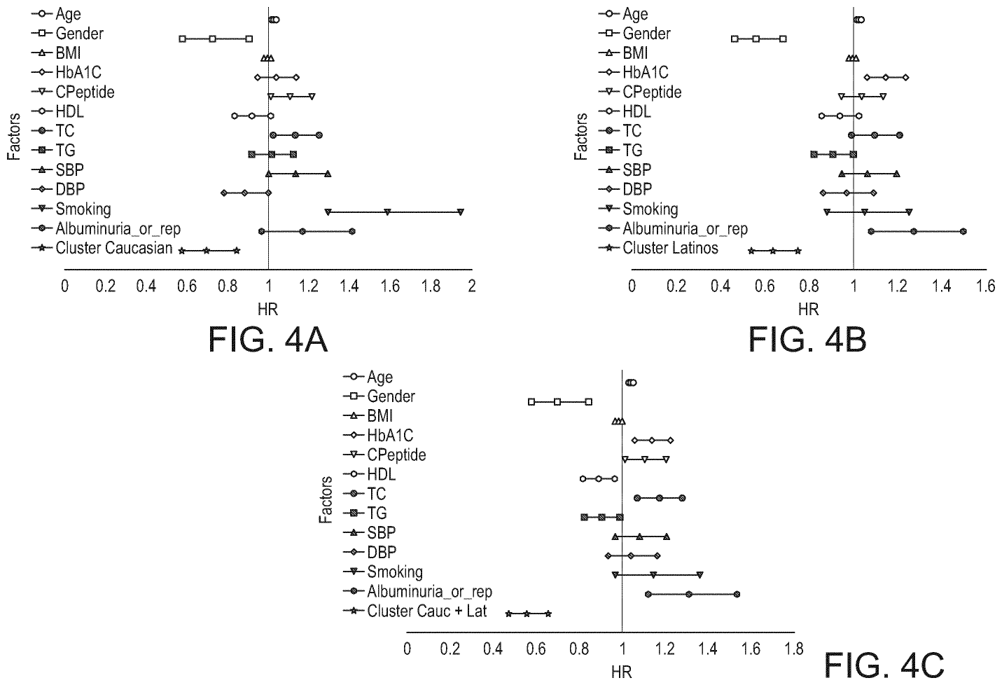

FIG. 4 shows cox-regression results presented as Hazard rations (HR) with confidence intervals. a-b, HR for the 2nd co-primary composite of CVO in Caucasians (a) and Latinos (b) for known CVO risk factors and cluster identity (as defined in FIG. 3, panel a-b). c, HR for the 1st co-primary composite of CVO for the same factors as above, considering the entire population (Caucasians plus Latinos). Cox regression models were significant P below 0.0000).

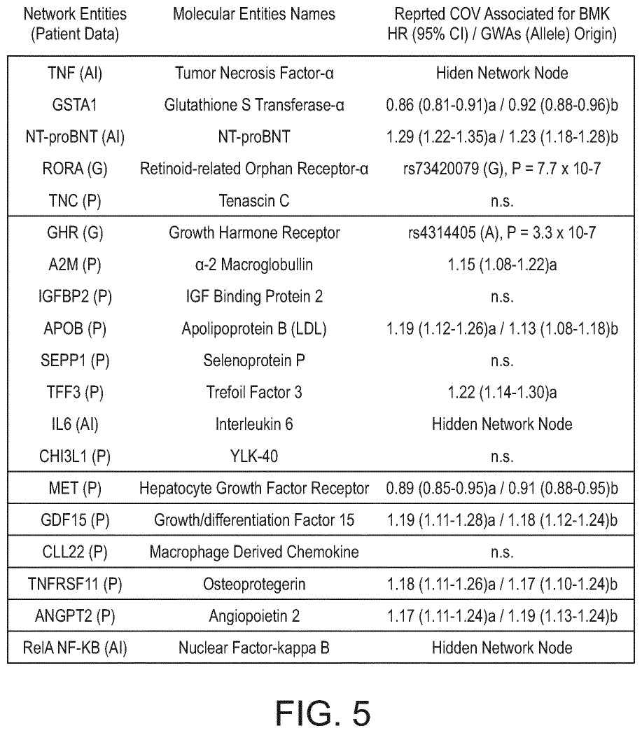

FIG. 5 shows the list of gene names from the identified disease network. Three main clusters (A, B, C in FIG. 3) of genes were identified (highlighted in red=lower activity, and shades of blue=higher activity of molecules in patients at higher risk vs. those at lower risk to CVO events). Individual patient data for these genes (G=genotype and P=protein levels) was used for the patient stratification workflow (FIG. 1). Al=molecular activity was inferred by Bayesian statistics. RORA and GHR variants were not previously reported to be associated with CVD, with the effect allele being rare in Europeans, but frequent for in Africans (MAF=0.41) and for the GHR variant in Asians (MAF=0.13). Results from standard BMK analyses for CVO in the ORIGIN cohort are reported for (a) 1st and (b) second co-primary cardiovascular composites, as previously published (CITE PUBLICATION). 1st co-primary endpoint=composite of CV death, or nonfatal MI or nonfatal stroke, and 2^{nd} co-primary endpoint=composite of 1st co-primary or revascularization procedure or hospitalization for heart failure). n.s.=biomarkers that were not significantly associated with CVO, but associated with death in the ORIGIN CVO trial.

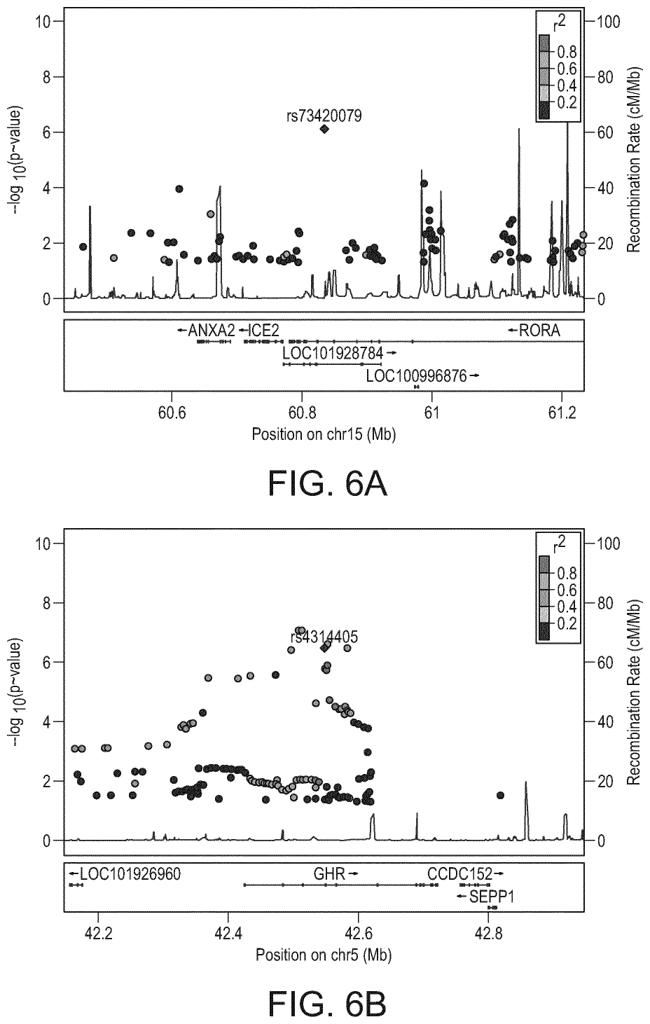

FIGS. 6a and 6b (s2) show locus zoom plots of the GWAs results from the ORIGIN study. SNPs are plotted on the x-axis according to their position on each chromosome against associations with CAD on the y-axis (shown as −log 10 P). The loci used for patient stratification (rs73420079, rs4314405; P<5×10-8) are indicated in the locus zoom plots b and c, respectively. MAF for these loci was around 1% in Europeans, except for MIR3169, which had a MAF=18%.

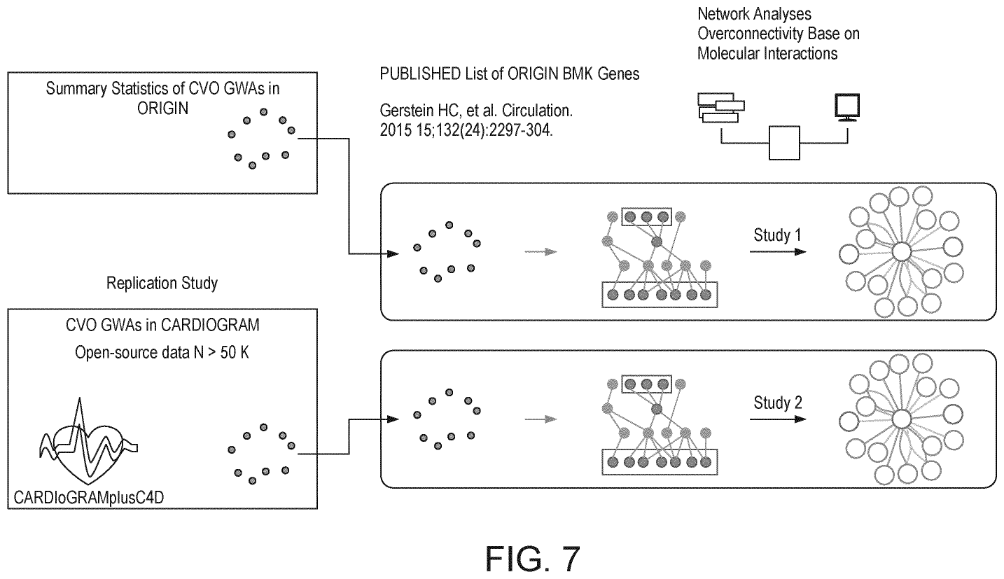

FIG. 7 shows the network analyses design. Summary statistics from ORIGIN CAD GWAs was used to identify most relevant genes association with CVO in this cohort. In a posterio analysis these gene plus a list of biomarkers (BMKs) encoding genes were used to produce networks of overconnected genes. This procedure was replicated using GWAs summary statistics for CAD outcome from the CARDIOGRAM consortium (CARDIoGRAMpIusC4D, Nikpey et al 2015) plus the identical list of BMKs associate with CAD outcome.

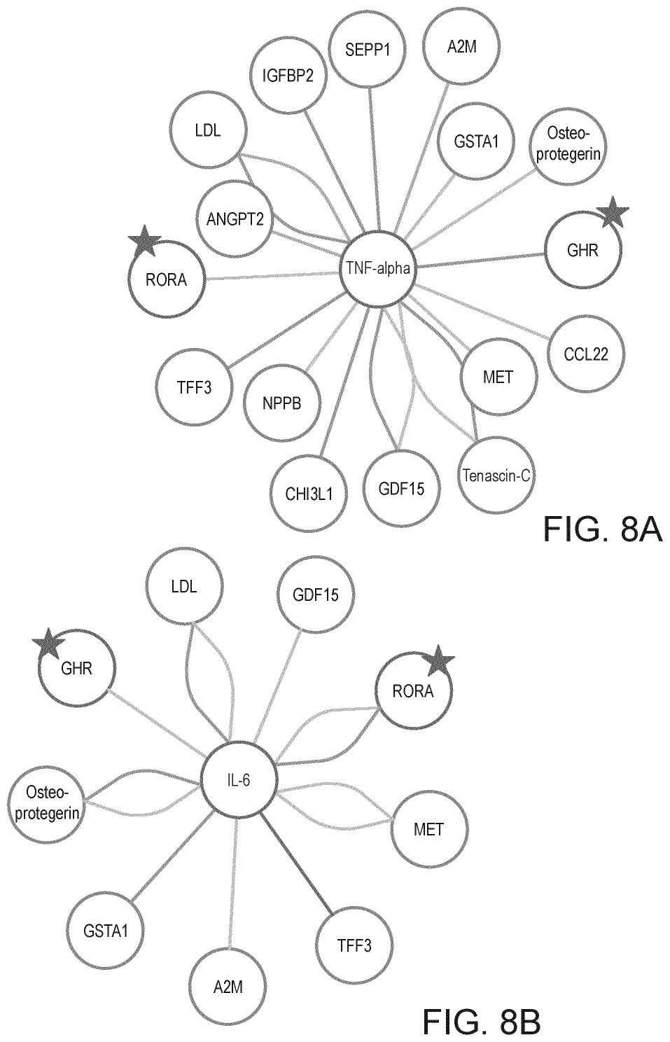

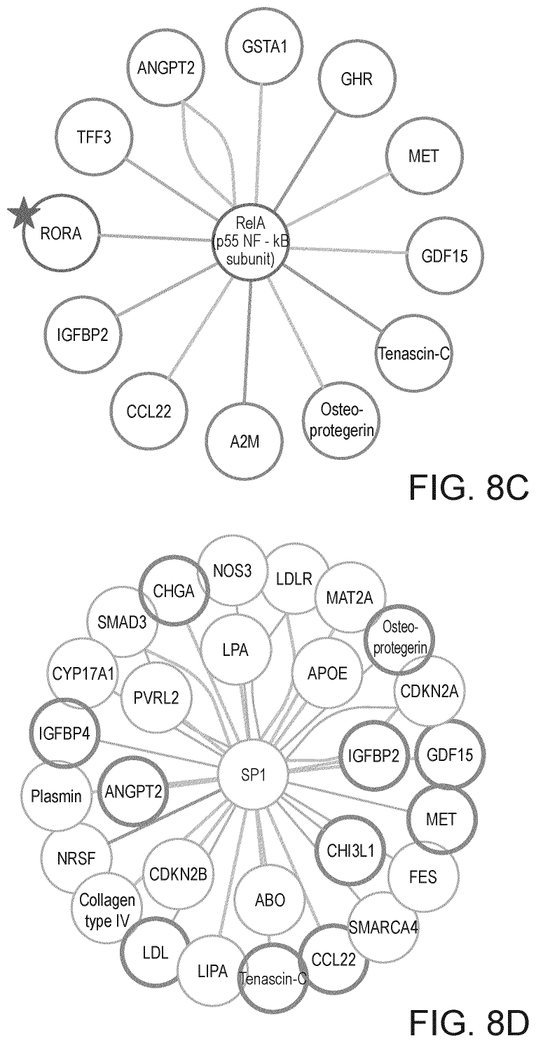

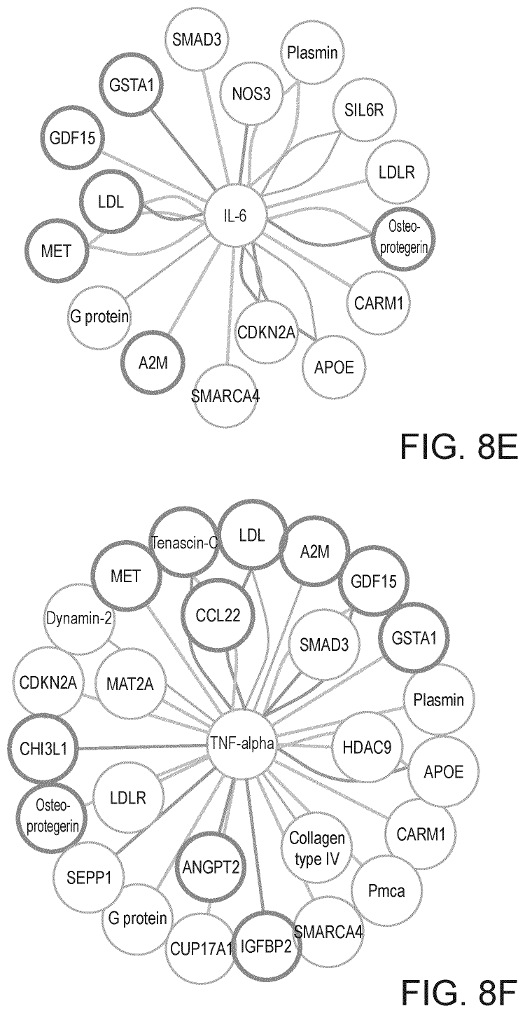

FIG. 8 shows networks of molecular entities that were prioritized in the overconnectivity analyses. a, b, c, First 3 top ranking networks in the discovery network analyses using candidates from GWAs and BMK analyses from the ORIGIN datasets. c, d, e Replication network analyses showing top sub-networks (ranking 1st, 4th and 8th) identified when using the CARDIOGRAM GWAs results plus BMK analyses results from ORIGIN. These most resembled the networks identified in a-c (replicated BMK in blue). Stars=genes associated with CVO in the ORIGIN GWAs.

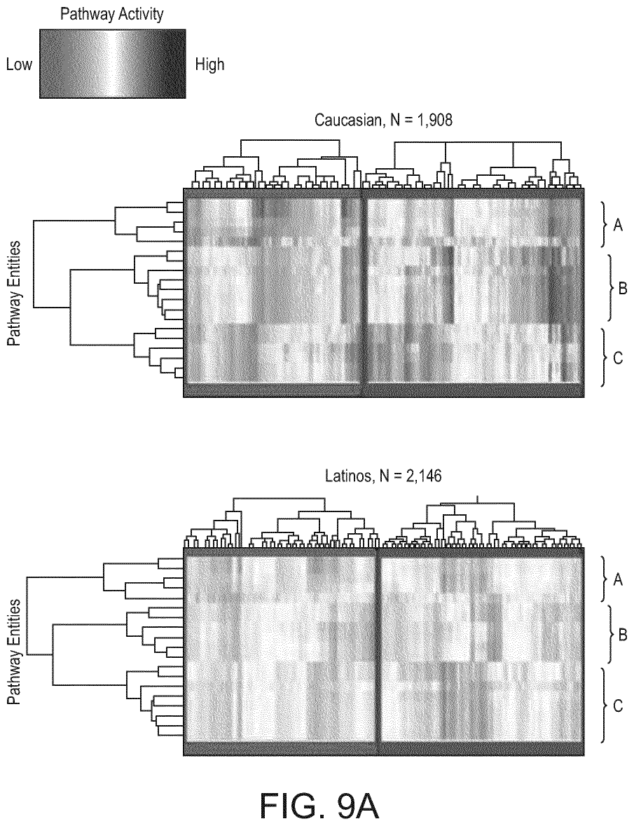

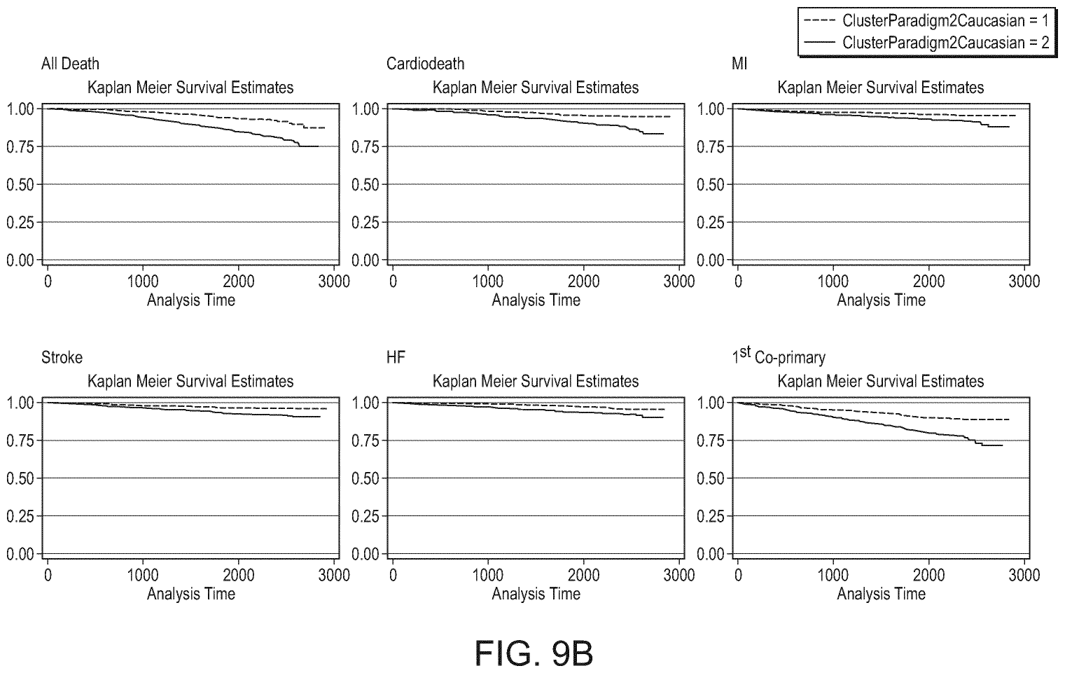

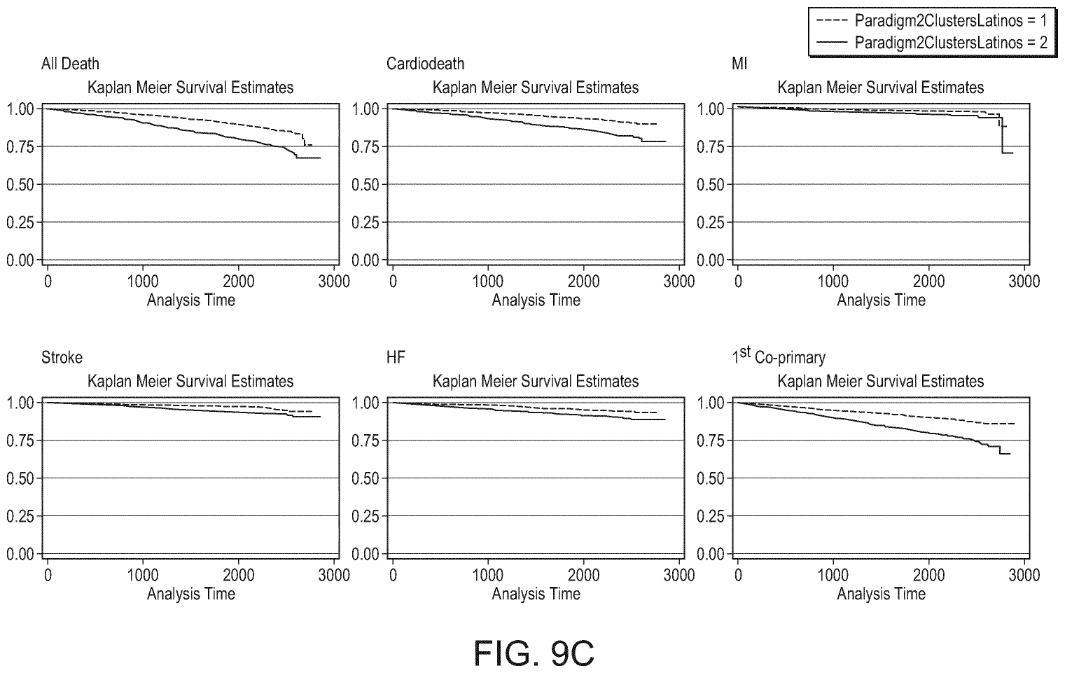

FIG. 9 shows Kaplan-Meier survival estimates by patient clusters for the measured CV outcomes in Caucasian and Latinos. (a), clusters identified and b-c, survival curves (red=cluster highlighted in red, blue=cluster highlighted in blue in the upper panels). (b) Caucasians and Latinos (c). There were significant differences in survival between clusters in all cases (Log-rank P<0.000 was the less significant difference).

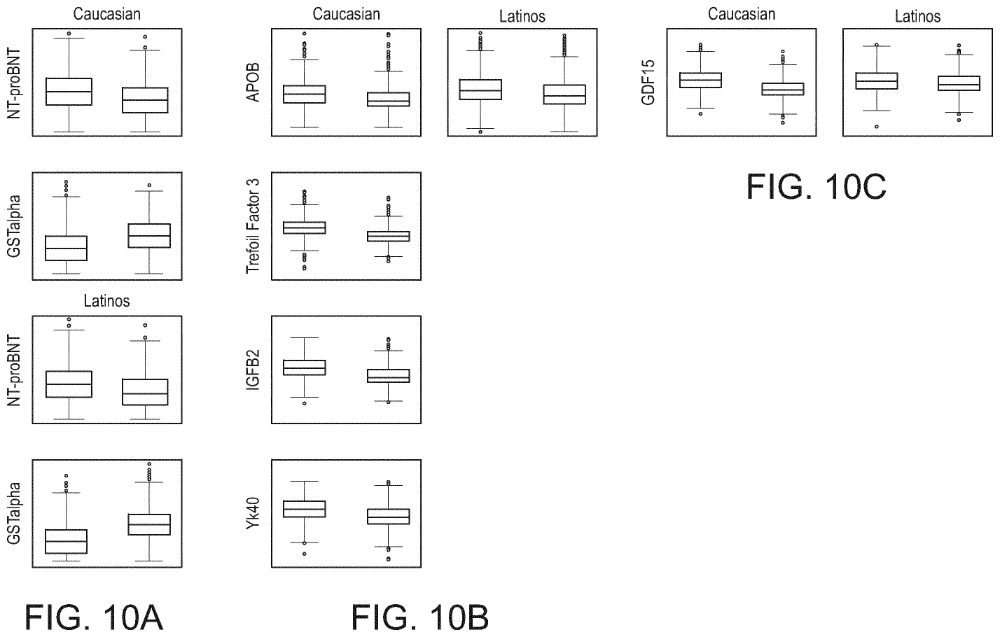

FIG. 10 Box-plots for levels of biomarkers comprising the clusters of genes (A, B, C) identified when clustering patient specific BMK activity (FIG. 2). Patients in the cluster at higher risk for CVO (left box-plot in each graphic) had higher BMKs levels, except for GSTalpha, which was lower in the higher risk group.

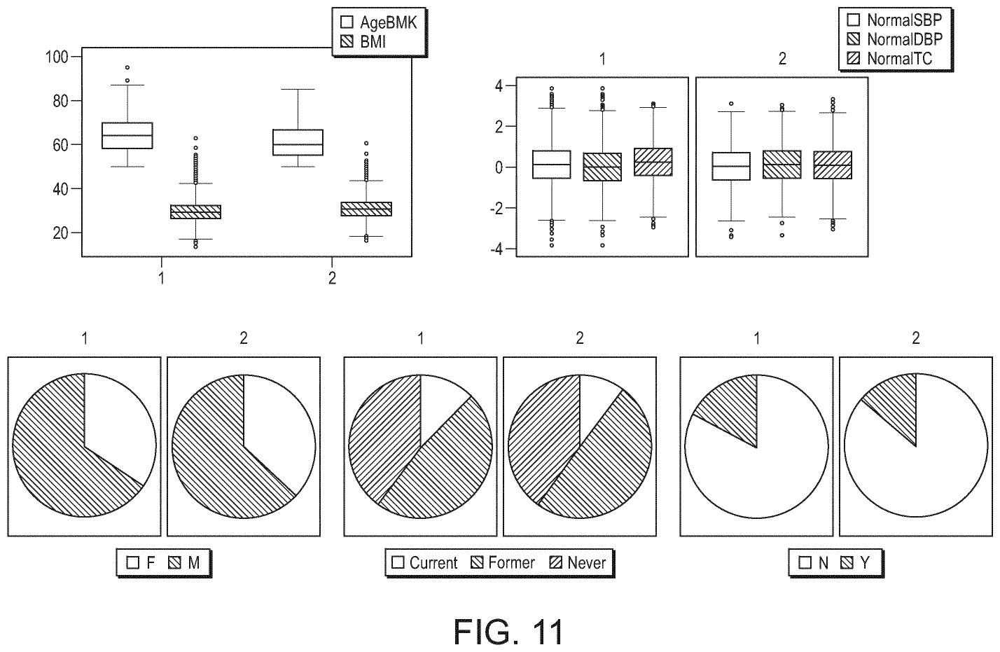

FIG. 11 shows visual inspections of cardiovascular risk factors in the high (1) versus low (2) CVO risk clusters (Caucasian plus Latino sub-populations). Upper panels, Box-plots for age, BMI and levels of BMK routinely measured in the clinic. Patients in the cluster at higher risk for CVO (left box-plots in each graphic) were in average similar to patients in the cluster with lower CVO risk. Lower panel, Categorical CVO risk variables such as sex (left lower panel), smoking (mid lower panel), and albuminuria (right lower panel) or reported albuminuria were slightly different between clusters. More details for these and other risk factors are given in Table 1. Statistical analyses for CVO risk (logistic regression) and survival (cox regression) included these as co-variates to calculate the risk and Hazard ratios per cluster as presented in the MS body. NormalTC=normalized Total Cholesterol; normalSBP/DBP=normalized systolic blood pressure/diastolic blood pressure.

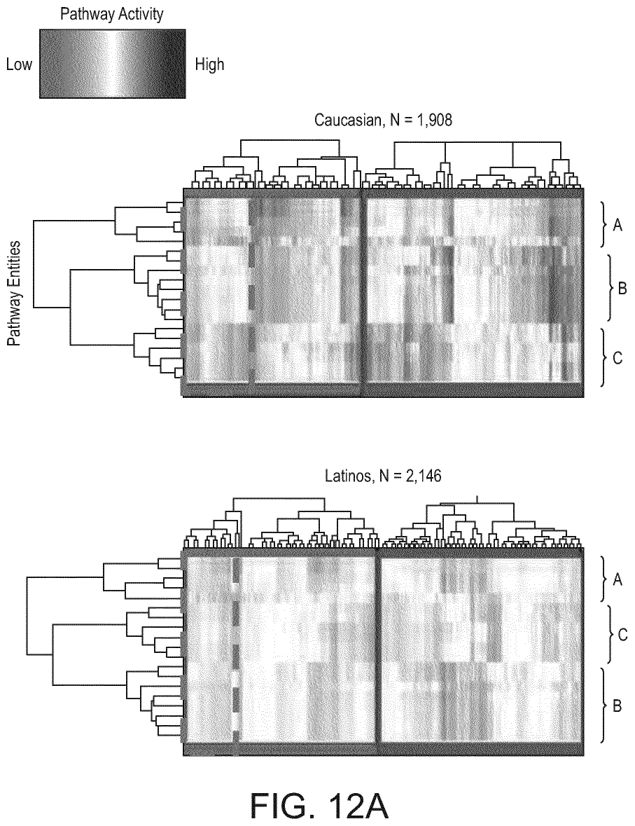

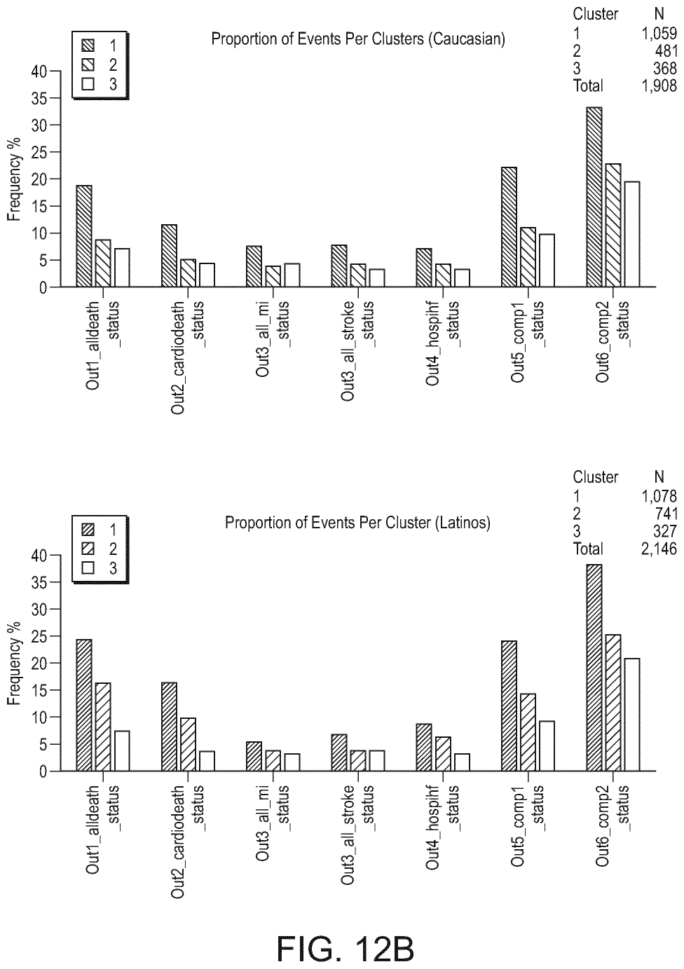

FIG. 12 shows sub-cluster analyses. (a) Sub-clusters within clusters (cluster 2 is now divided in 2 and 3) in both Caucasian and Latinos. (b) Proportion of CVO events per clusters 1 to 3 and the corresponding N of individuals.

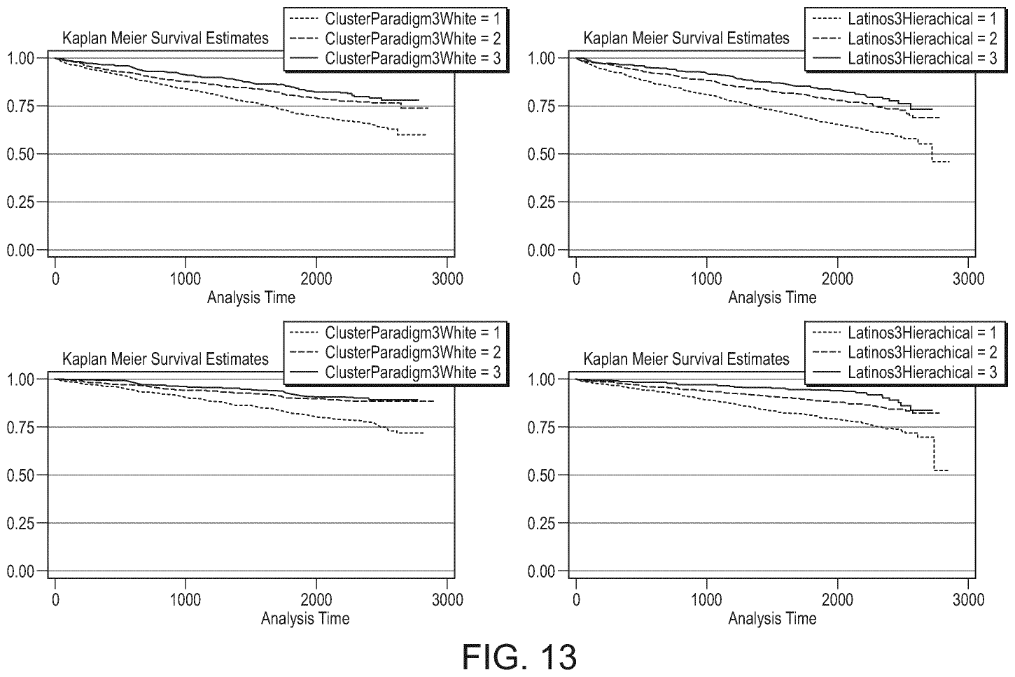

FIG. 13 shows Kaplan-Meier survival estimates for measured outcomes in Caucasian (left panels) and Latinos (right panels) for the 3 identified sub-clusters (as shown in FIG. 13). There were significant differences in survival between clusters in all cases (Log-rank P<0.0000).

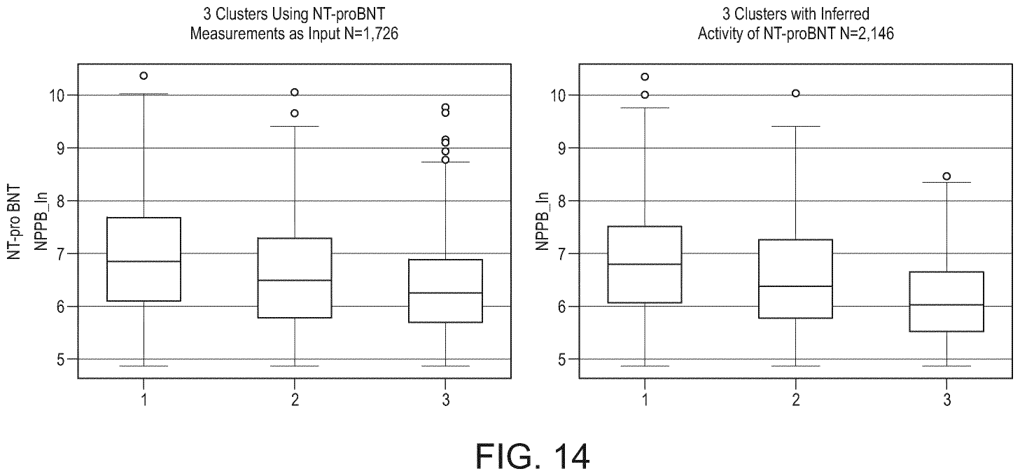

FIG. 14 (S10) shows NTproBNP biomarker levels in 3 clusters of patients obtained by either using NT-proBNT levels (left panel) or without using it (right panel) as input for the network calculations that generated these clusters in Latinos. There were no significant differences NT-proBNT levels when using the first or the later analyses results as classifier of patients (clusters 1 to 3).

### EXAMPLES

The inventors set out to identify biomarkers, and combinations of biomarkers, associated with CVD progression, with the aim of developing strategies to enable identification of individuals who are at risk from suffering from a CVD event, and thus enable early intervention to prevent, or reduce the risk of, the individual from suffering from a CVD event. Identification of suitable biomarkers and biomarker networks will also optimize clinical trials plans, drug efficacy, and optimize treatment.

### Example 1—Identification of Biomarkers and Biomarker Networks

### Materials and Methods

### ORIGIN Cohort

The participants of the ORIGIN biomarker sub-study (N=8,401, Supplementary Table S1) were chosen among the randomized patients who were treated by Lantus or placebo. A smaller sub-population of this sample was genotyped (5,078 samples). After quality control (see below), sample size was 4,390 individuals. Only these genotyped subjects entered the analyses described in this protocol for patient stratification (demographic characteristics in Supplementary Table S1).

### Biomarkers Quality Control and Normalization

The quality control for the protein biomarkers measured in the ORIGIN cohort was previously described (Gerstein et al., 2015). Normalization=biomarkers that were not normally distributed were log transformed. Standard biomarker analyses for CVO prediction in this cohort were described elsewhere (Gerstein et al., 2015).

Quantitative BMK measurements were also transformed to categorical variables (−1, 0, +1) based on percentiles of the distributions for each BMK, separately for Caucasian, Latinos and subjects of African origin. The algorithm PARADIGM cannot deal with continuous traits for clustering.

### Genotyping Quality Control and Population Stratification Analysis

Genotyping of the ORIGIN cohort, N=5,078 samples, was performed using Illumina HumanCore Exome DNA Analysis Bead Chip (Illumina Omni2.5). Over 540,000 genetic variants were called, including extensive coverage of coding variants, both common and rare. Single nucleotide polymorphisms (SNPs) were excluded with call rate <0.99, minor allele frequency <0.01, or deviation from Hardy-Weinberg equilibrium (P<1×10-6). Individuals were excluded if their self-reported sex, ethnicity and relatedness were not in concordance with their genetic information. After quality control, sample size was 4,390 individuals and 284,024 SNPs (note: SNPs excluded because of low allele frequency were accurately genotyped for the most part and will be included in future analyses). Genome-wide genotype imputation was performed using Impute v2.3.0. The inventors used the NCBI build 37 of Phase I integrated variant 1000 Genomes Haplotypes (SHAPEIT2) as the reference panel. Imputed SNPs were excluded with imputation certainty score <0.3. Final number of Imputed SNPs was 10,501,330.

Principal components were generated based on whole genome genotyping separately for Caucasian and Latinos. These were used as co-variates in genetic analysis for the genome-wide association study (GWAs) from the ORIGIN study.

Due to population ethnic substructure, subpopulations were defined by ethnic groups (Caucasian and Latinos) and analyzed separately. HWE (P>0.001) for the SNPs entering the analyses was tested as part of the quality control to define if the population subgroups met expected genotypes distributions. Subpopulations were meta-analyzed and association results were visualized in Manhattan and QQ plots (FIG. 6).

### Genome-Wide Association Analyses

GWA analyses were conducted separately for Caucasian (N=1,931) and Latinos (N=2,216). Genotypes consisting of both directly typed and imputed SNPs (N=4.9-9

Mio) entered the GWA analyses. To avoid over-inflation of test statistics due to population structure or relatedness, the inventors applied genomic controls for the GWA analysis. Principal components were generated based on whole genome genotyping separately for Caucasian and Latinos. These were used as co-variates in the genome-wide association analyses from the ORIGIN study.

Linear regression (PLINK) for associations with normalization was performed under an additive model, with SNP allele dosage as predictor and with age, gender.

Meta-analysis was performed. Corresponding to Bonferroni adjustment for one million independent tests, the inventors specified a threshold of P for genome-wide significance. The CARDIOGRAM consortium dataset used was the CARDIoGRAMplusC4D 1000 Genomes-based GWAS meta-analysis summary statistics. It comprised GWAS studies of mainly European, South Asian, and East Asian, descents imputed using the 1000 Genomes phase 1 v3 training set with 38 million variants. The study interrogated 9.4 million variants and involved 60,801 CVD cases and 123,504 controls (Nikpey et al., 2015). To assess the number of independent loci associated with CVD, correlated SNPs were grouped using a LD-based result clumping procedure (PLINK, Purcell et al, 2007). This procedure was used for gene mapping of loci (Supplementary Table S2) entering the overconnectivity network analysis. Variants associated with CVD at a p-value below 10-6 with proxies at a p-value below or equal 10-5 in ORIGIN cohort and p-value below 10-7 with proxies at a p-value below equal 10-6 in the CARDIOGRAM cohort were mapped to genes, so that these could be considered in the network analyses. The inventors excluded alleles with a MAF below 1 percent and poor imputation quality (Info below 0.4) from the clumping procedure.

### Workflow

All steps of the workflow (FIG. 1) are as follows:
(1) Candidate molecules selection
   A list of entities included in the discovery and replication studies is presented in Table S3.
(2) Gene mapping
   SNPs associated with CAD in ORIGIN and in CARDIOGRAM were mapped to genes using the clustering procedure available in PLINK (Purcell et al, 2007) based on empirical estimates of linkage disequilibrium (LD) between single nucleotide polymorphisms (SNPs). The inventors used 1000 Genomes (phase 1 release v3) as the reference dataset to estimate the LD between variants; the clumping analysis was performed using LD r2>0.8 (—clump-r2 0.8) to clump variants to an index SNP within a range of 250 kb (—clump-kb 250). To identify clumped genomic regions corresponding to genes The inventors used the—clump-range function with a gene list (hg19).(Supplementary Table S2);
(3) Network analysis for disease network identification
   The inventors used overconnectivity analyses to build a network representative of cardiovascular disease, as described in more detail below.
(4) Single level patient data curation
   To initiate the patient stratification workflow (FIG. 1), the inventors generated first the disease network and used the patient specific data for the prioritized molecules (Table 1). Patient specific data quality control is described above.
(5) Probabilistic graphical model analysis for identification of patient specific pathway activity
   The method is explained in the manuscript and below in more detail.
(6) Clustering
   The inventors used an R package as described below.
(7) Linking the identified patient clusters to CVO
   More details about the analyses, outcomes and co-variates are described below.
(8) Single biomarkers comparisons
   These were done by visual inspection of box-plots and median comparisons.
(9) Characterization of clustered populations.

### Disease Network Identification

A disease network was identified using the overconnectivity algorithm, as implemented in the R based Computational Biology for Drug discovery (CDDD) package developed by Clarivate Analytics. The specificity of the network for the disease relies on the disease linked molecules chosen to produce the network (FIG. 7) and on the underneath libraries of protein-protein interactions in humans. For this purpose, the inventors extracted high trust interaction manually curated systems biology knowledge bases from Ingenuity (IPA from QIAGEN Inc.) and Metabase (Metacore from Clarivate Analytics) to be used as libraries for the CBDD package. To create the CVD disease network, the inventors: i) made use of topological characteristics of human protein interaction networks to identify one-step away direct regulators of the dataset that are statistically overconnected with the objects from the data set (hypergeometric distribution), ii) used as input datasets, names of genes having evidence of association to CVD (see Baysian network analyses below and in FIG. 7). As input data for the overconnectivity analyses, the inventors used in the discovery analyses, names of genes corresponding to 16 protein BMKs and, from 8 loci associated with CVO in the Origin cohort GWAs (Table S3). To validate the initially identified network, the inventors re-ran these analyses replacing genetic associated loci from the ORIGIN study, by other 90 genes from an independent large GWAs meta-analysis (CARDIoGRAMpIusC4D, cite Nikpey et al 20115) from the CARDIOGRAM consortium (FIG. 7, Table S3).

### Bayesian Network Analyses for Data Integration

PARADIGM is a data integration approach based on probabilistic graphical models. It renders a pathway or network as a probabilistic graphical model (PGM), learning its parameters from supplied omics data sets. The model allows inference of true activity score for each node in the pathway given the different omics measurements for the nodes. PARADIGM allows prediction on the level of individual patients and is capable of accommodating such data types as gene/protein expression, copy number changes, metabolomics, direct protein activity assays such as kinase activity measurements. PARADIGM combines multiple genome-scale measurements at the sample level to infer the activities of genes, products and abstract process within a pathway or subnetwork. Edges of original network connect hidden variables of different nodes (e.g. activity hidden variable of node A affects protein or DNA hidden variable of node B, depending on mechanism of A-B link).

Each node is assigned a conditional probability distribution when the model is created. The distribution tells how likely it is to observe a node in particular state given states of its parents in the model Three states are allowed for each node (activated, repressed, unchanged). The distributions for hidden variables are defined at the first step. Distributions for observed variables of each molecular level are learned by EM algorithm using the input data. After model is complete, inference can be made about probabilities of observing hidden nodes in a particular state—either without observed data (prior probability) or taking data into account (posterior probability).

The main output is a matrix of integrated pathway activities (IPAs) A where A_ij represents the inferred activity of entity i in patient sample j. The values in A are signed and are non-zero if the patient data makes the activation or inhibition of the hidden node more likely compared to prior. The A is supposed to be used instead of original data sets for purposes such as patient stratification or association analysis to reveal biological entities with activity associated to clinical traits.

The output is a matrix of activity scores for each node in the network and each sample. The activity score represents signed log likelihood ratio (positive when the node is predicted to be active, negative when node is predicted to be repressed). A pathway is converted into a probabilistic graphical model that includes both hidden states for each node and observed states for the nodes which can correspond to the input data sets. There are two possible modes to assess IPA significance. Both involve permutation—calculation of IPA scores on many randomized samples.

For the ‘within’ permutation, a permuted data sample is created by creating new set of evidence (i.e. states for observed variables at gene expression and gene copy number) by assigning a value of the random node in pathway/subnetwork and random sample to each observed node.

For the ‘any’ permutation, the procedure is the same, but the random node selection step could choose a node from anywhere in the input data (regardless of whether a particular pathway/subnetwork contains such a node).

For both permutation types, iterations permuted samples are created, and the IPA scores for each permuted sample is calculated. The distribution of scores from permuted data is used as a null distribution to estimate the significance of IPA scores in real data set. SCRIPT: Paradigm (Vaske et al., 2010) in R with CBDD package developed by CLARIVATES.

Clusters were produced separately for the discovery (Caucasian; N=1908) and replication (Latinos; N=2146 sub-populations.
1. Network: Interactome (Sanofi network from IPA and Metabase) human high quality.
2. Matrix: Patient specific information form 13 protein biomarkers and genotypes from 2 GWAs genes were used to run paradigm. These molecular entities appeared in the first 3 subnetworks obtained from the network analysis with the overconnectivity algorithm. Proteins coded as −1, 0, 1 (accordingly with the distribution) per patient. A matrix containing the 3 subnetworks obtained with the OVERCONNECTIVITY algorithm was also used as input for paradigm (though it contained molecular entities from which the inventors did not provide BMK measurements as input).
3. Levels: DNA and protein

### List of Proteins/Genes Included in the Analysis:

### Phenotypic Matrix (Proteins)

1) Alpha 2 macroglobulin (1st co-primary and death)
2) Angiopoietin 2 (1st, 2nd CVO and death)
3) Apolipoprotein B (1st, 2nd CVO and death)
4) YLK-40 (CHI3L1) (death)
5) Glutathione S transferase alpha (1st, 2nd CVO and death)
6) Tenascin c (death)
7) IGF binding protein 2 (death)
8) Hepatocyte growth factor receptor (MET) (1st, 2nd CVO and death)
9) Osteoprotegerin (TNFRSF11) (1st, 2nd CVO and death)
10) Macrophage derived chemokine (CCL22) (death)
11) Selenoprotein P (death)
12) Trefoil factor 3 (1st CVO and death)
13) GDF15 (1st, 2nd CVO and death)
   Genotypes from Genes:
1) RORA (rs73420079 CAD effect allele G-AA=−1, AG=0, GG=1),
2) GHR (rs4314405 CAD effect allele A-GG=−1, AG=0, AA=1).

Biomarkers to be predicted as key members of the input networks:

TNF-alpha, IL6, ReIA NF-KB Subunit, NT-proBNP (NPPB)

### Patient Stratification

The inventors initiated the patient stratification analyses based on the disease sub-networks identified in the ORIGIN cohort (FIG. 2) and using the respective patient specific protein or genotypes data (workflow FIG. 1; FIG. 2 and Table 1). Due to population ethnic background. Two main subpopulation with protein biomarker and genotypes available were considered (Caucasian and Latinos), with similar sample sizes (N=1,908 and N=2,146; respectively) and sample characteristics for CVO risk factors (Supplementary Table S1). These were thereby considered as discovery and validation samples for i) Bayesian network analyses, ii) hierarchical clustering, and iii) CVO risk and survival analyses. The Bayesian network analyses were conducted using the PARADIGM algorithm (Vaske et al), implemented in R as part of the CDDD package developed by Clarivate Analytics. The data integration approach is based on probabilistic graphical models. It renders a pathway or network as a probabilistic graphical model (PGM), learning its parameters from supplied omics data sets (FIG. 2. The model allows inference of true activity score for each node (molecule) in the pathway given the different omics measurements (per patient) for the nodes (FIG. 2; Table 1). Each node is assigned a conditional probability distribution when the model is created. The distribution tells how likely it is to observe a node in particular state given states of its parents in the model. Three states are allowed for each node (activated, repressed, unchanged). The distributions for hidden nodes are defined at the first step (e.g., a transcription factor regulating a network of genes, see FIG. 1). Distributions for observed variables of each molecular level are learned by EM algorithm using the input data. After model is complete, inference can be made about probabilities of observing hidden nodes in a particular state—either without observed data (prior probability) or taking data into account (posterior probability). The output is a matrix of activity scores for each node in the network and each sample. The activity score represents signed log likelihood ratio (positive when the node is predicted to be active, negative when node is predicted to be repressed). A pathway is converted into a probabilistic graphical model that includes both hidden states for each node and observed states for the nodes which can correspond to the input data sets. This function takes a matrix of activity scores and calculates p-values for each value using permutation approach. Hierarchical clustering of the calculated pathway activity per patient and molecule was done using the R hclust function. The dissimilarities between clusters was computed using squared Euclidean distance.

### Linking the Identified Patient Clusters to CVO

To verify the relevance of the identified patient clusters in both the Caucasian and Latino populations (FIG. 3) to the CV outcomes measured in the ORIGIN cohort, the inventors used logistic regression, Cox proportional hazard models, and survival analyses. To confirm consistency of results, these analyses were repeated for sub-clusters within the initially identified clusters (FIGS. 12 and 13). Logistic regression was used to estimate if known CVO risk factors could alone distinguish patients from the higher versus lower CVO risk clusters identified by molecular signatures. Cox proportional hazard models were used to estimate the association between clusters and time-to-event, as variation in cumulative disease incidence could be accessed in the prospective study. Kaplan-Meier survival estimates were used to visualize the relevance of the identified clusters to disease progression and log-rank test was used to test the equality of survivor functions. CV outcomes used for the analyses were: myocardial infarction (MI), stroke, cardiovascular death and heart failure (HF) with hospitalization, beyond death for all causes (FIGS. 9 and 12). Two additional MACEs were used, (i) 1st co-primary endpoint (═CV composite): composite of CV death, or nonfatal MI or nonfatal stroke, and (ii) 2nd co-primary endpoint (=expanded CV composite): composite of 1st co-primary or revascularization procedure or hospitalization for heart failure (FIG. 3). Co-variates used in the models were: age, sex, BMI (kg/m2), HbA1c (%), c-Peptide, HDL-C (mmol/L), LDL-C (mmol/L), TG (mmol/L), TC (mmol/L), SBP (mm Hg), DBP (mm Hg), smoking status, albuminuria or reported albuminuria. All continuous variants were normalized by inverse normal transformation. For sample characteristics for the higher versus lower CVO risk identified clusters in the Caucasian and Latino see Supplementary Table S1 and FIG. 10. Box-plots of the original biomarker levels were produced for visual inspection of differences in BMK levels between clusters (FIG. 9). Analyses were done using STATA Version 15.

### Results

### ORIGIN GWAs Results

In the GWAs the inventors conducted with the ORIGIN cohort, the inventors identified a few variants associated or borderline associated to CVD (FIG. 11).

These were mostly rare variants (MAF=0.01 in EUR), or did not map to a gene region (Supplementary Table S2). These variants were not present in the CARDIOGRAM dataset, and the inventors could not validate these findings using the Cardiovascular Disease Knowledge Portal (http://broadcvdi.org/home/portalHome). The inventors mapped GWAs results to nearby loci (Supplementary Tables S2) to inform the network analysis, identifying biological interactions of these loci to other CVD biomarkers (Supplementary Table S3). Results from the GWA analyses are shown in Table 1 for loci that were prioritized in the network analyses, all other findings are reported in FIG. 6 and Supplementary Table S2.

### CVD Disease Network Prioritization

The inventors built a CVD network (FIG. 7) based on proteins reported to be associated with CVO or death and the loci associated with CVD in the ORIGIN CVO trial (Supplementary Table S3), with the purpose to identify direct regulators of disease that are statistically overconnected. From the 8 top ranking loci (GHR, RORA, CLINT1, GRM8, LOC101928784, MYH16, SPOCK1, and TMTC2) from the CVD GWAs in the ORIGIN cohort (Supplementary Table S2), only 2 (RORA and GHR; FIG. 6 were prioritized in the overconnectivity analyses (FIG. 2). To validate the main networks identified (FIG. 2), the inventors re-ran these analyses with an independent GWAs dataset from the CARDIOGRAM consortium (Supplementary Table S2 and FIG. 7 for study design). Overconnectivity analysis pointed to 14 (12 in the replication study; (FIG. 8) out of the 16 protein BMKs (Supplementary Table S3) that were earlier identified to be associated with CVO in the ORIGIN cohort (Table 1). These were part of the top ranking significant networks (Supplementary Table S4) in the discovery and replication analyses (FIGS. 7 and 8). To note, though 90 other candidate genes were fed as input to the network calculations in the replication study (Supplementary Table S3), only 2 protein BMKs (NPPB and TFF3) and two genetically associated genes (RORA and GHR) were specific to the sub-network identified in the discovery analyses (FIG. 8).

### Patient Stratification Linked to CVO Progression

The prioritized sub-networks and their molecular directional interactions (FIG. 2; Supplementary Table S5 for interactions with the respective supporting references) were used to inform the subsequent patient stratification analyses, as specified in the workflow (FIG. 1; see Methods). The clustering of the calculated molecular activities was agnostic to CVO: 3 main gene clusters (A, B, C in FIG. 3, and panel a) and 2 main patient clusters were identified (FIG. 3, panel a) in Caucasians (with 1,059 and 849 patients) and replicated in Latinos (with 1,078 and 1,068 patients). The pathway activity for the higher CVO risk patients was clearly repressed in gene cluster A, and activated in gene cluster B, as for most of the patients in cluster C (FIG. 3 and Table 1). The protein BMKs were previously reported to be associated with CVO or death for all causes in the ORIGIN cohort (Gerstein et al., 2015).

A relation of the clusters to 1st and 2nd co-primary composites of CVO was found in a second step through survival analyses (Kaplan-Maier survival estimates and Log rank analyses to test for equality of survival function) and Cox-regression models adjusted for CVO risk factors (FIG. 3, panels b-c; and FIG. 4, panels a-c). Similarly, patient clusters also associated to myocardial infarction, stroke, cardiovascular death, heart failure with hospitalization, beyond death for all causes (FIG. 9 for Kaplan-Maier survival estimates), with the higher risk cluster having in average about 2 times greater chances of event occurrence than the lower risk cluster (FIG. 3, panels d-e).

The distribution of CVO risk factors (Supplementary Table 1 and FIG. 11) was mostly similar between the higher versus lower CVO risk patients cluster in both Caucasian and Latinos (Supplementary Table 1). In a logistic regression model including age, sex, BMI, HbA1c, c-Peptide, HDL-C, LDL-C, TG, TC, SBP, DBP, smoking status, albuminuria or reported albuminuria; only albuminuria, total cholesterol and age were relevant to separate lower versus higher CVO risk clusters in both Caucasians and Latinos (Supplementary Table 6). Smoking habits were just distinct for clusters identified in Caucasians, while Gender and BMI only contributed to separate clusters in Latinos (Supplementary Table 6). To test how relevant the clusters were to predict CVO after adjusting for these known CVO risk factors, the inventors used Cox-regression models (FIG. 3 and in more detail in FIG. 4). Hazard ratios (HR) to the 2nd co-primary composites of CVO were largest for gender (women had a reduced HR in relation to man) and cluster (lower CVO risk cluster was protective) in both the Caucasian and Latino group (FIG. 4, a-b). Smoking had a large effect in Caucasians, but a modest one in Latinos. Consistently across populations, HDL, TC, SBP and albuminuria were also relevant, but contributed to a lesser extent than Gender and Cluster. Above 80 percent of the population was diabetic (Supplementary Table 1); HbA1C was more relevant for the HR in Latinos, while was C-Peptide in Caucasians (FIG. 4, a-b). The inventors repeated this analysis for all outcomes (FIG. 3, d-e) for the combined Caucasian and Latino populations, as presented in FIG. 4 (panel c) for the 1 st co-primary composites of CVO—which results were similar to analyses with the 2nd co-primary composites of CVO. To test if the clustering was not randomly associated with CVO survival, the inventors repeated the analyses shown in FIG. 3 for sub-clusters of the initially identified clusters, with consistent results (Supplementary FIGS. 12 and 13). Average levels of protein BMKs in the higher versus lower CVO risk cluster (FIG. 10) were as expected, corresponding to the direction of associations of these proteins to CVO in the ORIGIN cohort (Table 1). Also, measured levels of protein BMKs in the higher versus lower CVO risk cluster mostly corresponded to higher versus lower calculated activity for the respective molecules (FIG. 3, panel a; Table 1), except for NT-proBNP. Thereby, calculated activities does not necessary reflects levels of BMKs, as activity will be inferred (e.g., how likely the protein x activity is de-activated?) based on known molecular interactions. Although NT-proBNP levels were higher in the higher CVO risk patient cluster compared to the lower CVO risk patients (FIG. 10), the inferred activity for this gene in the network was lower in the higher CVO risk patient group, than in the lower risk patient group (Table 1). When recalculating the network activities using NT-proBNP levels as input for a sub-set of patients who had its levels measured, levels of the protein per cluster were equivalent to its levels in similar clusters when calculating the network activity without using NTproBNP levels (FIG. 14). Similarly, in the absence of TNFalpha measurements for the ORIGIN trial, the network activity was calculated taking into consideration only the neighboring molecules, which indicated that TNFalpha was less activity in higher CVO risk patients, although levels of the actual protein could have been high. The molecular activities of IL6 and ReIA NF-KB, also upstream regulators of the networks, were estimated to be higher in the higher CVO risk group (FIG. 3, Table 1). These networks were less informative than TNFalpha, which contained already all interacting molecules (FIG. 2), but as IL6 and ReIA NF-KB were central to those their activities could be calculated.

### DISCUSSION

Here, the inventors developed a workflow that goes from the identification of a CVD network to the proof of concept that the molecular interactions identified can be used to stratify patients with regard to disease progression. The inventor's computational biology approach has identified the group of biomarkers associated with CVD outcomes and has associated, for the first time, SNPs associated with CVD risk. The inventor's work has identified molecular interactions and shows interdependencies of molecular activities that associate with different stages of CVD progression.

These results may enable the identification of individuals who are at risk from suffering from a CVD event, and thus enables early intervention to prevent, or reduce the risk of, the individual from suffering from a CVD event. In particular, detection of each biomarker in isolation enables the identification of individuals who are at risk from suffering from a CVD event, and detection of multiple biomarkers, i.e. the biomarker signature provides a particularly effective means of enabling early intervention to prevent, or reduce the risk of, the individual from suffering from a CVD event.

This is the first evidence for the reported GHR and RORA variants to be associated with CVD, and Example 2 sets out the method of determining a subject's risk of cardiovascular disease based on detections of SNPs in GHR and RORA genes. Though the genetic evidence of association with CVD is not strong, RORA is known to regulate a number of genes involved in lipid metabolism such as apolipoproteins Al, APOA5, CIII, CYP71 and PPARgamma, possibly working as a receptor for cholesterol or one of its derivatives cite Uniprot). Additionally, overexpression of RORA isoforms suppresses TNFalpha induced expression of adhesion molecules in human umbilical vein endothelial cells, regulating inflammatory response (Migita et al., 2004). The inventor's network analyses shows that RORA and GHR interact with main regulatory hubs of the identified CVD network of BMKs. Consistent with that, their genotypes contributed to calculations of the network activity and to the clustering of patients, which were in turn related to CVD progression.

RORA and GHR connected to the main networks through upstream regulators (TNFalpha and IL6) common to the prioritized molecules. Though, TNFalpha and IL6 were not part of the input information to create the network, the inventors could uncover these as hidden main regulators of the input molecules. Links of TNFalpha and IL6 to CVD are known (Lopez-Candales et al., 2017)—but, the network allows the visualization of the directions of the molecular interactions (FIG. 2). These were used to inform the Bayesian statistics method to calculate the patient specific pathway activity, providing thereby, a summarization of different omics layers, as more robust signatures for elucidating meaningful patient subgroups and understanding mechanistic interactions.

One of the advantages of the Bayesian network analyses over single BMK analyses, is that the activity of BMKs that were not measured or had poor measurement quality in the patient sample (for instance TNFalpha, and IL6 in the ORIGIN trial) can be estimated, in special if these are central to the disease network. This can lead to the discovery of new BMKs of disease progression. TNFalpha and IL6 have been intensively studies in CVD models, but in humans their protein detection is somewhat cumbersome and their levels are also influenced by physical activity (Vijayaraghava et al., 2017).

Here, the inventors could infer their activities based on downstream interacting protein, from which measured levels were fed into the Bayesian model (FIG. 2). The calculated activities may be interpreted as activation of the molecule, rather than actually a measure of levels of the protein, i.e., a transcription factor is calculated to be active or not based on downstream targets, not only of the effective measured protein levels. For instance, the TNFalpha pathway was less active in patients at higher risk to CV events. It may appear at first contradictory, as in functional studies inhibition of TNFalpha had beneficial effects on cardiac function and outcome. Nevertheless, most prospective studies with TNFalpha inhibitors bind, which to TNFalpha directly, reported disappointing and inconsistent results for CVO risk. For instance, in rheumatoid arthritis (RA) patients, it is rather the control of the disease itself (e.g., inflammatory pathway) than TNFalpha levels reduction per se that is necessary for CVO prevention (Coblyn et al., 2016). Thereby, specific protein interactions should be relevant to the inflammatory process and TNFalpha is just a peace of the puzzle. In this context, IL6, which is also an inflammatory marker, was predicted to be more active in the higher CVO risk cluster in the inventors study. The transcription factor nuclear factor kappa-B, which was predicted to be more active in the higher CVO risk cluster in the inventors study, regulates expression of many proinflammatory cytokines and is cardioprotective during acute hypoxia and reperfusion injury. As for TNFalpha, the activity of NTproBNT, a cardiac hormone that may function as a paracrine antifibrotic factor in the heart, was predicted to be lower in patients at higher CVO risk. Here, the inventors could confirm that the majority of these patients had actually higher levels of protein, than those less prone to CVO. BNP and NTproBNP peptides are released into the blood circulation in response to pressure and volume overload of the cardiac chambers. Cleavage of pre-proBNP precursor within cardiomyocytes leads to the formation of proBNP, which is subsequently cleaved into Nterminal (NTproBNP) and C-terminal (BNP) fragments. Most biological effects of BNP are the result of its binding to the natriuretic peptide receptor (Dhingra 2002). Thereby, NTproBNP calculated activity reflects rather molecular interactions that indicate it is active than the actual protein levels. The inventors, without wishing to be bound to any particularly theory, hypothesize that though NTproBNP levels are high in patients at higher CVO risk, it is being not effective as it should be.

NPPB and TFF3 were the top ranking associated BMKs by conventional statistics in the ORIGIN cohort, but did not appear in the network constructed with the CARDIOGRAM gene set. As the overconnectivity algorithm identifies the shortest path connecting genes, the inventors, without wishing to be bound to any particular theory, can conclude that: i) there is an active biological network that connects GWAs associated genes to protein biomarkers that had been associated to CVD, to which TNFalpha plays a central role; ii) though NPPB and TFF3 clearly are relevant biomarkers of CVD, these appear to be more downstream to the cascade of events related to the TNFalpha than the gene set identified in the CARDIOGRAM study. Nevertheless, for the purpose of identifying a disease related network for BMK discovery, protein BMKs should be more relevant than loci associated with CVD, as GWA results often map to genes that do not encode circulating proteins. In the patient stratification analyses, the inventors used mainly circulating proteins (N=13 plus 2 genetic markers), making it feasible for translational application, as tissue specific samples are often difficult or not feasible to obtain in standard clinical practice.

Though the complexity of this computational approach does not make it a straight forward process to be used in the clinic, the obtained patient strata can be further investigated to identify ideal BMKs (including known clinical parameters) combinations and ratios that would represent different stages of disease progression.

Identifying clusters of sub-populations progressing differently towards CVO will be informative to: i) define how the molecular signatures of each cluster translate into combinations of biomarkers with prognostic value, and iii) to define how these markers will respond to treatment in an additional cohort. Thereby, molecular signatures of disease progression should lead to strategies to optimize clinical trials plans, and drug efficacy, and so to optimize treatment.

Supplementary TABLE S1. Study sample characteristics for the total sample, and high versus low CVO risk clusters for the Caucasian and Latino sub-populations.

Supplementary TABLE S2. List of molecular entities used in the overconnectivity analyses. Results from the GWA analyses conducted in ORIGIN and in the CARDIOGRAM consortium were assigned to loci (see Methods) and protein biomarkers to their respective genes.

Supplementary TABLE S3. List of associated loci included in the overconnectivity network analyses. Identified loci at P<10-6, in the ORIGIN cohort, and P<10-8, in the CARDIOGRAM consortium, were entered in the analyses. Attached excel file.

Supplementary TABLE S4. List of sub-networks identified in the overconnectivity analyses with their respective p-values.

Supplementary TABLE S5. Overconnectivity network result table. Nodes and relationships of entities comprising the networks used for the Bayesian statistics based network analyses.

Supplementary TABLE S6. Logistic regression model to identify standard CVO risk factors (age, sex, BMI, HbA1c, c-Peptide, HDL-C, LDL-C, TG, TC, SBP, DBP, smoking status, albuminuria or reported albuminuria) contributing to clusters separation in Caucasian and Latinos.

### Example 2—Detection of SNPs Associated with CVD Risk

Having identified two SNPs associated with an individual having an increased risk in cardiovascular disease (CVD), i.e. Reference SNP cluster ID: rs73420079 (SEQ ID No: 3) and Reference SNP cluster ID: rs4314405 (SEQ ID No: 40), the inventors work enables the identification of individuals with an increased risk of CVD using oligonucleotide probes designed to detected the SNPs.

Oligonucleotide probes for detecting the presence of the SNPs can be produced and synthesized by any available oligonucleotide probe design tool, based on the SNP rs number. For example, probes of the SNPs can be sent to Illumina's® Illumina Assay Design Tool for scoring, based on the rs number format, to produce an assay ready probe.

A sample may be isolated from the patient, the sample can be a blood sample. The individual's nucleic acid is isolated from the sample. The isolation may occur by any means convenient to the practitioner. For instance, the isolation may occur by first lysing the cell using detergents, enzymatic digestion or physical disruption. The contaminating material is then removed from the nucleic acids by use of, for example, enzymatic digestion, organic solvent extraction, or chromatographic methods.

The individual's nucleic acid may be purified and/or concentrated by any means, including precipitation with alcohol, centrifugation and/or dialysis. The individual's nucleic acid is then assayed for presence or absence of one or more of the SNPs using the oligonucleotide probes that are capable of hybridizing to a nucleic acid sequence comprising one or more of the SNPs.

The detection of Reference SNP cluster ID: rs73420079 (SEQ ID No: 3) and/or Reference SNP cluster ID: rs4314405 (SEQ ID No: 40) in the sample indicates that the individual is at in increased risk of CVD.

1. A method of determining, diagnosing and/or prognosing an individual's risk of suffering from cardiovascular disease, the method comprising;
a. detecting, in a sample obtained from an individual, the expression level, amount and/or activity of two or more biomarkers selected from a group consisting of: Tumour Necrosis Factor (TNF)-□□ Glutathione S-Transferase Alpha 1 (GSTA1); N-terminal-pro hormone BNP (NT-proBNP); Retinoic Acid Receptor-Related Orphan Receptor Alpha (RORA); Tenascin C (TNC); Growth Hormone Receptor (GHR); Alpha-2-Macroglobulin (A2M); Insulin Like Growth Factor Binding Protein 2 (IGFBP2); Apolipoprotein B (APOB); Selenoprotein P (SEPP1); Trefoil Factor (TFF3); Interleukin 6 (IL6); Chitinase 3 Like 1 (CHI3L1); Hepatocyte Growth Factor Receptor (MET); Growth Differentiation Factor 15 (GDF15); Chemokine (C-C Motif) Ligand 22 (CCL22); Tumour Necrosis Factor Receptor Superfamily, Member 11 (TNFRSF11); Angiopoietin 2 (ANGPT2); and v-Rel Avian Reticuloendotheliosis Viral Oncogene Homolog A Nuclear Factor-kappa B (ReLA NF-KB);
b. comparing the expression level, amount and/or activity of the biomarker with a reference from a healthy control population; and
c. determining, diagnosing and/or prognosing the risk of an individual suffering from cardiovascular disease if the expression level, amount, and/or activity of the biomarker deviates from the reference from a healthy control population.

2. The method of claim 1, wherein a decrease in expression, amount and/or activity of TNF-□, GSTA1, NT-proBNP, RORA and/or TNC, when compared to the reference, is indicative of an individual having a higher risk of suffering from cardiovascular disease or a negative prognosis.

3. The method of either claim 1 or claim 2, wherein an increase in expression, amount and/or activity of GHR, A2M, IGFBP2, APOB, SEPP1, TFF3, IL6 and/or CHI3L1, when compared to the reference, is indicative of an individual having a higher risk of suffering from cardiovascular disease or a negative prognosis.

4. The method of any preceding claim, wherein a decrease in expression, amount and/or activity of MET, GDF15, CCL22, TNFRSF11, ANGPT2 and/or ReIA NF-KB, when compared to the reference, is indicative of an individual having a lower risk of suffering from cardiovascular disease or a positive prognosis.

5. The method according to any preceding claim, wherein step a) comprises detecting, in a sample obtained from the individual, the expression levels, amount and/or activities of at least 3, at least 4, at least 5, at least 6, at least 7, at least 8, at least 9, at least 10, at least 11, at least 12, at least 13, at least 14, at least 15, at least 16, at least 17 or at least 18 of the biomarkers selected from the group consisting of: TNF-1; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2; and ReIA NF-KB.

6. The method according to any proceeding claim, wherein the cardiovascular disease is selected from the group consisting of: cardiovascular death; myocardial infarction; stroke; and heart failure.

7. The method according to any proceeding claim, wherein the sample comprises blood, urine or tissue.

8. A kit for determining, diagnosing and/or prognosing the risk of an individual suffering from cardiovascular disease, the kit comprising:
a. detection means for detecting, in a sample obtained from a test subject, the expression level, amount and/or activity of two or more biomarkers selected from the group consisting of TNF-a; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB; and
b. a reference value from a healthy control population for expression level, amount and/or activity of two or more a biomarkers selected from the group consisting of TNF-a; GSTA1; NT-proBNP; RORA; TNC; GHR; A2M; IGFBP2; APOB; SEPP1; TFF3; IL6; CHI3L1; MET; GDF15; CCL22; TNFRSF11; ANGPT2 and ReIA NF-KB,
wherein the kit is used to identify:
i) a decrease in expression, amount and/or activity of TNF-a; GSTA1; NT-proBNP; RORA and/or TNC when compared to the reference; and/or an increase in expression, amount and/or activity of GHR; A2M; IGFBP2; APOB; SEPP1; TFF3; IL6 and/or CHI3L1; when compared to the reference to determine, diagnose and/or prognose that an individual has a higher risk of suffering from cardiovascular disease; and/or
ii) a decrease expression, amount and/or activity of MET; GDF15; CCL22; TNFRSF11; ANGPT2 and/or ReIA NF-KB when compared to the reference to determine, diagnose and/or prognose that an individual has a lower risk of suffering from cardiovascular disease.

9. A method of determining, diagnosing and/or prognosing an individual's risk of suffering from cardiovascular disease, the method comprising detecting, in a sample obtained from an individual, a single nucleotide polymorphism (SNP) in the RORA gene, wherein the presence of the SNP is indicative of an individual having an increased risk of suffering from cardiovascular disease.

10. The method according to claim 9, wherein the SNP is Reference SNP cluster ID: rs73420079.

11. A method of determining, diagnosing and/or prognosing an individual's risk of suffering from cardiovascular disease, the method comprising detecting, in a sample obtained from an individual, a single nucleotide polymorphism (SNP) in the GHR gene, wherein the presence of the SNP is indicative of an individual having an increased risk of suffering from cardiovascular disease.

12. The method according to claim 11, wherein the SNP is Reference SNP cluster ID: rs4314405.

13. GHR and/or RORA, for use in diagnosis or prognosis.

14. GHR and/or RORA, for use in diagnosing or prognosing cardiovascular disease.

15. GHR and/or RORA for use according to claim 13 or claim 14, wherein RORA comprises an SNP as defined in claim 10 and/or GHR comprise an SNP as defined in claim 12.
